(19) United States
(12) Patent Application Publication
(10) Pub. No.: US 2013/0337564 A1
(43) Pub. Date: Dec. 19, 2013

(54) Treatment of Pluripotent Cells

(71) Applicant: Janssen Biotech, Inc., Raritan, NJ (US)

(72) Inventors: Janet Davis, Raritan, NJ (US); Jiajian Liu, Princeton, NJ (US)

(21) Appl. No.: 13/917,109

(22) Filed: Jun. 13, 2013

Related U.S. Application Data
(60) Provisional application No. 61/741,776, filed on Jun. 14, 2012.

(51) Int. Cl. C12N 5/071

(52) U.S. Cl. CPC C12N 5/0676
USPC 435/377

(57) ABSTRACT

The present invention is directed to methods to treat pluripotent cells, whereby the pluripotent cells can be efficiently expanded in culture and differentiated by treating the pluripotent cells with an inhibitor of GSK-3B enzyme activity.

### CROSS REFERENCE TO RELATED APPLICATION

The present application claims the benefit of U.S. Provisional Patent Application Ser. No. 61/741,776, filed Jun. 14, 2012, which is incorporated herein by reference in its entirety for all purpose.

### FIELD OF THE INVENTION

The present invention is directed to methods to treat pluripotent cells, whereby the pluripotent cells can be efficiently expanded in culture and differentiated by treating the pluripotent cells with an inhibitor of GSK-3B enzyme activity.

### BACKGROUND

Advances in cell-replacement therapy for Type I diabetes mellitus and a shortage of transplantable islets of Langerhans have focused interest on developing sources of insulin-producing cells, or β cells, appropriate for engraftment. One approach is the generation of functional β cells from pluripotent cells, such as, for example, embryonic stem cells.

In vertebrate embryonic development, a pluripotent cell gives rise to a group of cells comprising three germ layers (ectoderm, mesoderm, and endoderm) in a process known as gastrulation. Tissues such as, for example, thyroid, thymus, pancreas, gut, and liver, will develop from the endoderm, via an intermediate stage. The intermediate stage in this process is the formation of definitive endoderm. Definitive endoderm cells express a number of markers, such as, HNF-3 beta, GATA-4, Mix11, CXCR4 and SOX-17.

Formation of the pancreas arises from the differentiation of definitive endoderm into pancreatic endoderm. Cells of the pancreatic endoderm express the pancreatic-duodenal homeobox gene, PDX-1. In the absence of PDX-1, the pancreas fails to develop beyond the formation of ventral and dorsal buds. Thus, PDX-1 expression marks a critical step in pancreatic organogenesis. The mature pancreas contains, among other cell types, exocrine tissue and endocrine tissue. Exocrine and endocrine tissues arise from the differentiation of pancreatic endoderm.

The generation of a sufficient amount of cellular material for transplantation requires a source of the cellular material that can be efficiently expanded in culture, and efficiently differentiated into the tissue of interest, for example, functional β cells.

Current methods to culture human embryonic stem cells are complex; they require the use of exogenous factors, or chemically defined media in order for the cells to proliferate without loosing their pluripotency. Furthermore differentiation of embryonic stem cells often results in a decrease in the cells to expand in culture.

In one example, Cheon et al (BioReprod DOI:10.1095/biolreprod.105.046870, Oct. 19, 2005) disclose a feeder-free, serum-free culture system in which embryonic stem cells are maintained in unconditioned serum replacement (SR) medium supplemented with different growth factors capable of triggering embryonic stem cell self-renewal.

In another example, US20050233446 discloses a defined media useful in culturing stem cells, including undifferentiated primate primordial stem cells. In solution, the media is substantially isotonic as compared to the stem cells being cultured. In a given culture, the particular medium comprises a base medium and an amount of each of bFGF, insulin, and ascorbic acid necessary to support substantially undifferentiated growth of the primordial stem cells.

In another example, WO2005086845 discloses a method for maintenance of an undifferentiated stem cell, said method comprising exposing a stem cell to a member of the transforming growth factor-beta (TGFβ) family of proteins, a member of the fibroblast growth factor (FGF) family of proteins, or nicotinamide (NIC) in an amount sufficient to maintain the cell in an undifferentiated state for a sufficient amount of time to achieve a desired result.

Inhibitors of glycogen synthase kinase-3 (GSK-3) are known to promote proliferation and expansion of adult stem cells. In one example, Tateishi et al. (Biochemical and Biophysical Research Communications (2007) 352: 635) show that inhibition of GSK-3 enhances growth and survival of human cardiac stem cells (hCSCs) recovered from the neonatal or adult human heart and having mesenchymal features.

For example, Rulifson et al (PNAS 144, 6247-6252, (2007)) states “Wnt signaling stimulates islet β cell proliferation.

In another example, WO2007016485 reports that addition of GSK-3 inhibitors to the culture of non-embryonic stem cells, including multipotent adult progenitor cells, leads to the maintenance of a pluripotent phenotype during expansion and results in a more robust differentiation response.

In another example, US2006030042 uses a method of inhibiting GSK-3, either by addition of Wnt or a small molecule inhibitor of GSK-3 enzyme activity, to maintain embryonic stem cells without the use of a feeder cell layer.

In another example, WO2006026473 reports the addition of a GSK-3B inhibitor, to stabilize pluripotent cells through transcriptional activation of c-myc and stabilization of c-myc protein.

In another example, WO2006100490 reports the use of a stem cell culture medium containing a GSK-3 inhibitor and a gp130 agonist to maintain a self-renewing population of pluripotent stem cells, including mouse or human embryonic stem cells.

In another example, Sato et al. (Nature Medicine (2004) 10:55-63) show that inhibition of GSK-3 with a specific pharmacological compound can maintain the undifferentiated phenotype of embryonic stem cells and sustain expression of pluripotent state-specific transcription factors such as Oct-3/4, Rex-1, and Nanog.

In another example, Maurer et al (Journal of Proteome Research (2007) 6:1198-1208) show that adult, neuronal stem cells treated with a GSK-3 inhibitor show enhanced neuronal differentiation, specifically by promoting transcription of β-catenin target genes and decreasing apoptosis.

In another example, Gregory et al (Annals of the New York Academy of Sciences (2005) 1049:97-106) report that inhibitors of GSK-3B enhance in vitro osteogenesis.

In another example, Feng et al (Biochemical and Biophysical Research Communications (2004) 324:1333-1339) show that hematopoietic differentiation from embryonic stem cells is associated with down-regulation of the Wnt/β-catenin pathway, where Wnt is a natural inhibitor of GSK3.

Therefore, there still remains a significant need to develop methods for treating pluripotent stem cell such that they can be expanded to address the current clinical needs, while retaining the potential to differentiate into pancreatic endocrine cells, pancreatic hormone expressing cells, or pancreatic hormone secreting cells.

### SUMMARY

The present invention provides a method to expand and differentiate pluripotent cells by treating the pluripotent cells with an inhibitor of GSK-3B enzyme activity.

In one embodiment, the present invention provides a method to expand and differentiate pluripotent cells, comprising the steps of:
a. Culturing pluripotent cells, and
b. Treating the pluripotent cells with an inhibitor of GSK-3B enzyme activity.

In one embodiment, the pluripotent cells are differentiated into cells expressing markers characteristic of the definitive endoderm lineage.

The pluripotent cells may be human embryonic stem cells, or they may be cells expressing pluripotency markers derived from human embryonic stem cells, according to the methods disclosed in 60/913,475.

In one embodiment, the inhibitor of GSK-3B enzyme activity is a compound of the Formula (I):

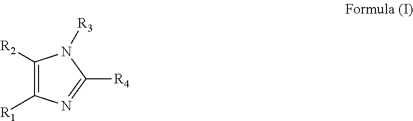

In one embodiment, the inhibitor of GSK-3B enzyme activity is a compound of the Formula (II):

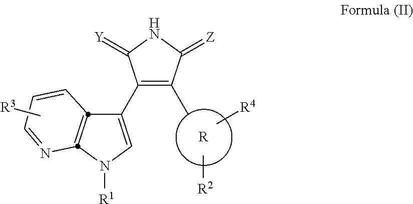

In one embodiment, the inhibitor of GSK-3B enzyme activity is a compound of the Formula (III):

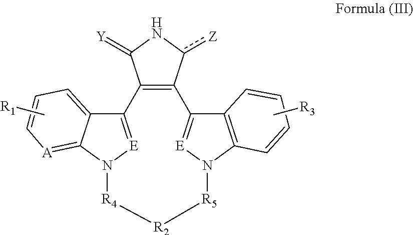

### BRIEF DESCRIPTION OF THE FIGURES

FIG. 1 shows the effect of a range of concentrations of the compound #221 on cell number, as determined by the number of nuclei observed (FIG. 1A) and Sox-17 expression, as determined by intensity of immunofluorescent staining (FIG. 1B). Results were obtained from cells of the human embryonic stem cell line H1 (white bars), or cells of the human embryonic stem cell line H9 (black bars), using the IN Cell Analyzer 1000 (GE Healthcare).

FIG. 2 shows the effect of a range of concentrations of the compound #206 on cell number, as determined by the number of nuclei observed (FIG. 2A) and Sox-17 expression, as determined by intensity of immunofluorescent staining (FIG. 2B). Results were obtained from cells of the human embryonic stem cell line H1 (white bars), or cells of the human embryonic stem cell line H9 (black bars), using the IN Cell Analyzer 1000 (GE Healthcare).

FIG. 3 shows the effect of a range of concentrations of the compound #223 on cell number, as determined by the number of nuclei observed (FIG. 3A) and Sox-17 expression, as determined by intensity of immunofluorescent staining (FIG. 3B). Results were obtained from cells of the human embryonic stem cell line H1 (white bars), or cells of the human embryonic stem cell line H9 (black bars), using the IN Cell Analyzer 1000 (GE Healthcare).

FIG. 4 shows the effect of a range of concentrations of the compound #47 on cell number, as determined by the number of nuclei observed (FIG. 4A) and Sox-17 expression, as determined by intensity of immunofluorescent staining (FIG. 4B). Results were obtained from cells of the human embryonic stem cell line H1 (white bars), or cells of the human embryonic stem cell line H9 (black bars), using the IN Cell Analyzer 1000 (GE Healthcare).

FIG. 5 shows the effect of a range of concentrations of the compound #103 on cell number, as determined by the number of nuclei observed (FIG. 5A) and Sox-17 expression, as determined by intensity of immunofluorescent staining (FIG. 5B). Results were obtained from cells of the human embryonic stem cell line H1 (white bars), or cells of the human embryonic stem cell line H9 (black bars), using the IN Cell Analyzer 1000 (GE Healthcare).

FIG. 6 shows the effect of a range of concentrations of the compound #133 on cell number, as determined by the number of nuclei observed (FIG. 6A) and Sox-17 expression, as determined by intensity of immunofluorescent staining (FIG. 6B). Results were obtained from cells of the human embryonic stem cell line H1 (white bars), or cells of the human embryonic stem cell line H9 (black bars), using the IN Cell Analyzer 1000 (GE Healthcare).

FIG. 7 shows the effect of a range of concentrations of the compound #136 on cell number, as determined by the number of nuclei observed (FIG. 7A) and Sox-17 expression, as determined by intensity of immunofluorescent staining (FIG. 7B). Results were obtained from cells of the human embryonic stem cell line H1 (white bars), or cells of the human embryonic stem cell line H9 (black bars), using the IN Cell Analyzer 1000 (GE Healthcare).

FIG. 8 shows the effect of a range of concentrations of the compound #198 on cell number, as determined by the number of nuclei observed (FIG. 8A) and Sox-17 expression, as determined by intensity of immunofluorescent staining (FIG. 8B). Results were obtained from cells of the human embryonic stem cell line H1 (white bars), or cells of the human embryonic stem cell line H9 (black bars), using the IN Cell Analyzer 1000 (GE Healthcare).

FIG. 9 shows the expression of CXCR4 on the surface of cells, as determined by immunofluorescent staining and flow cytometric analysis, on cells treated with the compounds shown, according to the methods described in Example 8.

FIG. 10 shows the expression of CXCR4 (FIG. 10A), HNF-3 beta (FIG. 10B), and Sox-17 (FIG. 10C), as determined by real-time PCR, in cells treated with the compounds shown, according to the methods described in Example 8.

FIG. 11 shows the effect of a range of concentrations of the compounds shown on cell number, as determined by the number of nuclei observed (FIG. 11A) and Pdx-1 expression, as determined by intensity of immunofluorescent staining (FIG. 11B), using the IN Cell Analyzer 1000 (GE Healthcare). Cells were treated according to the methods described in Example 9.

FIG. 12 shows the effect of a range of concentrations of the compounds shown on Pdx-1 expression (white bars) and HNF-6 (black bars), as determined by real-time PCR. Cells were treated according to the methods described in Example 9.

FIG. 13 shows the effect of a range of concentrations of the compounds shown on cell number, as determined by the number of nuclei observed (FIG. 13A) and insulin expression, as determined by intensity of immunofluorescent staining (FIG. 13B), using the IN Cell Analyzer 1000 (GE Healthcare). Cells were treated according to the methods described in Example 10.

FIG. 14 shows effect of a range of concentrations of the compounds shown on Pdx-1 expression (white bars) and insulin (black bars), as determined by real-time PCR. Cells were treated according to the methods described in Example 10.

FIG. 15 shows the effect of a range of concentrations of the compounds shown on cell number, as determined by the number of nuclei observed (FIG. 15A) and insulin expression, as determined by intensity of immunofluorescent staining (FIG. 15B), using the IN Cell Analyzer 1000 (GE Healthcare). Cells were treated according to the methods described in Example 11.

### DETAILED DESCRIPTION

For clarity of disclosure, and not by way of limitation, the detailed description of the invention is divided into the following subsections that describe or illustrate certain features, embodiments, or applications of the present invention.

### DEFINITIONS

Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent, meaning able to give rise to all embryonic and extraembryonic cell types; (2) pluripotent, meaning able to give rise to all embryonic cell types; (3) multipotent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multipotent stem cells; and (5) unipotent, meaning able to give rise to a single cell lineage (e.g., spermatogenic stem cells).

Differentiation is the process by which an unspecialized (“uncommitted”) or less specialized cell acquires the features of a specialized cell such as, for example, a nerve cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

“β-cell lineage” refer to cells with positive gene expression for the transcription factor PDX-1 and at least one of the following transcription factors: NGN-3, Nkx2.2, Nkx6.1, NeuroD, Isl-1, HNF-3 beta, MAFA, Pax4, and Pax6. Cells expressing markers characteristic of the β cell lineage include β cells.

“Cells expressing markers characteristic of the definitive endoderm lineage” as used herein refer to cells expressing at least one of the following markers: SOX-17, GATA-4, HNF-3 beta, GSC, Cer 1, Noda1, FGF8, Brachyury, Mix-like homeobox protein, FGF4 CD48, eomesodermin (EOMES), DKK4, FGF17, GATA-6, CXCR4, C-Kit, CD99, or OTX2. Cells expressing markers characteristic of the definitive endoderm lineage include primitive streak precursor cells, primitive streak cells, mesendoderm cells and definitive endoderm cells.

“Cells expressing markers characteristic of the pancreatic endoderm lineage” as used herein refer to cells expressing at least one of the following markers: PDX-1, HNF-1beta, PTF-1 alpha, HNF-6, or HB9. Cells expressing markers characteristic of the pancreatic endoderm lineage include pancreatic endoderm cells.

“Cells expressing markers characteristic of the pancreatic endocrine lineage” as used herein refer to cells expressing at least one of the following markers: NGN-3, NeuroD, Islet-1, PDX-1, NKX6.1, Pax-4, Ngn-3, or PTF-1 alpha. Cells expressing markers characteristic of the pancreatic endocrine lineage include pancreatic endocrine cells, pancreatic hormone expressing cells, and pancreatic hormone secreting cells, and cells of the β-cell lineage.

“Definitive endoderm” as used herein refers to cells which bear the characteristics of cells arising from the epiblast during gastrulation and which form the gastrointestinal tract and its derivatives. Definitive endoderm cells express the following markers: HNF-3 beta, GATA-4, SOX-17, Cerberus, OTX2, goosecoid, C-Kit, CD99, and Mix11.

“Extraembryonic endoderm” as used herein refers to a population of cells expressing at least one of the following markers: SOX-7, AFP, and SPARC.

“Markers” as used herein, are nucleic acid or polypeptide molecules that are differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

“Mesendoderm cell” as used herein refers to a cell expressing at least one of the following markers: CD48, eomesodermin (EOMES), SOX-17, DKK4, HNF-3 beta, GSC, FGF17, GATA-6.

“Pancreatic endocrine cell”, or “pancreatic hormone expressing cell” as used herein refers to a cell capable of expressing at least one of the following hormones: insulin, glucagon, somatostatin, and pancreatic polypeptide.

“Pancreatic hormone secreting cell” as used herein refers to a cell capable of secreting at least one of the following hormones: insulin, glucagon, somatostatin, and pancreatic polypeptide.

“Pre-primitive streak cell” as used herein refers to a cell expressing at least one of the following markers: Noda1, or FGF8

“Primitive streak cell” as used herein refers to a cell expressing at least one of the following markers: Brachyury, Mix-like homeobox protein, or FGF4.

In one embodiment, the present invention provides a method for the expansion and differentiation of pluripotent cells comprising treating the pluripotent cells with an inhibitor of GSK-3B enzyme activity.

In one embodiment, the present invention provides a method to expand and differentiate pluripotent cells, comprising the steps of:
c. Culturing pluripotent cells, and
d. Treating the pluripotent cells with an inhibitor of GSK-3B enzyme activity.

In one embodiment, the pluripotent cells are differentiated into cells expressing markers characteristic of the definitive endoderm lineage.

Markers characteristic of the definitive endoderm lineage are selected from the group consisting of SOX17, GATA4, Hnf-3beta, GSC, Cer 1, Noda1, FGF8, Brachyury, Mix-like homeobox protein, FGF4 CD48, eomesodermin (EOMES), DKK4, FGF17, GATA6, CXCR4, C-Kit, CD99, and OTX2. Contemplated in the present invention is a cell, derived from a pluripotent cell that expresses at least one of the markers characteristic of the definitive endoderm lineage. In one aspect of the present invention, a cell expressing markers characteristic of the definitive endoderm lineage is a primitive streak precursor cell. In an alternate aspect, a cell expressing markers characteristic of the definitive endoderm lineage is a mesendoderm cell. In an alternate aspect, a cell expressing markers characteristic of the definitive endoderm lineage is a definitive endoderm cell.

The pluripotent cells may be treated with the inhibitor of GSK-3B enzyme activity for about one to about 72 hours. Alternatively, the pluripotent cells may be treated with the inhibitor of GSK-3B enzyme activity for about 12 to about 48 hours. Alternatively, the pluripotent cells may be treated with the inhibitor of GSK-3B enzyme activity for about 48 hours.

In one embodiment, the inhibitor of GSK-3B enzyme activity is used at a concentration of about 100 nM to about 100 μM. Alternatively, the inhibitor of GSK-3B enzyme activity is used at a concentration of about 1 μM to about 10 μM. Alternatively, the inhibitor of GSK-3B enzyme activity is used at a concentration of about 10 μM.

### Compounds Suitable for Use in the Methods of the Present Invention

In one embodiment, the inhibitor of GSK-3B enzyme activity is a compound of the Formula (I):

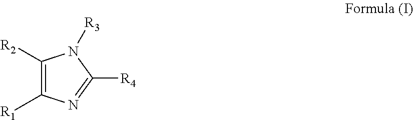

wherein:

R₁ is phenyl, substituted phenyl wherein the phenyl substituents are selected from the group consisting of C₁₋₅alkyl, halogen, nitro, trifluoromethyl and nitrile, or pyrimidinyl;

R₂ is phenyl, substituted phenyl wherein the phenyl substituents are selected from the group consisting of C₁₋₅alkyl, halogen, nitro, trifluoromethyl and nitrile, or pyrimidinyl which is optionally C₁₋₄alkyl substituted, and at least one of R₁ and R₂ is pyrimidinyl;

R₃ is hydrogen, 2-(trimethylsilyl)ethoxymethyl, C₁₋₅alkoxycarbonyl, aryloxycarbonyl, arylC₁₋₅alkyloxycarbonyl, arylC₁₋₅alkyl, substituted arylC₁₋₅alkyl wherein the one or more aryl substituents are independently selected from the group consisting of C₁₋₅alkyl, C₁₋₅alkoxy, halogen, amino, C₁₋₅alkylamino, and diC₁₋₅alkylamino, phthalimidoC₁₋₅alkyl, aminoC₁₋₅alkyl, diaminoC₁₋₅alkyl, succinimidoC₁₋₅alkyl, C₁₋₅alkylcarbonyl, arylcarbonyl, C₁₋₅alkylcarbonylC₁₋₅alkyl and aryloxycarbonylC₁₋₅alkyl;

R₄ is -(A)-(CH₂)_{q}—X;

A is vinylene, ethynylene or

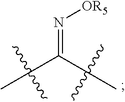

R₅ is selected from the group consisting of hydrogen, C₁₋₅alkyl, phenyl and phenylC₁₋₅alkyl;

q is 0-9;

X is selected from the group consisting of hydrogen, hydroxy, vinyl, substituted vinyl wherein one or more vinyl substituents are each selected from the group consisting of fluorine, bromine, chlorine and iodine, ethynyl, substituted ethynyl wherein the ethynyl substituents are selected from the group consisting of fluorine, bromine chlorine and iodine, C₁₋₅alkyl, substituted C₁₋₅alkyl wherein the one or more alkyl substituents are each selected from the group consisting of C₁₋₅alkoxy, trihaloalkyl, phthalimido and amino, C₃₋₇cycloalkyl, C₁₋₅alkoxy, substituted C₁₋₅alkoxy wherein the alkyl substituents are selected from the group consisting of phthalimido and amino, phthalimidooxy, phenoxy, substituted phenoxy wherein the one or more phenyl substituents are each selected from the group consisting of C₁₋₅alkyl, halogen and C₁₋₅alkoxy, phenyl, substituted phenyl wherein the one or more phenyl substituents are each selected from the group consisting of C₁₋₅alkyl, halogen and C₁₋₅alkoxy, arylC₁₋₅alkyl, substituted arylC₁₋₅alkyl wherein the one or more aryl substituents are each selected from the group consisting of C₁₋₅alkyl, halogen and C₁₋₅alkoxy, aryloxyC₁₋₅alkylamino, C₁₋₅alkylamino, diC₁₋₅ alkylamino, nitrile, oxime, benxyloxyimino, C₁₋₅alkyloxyimino, phthalimido, succinimido, C₁₋₅alkylcarbonyloxy, phenylcarbonyloxy, substituted phenylcarbonyloxy wherein the one or more phenyl substituents are each selected from the group consisting of C₁₋₅alkyl, halogen and C₁₋₅alkoxy, phenylC₁₋₅alkylcarbonyloxy wherein the one or more phenyl substituents are each selected from the group consisting of C₁₋₅alkyl, halogen and C₁₋₅alkoxy, aminocarbonyloxy, C₁₋₅alkylaminocarbonyloxy, diC₁₋₅alkylaminocarbonyloxy, C₁₋₅alkoxycarbonyloxy, substituted C₁₋₅alkoxycarbonyloxy wherein the one or more alkyl substituents are each selected from the group consisting of methyl, ethyl, isopropyl and hexyl, phenoxycarbonyloxy, substituted phenoxycarbonyloxy wherein the one or more phenyl substituents are each selected from the group consisting of C₁₋₅alkyl, C₁₋₅alkoxy and halogen, C₁₋₅alkylthio, substituted C₁₋₅alkylthio wherein the alkyl substituents are selected from the group consisting of hydroxy and phthalimido, C₁₋₅alkylsulfonyl, phenylsulfonyl, substituted phenylsulfonyl wherein the one or more phenyl substituents are each selected from the group consisting of bromine, fluorine, chloride, C₁₋₅alkoxy and trifluoromethyl; with the proviso that if A is

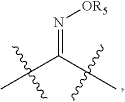

q is 0 and X is H, then R₃ may not be 2-(trimethylsilyl)ethoxymethyl; and pharmaceutically acceptable salts thereof.

An example of the invention includes a compound of Formula (I) wherein R₁ is substituted phenyl and R₂ is pyrimidin-3-yl.

An example of the invention includes a compound of Formula (I) wherein R₁ is 4-fluorophenyl.

An example of the invention includes a compound of Formula (I) wherein R₃ is hydrogen, arylC₁₋₅alkyl, or substituted arylC₁₋₅alkyl.

An example of the invention includes a compound of Formula (I) wherein R₃ is hydrogen or phenylC₁₋₅alkyl.

An example of the invention includes a compound of Formula (I) wherein A is ethynylene and q is 0-5.

An example of the invention includes a compound of Formula (I) wherein X is succinimido, hydroxy, methyl, phenyl, C₁₋₅alkylsulfonyl, C₃₋₆cycloalkyl, C₁₋₅alkylcarbonyloxy, C₁₋₅alkoxy, phenylcarbonyloxy, C₁₋₅alkylamino, diC₁₋₅alkylamino or nitrile.

Compounds of Formula (I) are disclosed in commonly assigned U.S. Pat. No. 6,214,830, the complete disclosure of which is herein incorporated by reference.

An example of the invention includes a compound of Formula (I) wherein the compound is selected from the group consisting of the compounds listed in Table A, below:

TABLE A
Compounds of Formula (I)

| Compound | Name |
|---|---|
| A-1 | 4-[5-(4-Fluorophenyl)-1H-imidazol-4-yl]pyridine |
| A-2 | 4-[4-(4-Fluorophenyl)-1-(3-phenylpropyl)-1H-imidazol-5-yl]pyridine |
| A-3 | 4-[5-(4-Fluorophenyl)-1-(3-phenylpropyl)-1H-imidazol-4-yl]pyridine |
| A-4 | 4-[4-(4-Fluorophenyl)-2-iodo-1-(3-phenylpropyl)-1H-imidazol-5-yl]pyridine |
| A-5 | 4-[4-(4-Fluorophenyl)-1-(3-phenylpropyl)-5-pyridin-4-yl-1H-imidazol-2-yl]but-3-yn-1-ol |
| A-6 | 4-[4-(4-Fluorophenyl)-1-({[2-(trimethylsilyl)ethyl]oxy}methyl)-1H-imidazol-5-yl]pyridine |
| A-7 | 4-[5-(4-Fluorophenyl)-1-({[2-(trimethylsilyl)ethyl]oxy}methyl)-1H-imidazol-4-yl]pyridine |
| A-8 | 5-(4-fluorophenyl)-2-iodo-4-(4-pyridyl)-1-[2-(trimethylsilyl)ethoxymethyl]-imidazole |
| A-9 | 5-(4-fluorophenyl)-4-(4-pyridyl)-2-(trimethylsilyl)ethinyl-1- |
| | [2-(trimethylsilyl)ethoxymethyl]-imidazole |
| A-10 | 2-(2-chlorovinyl)-5-(4-fluorophenyl)-4-(4-pyridyl)-imidazole |
| A-11 | 5-(4-Fluorophenyl)-4-pyridin-4-yl-1-({[2- |
| | (trimethylsilyl)ethyl]oxy}methyl)-1H-imidazole-2-carbaldehyde |
| A-12 | 4-[2-(2,2-Dibromoethenyl)-5-(4-fluorophenyl)-1-({[2- |
| | (trimethylsilyl)ethyl]oxy}methyl)-1H-imidazol-4-yl]pyridine |
| A-13 | 3-[4-(4-Fluorophenyl)-5-pyridin-4-yl-1H-imidazol-2-yl]-1-phenylprop-2-yn-1-ol |
| A-14 | 5-(4-Fluorophenyl)-4-pyridin-4-yl-1-{[2- |
| | (trimethylsilyl)ethoxy]methyl}-1H-imidazole-2-carbaldehyde oxime |
| A-15 | 5-(4-fluorophenyl)-4-(4-pyridyl)-2-imidazole oxime |
| A-16 | 4-[2-(5-Chloropent-1-yn-1-yl)-4-(4-fluorophenyl)-1-(3-phenylpropyl)- |
| | 1H-imidazol-5-yl]pyridine |
| A-17 | 4-[4-(4-Fluorophenyl)-1-(3-phenylpropyl)-5-pyridin-4-yl-1H-imidazol- |
| | 2-yl]but-3-yn-1-yl phenylcarbamate |
| A-18 | 4-[2-(4-Chlorobut-1-yn-1-yl)-4-(4-fluorophenyl)-1-(3-phenylpropyl)- |
| | 1H-imidazol-5-yl]pyridine |
| A-19 | 4-[4-(4-Fluorophenyl)-1-(3-phenylpropyl)-5-pyridin-4-yl-1H-imidazol- |
| | 2-yl]-N,N-dimethylbut-3-yn-1-amine |

An example of the invention includes a compound of Formula (I) wherein the compound is Compound A-5 of the formula:

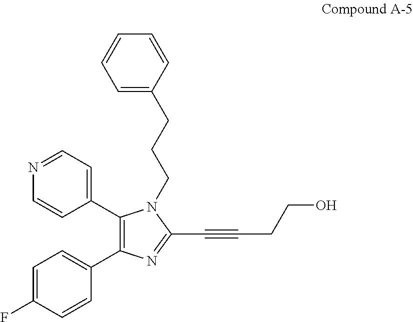 [SMILES: OCCC#Cc1nc(-c2ccc(F)cc2)c(-c2ccncc2)n1CCCc1ccccc1]

In one embodiment, the inhibitor of GSK-3B enzyme activity is a compound of the Formula (II):

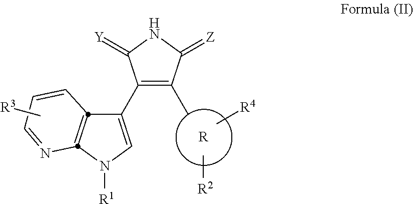

### Wherein:

R is selected from the group consisting of Rₐ, —C₁₋₈alkyl-Rₐ, —C₂₋₈alkenyl-Rₐ, —C₂₋₈alkynyl-Rₐ and cyano;

Rₐ is selected from the group consisting of cycloalkyl, heterocyclyl, aryl and heteroaryl;

R¹ is selected from the group consisting of hydrogen, —C₁₋₈alkyl-R⁵, —C₂₋₈alkenyl-R⁵, —C₂₋₈alkynyl-R⁵, —C(O)—(C₁₋₈)alkyl-R⁹, —C(O)-aryl-R⁸, —C(O)—O—(C₁₋₈)alkyl-R⁹, —C(O)—O-aryl-R⁸, —C(O)—NH(C₁₋₈alkyl-R⁹), —C(O)—NH(aryl-R⁸), —C(O)—N(C₁₋₈alkyl-R⁹)₂, —SO₂—(C₁₋₈)alkyl-R⁹, —SO₂-aryl-R⁸, -cycloalkyl-R⁶, -heterocyclyl-R⁶, -aryl-R⁶ and -heteroaryl-R⁶; wherein heterocyclyl and heteroaryl are attached to the azaindole nitrogen atom in the one position via a heterocyclyl or heteroaryl ring carbon atom;

R⁵ is 1 to 2 substituents independently selected from the group consisting of hydrogen, —O—(C₁₋₈)alkyl, —O—(C₁₋₈)alkyl-OH, —O—(C₁₋₈)alkyl-O—(C₁₋₈)alkyl, —O—(C₁₋₈)alkyl-NH₂, —O—(C₁₋₈)alkyl-NH(C₁₋₈alkyl), —O—(C₁₋₈)alkyl-N(C₁₋₅alkyl)₂, —O—(C₁₋₈)alkyl-S—(C₁₋₈)alkyl, —O—(C₁₋₈)alkyl-SO₂—(C₁₋₈)alkyl, —O—(C₁₋₈)alkyl-SO₂—NH₂, —O—(C₁₋₈)alkyl-SO₂—NH(C₁₋₅alkyl), —O—(C₁₋₈)alkyl-SO₂—N(C₁₋₅alkyl)₂, —O—C(O)H, —O—C(O)—(C₁₋₈)alkyl, —O—C(O)—NH₂, —O—C(O)—NH(C₁₋₅alkyl), —O—C(O)—N(C₁₋₅alkyl)₂, —O—(C₁₋₈)alkyl-C(O)H, —O—(C₁₋₈)alkyl-C(O)—(C₁₋₈)alkyl, —O—(C₁₋₈)alkyl-CO₂H, —O—(C₁₋₈)alkyl-C(O)—O—(C₁₋₈)alkyl, —O—(C₁₋₈)alkyl-C(O)—NH₂, —O—(C₁₋₈)alkyl-C(O)—NH(C₁₋₈alkyl), —O—(C₁₋₈)alkyl-C(O)—N(C₁₋₈alkyl)₂, —C(O)H, —C(O)—(C₁₋₈)alkyl, —CO₂H, —C(O)—O—(C₁₋₈)alkyl, —C(O)—NH₂, —C(NH)—NH₂, —C(O)—NH(C₁₋₅alkyl), —C(O)—N(C₁₋₈alkyl)₂, —SH, —S—(C₁₋₈)alkyl, —S—(C₁₋₈)alkyl-S—(C₁₋₈)alkyl, —S—(C₁₋₈)alkyl-O—(C₁₋₈)alkyl, —S—(C₁₋₈)alkyl-O—(C₁₋₈)alkyl-OH, —S—(C₁₋₈)alkyl-O—(C₁₋₈)alkyl-NH₂, —S—(C₁₋₈)alkyl-O—(C₁₋₈)alkyl-NH(C₁₋₅alkyl), —S—(C₁₋₈)alkyl-O—(C₁₋₈)alkyl-N(C₁₋₅alkyl)₂, —S—(C₁₋₈)alkyl-NH(C₁₋₅alkyl), —SO₂—(C₁₋₈)alkyl, —SO₂—NH₂, —SO₂—NH(C₁₋₅alkyl), —SO₂—N(C₁₋₅alkyl)₂, —N—R⁷, cyano, (halo)₁₋₃, hydroxy, nitro, oxo, -cycloalkyl-R⁶, -heterocyclyl-R⁶, -aryl-R⁶ and -heteroaryl-R⁶;

R⁶ is 1 to 4 substituents attached to a carbon or nitrogen atom independently selected from the group consisting of hydrogen, —C₁₋₈alkyl, —C₂₋₈alkenyl, —C₂₋₈alkynyl, —C(O)H, —C(O)—(C₁₋₈)alkyl, —CO₂H, —C(O)—O—(C₁₋₈)alkyl, —C(O)—NH₂, —C(NH)—NH₂, —C(O)—NH(C₁₋₈alkyl), —C(O)—N(C₁₋₈)alkyl)₂, —SO₂—(C₁₋₈)alkyl, —SO₂—NH₂, —SO₂—NH(C₁₋₅alkyl), —SO₂—N(C₁₋₅alkyl)₂, —(C₁₋₈)alkyl-N—R⁷, —(C₁₋₈)alkyl-(halo)₁₋₃, —(C₁₋₈)alkyl-OH, -aryl-R⁸, —(C₁₋₈)alkyl-aryl-R⁸ and —(C₁₋₈)alkyl-heteroaryl-R⁸; with the proviso that, when R⁶ is attached to a carbon atom, R⁶ is further selected from the group consisting of —C₁₋₈alkoxy, —(C₁₋₈)alkoxy-(halo)₁₋₃, —SH, —S—(C₁₋₈)alkyl, —N—R⁷, cyano, halo, hydroxy, nitro, oxo and -heteroaryl-R⁸;

R⁷ is 2 substituents independently selected from the group consisting of hydrogen, —C₁₋₈alkyl, —C₂₋₈alkenyl, —C₂₋₈alkynyl, —(C₁₋₈)alkyl-OH, —(C₁₋₈)alkyl-O—(C₁₋₈)alkyl, —(C₁₋₈)alkyl-NH₂, —(C₁₋₈)alkyl-NH(C₁₋₅alkyl), —(C₁₋₈)alkyl-N(C₁₋₅alkyl)₂, —(C₁₋₈)alkyl-S—(C₁₋₈)alkyl, —C(O)H, —C(O)—(C₁₋₈)alkyl, —C(O)—O—(C₁₋₈)alkyl, —C(O)—NH₂, —C(O)—NH(C₁₋₅alkyl), —C(O)—N(C₁₋₈alkyl)₂, —SO₂—(C₁₋₈)alkyl, —SO₂—NH₂, —SO₂—NH(C₁₋₈alkyl), —SO₂—N(C₁₋₈alkyl)₂, —C(N)—NH₂, -cycloalkyl-R⁸, —(C₁₋₈)alkyl-heterocyclyl-R⁸, -aryl-R⁸, —(C₁₋₈)alkyl-aryl-R⁸ and —(C₁₋₈)alkyl-heteroaryl-R⁸;

R⁸ is 1 to 4 substituents attached to a carbon or nitrogen atom independently selected from the group consisting of hydrogen, —C₁₋₈alkyl, —(C₁₋₈)alkyl-(halo)₁₋₃ and —(C₁₋₈)alkyl-OH; with the proviso that, when R⁸ is attached to a carbon atom, R⁸ is further selected from the group consisting of —C₁₋₈alkoxy, —NH₂, —NH(C₁₋₅alkyl), —N(C₁₋₈alkyl)₂, cyano, halo, —(C₁₋₈)alkoxy-(halo)₁₋₃, hydroxy and nitro;

R⁹ is 1 to 2 substituents independently selected from the group consisting of hydrogen, —C₁₋₈alkoxy, —NH₂, —NH(C₁₋₅alkyl), —N(C₁₋₅alkyl)₂, cyano, (halo)₁₋₃, hydroxy and nitro;

R² is one substituent attached to a carbon or nitrogen atom selected from the group consisting of hydrogen, —C₁₋₈alkyl-R⁵, —C₂₋₈alkenyl-R⁵, —C₂₋₈alkynyl-R⁵, —C(O)H, —C(O)—(C₁₋₈)alkyl-R⁹, —C(O)—NH₂, —C(O)—NH(C₁₋₈alkyl-R⁹), —C(O)—N(C₁₋₈alkyl-R⁹)₂, —C(O)—NH(aryl-R⁸), —C(O)-cycloalkyl-R⁸, —C(O)-heterocyclyl-R⁸, —C(O)-aryl-R⁸, —C(O)-heteroaryl-R⁸, —CO₂H, —C(O)—O—(C₁₋₈)alkyl-R⁹, —C(O)—O-aryl-R⁸, —SO₂—(C₁₋₈)alkyl-R⁹, —SO₂-aryl-R⁸, -cycloalkyl-R⁶, -aryl-R⁶ and —(C₁₋₈)alkyl-N—R⁷; with the proviso that, when R² is attached to a carbon atom, R² is further selected from the group consisting of —C₁₋₈alkoxy-R⁵, —N—R⁷, cyano, halogen, hydroxy, nitro, oxo, -heterocyclyl-R⁶ and -heteroaryl-R⁶;

R³ is 1 to 3 substituents attached to a carbon atom independently selected from the group consisting of hydrogen, —C₁₋₈alkyl-R¹⁰, —C₂₋₈alkenyl-R¹⁰, —C₂₋₈alkynyl-R¹⁰, —C₁₋₈alkoxy-R¹⁰, —C(O)H, —C(O)—(C₁₋₈)alkyl-R⁹, —C(O)—NH₂, —C(O)—NH(C₁₋₈alkyl-R⁹), —C(O)—N(C₁₋₈alkyl-R⁹)₂, —C(O)-cycloalkyl-R⁸, —C(O)-heterocyclyl-R⁸, —C(O)-aryl-R⁸, —C(O)-heteroaryl-R⁸, —C(NH)—NH₂, —CO₂H, —C(O)—O—(C₁₋₈)alkyl-R⁹, —C(O)—O-aryl-R⁸, —SO₂—(C₁₋₈)alkyl-R⁹, —SO₂-aryl-R⁸, —N—R⁷, cyano, halogen, hydroxy, nitro, -cycloalkyl-R⁸, -heterocyclyl-R⁸, -aryl-R⁸ and -heteroaryl-R⁸;

R⁴ is 1 to 4 substituents attached to a carbon atom independently selected from the group consisting of hydrogen, —C₁₋₅alkyl-R¹⁰, —C₂₋₈alkenyl-R¹⁰, —C₂₋₈alkynyl-R¹⁰, —C₁₋₈alkoxy-R¹⁰, —C(O)H, —C(O)—(C₁₋₈)alkyl-R⁹, —C(O)—NH₂, —C(O)—NH(C₁₋₈alkyl-R⁹), —C(O)—N(C₁₋₈alkyl-R⁹)₂, —C(O)-cycloalkyl-R⁸, —C(O)-heterocyclyl-R⁸, —C(O)-aryl-R⁸, —C(O)-heteroaryl-R⁸, —C(NH)—NH₂, —CO₂H, —C(O)—O—(C₁₋₅)alkyl-R⁹, —C(O)—O-aryl-R⁸, —SH, —S—(C₁₋₅)alkyl-R¹⁰, —SO₂—(C₁₋₈)alkyl-R⁹, —SO₂-aryl-R⁸, —SO₂—NH₂, —SO₂—NH(C₁₋₈alkyl-R⁹), —SO₂—N(C₁₋₈alkyl-R⁹)₂, —N—R⁷, cyano, halogen, hydroxy, nitro, -cycloalkyl-R⁸, -heterocyclyl-R⁸, -aryl-R⁸ and -heteroaryl-R⁸;

R¹⁰ is 1 to 2 substituents independently selected from the group consisting of hydrogen, —NH₂, —NH(C₁₋₈alkyl), —N(C₁₋₈alkyl)₂, cyano, (halo)₁₋₃, hydroxy, nitro and oxo; and,

Y and Z are independently selected from the group consisting of O, S, (H,OH) and (H,H); with the proviso that one of Y and Z is O and the other is selected from the group consisting of O, S, (H,OH) and (H,H); and pharmaceutically acceptable salts thereof.

Embodiments of the present invention include compounds of Formula (II) wherein, R is selected from the group consisting of Rₐ, —C₁₋₄alkyl-Rₐ, —C₂₋₄alkenyl-Rₐ, —C₂₋₄alkynyl-Rₐ and cyano.

Embodiments of the present invention include compounds of Formula (II) wherein, Rₐ is selected from the group consisting of heterocyclyl, aryl and heteroaryl.

In one embodiment, Rₐ is selected from the group consisting of dihydro-pyranyl, phenyl, naphthyl, thienyl, pyrrolyl, imidazolyl, pyrazolyl, pyridinyl, azaindolyl, indazolyl, benzofuryl, benzothienyl, dibenzofuryl and dibenzothienyl.

Embodiments of the present invention include compounds of Formula (II) wherein, R¹ is selected from the group consisting of hydrogen, —C₁₋₄alkyl-R⁵, —C₂₋₄alkenyl-R⁵, —C₂₋₄alkynyl-R⁵, —C(O)—(C₁₋₄)alkyl-R⁹, —C(O)-aryl-R⁸, —C(O)—O—(C₁₋₄)alkyl-R⁹, —C(O)—O-aryl-R⁸, —C(O)—NH(C₁₋₄alkyl-R⁹), —C(O)—NH(aryl-R⁸), —C(O)—N(C₁₋₄alkyl-R⁹)₂, —SO₂—(C₁₋₄)alkyl-R⁹, —SO₂-aryl-R⁸, -cycloalkyl-R⁶, -heterocyclyl-R⁶, -aryl-R⁶ and -heteroaryl-R⁶; wherein heterocyclyl and heteroaryl are attached to the azaindole nitrogen atom in the one position via a heterocyclyl or heteroaryl ring carbon atom.

In one embodiment, R¹ is selected from the group consisting of hydrogen, —C₁₋₄alkyl-R⁵, -aryl-R⁶ and -heteroaryl-R⁶; wherein heteroaryl is attached to the azaindole nitrogen atom in the one position via a heteroaryl ring carbon atom.

In one embodiment, R¹ is selected from the group consisting of hydrogen, —C₁₋₄alkyl-R⁵ and -naphthyl-R⁶.

Embodiments of the present invention include compounds of Formula (II) wherein, R⁵ is 1 to 2 substituents independently selected from the group consisting of hydrogen, —O—(C₁₋₄)alkyl, —O—(C₁₋₄)alkyl-OH, —O—(C₁₋₄)alkyl-O—(C₁₋₄)alkyl, —O—(C₁₋₄)alkyl-NH₂, —O—(C₁₋₄)alkyl-NH(C₁₋₄alkyl), —O—(C₁₋₄alkyl-N(C₁₋₄alkyl)₂, —O—(C₁₋₄)alkyl-S—(C₁₋₄)alkyl, —O—(C₁₋₄)alkyl-SO₂—(C₁₋₄)alkyl, —O—(C₁₋₄)alkyl-SO₂—NH₂, —O—(C₁₋₄)alkyl-SO₂—NH(C₁₋₄alkyl), —O—(C₁₋₄)alkyl-SO₂—N(C₁₋₄alkyl)₂, —O—C(O)H, —O—C(O)—(C₁₋₄)alkyl, —O—C(O)—NH₂, —O—C(O)—NH(C₁₋₄alkyl), —O—C(O)—N(C₁₋₄alkyl)₂, —O—(C₁₋₄)alkyl-C(O)H, —O—(C₁₋₄)alkyl-C(O)—(C₁₋₄)alkyl, —O—(C₁₋₄)alkyl-CO₂H, —O—(C₁₋₄)alkyl-C(O)—O—(C₁₋₄)alkyl, —O—(C₁₋₄)alkyl-C(O)—NH₂, —O—(C₁₋₄)alkyl-C(O)—NH(C₁₋₄alkyl), —O—(C₁₋₄alkyl-C(O)—N(C₁₋₄alkyl)₂, —C(O)H, —C(O)—(C₁₋₄)alkyl, —CO₂H, —C(O)—O—(C₁₋₄)alkyl, —C(O)—NH₂, —C(NH)—NH₂, —C(O)—NH(C₁₋₄alkyl), —C(O)—N(C₁₋₄alkyl)₂, —SH, —S—(C₁₋₄alkyl, —S—(C₁₋₄)alkyl-S—(C₁₋₄)alkyl, —S—(C₁₋₄)alkyl-O—(C₁₋₄alkyl, —S—(C₁₋₄)alkyl-O—(C₁₋₄)alkyl-OH, —S—(C₁₋₄)alkyl-O—(C₁₋₄)alkyl-NH₂, —S—(C₁₋₄)alkyl-O—(C₁₋₄)alkyl-NH(C₁₋₄alkyl), —S—(C₁₋₄)alkyl-O—(C₁₋₄)alkyl-N(C₁₋₄alkyl)₂, —S—(C₁₋₄alkyl-NH(C₁₋₄alkyl), —SO₂—(C₁₋₄)alkyl, —SO₂—NH₂, —SO₂—NH(C₁₋₄alkyl), —SO₂—N(C₁₋₄alkyl)₂, —N—R⁷, cyano, (halo)₁₋₃, hydroxy, nitro, oxo, -cycloalkyl-R⁶, -heterocyclyl-R⁶, -aryl-R⁶ and -heteroaryl-R⁶.

In one embodiment, R⁵ is 1 to 2 substituents independently selected from the group consisting of hydrogen, —O—(C₁₋₄)alkyl, —N—R⁷, hydroxy and -heteroaryl-R⁶.

In one embodiment, R⁵ is 1 to 2 substituents independently selected from the group consisting of hydrogen, —O—(C₁₋₄)alkyl, —N—R⁷, hydroxy, -imidazolyl-R⁶, -triazolyl-R⁶ and -tetrazolyl-R⁶.

Embodiments of the present invention include compounds of Formula (II) wherein, R⁶ is 1 to 4 substituents attached to a carbon or nitrogen atom independently selected from the group consisting of hydrogen, —C₁₋₄alkyl, —C₂₋₄alkenyl, —C₂₋₄alkynyl, —C(O)H, —C(O)—(C₁₋₄alkyl, —CO₂H, —C(O)—O—(C₁₋₄alkyl, —C(O)—NH₂, —C(NH)—NH₂, —C(O)—NH(C₁₋₄alkyl), —C(O)—N(C₁₋₄)alkyl)₂, —SO₂—(C₁₋₄)alkyl, —SO₂—NH₂, —SO₂—NH(C₁₋₄alkyl), —SO₂—N(C₁₋₄alkyl)₂, —(C₁₋₄)alkyl-N—R⁷, —(C₁₋₄)alkyl-(halo)₁₋₃, —(C₁₋₄alkyl-OH, -aryl-R⁸, —(C₁₋₄)alkyl-aryl-R⁸ and —(C₁₋₄)alkyl-heteroaryl-R⁸; with the proviso that, when R⁶ is attached to a carbon atom, R⁶ is further selected from the group consisting of —C₁₋₄alkoxy, —(C₁₋₄)alkoxy-(halo)₁₋₃, —SH, —S—(C₁₋₄alkyl, —N—R⁷, cyano, halo, hydroxy, nitro, oxo and -heteroaryl-R⁸.

In one embodiment, R⁶ is hydrogen.

Embodiments of the present invention include compounds of Formula (II) wherein, R⁷ is 2 substituents independently selected from the group consisting of hydrogen, —C₁₋₄alkyl, —C₂₋₄alkenyl, —C₂₋₄alkynyl, —(C₁₋₄)alkyl-OH, —(C₁₋₄)alkyl-O—(C₁₋₄)alkyl, —(C₁₋₄)alkyl-NH₂, —(C₁₋₄)alkyl-NH(C₁₋₄alkyl), —(C₁₋₄)alkyl-N(C₁₋₄alkyl)₂, —(C₁₋₄)alkyl-S—(C₁₋₄)alkyl, —C(O)H, —C(O)—(C₁₋₄alkyl, —C(O)—O—(C₁₋₄alkyl, —C(O)—NH₂, —C(O)—NH(C₁₋₄alkyl), —C(O)—N(C₁₋₄alkyl)₂, —SO₂—(C₁₋₄)alkyl, —SO₂—NH₂, —SO₂—NH(C₁₋₄alkyl), —SO₂—N(C₁₋₄alkyl)₂, —C(N)—NH₂, -cycloalkyl-R⁸, —(C₁₋₄)alkyl-heterocyclyl-R⁸, -aryl-R⁸, —(C₁₋₄)alkyl-aryl-R⁸ and —(C₁₋₄)alkyl-heteroaryl-R⁸.

In one embodiment R² is 2 substituents independently selected from the group consisting of hydrogen, —C₁₋₄alkyl, —C(O)H, —C(O)—(C₁₋₄)alkyl, —C(O)—O—(C₁₋₄)alkyl, —SO₂—NH₂, —SO₂—NH(C₁₋₄alkyl) and —SO₂—N(C₁₋₄alkyl)₂.

Embodiments of the present invention include compounds of Formula (II) wherein, R⁸ is 1 to 4 substituents attached to a carbon or nitrogen atom independently selected from the group consisting of hydrogen, —C₁₋₄alkyl, —(C₁₋₄)alkyl-(halo)₁₋₃ and —(C₁₋₄)alkyl-OH; with the proviso that, when R⁸ is attached to a carbon atom, R⁸ is further selected from the group consisting of —C₁₋₄alkoxy, —NH₂, —NH(C₁₋₄alkyl), —N(C₁₋₄alkyl)₂, cyano, halo, —(C₁₋₄)alkoxy-(halo)₁₋₃, hydroxy and nitro.

In one embodiment, R⁸ is hydrogen.

Embodiments of the present invention include compounds of Formula (II) wherein, R⁹ is 1 to 2 substituents independently selected from the group consisting of hydrogen, —C₁₋₄alkoxy, —NH₂, —NH(C₁₋₄alkyl), —N(C₁₋₄alkyl)₂, cyano, (halo)₁₋₃, hydroxy and nitro.

In one embodiment, R⁹ is hydrogen.

Embodiments of the present invention include compounds of Formula (II) wherein, R² is one substituent attached to a carbon or nitrogen atom selected from the group consisting of hydrogen, —C₁₋₄alkyl-R⁵, —C₂₋₄alkenyl-R⁵, —C₂₋₄alkynyl-R⁵, —C(O)H, —C(O)—(C₁₋₄)alkyl-R⁹, —C(O)—NH₂, —C(O)—NH(C₁₋₄)alkyl-R⁹), —C(O)—N(C₁₋₄)alkyl-R⁹)₂, —C(O)—NH(aryl-R⁸), —C(O)-cycloalkyl-R⁸, —C(O)-heterocyclyl-R⁸, —C(O)-aryl-R⁸, —C(O)-heteroaryl-R⁸, —CO₂H, —C(O)—O—(C₁₋₄)alkyl-R⁹, —C(O)—O-aryl-R⁸, —SO₂—(C₁₋₄)alkyl-R⁹, —SO₂-aryl-R⁸, -cycloalkyl-R⁶, -aryl-R⁶ and —(C₁₋₄)alkyl-N—R⁷; with the proviso that, when R² is attached to a carbon atom, R² is further selected from the group consisting of —C₁₋₄alkoxy-R⁵, —N—R⁷, cyano, halogen, hydroxy, nitro, oxo, -heterocyclyl-R⁶ and -heteroaryl-R⁶.

In one embodiment, R² is one substituent attached to a carbon or nitrogen atom selected from the group consisting of hydrogen, —C₁₋₄alkyl-R⁵, —C₂₋₄alkenyl-R⁵, —C₂₋₄alkynyl-R⁵, —CO₂H, —C(O)—O—(C₁₋₄)alkyl-R⁹, -cycloalkyl-R⁶, -aryl-R⁶ and —(C₁₋₄)alkyl-N—R⁷; with the proviso that, when R² is attached to a nitrogen atom, a quaternium salt is not formed; and, with the proviso that, when R² is attached to a carbon atom, R² is further selected from the group consisting of —C₁₋₄alkoxy-R⁵, —N—R⁷, cyano, halogen, hydroxy, nitro, oxo, -heterocyclyl-R⁶ and -heteroaryl-R⁶.

In one embodiment, R² is one substituent attached to a carbon or nitrogen atom selected from the group consisting of hydrogen, —C₁₋₄alkyl-R⁵ and -aryl-R⁶; with the proviso that, when R² is attached to a nitrogen atom, a quaternium salt is not formed; and, with the proviso that when R² is attached to a carbon atom, R² is further selected from the group consisting of —N—R⁷, halogen, hydroxy and -heteroaryl-R⁶.

Embodiments of the present invention include compounds of Formula (II) wherein, R³ is 1 to 3 substituents attached to a carbon atom independently selected from the group consisting of hydrogen, —C₁₋₄alkyl-R¹⁰, —C₂₋₄alkenyl-R¹⁰, —C₂₋₄alkynyl-R¹⁰, —C₁₋₄alkoxy-R¹⁰, —C(O)H, —C(O)—(C₁₋₄)alkyl-R⁹, —C(O)—NH₂, —C(O)—NH(C₁₋₄)alkyl-R⁹), —C(O)—N(C₁₋₄)alkyl-R⁹)₂, —C(O)-cycloalkyl-R⁸, —C(O)-heterocyclyl-R⁸, —C(O)-aryl-R⁸, —C(O)-heteroaryl-R⁸, —C(NH)—NH₂, —CO₂H, —C(O)—O—(C₁₋₄)alkyl-R⁹, —C(O)—O-aryl-R⁸, —SO₂—(C₁₋₈)alkyl-R⁹, —SO₂-aryl-R⁸, —N—R⁷, —(C₁₋₄)alkyl-N—R⁷, cyano, halogen, hydroxy, nitro, -cycloalkyl-R⁸, -heterocyclyl-R⁸, -aryl-R⁸ and -heteroaryl-R⁸.

In one embodiment, R³ is one substituent attached to a carbon atom selected from the group consisting of hydrogen, —C₁₋₄alkyl-R¹⁰, —C₂₋₄alkenyl-R¹⁰, —C₂₋₄alkynyl-R¹⁰, —C₁₋₄alkoxy-R¹⁰, —C(O)H, —CO₂H, —NH₂, —NH(C₁₋₄alkyl), —N(C₁₋₄alkyl)₂, cyano, halogen, hydroxy and nitro.

In one embodiment, R³ is one substituent attached to a carbon atom selected from the group consisting of hydrogen, —C₁₋₄alkyl-R¹⁰, —NH₂, —NH(C₁₋₄alkyl), —N(C₁₋₄alkyl)₂, halogen and hydroxy.

Embodiments of the present invention include compounds of Formula (II) wherein, R⁴ is 1 to 4 substituents attached to a carbon atom independently selected from the group consisting of hydrogen, —C₁₋₄alkyl-R¹⁰, —C₂₋₄alkenyl-R¹⁰, —C₂₋₄alkynyl-R¹⁰, —C₁₋₄alkoxy-R¹⁰, —C(O)H, —C(O)—(C₁₋₄)alkyl-R⁹, —C(O)—NH₂, —C(O)—NH(C₁₋₄)alkyl-R⁹), —C(O)—N(C₁₋₄)alkyl-R⁹)₂, —C(O)-cycloalkyl-R⁸, —C(O)-heterocyclyl-R⁸, —C(O)-aryl-R⁸, —C(O)-heteroaryl-R⁸, —C(NH)—NH₂, —CO₂H, —C(O)—O—(C₁₋₄)alkyl-R⁹, —C(O)—O-aryl-R⁸, —SH, —S—(C₁₋₄)alkyl-R¹⁰, —SO₂—(C₁₋₄)alkyl-R⁹, —SO₂-aryl-R⁸, —SO₂—NH₂, —SO₂—NH(C₁₋₄alkyl-R⁹), —SO₂—N(C₁₋₄)alkyl-R⁹)₂, —N—R⁷, cyano, halogen, hydroxy, nitro, -cycloalkyl-R⁸, -heterocyclyl-R⁸, -aryl-R⁸ and -heteroaryl-R⁸.

In one embodiment, R⁴ is 1 to 4 substituents attached to a carbon atom independently selected from the group consisting of hydrogen, —C₁₋₄alkyl-R¹⁰, —C₂₋₄alkenyl-R¹⁰, —C₂₋₄alkynyl-R¹⁰, —C₁₋₄alkoxy-R¹⁰, —C(O)H, —CO₂H, —NH₂, —NH(C₁₋₄alkyl), —N(C₁₋₄alkyl)₂, cyano, halogen, hydroxy, nitro, -cycloalkyl, -heterocyclyl, -aryl and -heteroaryl.

In one embodiment, R⁴ is 1 to 4 substituents attached to a carbon atom independently selected from the group consisting of hydrogen, C₁₋₄alkyl-R¹⁰, C₁₋₄alkoxy-R¹⁰, —NH₂, —NH(C₁₋₄alkyl), —N(C₁₋₄alkyl)₂, halogen and hydroxy.

In one embodiment, R⁴ is 1 to 4 substituents attached to a carbon atom independently selected from the group consisting of hydrogen, C₁₋₄alkyl-R¹⁰, C₁₋₄alkoxy-R¹⁰, —NH₂, —NH(C₁₋₄alkyl), —N(C₁₋₄alkyl)₂, chlorine, fluorine and hydroxy.

Embodiments of the present invention include compounds of Formula (II) wherein, R¹⁰ is 1 to 2 substituents independently selected from the group consisting of hydrogen, —NH₂, —NH(C₁₋₄alkyl), —N(C₁₋₄alkyl)₂, cyano, (halo)₁₋₃, hydroxy, nitro and oxo.

In one embodiment, R¹⁰ is 1 to 2 substituents independently selected from the group consisting of hydrogen and (halo)₁₋₃.

In one embodiment, R¹⁰ is 1 to 2 substituents independently selected from the group consisting of hydrogen and (fluoro)₃.

Embodiments of the present invention include compounds of Formula (II) wherein, Y and Z are independently selected from the group consisting of O, S, (H,OH) and (H,H); with the proviso that one of Y and Z is O and the other is selected from the group consisting of O, S, (H,OH) and (H,H).

In one embodiment, Y and Z are independently selected from the group consisting of O and (H,H); with the proviso that one of Y and Z is O, and the other is selected from the group consisting of O and (H,H).

In one embodiment, Y and Z are independently selected from O.

Compounds of Formula (II) are disclosed in commonly assigned U.S. Pat. No. 7,125,878, the complete disclosure of which is herein incorporated by reference.

An example of the invention includes a compound of Formula (II) wherein the compound is selected from the group consisting of the compounds listed in Table B, below:

TABLE B
Compounds of Formula (II)

| Compound | Name |
|---|---|
| B-1 | 3-(2-Chlorophenyl)-4-[1-(3-hydroxypropyl)-1H-pyrrolo[2,3-b]pyridin-3- |
| | yl]-1H-pyrrole-2,5-dione |
| B-2 | 3-(2-Chlorophenyl)-4-{1-[3-(dimethylamino)propyl]-1H-pyrrolo[2,3- |
| | b]pyridin-3-yl}-1H-pyrrole-2,5-dione |
| B-3 | 3-[1-(3-Hydroxypropyl)-1H-pyrrolo[2,3-b]pyridin-3-yl]-4-naphthalen-1- |
| | yl-1H-pyrrole-2,5-dione |
| B-4 | 3-{1-[3-(Dimethylamino)propyl]-1H-pyrrolo[2,3-b]pyridin-3-yl}-4- |
| | naphthalen-1-yl-1H-pyrrole-2,5-dione |
| B-5 | 3-(5-Chloro-1-benzothiophen-3-yl)-4-[1-(3-hydroxypropyl)-1H- |
| | pyrrolo[2,3-b]pyridin-3-yl]-1H-pyrrole-2,5-dione |
| B-6 | 3-[1-(3-Hydroxypropyl)-1H-pyrrolo[2,3-b]pyridin-3-yl]-4-(1H-indazol-3- |
| | yl)-1H-pyrrole-2,5-dione |
| B-7 | 3-(1-Ethyl-1H-pyrrolo[2,3-b]pyridin-3-yl)-4-[1-(3-hydroxypropyl)-1H- |
| | pyrrolo[2,3-b]pyridin-3-yl]-1H-pyrrole-2,5-dione |
| B-8 | 3-[1-(3-Hydroxypropyl)-1H-pyrrolo[2,3-b]pyridin-3-yl]-4-(2- |
| | methoxyphenyl)-1H-pyrrole-2,5-dione |
| B-9 | 3-[1-(3-Hydroxypropyl)-1H-pyrrolo[2,3-b]pyridin-3-yl]-4-(3- |
| | methoxyphenyl)-1H-pyrrole-2,5-dione |
| B-10 | 3-(2-Chloro-4-fluorophenyl)-4-[1-(3-hydroxypropyl)-1H-pyrrolo[2,3- |
| | b]pyridin-3-yl]-1H-pyrrole-2,5-dione |
| B-11 | 3-[1-(3-Hydroxypropyl)-1H-pyrrolo[2,3-b]pyridin-3-yl]-4-[2- |
| | (trifluoromethyl)phenyl]-1H-pyrrole-2,5-dione |
| B-12 | 3-[1-(3-Hydroxypropyl)-1H-pyrrolo[2,3-b]pyridin-3-yl]-4-pyridin-2-yl- |
| | 1H-pyrrole-2,5-dione |
| B-13 | 3-[3-Chloro-5-(trifluoromethyl)pyridin-2-yl]-4-[1-(3-hydroxypropyl)-1H- |
| | pyrrolo[2,3-b]pyridin-3-yl]-1H-pyrrole-2,5-dione |
| B-14 | 3-[1-(3-Hydroxypropyl)-1H-pyrrolo[2,3-b]pyridin-3-yl]-4-thiophen-2-yl- |
| | 1H-pyrrole-2,5-dione |
| B-15 | 3-(2,5-Dichlorothiophen-3-yl)-4-[1-(3-hydroxypropyl)-1H-pyrrolo[2,3- |
| | b]pyridin-3-yl]-1H-pyrrole-2,5-dione |
| B-16 | 3-[1-(3-Hydroxypropyl)-1H-pyrazol-3-yl]-4-[1-(3-hydroxypropyl)-1H- |
| | pyrrolo[2,3-b]pyridin-3-yl]-1H-pyrrole-2,5-dione |
| B-17 | 3-[1-(3-Hydroxypropyl)-1H-pyrrolo[2,3-b]pyridin-3-yl]-4-(1H-imidazol- |
| | 2-yl)-1H-pyrrole-2,5-dione |
| B-18 | 3-[1-(3-Hydroxypropyl)-1H-imidazol-4-yl]-4-[1-(3-hydroxypropyl)-1H- |
| | pyrrolo[2,3-b]pyridin-3-yl]-1H-pyrrole-2,5-dione |
| B-19 | 3-[1-(2-Hydroxyethyl)-1H-imidazol-4-yl]-4-[1-(3-hydroxypropyl)-1H- |
| | pyrrolo[2,3-b]pyridin-3-yl]-1H-pyrrole-2,5-dione |
| B-20 | 3-{1-[3-(Dimethylamino)propyl]-1H-indazol-3-yl}-4-(1-naphthalen-2-yl- |
| | 1H-pyrrolo[2,3-b]pyridin-3-yl)-1H-pyrrole-2,5-dione |
| B-21 | 3-[1-(3-Hydroxypropyl)-1H-indazol-3-yl]-4-(1-naphthalen-2-yl-1H- |
| | pyrrolo[2,3-b]pyridin-3-yl)-1H-pyrrole-2,5-dione |
| B-22 | 3-[(E)-2-(4-Fluorophenyl)ethenyl]-4-[1-(3-hydroxypropyl)-1H- |
| | pyrrolo[2,3-b]pyridin-3-yl]-1H-pyrrole-2,5-dione |
| B-23 | 3-(3,4-Dihydro-2H-pyran-6-yl)-4-[1-(3-hydroxypropyl)-1H-pyrrolo[2,3- |
| | b]pyridin-3-yl]-1H-pyrrole-2,5-dione |
| B-24 | 4-[1-(3-Hydroxypropyl)-1H-pyrrolo[2,3-b]pyridin-3-yl]-1H,1′H-3,3′- |
| | bipyrrole-2,5-dione |
| B-25 | 3-(1-Benzofuran-2-yl)-4-[1-(3-hydroxypropyl)-1H-pyrrolo[2,3-b]pyridin- |
| | 3-yl]-1H-pyrrole-2,5-dione |
| B-26 | 3-[1-(3-Hydroxypropyl)-1H-pyrrolo[2,3-b]pyridin-3-yl]-4-(1-methyl-1H- |
| | pyrazol-3-yl)-1H-pyrrole-2,5-dione |
| B-27 | 4-[1-(3-Hydroxypropyl)-1H-pyrrolo[2,3-b]pyridin-3-yl]-2,5-dioxo-2,5- |
| | dihydro-1H-pyrrole-3-carbonitrile |
| B-28 | 3-Dibenzo[b,d]thien-4-yl-4-[1-(3-hydroxypropyl)-1H-pyrrolo[2,3- |
| | b]pyridin-3-yl]-1H-pyrrole-2,5-dione |
| B-29 | 3-Dibenzo[b,d]furan-4-yl-4-[1-(3-hydroxypropyl)-1H-pyrrolo[2,3- |
| | b]pyridin-3-yl]-1H-pyrrole-2,5-dione |
| B-30 | 3-(2-Hydroxyphenyl)-4-{1-[3-(methyloxy)propyl]-1H-pyrrolo[2,3- |
| | b]pyridin-3-yl}-1H-pyrrole-2,5-dione |
| B-31 | 3-[3,4-Bis(methyloxy)phenyl]-4-{1-[3-(methyloxy)propyl]-1H- |
| | pyrrolo[2,3-b]pyridin-3-yl}-1H-pyrrrole-2,5-dione |
| B-32 | 3-(3,4-Dihydroxyphenyl)-4-[1-(3-hydroxypropyl)-1H-pyrrolo[2,3- |
| | b]pyridin-3-yl]-1H-pyrrole-2,5-dione |
| B-33 | 3-[2-(Methyloxy)phenyl]-4-(1-naphthalen-2-yl-1H-pyrrolo[2,3-b]pyridin- |
| | 3-yl)-1H-pyrrole-2,5-dione |
| B-34 | 1,1-Dimethylethyl [3-(3-{4-[2-(methyloxy)phenyl]-2,5-dioxo-2,5- |
| | dihydro-1H-pyrrol-3-yl}-1H-pyrrolo[2,3-b]pyridin-1-yl)propyl]carbamate |
| B-35 | 3-[1-(3-Aminopropyl)-1H-pyrrolo[2,3-b]pyridin-3-yl]-4-[2- |
| | (methyloxy)phenyl]-1H-pyrrole-2,5-dione |
| B-36 | N-[3-(3-{4-[2-(Methyloxy)phenyl]-2,5-dioxo-2,5-dihydro-1H-pyrrol-3- |
| | yl}-1H-pyrrolo[2,3-b]pyridin-1-yl)propyl]acetamide |
| B-37 | N-[3-(3-{4-[2-(Methyloxy)phenyl]-2,5-dioxo-2,5-dihydro-1H-pyrrol-3- |
| | yl}-1H-pyrrolo[2,3-b]pyridin-1-yl)propyl]sulfamide |
| B-38 | 3-(2-methoxyphenyl)-4-[1-[3-(1H-tetrazol-1-yl)propyl]-1H-pyrrolo[2,3- |
| | b]pyridine-3-yl]-1H-pyrrole-2,5-dione |
| B-39 | 3-(2-methoxyphenyl)-4-[1-[3-(2H-tetrazol-2-yl)propyl]-1H-pyrrolo[2,3- |
| | b]pyridine-3-yl]-1H-pyrrole-2,5-dione |
| B-40 | 3-[1-(3-Hydroxypropyl)-1H-pyrrolo[2,3-b]pyridin-3-yl]-4-pyrazin-2-yl- |
| | 1H-pyrrole-2,5-dione |
| B-41 | 3-[2,4-Bis(methyloxy)pyrimidin-5-yl]-4-[1-(3-hydroxypropyl)-1H- |
| | pyrrolo[2,3-b]pyridin-3-yl]-1H-pyrrole-2,5-dione |
| B-42 | 4-(3-{4-[2,4-Bis(methyloxy)pyrimidin-5-yl]-2,5-dioxo-2,5-dihydro-1H- |
| | pyrrol-3-yl}-1H-pyrrolo[2,3-b]pyridin-1-yl)butanenitrile |
| B-43 | 4-{3-[4-(1-Methyl-1H-pyrazol-3-yl)-2,5-dioxo-2,5-dihydro-1H-pyrrol-3- |
| | yl]-1H-pyrrolo[2,3-b]pyridin-1-yl}butanenitrile |
| B-44 | 3-[2,4-Bis(methyloxy)pyrimidin-5-yl]-4-[1-(2-phenylethyl)-1H- |
| | pyrrolo[2,3-b]pyridin-3-yl]-1H-pyrrole-2,5-dione |

An example of the invention includes a compound of Formula (II) wherein the compound is selected from the group consisting of:

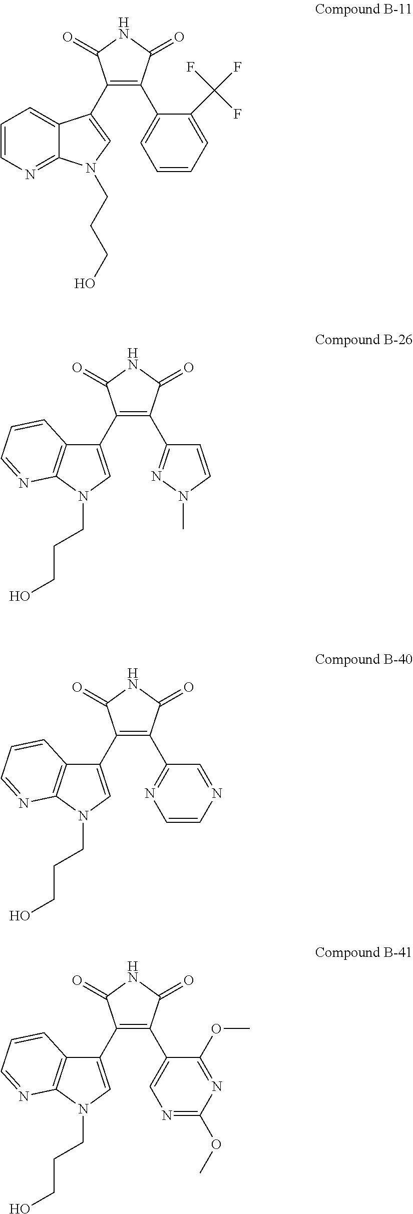 [SMILES: O=C1NC(=O)C(c2cn(CCCO)c3ncccc23)=C1c1ccccc1C(F)(F)F ; Cn1ccc(C2=C(c3cn(CCCO)c4ncccc34)C(=O)NC2=O)n1 ; O=C1NC(=O)C(c2cn(CCCO)c3ncccc23)=C1c1cnccn1 ; COc1ncc(C2=C(c3cn(CCCO)c4ncccc34)C(=O)NC2=O)c(OC)n1]

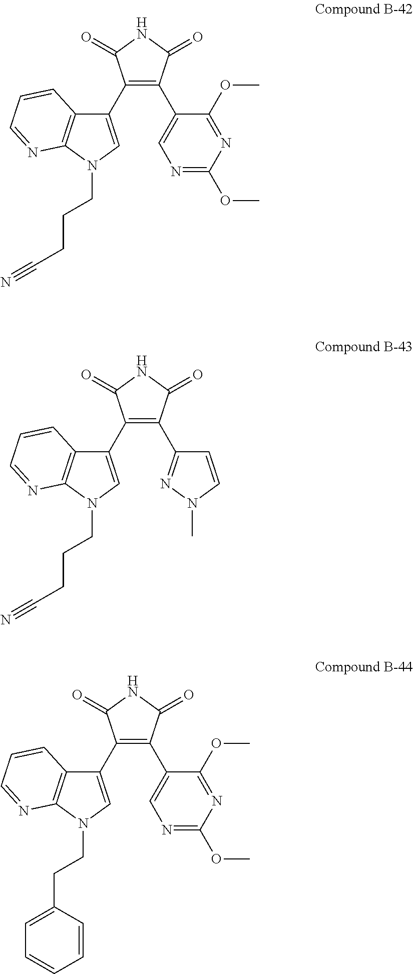 [SMILES: COc1ncc(C2=C(c3cn(CCCC#N)c4ncccc34)C(=O)NC2=O)c(OC)n1 ; Cn1ccc(C2=C(c3cn(CCCC#N)c4ncccc34)C(=O)NC2=O)n1 ; COc1ncc(C2=C(c3cn(CCc4ccccc4)c4ncccc34)C(=O)NC2=O)c(OC)n1]

In one embodiment, the inhibitor of GSK-3B enzyme activity is a compound of the Formula (III):

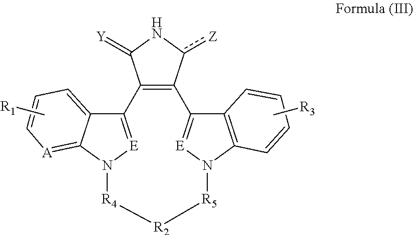

wherein

A and E are independently selected from the group consisting of a hydrogen substituted carbon atom and a nitrogen atom; wherein

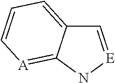

is independently selected from the group consisting of 1H-indole, 1H-pyrrolo[2,3-b]pyridine, 1H-pyrazolo[3,4-b]pyridine and 1H-indazole;

Z is selected from O; alternatively, Z is selected from dihydro; wherein each hydrogen atom is attached by a single bond;

R₄ and R₅ are independently selected from C₁₋₈alkyl, C₂₋₈alkenyl and C₂₋₈alkynyl optionally substituted with oxo;

R₂ is selected from the group consisting of —C₁₋₈alkyl-, —C₂₋₈alkenyl-, —C₂₋₈alkynyl-, —O—(C₁₋₈)alkyl-O—, —O—(C₂₋₈)alkenyl-O—, —O—(C₂₋₈)alkynyl-O—, —C(O)—(C₁₋₈)alkyl-C(O)— (wherein any of the foregoing alkyl, alkenyl and alkynyl linking groups are straight carbon chains optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, —C(O)O—(C₁₋₈)alkyl, —C₁₋₈alkyl-C(O)O—(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy, hydroxy(C₁₋₈)alkyl and oxo; and, wherein any of the foregoing alkyl, alkenyl and alkynyl linking groups are optionally substituted with one to two substituents independently selected from the group consisting of heterocyclyl, aryl, heteroaryl, heterocyclyl(C₁₋₈)alkyl, aryl(C₁₋₈)alkyl, heteroaryl(C₁₋₈)alkyl, spirocycloalkyl and spiroheterocyclyl (wherein any of the foregoing cycloalkyl, heterocyclyl, aryl and heteroaryl substituents are optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl; and, wherein any of the foregoing heterocyclyl substituents are optionally substituted with oxo)), cycloalkyl, heterocyclyl, aryl, heteroaryl (wherein cycloalkyl, heterocyclyl, aryl and heteroaryl are optionally substituted with one to four substituents independently selected from the group consisting of C₁₋₅alkyl, C₁₋₅alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl; and, wherein heterocyclyl is optionally substituted with oxo), —(O—(CH₂)₁₋₆)₀₋₅—O—, —O—(CH₂)₁₋₆—O—(CH₂)₁₋₆—O—, —O—(CH₂)₁₋₆—O—(CH₂)₁₋₆—O—(CH₂)₁₋₆—O—, —(O—(CH₂)₁₋₆)₀₋₅—NR₆—, —O—(CH₂)₁₋₆—NR₆—(CH₂)₁₋₆—O—, —O—(CH₂)₁₋₆—O—(CH₂)₁₋₆—NR₆—, —(O—(CH₂)₁₋₆)₀₋₅—S—, —O—(CH₂)₁₋₆—S—(CH₂)₁₋₆—O—, —O—(CH₂)₁₋₆—O—(CH₂)₁₋₆—S—, —NR₆—, —NR₆—NR₇—, —NR₆—(CH₂)₁₋₆—NR₂—, —NR₆—(CH₂)₁₋₆—NR₂—(CH₂)₁₋₆—NR₈—, —NR₆—C(O)—, —C(O)—NR₆—, —C(O)—(CH₂)₀₋₆—NR₆—(CH₂)₀₋₆—C(O)—, —NR₆—(CH₂)₀₋₆—C(O)—(CH₂)₁₋₆—C(O)—(CH₂)₀₋₆—NR₂—, —NR₆—C(O)—NR₇—, —NR₆—C(NR₇)—NR₈—, —O—(CH₂)₁₋₆—NR₆—(CH₂)₁₋₆—S—, —S—(CH₂)₁₋₆—NR₆—(CH₂)₁₋₆—O—, —S—(CH₂)₁₋₆—NR₆—(CH₂)₁₋₆—S—, —NR₆—(CH₂)₁₋₆—S—(CH₂)₁₋₆—NR₂— and —SO₂— (wherein R₆, R₇ and R₈ are independently selected from the group consisting of hydrogen, C₁₋₅alkyl, C₁₋₈alkoxy(C₁₋₈)alkyl, carboxyl(C₁₋₈)alkyl, amino(C₁₋₈)alkyl (wherein amino is substituted with a substituent independently selected from the group consisting of hydrogen and C₁₋₄alkyl), hydroxy(C₁₋₈)alkyl, heterocyclyl(C₁₋₅)alkyl, aryl(C₁₋₈)alkyl and heteroaryl(C₁₋₈)alkyl (wherein the foregoing heterocyclyl, aryl and heteroaryl substituents are optionally substituted with one to four substituents independently selected from the group consisting of C₁₋₅alkyl, C₁₋₅alkoxy, C₁₋₅alkoxy(C₁₋₅)alkyl, carboxyl, carboxyl(C₁₋₅)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl; and, wherein heterocyclyl is optionally substituted with oxo)); with the proviso that, if A and E are selected from a hydrogen substituted carbon atom, then R₂ is selected from the group consisting of —C₂₋₈alkynyl-, —O—(C₁₋₈)alkyl-O—, —O—(C₂₋₈)alkenyl-O—, —O—(C₂₋₈)alkynyl-O—, —C(O)—(C₁₋₈)alkyl-C(O)— (wherein any of the foregoing alkyl, alkenyl and alkynyl linking groups are straight carbon chains optionally substituted with one to four substituents independently selected from the group consisting of C₁₋₈alkyl, C₁₋₈alkoxy, C₁₋₈alkoxy(C₁₋₅)alkyl, carboxyl, carboxyl(C₁₋₈)alkyl, —C(O)O—(C₁₋₈)alkyl, —C₁₋₈alkyl-C(O)O—(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy, hydroxy(C₁₋₈)alkyl and oxo; and, wherein any of the foregoing alkyl, alkenyl and alkynyl linking groups are optionally substituted with one to two substituents independently selected from the group consisting of heterocyclyl, aryl, heteroaryl, heterocyclyl(C₁₋₈)alkyl, aryl(C₁₋₈)alkyl, heteroaryl(C₁₋₈)alkyl, spirocycloalkyl and spiroheterocyclyl (wherein any of the foregoing cycloalkyl, heterocyclyl, aryl and heteroaryl substituents are optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl; and, wherein any of the foregoing heterocyclyl substituents are optionally substituted with oxo)), cycloalkyl (wherein cycloalkyl is optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl), —(O—(CH₂)₁₋₆)₁₋₅—O—, —O—(CH₂)₁₋₆—O—(CH₂)₁₋₆—O—, —O—(CH₂)₁₋₆—O—(CH₂)₁₋₆—O—(CH₂)₁₋₆—O—, —O—(CH₂)₁₋₆)₁₋₅—NR₆—, —O—(CH₂)₁₋₆—NR₆—(CH₂)₁₋₆—O—, —O—(CH₂)₁₋₆—O—(CH₂)₁₋₆—NR₆—, —(O—(CH₂)₁₋₆)₀₋₅—S—, —O—(CH₂)₁₋₆—S—(CH₂)₁₋₆—O—, —O—(CH₂)₁₋₆—O—(CH₂)₁₋₆—S—, —NR₆—NR₇—, —NR₆—(CH₂)₁₋₆—NR₉—, —NR₆—(CH₂)₁₋₆—NR₂—(CH₂)₁₋₆—NR₈—, —NR₉—C(O)—, —C(O)—NR₉—, —C(O)—(CH₂)₀₋₆—NR₆—(CH₂)₀₋₆—C(O)—, —NR₆—(CH₂)₀₋₆—C(O)—(CH₂)₁₋₆—C(O)—(CH₂)₀₋₆—NR₂—, —NR₆—C(O)—NR₇—, —NR₆—C(NR₇)—NR₈—, —O—(CH₂)₁₋₆—NR₆—(CH₂)₁₋₆—S—, —S—(CH₂)₁₋₆—NR₆—(CH₂)₁₋₆—O—, —S—(CH₂)₁₋₆—NR₆—(CH₂)₁₋₆—S— and —NR₆—(CH₂)₁₋₆—S—(CH₂)₁₋₆—NR₂— (wherein R₆, R₇ and R₈ are independently selected from the group consisting of hydrogen, C₁₋₅alkyl, C₁₋₈alkoxy(C₁₋₈)alkyl, carboxyl(C₁₋₈)alkyl, amino(C₁₋₈)alkyl (wherein amino is substituted with a substituent independently selected from the group consisting of hydrogen and C₁₋₄alkyl), hydroxy(C₁₋₈)alkyl, heterocyclyl(C₁₋₈)alkyl, aryl(C₁₋₈)alkyl and heteroaryl(C₁₋₈)alkyl (wherein the foregoing heterocyclyl, aryl and heteroaryl substituents are optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl; and, wherein heterocyclyl is optionally substituted with oxo); and, wherein R₉ is selected from the group consisting of C₁₋₈alkyl, C₁₋₅alkoxy(C₁₋₈)alkyl, carboxyl(C₁₋₈)alkyl, amino(C₁₋₈)alkyl (wherein amino is substituted with a substituent independently selected from the group consisting of hydrogen and C₁₋₄alkyl), hydroxy(C₁₋₈)alkyl, heterocyclyl(C₁₋₈)alkyl, aryl(C₁₋₈)alkyl and heteroaryl(C₁₋₈)alkyl (wherein the foregoing heterocyclyl, aryl and heteroaryl substituents are optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl; and, wherein heterocyclyl is optionally substituted with oxo)); and,

R₁ and R₃ are independently selected from the group consisting of hydrogen, C₁₋₈alkyl, C₂₋₈alkenyl, C₂₋₈alkynyl (wherein alkyl, alkenyl and alkynyl are optionally substituted with a substituent selected from the group consisting of C₁₋₈alkoxy, alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), (halo)₁₋₃, (halo)₁₋₃(C₁₋₈)alkyl, (halo)₁₋₃(C₁₋₈)alkoxy, hydroxy, hydroxy(C₁₋₈)alkyl and oxo), C₁₋₈alkoxy, C₁₋₈alkoxycarbonyl, (halo)₁₋₃(C₁₋₈)alkoxy, C₁₋₈alkylthio, aryl, heteroaryl (wherein aryl and heteroaryl are optionally substituted with a substituent selected from the group consisting of C₁₋₈alkyl, C₁₋₈alkoxy, alkoxy(C₁₋₅)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl), amino (substituted with a substituent independently selected from the group consisting of hydrogen and C₁₋₄alkyl), cyano, halogen, hydroxy and nitro; and pharmaceutically acceptable salts thereof.

In one embodiment, a compound of Formula (III) is a compound selected from the group consisting of:

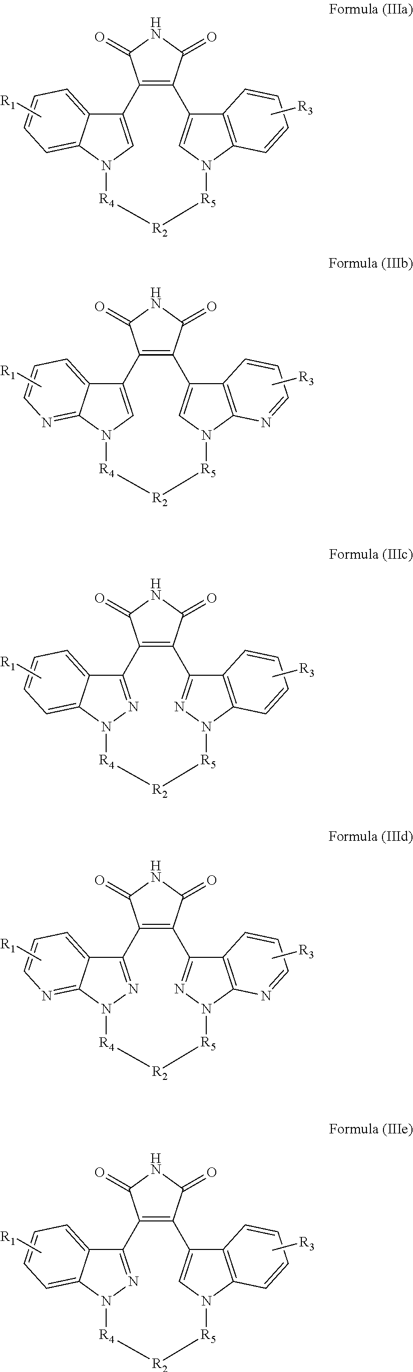

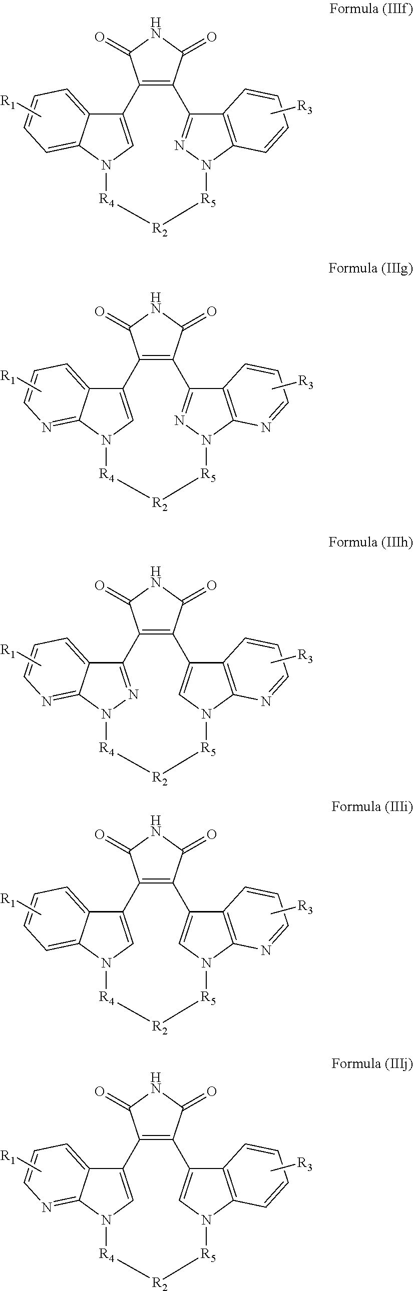

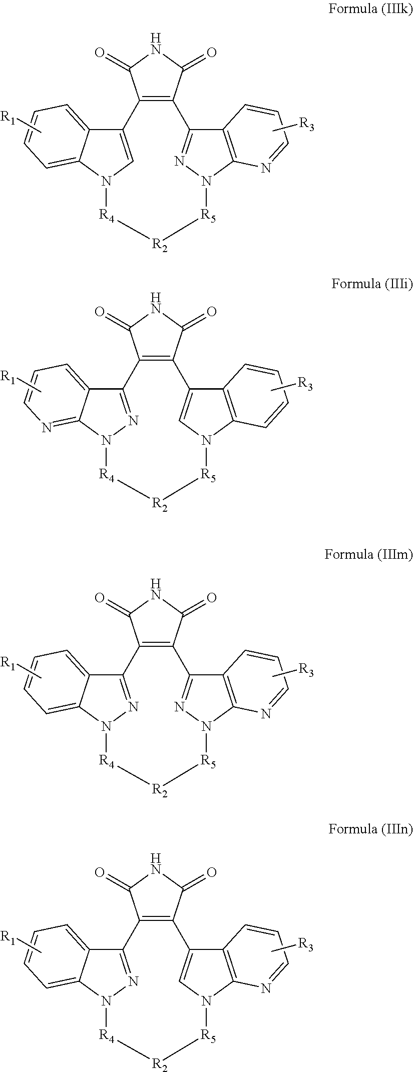

wherein all other variables are as previously defined; and, pharmaceutically acceptable salts thereof.

In one embodiment, a compound of Formula (III) is a compound selected from the group consisting of:

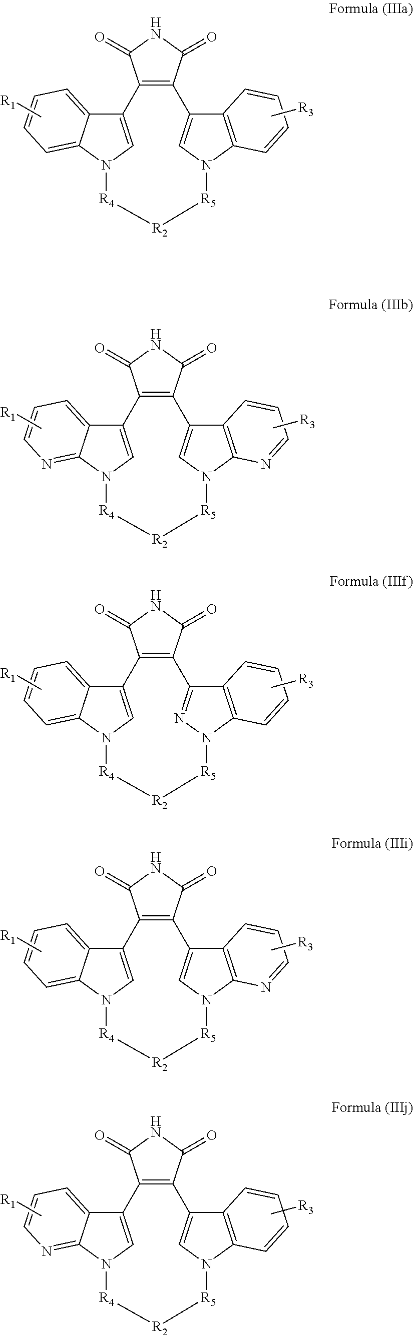

wherein all other variables are as previously defined; and, pharmaceutically acceptable salts thereof.

Compounds of Formula (III) are disclosed in commonly assigned U.S. Pat. No. 6,828,327, the complete disclosure of which is herein incorporated by reference.

An example of the invention includes a compound of Formula (III) wherein the compound is selected from the group consisting of compounds listed in Table C, below:

TABLE C
Compounds of Formula (III)

| Compound | Name |
|---|---|
| C-1 | 6,7,9,10,12,13,15,16-Octahydro-23H-5,26:17,22-di(metheno)dipyrido[2,3- |
| | k:3′,2′-q]pyrrolo[3,4-n][1,4,7,10,19]trioxadiazacyclohenicosine-23,25(24H)- |
| | dione |
| C-2 | 10,11,13,14,16,17,19,20,22,23-Decahydro-1H-9,4:24,29- |
| | di(metheno)dipyrido[2,3-n:3′,2′-t]pyrrolo[3,4- |
| | q][1,4,7,10,13,22]tetraoxadiazacyclotetracosine-1,3(2H)-dione |
| C-3 | 10,11,13,14,16,17,19,20,22,23,25,26-Dodecahydro-1H-9,4:27,32- |
| | di(metheno)dipyrido[2,3-q:3′,2′-w]pyrrolo[3,4- |
| | t][1,4,7,10,13,16,25]pentaoxadiazacycloheptacosine-1,3(2H)-dione |
| C-4 | 6,7,9,10,12,13-Hexahydro-20H-5,23:14,19- |
| | di(metheno)dibenzo[h,n]pyrrolo[3,4-k][1,4,7,16]dioxadiazacyclooctadecine- |
| | 20,22(21H)-dione |
| C-5 | 6,7,9,10,12,13,15,16-Octahydro-23H-5,26:17,22- |
| | di(metheno)dibenzo[k,q]pyrrolo[3,4-n][1,4,7,10,19]trioxadiazacyclohenicosine- |
| | 23,25(24H)-dione |
| C-6 | 10,11,13,14,16,17,19,20,22,23-Decahydro-1H-9,4:24,29- |
| | di(metheno)dibenzo[n,t]pyrrolo[3,4- |
| | q][1,4,7,10,13,22]tetraoxadiazacyclotetracosine-1,3(2H)-dione |
| C-7 | 10,11,13,14,16,17,19,20,22,23,25,26-Dodecahydro-1H-9,4:27,32- |
| | di(metheno)dibenzo[q,w]pyrrolo[3,4- |
| | t][1,4,7,10,13,16,25]pentaoxadiazacycloheptacosine-1,3(2H)-dione |
| C-8 | 4,12,14,22- |
| | Tetraazaheptacyclo[20.6.1.1~7,14~.1~16,20~.0~2,6~.0~8,13~.0~23,~28~]hentri |
| | aconta-1(29),2(6),7(31),8,10,12,16(30),17,19,23,25,27-dodecaene-3,5-dione |
| | (non-preferred name) |
| C-9 | 4,12,14,22,30- |
| | Pentaazaheptacyclo[20.6.1.1~7,14~.1~16,20~.0~2,6~.0~8,13~.0~23,28~]hentri |
| | aconta-1(29),2(6),7(31),8,10,12,16(30),17,19,23,25,27-dodecaene-3,5-dione |
| | (non-preferred name) |
| C-10 | 6,7,9,10,12,13-Hexahydro-20H-5,23:14,19-di(metheno)pyrido [2,3- |
| | k]pyrrolo[3,4-n][4,7,1,10]benzodioxadiazacyclooctadecine-20,22(21H)-dione |
| C-11 | 6,7,9,10,12,13,15,16-Octahydro-23H-5,26:17,22-di(metheno)pyrido[2,3- |
| | n]pyrrolo[3,4-q][4,7,10,1,13]benzotrioxadiazacylohenicosine- |
| | 23,25(24H)-dione |
| C-12 | 11-Ethyl-6,7,10,11,12,13,15,16-octahydro-9H,23H-5,26:17,22- |
| | di(metheno)dibenzo[k,q]pyrrolo[3,4- |
| | n][1,7,4,10,19]dioxatriazacyclohenicosine-23,25(24H)-dione |
| C-13 | 11-Methyl-6,7,10,11,12,13,15,16-octahydro-9H,23H-5,26:17,22- |
| | di(metheno)dibenzo[k,q]pyrrolo[3,4- |
| | n][1,7,4,10,19]dioxatriazacyclohenicosine-23,25(24H)-dione |
| C-14 | 11-(1-Methylethyl)-6,7,10,11,12,13,15,16-octahydro-9H,23H-5,26:17,22- |
| | di(metheno)dibenzo[k,q]pyrrolo[3,4- |
| | n][1,7,4,10,19]dioxatriazacyclohenicosine-23,25(24H)-dione |
| C-15 | 8,11,14-Trimethyl-7,8,9,10,11,12,13,14,15,16-decahydro-6H,23H- |
| | 5,26:17,22-di(metheno)dibenzo[n,t]pyrrolo[3,4- |
| | q][1,4,7,10,13]pentaazacyclohenicosine-23,25(24H)-dione |
| C-16 | 11-Methyl-6,7,10,11,12,13,15,16-octahydro-9H,23H-5,26-(azeno)-17,22- |
| | (metheno)dibenzo[k,q]pyrrolo[3,4- |
| | n][1,7,4,10,19]dioxatriazacyclohenicosine-23,25(24H)-dione |
| C-17 | 11-Ethyl-6,7,10,11,12,13,15,16-octahydro-9H,23H-5,26-(azeno)-17,22- |
| | (metheno)dibenzo[k,q]pyrrolo[3,4- |
| | n][1,7,4,10,19]dioxatriazacyclohenicosine-23,25(24H)-dione |
| C-18 | 11-Ethyl-6,7,10,11,12,13,15,16-octahydro-9H,23H-5,26:17,22- |
| | di(metheno)dipyrido[2,3-k:3′,2′-q]pyrrolo[3,4- |
| | n][1,7,4,10,19]dioxatriazacyclohenicosine-23,25(24H)-dione |
| C-19 | 6,7,9,10,12,13,15,16-Octahydro-23H-5,26:17,22- |
| | di(metheno)dipyrido[2,3-k:3′,2′-q]pyrrolo[3,4- |
| | n][1,7,4,10,19]dioxathiadiazacyclohenicosine-23,25(24H)-dione |
| C-20 | 7,8,9,10,11,12,13,14,15,16-Decahydro-6H,23H-5,26:17,22- |
| | di(metheno)dipyrido[2,3-n:3′,2′-t]pyrrolo[3,4- |
| | q][1,7,13]triazacyclohenicosine-23,25(24H)-dione |
| C-21 | 11-Ethyl-7,8,9,10,11,12,13,14,15,16-decahydro-6H,23H-5,26:17,22- |
| | di(metheno)dipyrido[2,3-n:3′,2′-t]pyrrolo[3,4- |
| | q][1,7,13]triazacyclohenicosine-23,25(24H)-dione |
| C-22 | 6,7,8,9,10,11,12,13,14,15-Decahydro-22H-5,25:16,21- |
| | di(metheno)dipyrido[2,3-m:3′,2′-s]pyrrolo[3,4- |
| | p][1,6,12]triazacycloicosine-22,24(23H)-dione |
| C-23 | 10-Ethyl-6,7,8,9,10,11,12,13,14,15-decahydro-22H-5,25:16,21- |
| | di(metheno)dipyrido[2,3-m:3′,2′-s]pyrrolo[3,4- |
| | p][1,6,12]triazacycloicosine-22,24(23H)-dione |
| C-24 | 6,7,9,10-Tetrahydro-17H-5,20-(azeno)-11,16- |
| | (metheno)dibenzo[e,k]pyrrolo[3,4-h][1,4,13]oxadiazacyclopentadecine- |
| | 17,19(18H)-dione |
| C-25 | 8,9,11,12,13,14,15,16-Octahydro-6H,23H-5,26:17,22- |
| | di(metheno)dipyrido[2,3-b:3′,2′-h]pyrrolo[3,4- |
| | e][1,10]diazacyclohenicosine-10,23,25(7H,24H)-trione |
| C-26 | 8,9,11,12,13,14-Hexahydro-6H,21H-5,24:15,20-di(metheno)dipyrido[2,3- |
| | b:3′,2′-h]pyrrolo[3,4-e][1,10]diazacyclononadecine-10,21,23(7H,22H)-trione |
| C-27 | (7R,14R)-7,14-Dihydroxy-6,7,8,9,10,11,12,13,14,15-decahydro-22H- |
| | 5,25:16,21-di(metheno)dipyrido[2,3-b:3′,2′-h]pyrrolo[3,4- |
| | e][1,10]diazacycloicosine-22,24(23H)-dione |
| C-28 | 6,7,9,10,12,13-Hexahydro-20H-5,23:14,19-di(metheno)dipyrido[2,3- |
| | h:3′,2′-n]pyrrolo[3,4-k][1,4,7,16]dioxadiazacyclooctadecine-20,22(21H)-dione |
| C-29 | 11-(2-Methoxyethyl)-6,7,10,11,12,13,15,16-octahydro-9H,23H-5,26- |
| | (azeno)-17,22-(metheno)dibenzo[k,q]pyrrolo[3,4- |
| | n][1,7,4,10,19]dioxatriazacyclohenicosine-23,25(24H)-dione |
| C-30 | 11-(2-Hydroxyethyl)-6,7,10,11,12,13,15,16-octahydro-9H,23H- |
| | 5,26:17,22-di(metheno)dibenzo[k,q]pyrrolo[3,4- |
| | n][1,7,4,10,19]dioxatriazacyclohenicosine-23,25(24H)-dione |
| C-31 | 14-Methyl-6,7,9,10,12,13,14,15,16,17-decahydro-24H-5,27:18,23-di(metheno) |
| | dibenzo[l,r]pyrrolo[3,4-o][1,4,7,11,20]dioxatriazacyclodocosine-24,26(25H)-dione |

An example of the invention includes a compound of Formula (III) wherein the compound is selected from the group consisting of:

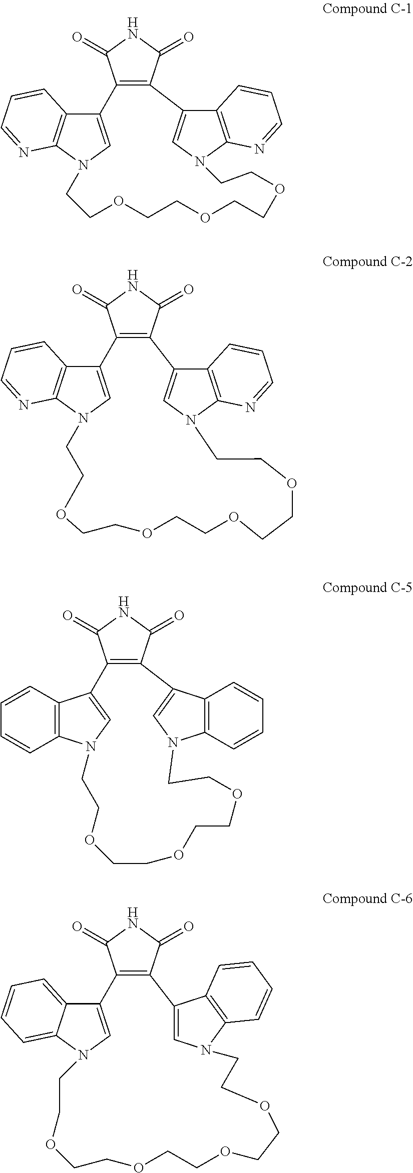 [SMILES: O=C1NC(=O)C2=C1c1cn(c3ncccc13)CCOCCOCCOCCn1cc2c2cccnc21 ; O=C1NC(=O)C2=C1c1cn(c3ncccc13)CCOCCOCCOCCOCCn1cc2c2cccnc21 ; O=C1NC(=O)C2=C1c1cn(c3ccccc13)CCOCCOCCOCCn1cc2c2ccccc21 ; O=C1NC(=O)C2=C1c1cn(c3ccccc13)CCOCCOCCOCCOCCn1cc2c2ccccc21]

Other examples of the invention include a compound selected from the group consisting of the compounds listed in Table D, below:

TABLE D
Additional Compounds

| Compound | Name |
|---|---|
| D-1a | 11-Ethyl-6,7,10,11,12,13,15,16-octahydro-9H,23H-5,26:17,22- |
| | di(metheno)dibenzo[k,q]pyrrolo[3,4- |
| | n][1,7,4,10,19]dioxatriazacyclohenicosine-23,25(24H)-dione |
| D-2a | 3-(1-{3-[(2-Hydroxyethyl)(methyl)amino]propyl}-1H-indazol-3-yl)-4-(1- |
| | pyridin-3-yl-1H-indol-3-yl)-1H-pyrrole-2,5-dione |
| D-3a | 3,5-Dichloro-N-[3-chloro-4-(3,4,12,12a-tetrahydro-1H-[1,4]thiazino[3,4- |
| | c][1,4]benzodiazepin-11(6H)-ylcarbonyl)phenyl]benzamide |
| D-4a | 3-[1-(2-Hydroxyethyl)-1H-indol-3-yl]-4-(1-pyridin-3-yl-1H-indol-3-yl)- |
| | 1H-pyrrole-2,5-dione |
| D-5a | 3-[2-(Methyloxy)phenyl]-4-(1-pyridin-3-yl-1H-indol-3-yl)-1H-pyrrole-2,5- |
| | dione |
| D-6a | 6-[(2-{[4-(2,4-Dichlorophenyl)-5-(4-methyl-1H-imidazol-2-yl)pyrimidin- |
| | 2-yl]amino}ethyl)amino]pyridine-3-carbonitrile |
| D-7a | 3-(5-Chloro-1-methyl-1H-indol-3-yl)-4-{1-[3-(1H-imidazol-1-yl)propyl]- |
| | 1H-indazol-3-yl}-1H-pyrrole-2,5-dione |
| D-8a | 3-(5-Chloro-1-methyl-1H-indol-3-yl)-4-{1-[3-(1H-1,2,3-triazol-1- |
| | yl)propyl]-1H-indazol-3-yl}-1H-pyrrole-2,5-dione |
| D-9a | 3-[1-(3-Hydroxypropyl)-1H-pyrrolo[2,3-b]pyridin-3-yl]-4-(1-menthyl-1H- |
| | pyrazol-3-yl)-1H-pyrrole-2,5-dione |
| D-10a | N-[3-(3-{4-[1-(1-Benzothien-3-yl)-1H-indol-3-yl]-2,5-dioxo-2,5-dihydro- |
| | 1H-pyrrol-3-yl}-1H-indazol-1-yl)propyl]sulfamide |
| D-11a | 3-[1-(3-Hydroxy-3-methylbutyl)-1H-indazol-3-yl]-4-(1-pyridin-3-yl-1H- |
| | indol-3-yl)-1H-pyrrole-2,5-dione |
| D-12a | 3-[1-(2-Hydroxyethyl)-1H-indazol-3-yl]-4-(1-pyrimidin-5-yl-1H-indol-3- |
| | yl)-1H-pyrrole-2,5-dione |
| D-13a | 3-[1-(2-Hydroxyethyl)-1H-indol-3-yl]-4-(1-pyrimidin-5-yl-1H-indol-3-yl)- |
| | 1H-pyrrole-2,5-dione |
| D-14a | (11Z)-8,9,10,13,14,15-Hexahydro-2,6:17,21-di(metheno)pyrrolo[3,4- |
| | h][1,15,7]dioxazacyclotricosine-22,24(1H,23H)-dione |
| D-15a | 3-(5-Chloro-1-pyridin-3-yl-1H-indol-3-yl)-4-[1-(3-hydroxypropyl)-1H- |
| | indazol-3-yl]-1H-pyrrole-2,5-dione |
| D-16a | 3-[2-(Methyloxy)phenyl]-4-{1-[3-(methyloxy)propyl]-1H-pyrrolo[3,2- |
| | c]pyridin-3-yl}-1H-pyrrole-2,5-dione |
| D-17a | 3-[1-(3-Hydroxypropyl)-1H-indazol-3-yl]-4-[1-(tetrahydro-2H-pyran-4- |
| | yl)-1H-indol-3-yl]-1H-pyrrole-2,5-dione |
| D-18a | 2-{3-[4-(5-Chloro-1-methyl-1H-indol-3-yl)-2,5-dioxo-2,5-dihydro-1H- |
| | pyrrol-3-yl]-1H-indazol-1-yl}-N-(2-hydroxyethyl)acetamide |
| D-19a | 4-(3-Chlorophenyl)-6-[3-(dimethylamino)propyl]-5,6- |
| | dihydropyrrolo[3′,4′:5,6]pyrido[3,4-b]indole-1,3(2H,4H)-dione |
| D-20a | 14-Ethyl-6,7,9,10,13,14,15,16-octahydro-12H,23H-5,26:17,22- |
| | di(metheno)dibenzo[k,q]pyrrolo[3,4- |
| | n][1,4,7,10,19]dioxatriacyclohenicosine-23,25(24H)-dione |
| D-21a | 14-(Phenylmethyl)-6,7,9,10,13,14,15,16-octahydro-12H,23H-5,26:17,22- |
| | di(metheno)dibenzo[k,q]pyrrolo[3,4- |
| | n][1,4,7,10,19]dioxatriacyclohenicosine-23,25(24H)-dione |
| D-22a | 3-[1-(2-Hydroxyethyl)-1H-indol-3-yl]-4-{1-[2-({2-[(2- |
| | hydroxyethyl)oxy]ethyl}oxy)ethyl]-1H-indol-3-yl}-1H-pyrrole-2,5-dione |
| D-23a | 8,11-Dimethyl-6,7,8,9,10,11,12,13-octahydro-20H-5,23:14,19- |
| | di(metheno)dibenzo[k,q]pyrrolo[3,4-n][1,4,7,10]tetraazacyclooctadecine- |
| | 20,22(21H)-dione |
| D-24a | 12,21-Dimethyl-11,12,13,14,16,17,20,21,22,23-decahydro-1H,10H,19H- |
| | 9,4:24,29-di(metheno)dibenzo[k,q]pyrrolo[3,4- |
| | n][1,4,7,10,19,22]dioxatetraazacyclotetracosine-1,3(2H)-dione |
| D-25a | 14-(Furan-2-ylmethyl)-6,7,9,10,13,14,15,16-octahydro-12H,23H- |
| | 5,26:17,22-di(metheno)dibenzo[k,q]pyrrolo[3,4- |
| | n][1,4,7,10,19]dioxatriazacyclohenicosine-23,25(24H)-dione |
| D-26a | 14-(2-Thienylmethyl)-6,7,9,10,13,14,15,16-octahydro-12H,23H- |
| | 5,26:17,22-di(metheno)dibenzo[k,q]pyrrolo[3,4- |
| | n][1,4,7,10,19]dioxatriazacyclohenicosine-23,25(24H)-dione |
| D-27a | 14-(Naphthalen-1-ylmethyl)-6,7,9,10,13,14,15,16-octahydro-12H,23H- |
| | 5,26:17,22-di(metheno)dibenzo[k,q]pyrrolo[3,4- |
| | n][1,4,7,10,19]dioxatriazacyclohenicosine-23,25(24H)-dione |
| D-28a | 14-(Pyridin-4-ylmethyl)-6,7,9,10,13,14,15,16-octahydro-12H,23H- |
| | 5,26:17,22-di(metheno)dibenzo[k,q]pyrrolo[3,4- |
| | n][1,4,7,10,19]dioxatriazacyclohenicosine-23,25(24H)-dione |
| D-29a | 3-{1-[2-(1,2,3,4-Tetrahydronaphthalen-1-ylamino)ethyl]-1H-indol-3-yl}-4- |
| | (1-{2-[(2-{[2-(1,2,3,4-tetrahydronaphthalen-1- |
| | ylamino)ethyl]oxy}ethyl)oxy]ethyl}-1H-indol-3-yl)-1H-pyrrole-2,5-dione |
| D-30a | 3-{1-[3-(Dimethylamino)phenyl]-1H-indol-3-yl}-4-[1-(2-hydroxyethyl)- |
| | 1H-indazol-3-yl]-1H-pyrrole-2,5-dione |
| D-31a | 3-{5-Chloro-1-[6-(dimethylamino)pyridin-3-yl]-1H-indol-3-yl}-4-[1-(2- |
| | hydroxyethyl)-1H-indazol-3-yl]-1H-pyrrole-2,5-dione |
| D-32a | Methyl 5-(5-chloro-3-{4-[1-(2-hydroxyethyl)-1H-indazol-3-yl]-2,5-dioxo- |
| | 2,5-dihydro-1H-pyrrol-3-yl}-1H-indol-1-yl)pyridine-3-carboxylate |

Other examples of the invention include a compound selected from the group consisting of:

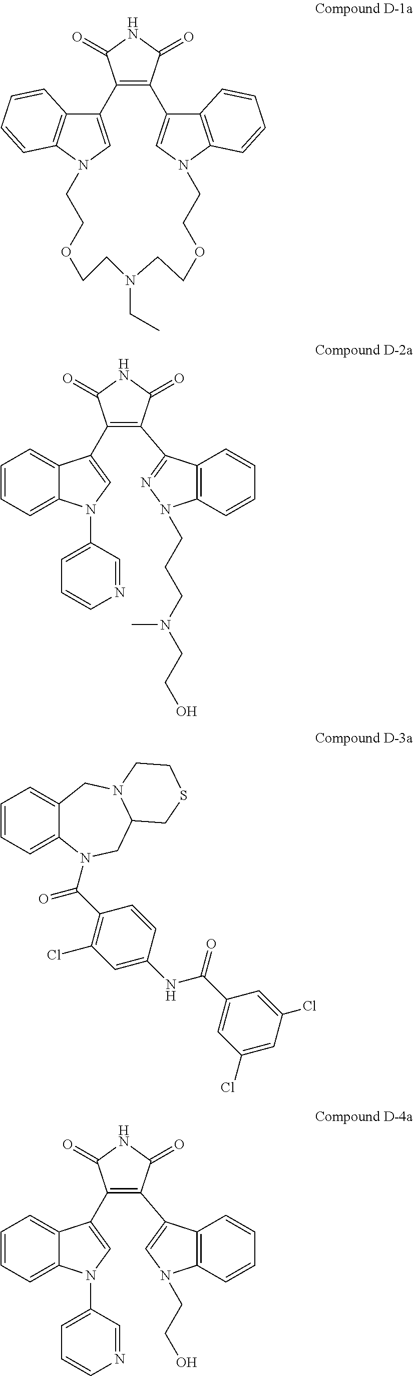 [SMILES: CCN1CCOCCn2cc(c3ccccc32)C2=C(C(=O)NC2=O)c2cn(c3ccccc23)CCOCC1 ; CN(CCO)CCCn1nc(C2=C(c3cn(-c4cccnc4)c4ccccc34)C(=O)NC2=O)c2ccccc21 ; O=C(Nc1ccc(C(=O)N2CC3CSCCN3Cc3ccccc32)c(Cl)c1)c1cc(Cl)cc(Cl)c1 ; O=C1NC(=O)C(c2cn(-c3cccnc3)c3ccccc23)=C1c1cn(CCO)c2ccccc12]

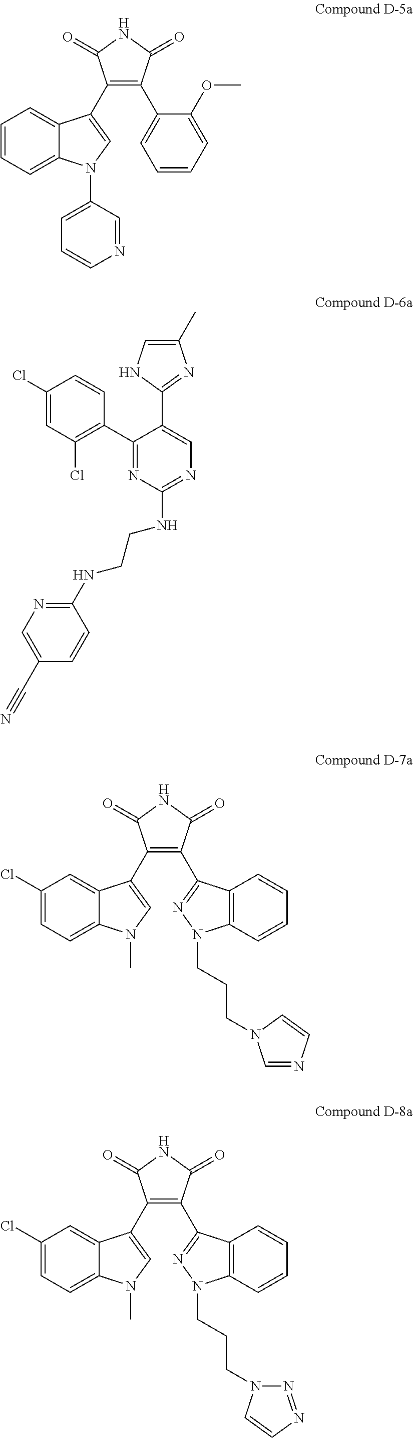 [SMILES: COc1ccccc1C1=C(c2cn(-c3cccnc3)c3ccccc23)C(=O)NC1=O ; Cc1c[nH]c(-c2cnc(NCCNc3ccc(C#N)cn3)nc2-c2ccc(Cl)cc2Cl)n1 ; Cn1cc(C2=C(c3nn(CCCn4ccnc4)c4ccccc34)C(=O)NC2=O)c2cc(Cl)ccc21 ; Cn1cc(C2=C(c3nn(CCCn4ccnn4)c4ccccc34)C(=O)NC2=O)c2cc(Cl)ccc21]

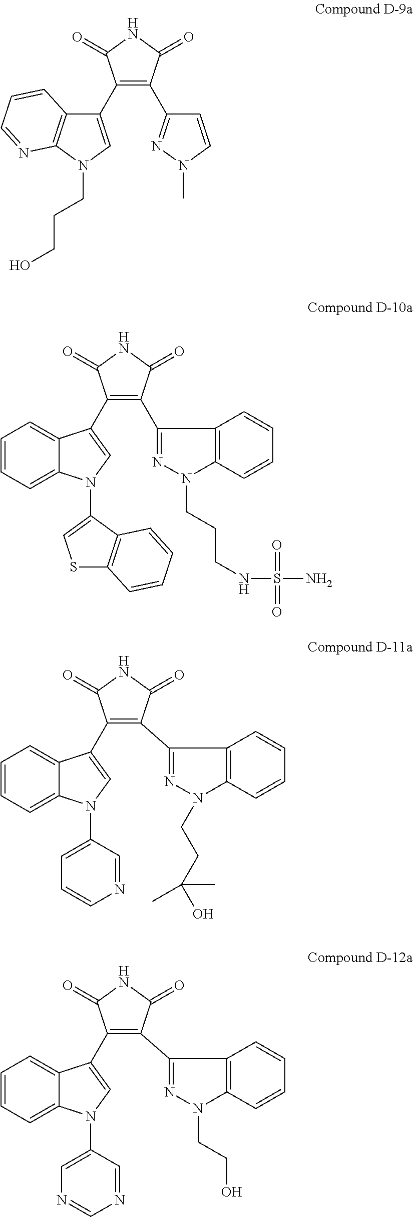 [SMILES: Cn1ccc(C2=C(c3cn(CCCO)c4ncccc34)C(=O)NC2=O)n1 ; NS(=O)(=O)NCCCn1nc(C2=C(c3cn(-c4csc5ccccc45)c4ccccc34)C(=O)NC2=O)c2ccccc21 ; CC(C)(O)CCn1nc(C2=C(c3cn(-c4cccnc4)c4ccccc34)C(=O)NC2=O)c2ccccc21 ; O=C1NC(=O)C(c2nn(CCO)c3ccccc23)=C1c1cn(-c2cncnc2)c2ccccc12]

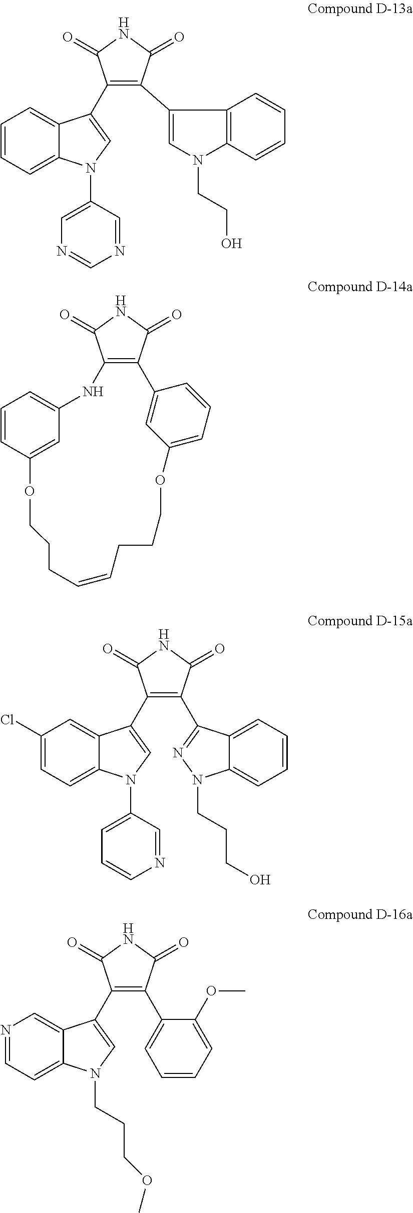 [SMILES: O=C1NC(=O)C(c2cn(-c3cncnc3)c3ccccc23)=C1c1cn(CCO)c2ccccc12 ; O=C1NC(=O)C2=C1Nc1cccc(c1)OCCC/C=C\CCCOc1cccc2c1 ; O=C1NC(=O)C(c2nn(CCCO)c3ccccc23)=C1c1cn(-c2cccnc2)c2ccc(Cl)cc12 ; COCCCn1cc(C2=C(c3ccccc3OC)C(=O)NC2=O)c2cnccc21]

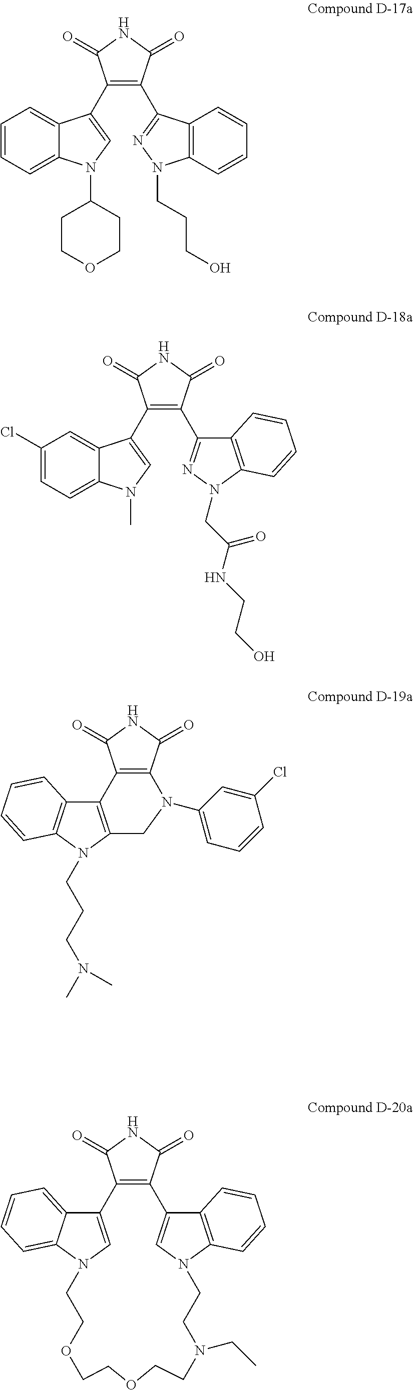 [SMILES: O=C1NC(=O)C(c2nn(CCCO)c3ccccc23)=C1c1cn(C2CCOCC2)c2ccccc12 ; Cn1cc(C2=C(c3nn(CC(=O)NCCO)c4ccccc34)C(=O)NC2=O)c2cc(Cl)ccc21 ; CN(C)CCCn1c2c(c3ccccc31)C1=C(C(=O)NC1=O)N(c1cccc(Cl)c1)C2 ; CCN1CCOCCOCCn2cc(c3ccccc32)C2=C(C(=O)NC2=O)c2cn(c3ccccc23)CC1]

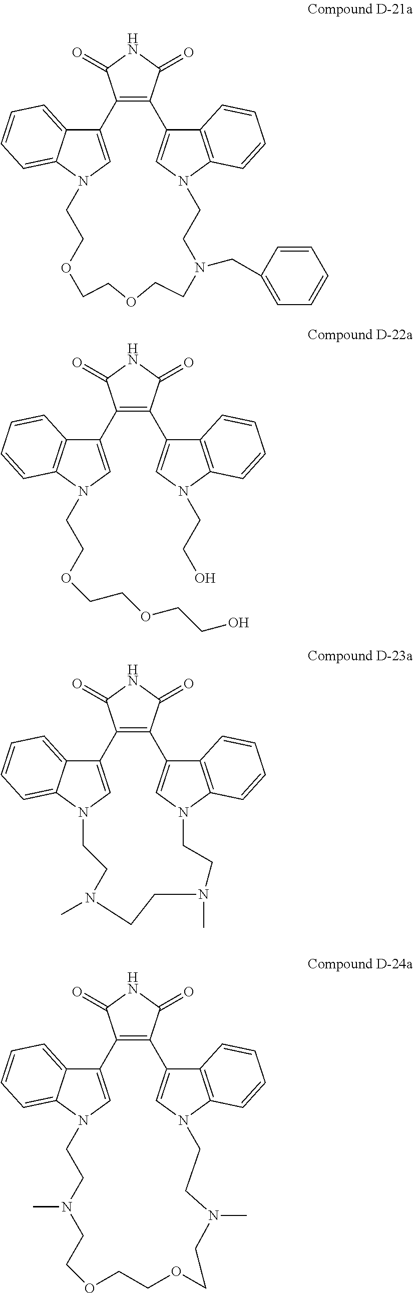 [SMILES: O=C1NC(=O)C2=C1c1cn(c3ccccc13)CCOCCOCCN(Cc1ccccc1)CCn1cc2c2ccccc21 ; O=C1NC(=O)C(c2cn(CCOCCOCCO)c3ccccc23)=C1c1cn(CCO)c2ccccc12 ; CN1CCN(C)CCn2cc(c3ccccc32)C2=C(C(=O)NC2=O)c2cn(c3ccccc23)CC1 ; CN1CCOCCOCCN(C)CCn2cc(c3ccccc32)C2=C(C(=O)NC2=O)c2cn(c3ccccc23)CC1]

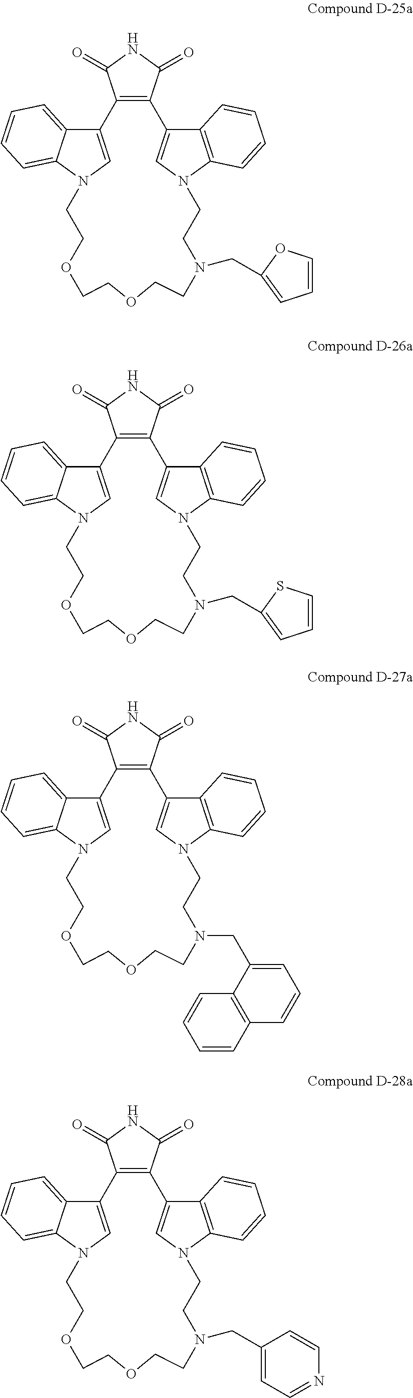 [SMILES: O=C1NC(=O)C2=C1c1cn(c3ccccc13)CCOCCOCCN(Cc1ccco1)CCn1cc2c2ccccc21 ; O=C1NC(=O)C2=C1c1cn(c3ccccc13)CCOCCOCCN(Cc1cccs1)CCn1cc2c2ccccc21 ; O=C1NC(=O)C2=C1c1cn(c3ccccc13)CCOCCOCCN(Cc1cccc3ccccc13)CCn1cc2c2ccccc21 ; O=C1NC(=O)C2=C1c1cn(c3ccccc13)CCOCCOCCN(Cc1ccncc1)CCn1cc2c2ccccc21]

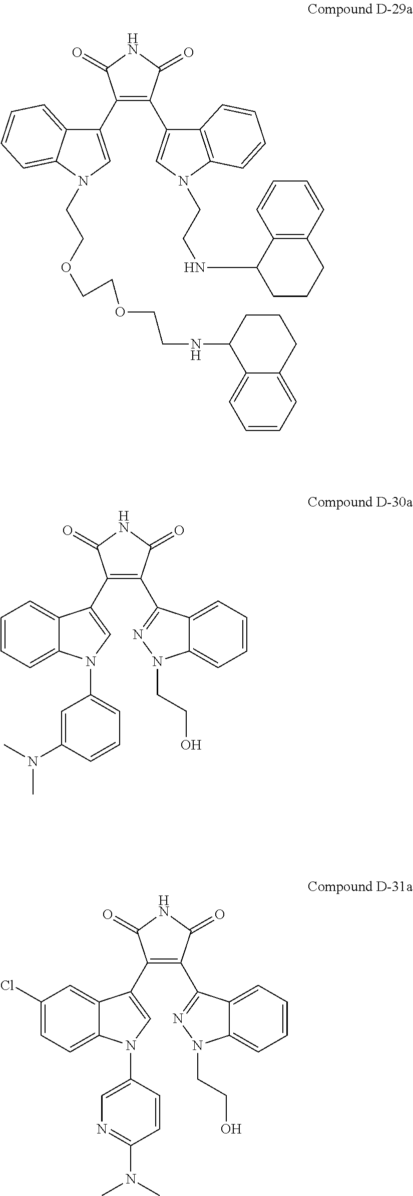 [SMILES: O=C1NC(=O)C(c2cn(CCOCCOCCNC3CCCc4ccccc43)c3ccccc23)=C1c1cn(CCNC2CCCc3ccccc32)c2ccccc12 ; CN(C)c1cccc(-n2cc(C3=C(c4nn(CCO)c5ccccc45)C(=O)NC3=O)c3ccccc32)c1 ; CN(C)c1ccc(-n2cc(C3=C(c4nn(CCO)c5ccccc45)C(=O)NC3=O)c3cc(Cl)ccc32)cn1]

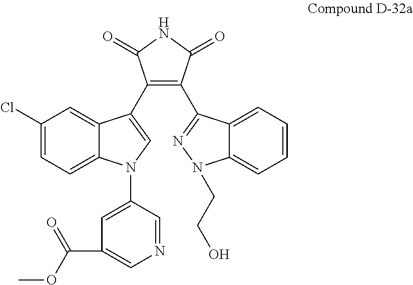 [SMILES: COC(=O)c1cncc(-n2cc(C3=C(c4nn(CCO)c5ccccc45)C(=O)NC3=O)c3cc(Cl)ccc32)c1]

### Cells Suitable for Treatment According to the Methods of the Present Invention

Pluripotent cells, suitable for use in the present invention express at least one of the following pluripotency markers selected from the group consisting of: ABCG2, cripto, FoxD3, Connexin43, Connexin45, Oct4, SOX-2, Nanog, hTERT, UTF-1, ZFP42, SSEA-3, SSEA-4, Tra1-60, and Tra1-81.

In one embodiment, the pluripotent cells are embryonic stem cells. In an alternate embodiment, the pluripotent cells are cells expressing pluripotency markers derived from embryonic stem cells. In one embodiment, the embryonic stem cells are human.

### Isolation, Expansion and Culture of Human Embryonic Stem Cells

Characterization of Human Embryonic Stem Cells:

Human embryonic stem cells may express one or more of the stage-specific embryonic antigens (SSEA) 3 and 4, and markers detectable using antibodies designated Tra-1-60 and Tra-1-81 (Thomson et al., Science 282:1145, 1998). Differentiation of human embryonic stem cells in vitro results in the loss of SSEA-4, Tra-1-60, and Tra-1-81 expression (if present) and increased expression of SSEA-1. Undifferentiated human embryonic stem cells typically have alkaline phosphatase activity, which can be detected by fixing the cells with 4% paraformaldehyde, and then developing with Vector Red as a substrate, as described by the manufacturer (Vector Laboratories, Burlingame Calif.) Undifferentiated pluripotent stem cells also typically express Oct-4 and TERT, as detected by RT-PCR.

Another desirable phenotype of propagated human embryonic stem cells is a potential to differentiate into cells of all three germinal layers: endoderm, mesoderm, and ectoderm tissues. Pluripotency of human embryonic stem cells can be confirmed, for example, by injecting cells into SCID mice, fixing the teratomas that form using 4% paraformaldehyde, and then examining them histologically for evidence of cell types from the three germ layers. Alternatively, pluripotency may be determined by the creation of embryoid bodies and assessing the embryoid bodies for the presence of markers associated with the three germinal layers.

Propagated human embryonic stem cell lines may be karyotyped using a standard G-banding technique and compared to published karyotypes of the corresponding primate species. It is desirable to obtain cells that have a “normal karyotype”, which means that the cells are euploid, wherein all human chromosomes are present and not noticeably altered.

Sources of Human Embryonic Stem Cells:

Types of human embryonic stem cells that may be used include established lines of human embryonic cells derived from tissue formed after gestation, including pre-embryonic tissue (such as, for example, a blastocyst), embryonic tissue, or fetal tissue taken any time during gestation, typically but not necessarily before approximately 10-12 weeks gestation. Non-limiting examples are established lines of human embryonic stem cells or human embryonic germ cells, such as, for example the human embryonic stem cell lines H1, H7, and H9 (WiCell). Also contemplated is use of the compositions of this disclosure during the initial establishment or stabilization of such cells, in which case the source cells would be primary pluripotent cells taken directly from the source tissues. Also suitable are cells taken from a pluripotent stem cell population already cultured in the absence of feeder cells. Also suitable are mutant human embryonic stem cell lines, such as, for example, BG01v (BresaGen, Athens, Ga.).

In one embodiment, Human embryonic stem cells are prepared as described by Thomson et al. (U.S. Pat. No. 5,843,780; Science 282:1145, 1998; Curr. Top. Dev. Biol. 38:133 ff., 1998; Proc. Natl. Acad. Sci. U.S.A. 92:7844, 1995).

Culture of Human Embryonic Stem Cells:

In one embodiment, human embryonic stem cells are cultured in a culture system that is essentially free of feeder cells, but nonetheless supports proliferation of human embryonic stem cells without undergoing substantial differentiation. The growth of human embryonic stem cells in feeder-free culture without differentiation is supported using a medium conditioned by culturing previously with another cell type. Alternatively, the growth of human embryonic stem cells in feeder-free culture without differentiation is supported using a chemically defined medium.

In an alternate embodiment, human embryonic stem cells are initially cultured layer of feeder cells that support the human embryonic stem cells in various ways. The human embryonic are then transferred to a culture system that is essentially free of feeder cells, but nonetheless supports proliferation of human embryonic stem cells without undergoing substantial differentiation.

Examples of conditioned media suitable for use in the present invention are disclosed in US20020072117, US6642048, WO2005014799, and Xu et al (Stem Cells 22: 972-980, 2004).

An example of a chemically defined medium suitable for use in the present invention may be found in US20070010011.

Suitable culture media may be made from the following components, such as, for example, Dulbecco's modified Eagle's medium (DMEM), Gibco #11965-092; Knockout Dulbecco's modified Eagle's medium (KO DMEM), Gibco #10829-018; Ham's F12/50% DMEM basal medium; 200 mM L-glutamine, Gibco #15039-027; non-essential amino acid solution, Gibco 11140-050; 13-mercaptoethanol, Sigma #M7522; human recombinant basic fibroblast growth factor (bFGF), Gibco #13256-029.

In one embodiment, the human embryonic stem cells are plated onto a suitable culture substrate that is treated prior to treatment according to the methods of the present invention. In one embodiment, the treatment is an extracellular matrix component, such as, for example, those derived from basement membrane or that may form part of adhesion molecule receptor-ligand couplings. In one embodiment, a the suitable culture substrate is Matrigel® (Becton Dickenson). Matrigel® is a soluble preparation from Engelbreth-Holm-Swarm tumor cells that gels at room temperature to form a reconstituted basement membrane.

Other extracellular matrix components and component mixtures are suitable as an alternative. This may include laminin, fibronectin, proteoglycan, entactin, heparan sulfate, and the like, alone or in various combinations.

The human embryonic stem cells are plated onto the substrate in a suitable distribution and in the presence of a medium that promotes cell survival, propagation, and retention of the desirable characteristics. All these characteristics benefit from careful attention to the seeding distribution and can readily be determined by one of skill in the art.

### Isolation, Expansion and Culture of Cells Expressing Pluripotency Markers that are Derived from Human Embryonic Stem Cells

In one embodiment, cells expressing pluripotency markers are derived from human embryonic stem cells by a method comprising the steps of:
a. Culturing human embryonic stem cells,
b. Differentiating the human embryonic stem cells into cells expressing markers characteristic of definitive endoderm cells, and
c. Removing the cells, and subsequently culturing them under hypoxic conditions, on a tissue culture substrate that is not pre-treated with a protein or an extracellular matrix prior to culturing the cells.

In one embodiment, cells expressing pluripotency markers are derived from human embryonic stem cells by a method comprising the steps of:
a. Culturing human embryonic stem cells, and
b. Removing the cells, and subsequently culturing them under hypoxic conditions, on a tissue culture substrate that is not pre-treated with a protein or an extracellular matrix.

### Cell Culture Under Hypoxic Conditions on a Tissue Culture Substrate that is Not Pre-Treated with a Protein or an Extracellular Matrix

In one embodiment, the cells are cultured under hypoxic conditions, on a tissue culture substrate that is not coated with an extracellular matrix for about 1 to about 20 days. In an alternate embodiment, the cells are cultured under hypoxic conditions, on a tissue culture substrate that is not coated with an extracellular matrix for about 5 to about 20 days. In an alternate embodiment, the cells are cultured under hypoxic conditions, on a tissue culture substrate that is not coated with an extracellular matrix for about 15 days.

In one embodiment, the hypoxic condition is about 1% O₂ to about 20% O₂. In an alternate embodiment, the hypoxic condition is about 2% O₂ to about 10% O₂. In an alternate embodiment, the hypoxic condition is about 3% O₂.

The cells may be cultured, under hypoxic conditions on a tissue culture substrate that is not pre-treated with a protein or an extracellular matrix, in medium containing serum, activin A, and a Wnt ligand. Alternatively, the medium may also contain IGF-1.

The culture medium may have a serum concentration in the range of about 2% to about 5%. In an alternate embodiment, the serum concentration may be about 2%.

Activin A may be used at a concentration from about 1 pg/ml to about 100 μg/ml. In an alternate embodiment, the concentration may be about 1 pg/ml to about 1 μg/ml. In another alternate embodiment, the concentration may be about 1 pg/ml to about 100 ng/ml. In another alternate embodiment, the concentration may be about 50 ng/ml to about 100 ng/ml. In another alternate embodiment, the concentration may be about 100 ng/ml.

The Wnt ligand may be selected from the group consisting of Wnt-1, Wnt-3a, Wnt-5a and Wnt-7a. In one embodiment, the Wnt ligand is Wnt-1. In an alternate embodiment, the Wnt ligand is Wnt-3a.

The Wnt ligand may be used at a concentration of about 1 ng/ml to about 1000 ng/ml. In an alternate embodiment, the Wnt ligand may be used at a concentration of about 10 ng/ml to about 100 ng/ml. In one embodiment, the concentration of the Wnt ligand is about 20 ng/ml.

IGF-1 may be used at a concentration of about 1 ng/ml to about 100 ng/ml. In an alternate embodiment, the IGF-1 may be used at a concentration of about 10 ng/ml to about 100 ng/ml. In one embodiment, the concentration of IGF-1 is about 50 ng/ml.

The cells expressing pluripotency markers derived by the methods of the present invention are capable of expansion in culture under hypoxic conditions, on tissue culture substrate that is not pre-treated with a protein or an extracellular matrix.

The cells expressing pluripotency markers derived by the methods of the present invention express at least one of the following pluripotency markers selected from the group consisting of: ABCG2, cripto, FoxD3, Connexin43, Connexin45, Oct4, SOX-2, Nanog, hTERT, UTF-1, ZFP42, SSEA-3, SSEA-4, Tra1-60, and Tra1-81.

### Further Differentiation of Cells Expressing Markers Characteristic of the Definitive Endoderm Lineage

Cells expressing markers characteristic of the definitive endoderm lineage may be differentiated into cells expressing markers characteristic of the pancreatic endoderm lineage by any method in the art.

For example, cells expressing markers characteristic of the definitive endoderm lineage may be differentiated into cells expressing markers characteristic of the pancreatic endoderm lineage according to the methods disclosed in D'Amour et al, Nature Biotechnology 24, 1392-1401 (2006).

For example, cells expressing markers characteristic of the definitive endoderm lineage are further differentiated into cells expressing markers characteristic of the pancreatic endoderm lineage, by treating the cells expressing markers characteristic of the definitive endoderm lineage with a fibroblast growth factor and KAAD-cyclopamine, then removing the medium containing the fibroblast growth factor and KAAD-cyclopamine and subsequently culturing the cells in medium containing retinoic acid, a fibroblast growth factor and KAAD-cyclopamine. An example of this method is disclosed in D′ Amour et al, Nature Biotechnology, 24: 1392-1401, (2006).

Markers characteristic of the pancreatic endoderm lineage are selected from the group consisting of Pdx1, HNF-1beta, PTF1a, HNF-6, HB9 and PROX1. Suitable for use in the present invention is a cell that expresses at least one of the markers characteristic of the pancreatic endoderm lineage. In one aspect of the present invention, a cell expressing markers characteristic of the pancreatic endoderm lineage is a pancreatic endoderm cell.

### Further Differentiation of Cells Expressing Markers Characteristic of the Pancreatic Endoderm Lineage

Cells expressing markers characteristic of the pancreatic endoderm lineage may be differentiated into cells expressing markers characteristic of the pancreatic endocrine lineage by any method in the art.

For example, cells expressing markers characteristic of the pancreatic endoderm lineage may be differentiated into cells expressing markers characteristic of the pancreatic endocrine lineage according to the methods disclosed in D'Amour et al, Nature Biotechnology 24, 1392-1401 (2006).

Markers characteristic of the pancreatic endocrine lineage are selected from the group consisting of NGN-3, NeuroD, Islet-1, Pdx-1, NKX6.1, Pax-4, Ngn-3, and PTF-1 alpha. In one embodiment, a pancreatic endocrine cell is capable of expressing at least one of the following hormones: insulin, glucagon, somatostatin, and pancreatic polypeptide. Suitable for use in the present invention is a cell that expresses at least one of the markers characteristic of the pancreatic endocrine lineage. In one aspect of the present invention, a cell expressing markers characteristic of the pancreatic endocrine lineage is a pancreatic endocrine cell. The pancreatic endocrine cell may be a pancreatic hormone expressing cell. Alternatively, the pancreatic endocrine cell may be a pancreatic hormone secreting cell.

In one aspect of the present invention, the pancreatic endocrine cell is a cell expressing markers characteristic of the β cell lineage. A cell expressing markers characteristic of the β cell lineage expresses Pdx1 and at least one of the following transcription factors: NGN-3, Nkx2.2, Nkx6.1, NeuroD, Isl-1, HNF-3 beta, MAFA, Pax4, and Pax6. In one aspect of the present invention, a cell expressing markers characteristic of the β cell lineage is a β cell.

### Detection of Cells Expressing Markers Characteristic of the Definitive Endoderm Linage

Formation of cells expressing markers characteristic of the definitive endoderm lineage may be determined by testing for the presence of the markers before and after following a particular protocol. Pluripotent stem cells typically do not express such markers. Thus, differentiation of pluripotent cells is detected when cells begin to express them.

The efficiency of differentiation may be determined by exposing a treated cell population to an agent (such as an antibody) that specifically recognizes a protein marker expressed by cells expressing markers characteristic of the definitive endoderm lineage.

Methods for assessing expression of protein and nucleic acid markers in cultured or isolated cells are standard in the art. These include quantitative reverse transcriptase polymerase chain reaction (RT-PCR), Northern blots, in situ hybridization (see, e.g., Current Protocols in Molecular Biology (Ausubel et al., eds. 2001 supplement)), and immunoassays such as immunohistochemical analysis of sectioned material, Western blotting, and for markers that are accessible in intact cells, flow cytometry analysis (FACS) (see, e.g., Harlow and Lane, Using Antibodies: A Laboratory Manual, New York: Cold Spring Harbor Laboratory Press (1998)).

Examples of antibodies useful for detecting certain protein markers are listed in Table IA and Table IB. It should be noted that alternate antibodies directed to the same markers that are recognized by the antibodies listed in Table IA and Table IB are available, or can be readily developed. Such alternate antibodies can also be employed for assessing expression of markers in the cells isolated in accordance with the present invention.

For example, characteristics of pluripotent stem cells are well known to those skilled in the art, and additional characteristics of pluripotent stem cells continue to be identified. Pluripotent stem cell markers include, for example, the expression of one or more of the following: ABCG2, cripto, FoxD3, Connexin43, Connexin45, Oct4, Sox2, Nanog, hTERT, UTF-1, ZFP42, SSEA-3, SSEA-4, Tra1-60, Tra1-81.

After treating pluripotent stem cells with the methods of the present invention, the differentiated cells may be purified by exposing a treated cell population to an agent (such as an antibody) that specifically recognizes a protein marker, such as CXCR4, expressed by cells expressing markers characteristic of the definitive endoderm lineage.

### Detection of Cells Expressing Markers Characteristic of the Pancreatic Endoderm Linage

Markers characteristic of the pancreatic endoderm lineage are well known to those skilled in the art, and additional markers characteristic of the pancreatic endoderm lineage continue to be identified. These markers can be used to confirm that the cells treated in accordance with the present invention have differentiated to acquire the properties characteristic of the pancreatic endoderm lineage. Pancreatic endoderm lineage specific markers include the expression of one or more transcription factors such as, for example, Hlxb9, PTF-1a, PDX-1, HNF-6, HNF-1beta.

The efficiency of differentiation may be determined by exposing a treated cell population to an agent (such as an antibody) that specifically recognizes a protein marker expressed by cells expressing markers characteristic of the pancreatic endoderm lineage.

Methods for assessing expression of protein and nucleic acid markers in cultured or isolated cells are standard in the art. These include quantitative reverse transcriptase polymerase chain reaction (RT-PCR), Northern blots, in situ hybridization (see, e.g., Current Protocols in Molecular Biology (Ausubel et al., eds. 2001 supplement)), and immunoassays such as immunohistochemical analysis of sectioned material, Western blotting, and for markers that are accessible in intact cells, flow cytometry analysis (FACS) (see, e.g., Harlow and Lane, Using Antibodies: A Laboratory Manual, New York: Cold Spring Harbor Laboratory Press (1998)).

Examples of antibodies useful for detecting certain protein markers are listed in Table IA and Table IB. It should be noted that alternate antibodies directed to the same markers that are recognized by the antibodies listed in Table IA and Table IB are available, or can be readily developed. Such alternate antibodies can also be employed for assessing expression of markers in the cells isolated in accordance with the present invention.

### Detection of Cells Expressing Markers Characteristic of the Pancreatic Endocrine Linage

Markers characteristic of cells of the pancreatic endocrine lineage are well known to those skilled in the art, and additional markers characteristic of the pancreatic endocrine lineage continue to be identified. These markers can be used to confirm that the cells treated in accordance with the present invention have differentiated to acquire the properties characteristic of the pancreatic endocrine lineage. Pancreatic endocrine lineage specific markers include the expression of one or more transcription factors such as, for example, NGN-3, NeuroD, Islet-1.

Markers characteristic of cells of the β cell lineage are well known to those skilled in the art, and additional markers characteristic of the β cell lineage continue to be identified. These markers can be used to confirm that the cells treated in accordance with the present invention have differentiated to acquire the properties characteristic of the β-cell lineage. β cell lineage specific characteristics include the expression of one or more transcription factors such as, for example, Pdx1 (pancreatic and duodenal homeobox gene-1), Nkx2.2, Nkx6.1, Isl1, Pax6, Pax4, NeuroD, Hnf1b, Hnf-6, Hnf-3beta, and MafA, among others. These transcription factors are well established in the art for identification of endocrine cells. See, e.g., Edlund (Nature Reviews Genetics 3: 524-632 (2002)).

The efficiency of differentiation may be determined by exposing a treated cell population to an agent (such as an antibody) that specifically recognizes a protein marker expressed by cells expressing markers characteristic of the pancreatic endocrine lineage. Alternatively, the efficiency of differentiation may be determined by exposing a treated cell population to an agent (such as an antibody) that specifically recognizes a protein marker expressed by cells expressing markers characteristic of the β cell lineage.

Methods for assessing expression of protein and nucleic acid markers in cultured or isolated cells are standard in the art. These include quantitative reverse transcriptase polymerase chain reaction (RT-PCR), Northern blots, in situ hybridization (see, e.g., Current Protocols in Molecular Biology (Ausubel et al., eds. 2001 supplement)), and immunoassays such as immunohistochemical analysis of sectioned material, Western blotting, and for markers that are accessible in intact cells, flow cytometry analysis (FACS) (see, e.g., Harlow and Lane, Using Antibodies: A Laboratory Manual, New York: Cold Spring Harbor Laboratory Press (1998)).

Examples of antibodies useful for detecting certain protein markers are listed in Table IA and Table IB. It should be noted that alternate antibodies directed to the same markers that are recognized by the antibodies listed in Table IA and Table IB are available, or can be readily developed. Such alternate antibodies can also be employed for assessing expression of markers in the cells isolated in accordance with the present invention.

The present invention is further illustrated, but not limited by, the following examples.

### Example 1

### Human Embryonic Stem Cell Culture

Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

The human embryonic stem cell lines H1, H7 and H9 were obtained from WiCell Research Institute, Inc., (Madison, Wis.) and cultured according to instructions provided by the source institute. Briefly, cells were cultured on mouse embryonic fibroblast (MEF) feeder cells in ES cell medium consisting of DMEM/F12 (Invitrogen/GIBCO) supplemented with 20% knockout serum replacement, 100 nM MEM nonessential amino acids, 0.5 mM beta-mercaptoethanol, 2 mM L-glutamine with 4 ng/ml human basic fibroblast growth factor (bFGF) (all from Invitrogen/GIBCO). MEF cells, derived from E13 to 13.5 mouse embryos, were purchased from Charles River. MEF cells were expanded in DMEM medium supplemented with 10% FBS (Hyclone), 2 mM glutamine, and 100 mM MEM nonessential amino acids. Sub-confluent MEF cell cultures were treated with 10 μg/ml mitomycin C (Sigma, St. Louis, Mo.) for 3 h to arrest cell division, then trypsinized and plated at 2×10⁴/cm² on 0.1% bovine gelatin-coated dishes. MEF cells from passage two through four were used as feeder layers. Human embryonic stem cells plated on MEF cell feeder layers were cultured at 37° C. in an atmosphere of 5% CO₂/within a humidified tissue culture incubator. When confluent (approximately 5-7 days after plating), human embryonic stem cells were treated with 1 mg/ml collagenase type IV (Invitrogen/GIBCO) for 5-10 min and then gently scraped off the surface using a 5-ml pipette. Cells were spun at 900 rpm for 5 min, and the pellet was resuspended and re-plated at a 1:3 to 1:4 ratio of cells in fresh culture medium.

In parallel, H1, H7, and H9 human embryonic stem cells were also seeded on plates coated with a 1:30 dilution of growth factor reduced MATRIGEL™ (BD Biosciences) and cultured in MEF-conditioned media supplemented with 8 ng/ml bFGF. The cells cultured on MATRIGEL™ were routinely passaged with collagenase IV (Invitrogen/GIBCO), Dispase (BD Biosciences) or Liberase enzyme (Source). Some of the human embryonic stem cell cultures were incubated under hypoxic conditions (approximately 3% O₂).

### Example 2

### Derivation and Culture of Cells Expressing Pluripotency Markers, Derived from Human Embryonic Stem Cells

Cells from the human embryonic stem cell lines H1 and H9 various passages (Passage 30-54) were cultured under hypoxic conditions (approximately 3% O₂) for at least three passages. The cells were cultured in MEF-CM supplemented with 8 ng/ml of bFGF and plated on MATRIGEL coated plates according to Example 1.

Cells were then treated with DMEM/F12 medium supplemented with 0.5% FBS, 20 ng/ml WNT-3a (Catalog#1324-WN-002, R&D Systems, MN), and 100 ng/ml Activin-A (R&D Systems, MN) for two days followed by treatment with DMEM/F12 media supplemented with 2% FBS and 100 ng/ml Activin-A (AA) for an additional 3 to 4 days. This protocol resulted in significant upregulation of definitive endoderm markers.

The cells were then treated with TrypLE™ Express solution (Invitrogen, CA) for 5 mins. Released cells were resuspended in DMEM-F12+2% FBS medium, recovered by centrifugation, and counted using a hemocytometer. The released cells were seeded at 1000-10,000 cells/cm² on tissue culture polystyrene (TCPS) treated flasks and cultured in DMEM-F12+2% FBS+100 ng/ml activin-A+20 ng/ml WNT-3A under hypoxic conditions (approximately 3% O₂) at 37° C. in standard tissue culture incubator. The TCPS flaks were not coated with MATRIGEL or other extracellular matrix proteins. The media was changed daily. In some cultures, the media was further supplemented with 10-50 ng/ml of IGF-I (insulin growth factor-I from R&D Systems, MN) or 1×ITS (Insulin, transferrin, and selenium from Invitrogen, Ca). In some of the culture conditions the basal media (DM-F12+2% FBS) was further supplemented with 0.1 mM mercaptoethanol (Invitrogen, CA) and non-essential amino acids (1×, NEAA from Invitrogen, CA).

Following 5 to 15 days of culturing, distinct cell colonies appeared surrounded by a large number of enlarged cells that appear to be in senescence. At approximately 50 to 60% confluency, the cultures were passaged by exposure to TrypLE™ Express solution for 5 mins at room temperature. The released cells were resuspended in DMEM-F12+2% FBS medium, recovered by centrifugation, and seeded at 10,000 cells/cm² on tissue culture polystyrene (TCPS) treated flasks in DMEM-F12+2% FBS+100 ng/ml activin-A+20 ng/ml WNT-3A+/−50 ng/ml of IGF-I. This media will be further referred to as the “growth media”.

### Example 3A

### Derivation of Cells Expressing Pluripotency Markers from a Single Cell Suspension of Human Embryonic Stem Cells

Cells from the human embryonic stem cell lines H1 P33 and H9 P45 were cultured under hypoxic conditions (approximately 3% O₂) for at least three passages. The cells were cultured in MEF-CM supplemented with 8 ng/ml of bFGF and plated on MATRIGEL coated plates according to Example 1. At approximately 60% confluency, the cultures were exposed to TrypLE™ Express solution (Invitrogen, CA) for 5 minutes. Released cells were resuspended in DMEM-F12+2% FBS medium, recovered by centrifugation, and counted using a hemocytometer. The released cells were seeded at 1000 to 10,000 cells/cm² on tissue culture polystyrene (TCPS) treated flasks and cultured in DM-F12+2% FBS+100 ng/ml activin-A+20 ng/ml WNT-3A+50 ng/ml of IGF-I+0.1 mM mercaptoethanol (Invitrogen, CA) and nonessential amino acids (1×, NEAA from Invitrogen, CA) under hypoxic conditions (approximately 3% O₂) at 37° C. in standard tissue culture incubator. The TCPS flasks were not coated with MATRIGEL or other extracellular matrix proteins. The media was changed daily. The first passage cells are referred to as P1.

### Example 3B

### Various Growth Media Useful for Expansion of Cells Expressing Pluripotency Markers Derived from Human Embryonic Stem Cells

Cells expressing pluripotency markers derived from human embryonic stem cells have been successfully cultured in the following media compositions for at least 2-30 passages:
1. DM-F12+2% FBS+100 ng/ml AA+20 ng/ml WNT-3A
2. DM-F12+2% FBS+100 ng/ml AA+20 ng/ml WNT-3A+50 ng/ml IGF-I
3. DM-F12+2% FBS+100 ng/ml AA+20 ng/ml WNT-3A+10 ng/ml IGF-I
4. DM-F12+2% FBS+50 ng/ml AA+20 ng/ml WNT-3A+50 ng/ml IGF-I
5. DM-F12+2% FBS+50 ng/ml AA+10 ng/ml WNT-3A+50 ng/ml IGF-I
6. DM-F12+2% FBS+50 ng/ml AA+20 ng/ml WNT-3A+10 ng/ml IGF-I
7. DM-F12+2% FBS+100 ng/ml AA+10 ng/ml WNT-3A+10 ng/ml IGF-I
8. HEScGRO defined media (Chemicon, CA)

The basal component of the above listed media may be replaced with similar media such as, RPMI, DMEM, CRML, Knockout™DMEM, and F12.

### Example 4

### Effects of Inhibitors of GSK-313 Enzyme Activity on the Viability of Cells Expressing Pluripotency Markers

Derivation and maintenance of cells expressing pluripotency makers was conducted as has been described in Example 2. Cells were grown in DMEM:F12 supplemented with 2% FCS (Invitrogen), 100 ng/ml Activin A, 20 ng/ml Wnt-3a, and 50 ng/ml IGF (R&D Biosystems). Cells were seeded at a density of 10,000 cells/cm² on Falcon polystyrene flasks and grown in monolayer culture at 37° C., 5% CO₂, low oxygen. After reaching 60-70% confluence, cells were passed by washing the monolayer with PBS and incubating with TrypLE (Invitrogen) for 3-5 minutes to allow detachment and single cell dispersal.

Screening was conducted using test compounds from a proprietary library of small molecules selected for their ability to inhibit GSK-3B enzyme activity. Compounds from this library were made available as 1 mM stocks, in a 96-well plate format in 50 mM HEPES, 30% DMSO. For assay, cells expressing pluripotency markers were washed, counted, and plated in normal culture medium at a seeding density of 20,000 cells per well in 96-well clear-bottom, dark-well plates (Costar). This seeding density was previously determined to yield optimal monolayer formation in overnight culture. On the following day, culture medium was removed, cell monolayers were rinsed three times with PBS, and test compounds were added to the wells in 80 μl aliquots, each diluted into assay medium at a final assay concentration of 10 μM. On day 2 of the assay, medium was removed from each well and replaced with a fresh aliquot of test compounds diluted into assay medium. Assay medium on days 1 and 2 of culture consisted of DMEM:F12 supplemented with 0.5% FCS and 100 ng/ml Activin A. On days 3 and 4 of culture, medium was removed from each well and replaced with DMEM:F12 supplemented with 2% FCS and 100 ng/ml Activin A (no test compound). On day 4 of assay, 15 μl of MTS (Promega) was added to each well and plates were incubated at 37° C. for 1.5 to 4 hours prior to reading optical density at 490 nm on a SpectraMax (Molecular Devices) instrument. Statistical measures consisting of mean, standard deviation, and coefficient of variation were calculated for each duplicate set. Toxicity was calculated for each test well relative to a positive control (wells treated with Activin A and Wnt3a on days 1 and 2 of culture).

Table II is a compilation of all screening results. Cells expressing pluripotency markers were plated initially as a confluent monolayer in this assay; hence, the results are representative of a toxicity measure over the four-day culture period. Results are expressed as percentage viability of control, and demonstrate variable toxicity for some compounds at the 10 μM screening concentration used. A larger proportion of the compounds have minimal or no measurable toxicity in this cell-based assay.

A small panel of select compounds was repeat tested over a narrow dose titration range, again using cells expressing pluripotency markers in a similar assay as described above. Table III is a summary of these results, demonstrating variable dose titration effects for a range of toxic and non-toxic compounds.

### Example 5

Effects of Inhibitors of GSK-313 Enzyme Activity on the Differentiation and Proliferation of Human Embryonic Stem Cells Determined using a High Content Screening Assay

Maintenance of human embryonic stem cells (H9 line) was conducted as described in Example 1. Colonies of cells were maintained in an undifferentiated, pluripotent state with passage on average every four days. Passage was performed by exposing cell cultures to a solution of collagenase (1 mg/ml; Sigma-Aldrich) for 10 to 30 minutes at 37° C. followed by gentle scraping with a pipette tip to recover cell clusters. Clusters were allowed to sediment by gravity, followed by washing to remove residual collagenase. Cell clusters were split at a 1:3 ratio for routine maintenance culture or a 1:1 ratio for immediate assay. The human embryonic stem cell lines used were maintained at passage numbers less than passage 50 and routinely evaluated for normal karyotypic phenotype and absence of mycoplasma contamination.

Cell clusters used in the assay were evenly resuspended in normal culture medium and plated onto MATRIGEL-coated 96-well Packard VIEWPLATES (PerkinElmer) in volumes of 100 μA/well. MEF conditioned medium supplemented with 8 ng/ml bFGF was used for initial plating and recovery. Daily feeding was conducted by aspirating spent culture medium from each well and replacing with an equal volume of fresh medium. Plates were maintained at 37° C., 5% CO₂ in a humidified box throughout the duration of assay.

Screening was conducted using test compounds from a proprietary library of small molecules selected for their ability to inhibit GSK-3B enzyme activity. Compounds from this library were made available as 1 mM stocks, in a 96-well plate format in 50 mM HEPES, 30% DMSO. Screening compounds were tested in triplicate or duplicate sets. Primary screening assays were initiated by aspirating culture medium from each well followed by three washes in PBS to remove residual growth factors and serum. Test volumes of 80 to 100 μl per well were added back containing DMEM:F12 base medium (Invitrogen) supplemented with 0.5% FCS (HyClone) and 100 ng/ml activin A (R&D Biosystems) plus 10 μM test compound. Positive control wells contained the same base medium, substituting 10-20 ng/ml Wnt3a (R&D Biosystems) for the test compound. Negative control wells contained base medium with 0.5% FCS and activin A alone (AA only) or alternatively, 0.5% FCS without activin A or Wnt3a (no treatment). Wells were aspirated and fed again with identical solutions on day 2 of assay. On days 3 and 4, all assay wells were aspirated and converted to DMEM:F12 supplemented with 2% FCS and 100 ng/ml activin A (without test compound or Wnt3a); parallel negative control wells were maintained in DMEM:F12 base medium with 2% FCS and activin A (AA only) or alternatively, 2% FCS without activin A (no treatment).

At the end of culture, cells in 96-well plates were fixed with 4% paraformaldehyde at room temperature for 20 minutes, washed three times with PBS, and then permeabilized with 0.5% Triton X-100 for 20 minutes at room temperature. Alternatively, cells were fixed with ice cold 70% ethanol overnight at −20° C., washed three times with PBS, and then permeabilized with Triton X-100 for 5 minutes at 4° C. After fixing and permeabilizing, cells were washed again three times with PBS and then blocked with 4% chicken serum (Invitrogen) in PBS for 30 minutes at room temperature. Primary antibodies (goat anti-human Sox17 and goat anti-human HNF-3beta; R&D Systems) were diluted 1:100 in 4% chicken serum and added to cells for one hour at room temperature. Alexa Fluor 488 conjugated secondary antibody (chicken anti-goat IgG; Molecular Probes) was diluted 1:200 in PBS and added after washing the cells three times with PBS. To counterstain nuclei, 5 mM Draq5 (Alexis Biochemicals) was added for five minutes at room temperature. Cells were washed once with PBS and left in 100 ml/well PBS for imaging.

Cells were imaged using an IN Cell Analyzer 1000 (GE Healthcare) utilizing the 51008bs dichroic for cells stained with Draq5 and Alexa Fluor 488. Exposure times were optimized using a positive control wells and wells with secondary only for untreated negative controls. Twelve fields per well were obtained to compensate for any cell loss during the treatment and staining procedures. Total cell numbers and total cell intensity for Sox-17 and HNF-3beta were measured using the IN Cell Developer Toolbox 1.6 (GE Healthcare) software. Segmentation for the nuclei was determined based on grey-scale levels (baseline range 100-300) and nuclear size. Averages and standard deviations were calculated for replicates. Total protein expression was reported as total intensity or integrated intensity, defined as total fluorescence of the cell times area of the cell. Background was eliminated based on acceptance criteria of grey-scale ranges between 300 to 3000 and form factors greater than or equal to 0.4. Total intensity data were normalized by dividing the total intensities for each well by the average total intensity for the Wnt3a/Activin A positive control. Normalized data was calculated for averages and standard deviation for each replicate set.

Table IV is a representative summary of all screening results. Table V is a list of hits from this screening. Strong hits are defined as greater than or equal to 120% of control values; moderate hits are defined as falling within the interval of 60-120% of control values. A significant number of compounds induce both a proliferative response in this assay. In parallel, a significant number of compounds induce differentiation in this assay, as measured by the protein expression of Sox17 and Hnf-3b transcription factors.

### Example 6

### Effects of Inhibitors of GSK-313 Enzyme Activity on the Proliferation of Human Embryonic Stem Cells Determined using a Plate Reader Assay

Maintenance of human embryonic stem cells (H9 or H1 lines) was conducted as described in Example 1. Colonies of cells were maintained in an undifferentiated, pluripotent state with passage on average every four days. Passage was performed by exposing cell cultures to a solution of collagenase (1 mg/ml; Sigma-Aldrich) for 10 to 30 minutes at 37° C. followed by gentle scraping with a pipette tip to recover cell clusters. Clusters were allowed to sediment and washed to remove residual collagenase. Cell clusters were split at a ratio of 1:3 monolayer area for routine culture or a 1:1 ratio for immediate assay. The human embryonis stem cell lines used for these examples were maintained at passage numbers less than 50 and routinely evaluated for normal karyotypic phenotype as well as absence of mycoplasm contamination.

Cell clusters used in assay were evenly resuspended in normal culture medium and plated into MATRIGEL-coated 96-well Packard VIEWPLATES (PerkinElmer) in volumes of 100 μl/well. MEF conditioned medium supplemented with 8 ng/ml bFGF) was used for initial plating and recovery. Daily feeding was conducted by aspirating spent culture medium from each well and replacing with an equal volume of fresh medium. Plates were maintained at 37° C. in a humidified box, 5% CO₂ throughout the duration of assay.

Primary screening assays were initiated by aspirating culture medium from each well followed by three washes in PBS to remove residual growth factors and serum. Test volumes of 80-100 μl per well were added back containing DMEM:F12 base medium (Invitrogen) supplemented with 0.5% FCS (HyClone) and 100 ng/ml activin A (R&D Biosystems) and 10 μM test compound. Positive control wells contained the same medium substituting 10-20 ng/ml Wnt3a (R&D Biosystems). Negative control wells contained base medium with 0.5% FCS without activin A or Wnt3a. Screening compounds were tested in triplicate. Wells were aspirated and fed again with identical solutions on day 2 of the assay. On days 3 and 4, all assay wells were aspirated and converted to DMEM:F 12 supplemented with 2% FCS and 100 ng/ml activin A with the exception of negative control wells which were maintained in DMEM:F12 base medium with 2% FCS.

On day 4 of assay, 15-20 μl of MTS (Promega) was added to each well and plates were incubated at 37° C. for 1.5 to 4 hours. Densitometric readings at OD490 were determined using a Molecular Devices spectrophotometer plate reader. Average readings for replicate sets were calculated along with standard deviation and coefficient of variation. Experimental wells were compared to the Activin A/Wnt3a positive control to calculate a percent control value as a measure of proliferation.

Table VI is a representative summary of all screening results. Table VII is a list of hits from this screening. Strong hits are defined as greater than or equal to 120% of control values; moderate hits are defined as falling within the interval of 60-120% of control values. A significant number of compounds induce a proliferative response in this assay.

### Example 7

### Effects of GSK-313 Enzyme Inhibitors on the Differentiation and Proliferation of Human Embryonic Stem Cells: Dose Titration of Lead Compounds

It was important to confirm the activity of hits identified from primary screening and further analyze the range of activity by dose titration. New samples of a selective subset of primary screening hits were obtained as dry powders, solubilized to make fresh stock reagents, and diluted into secondary confirmation assays to evaluate effects on human embryonic stem cells.

Culture of two human embryonic stem cells (H1 and H9) was conducted as described in Example 1. Colonies of cells were maintained in an undifferentiated, pluripotent state on Matrigel™ (Invitrogen) coated polystyrene plastic, using a 1:30 dilution of Matrigel™ in DMEM:F12 to coat the surface. Cells were split by enzymatic passage every four days on average. Passage was performed by exposing cell monolayers to a solution of collagenase (1 mg/ml; Sigma-Aldrich) for 10 to 60 minutes at 37° C. followed by gentle scraping with a pipette tip to recover cell clusters. Clusters were allowed to sediment by gravity, then washed to remove residual collagenase. Cell clusters were split at a 1:3 ratio for maintenance culture or a 1:1 ratio for subsequent assay. The human embryonic stem cell lines were maintained at less than passage 50 and routinely evaluated for normal karyotypic phenotype and absence of mycoplasma contamination.

Preparation of Cells for Assay:

Cell clusters of the H1 or H9 human embryonic stem cell lines used in the assay were evenly resuspended in culture medium and plated onto Matrigel™-coated 96-well Packard VIEWPLATES (PerkinElmer) in volumes of 100 μl/well. MEF conditioned medium supplemented with 8 ng/ml bFGF was used for initial plating and expansion. Daily feeding was conducted by aspirating spent culture medium from each well and replacing with an equal volume of fresh medium. Cultures were allowed to expand one to three days after plating prior to initiating assay. Plates were maintained at 37° C., 5% CO₂ in a humidified box for the duration of assay.

Preparation of Compounds and Assay Medium:

A subset of hits resulting from primary screening was used for follow-up study and subsequent secondary assays. Twenty compounds available as dry powders were solubilized as 10 mM stocks in DMSO and stored dessicated at 20° C. until use Immediately prior to assay, compound stocks were diluted 1:1000 to make 10 μM test compound in DMEM:F12 base medium (Invitrogen) supplemented with 0.5% FCS (HyClone) and 100 ng/ml Activin A (R&D Biosystems). This was further diluted two-fold in series to make a seven point dilution curve for each compound, also in DMEM:F12 base medium with 0.5% FCS and 100 ng/ml Activin A.

Secondary Screening Assay:

Assay was initiated by aspirating culture medium from cell monolayers in each well followed by three washes in PBS to remove residual growth factors and serum. Test volumes of 100 μl per well were added back containing medium with 0.5% FCS and different concentrations of inhibitor compounds with 100 ng/ml Activin A, without Wnt3a. Positive control wells contained the same base medium with 0.5% FCS and with 20 ng/ml Wnt3a (R&D Biosystems) in the absence of test compound. Negative control wells contained the same base medium with 0.5% FCS, in the absence of Activin A, Wnt3a, or test compound. Assay wells were aspirated and fed again with identical concentrations of test compound or control solutions on day 2 of assay. On days 3 and 4, all assay wells were aspirated and fed with DMEM:F12 supplemented with 2% FCS and 100 ng/ml Activin A in the absence of both test compound or Wnt3a. Parallel negative control wells were maintained on days 3 and 4 in DMEM:F12 base medium with 2% FCS.

Assay Evaluation:

At the end of culture, cells in 96-well plates were washed twice with PBS then fixed with 4% paraformaldehyde at room temperature for 20 minutes, washed three times more with PBS, and then permeabilized with 0.5% Triton X-100 for 20 minutes at room temperature. After fixing and permeabilizing, cells were washed again three times with PBS and then blocked with 4% chicken serum (Invitrogen) in PBS for 30 minutes at room temperature. Primary antibodies (goat anti-human Sox17; R&D Systems) were diluted 1:100 in 4% chicken serum and added to the cells for one hour at room temperature. Alexa Fluor 488 conjugated secondary antibody (chicken anti-goat IgG; Molecular Probes) was diluted 1:200 in PBS and added to each well after washing the cells three times with PBS. To counterstain nuclei, 2 μg/ml Hoechst 33342 (Invitrogen) was added for ten minutes at room temperature. Cells were washed once with PBS and left in 100 μA/well PBS for imaging.

Cells were imaged using an IN Cell Analyzer 1000 (GE Healthcare) utilizing the 51008bs dichroic for cells stained with Hoechst 33342 and Alexa Fluor 488. Exposure times were optimized using positive control wells and wells stained with secondary antibody alone as an untreated negative control. Images from 15 fields per well were acquired to compensate for any cell loss during the treatment and staining procedures. Measurements for total cell number and total Sox-17 intensity were obtained for each well using IN Cell Developer Toolbox 1.7 (GE Healthcare) software. Segmentation for the nuclei was determined based on grey-scale levels (baseline range 100-300) and nuclear size. Averages and standard deviations were calculated for each replicate data set. Total Sox17 protein expression was reported as total intensity or integrated intensity, defined as total fluorescence of the cell times area of the cell. Background was eliminated based on acceptance criteria of grey-scale ranges between 300 to 3000 and form factors greater than or equal to 0.4. Total intensity data were normalized by dividing the total intensities for each well by the average total intensity for the Wnt3a/Activin A positive control. Normalized data were calculated for averages and standard deviations for each replicate set.

### Results

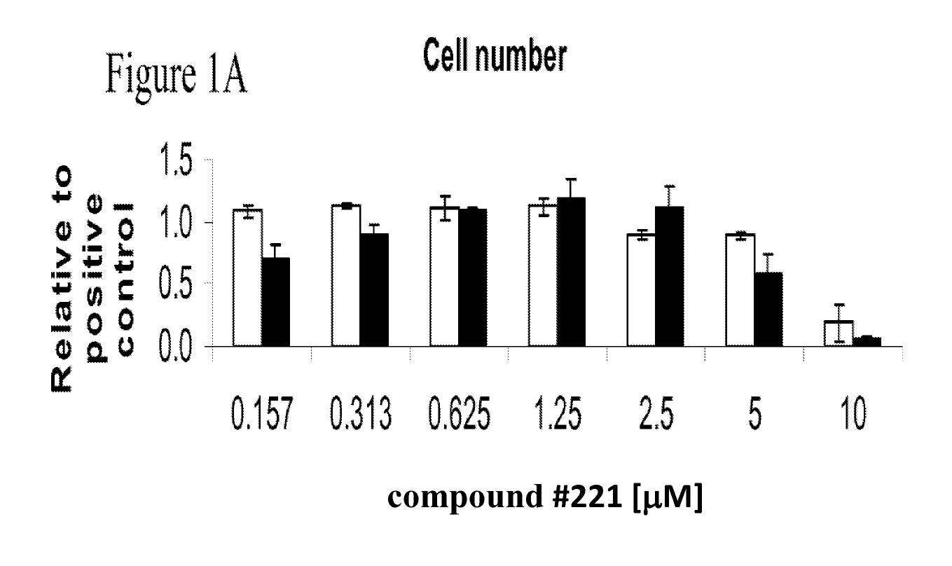

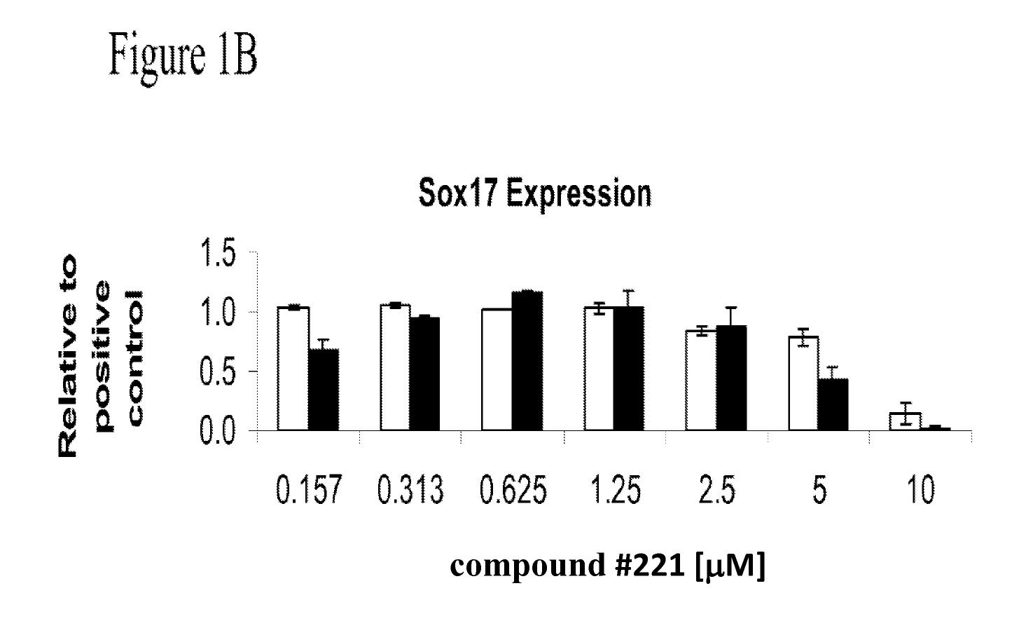

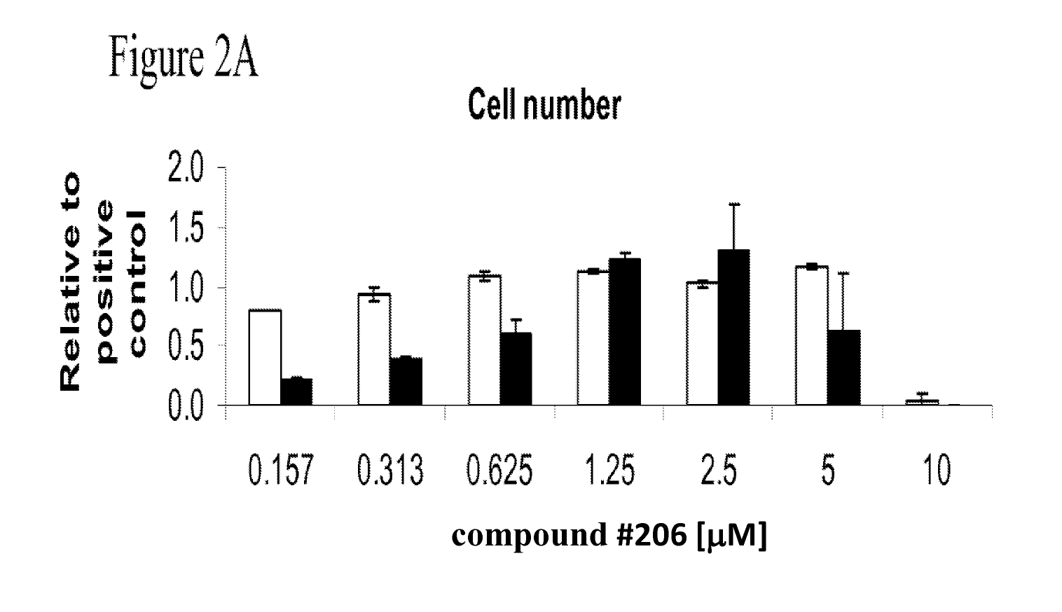

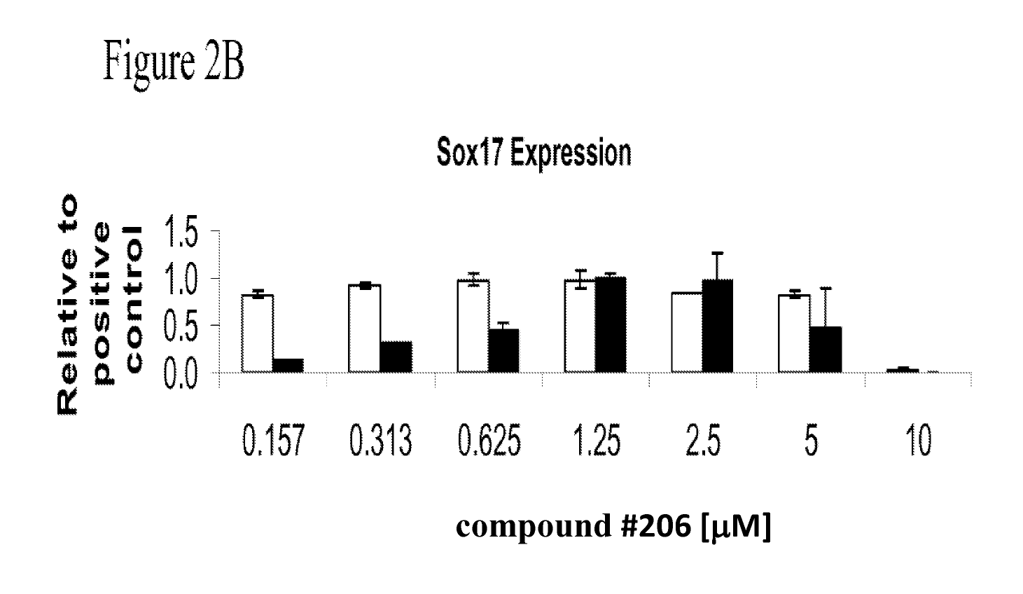

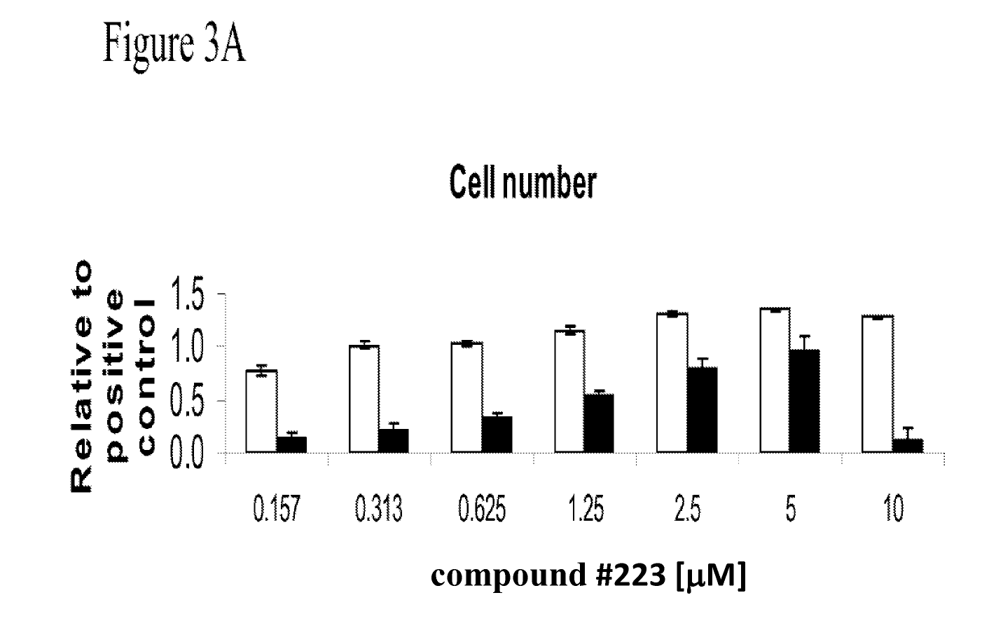

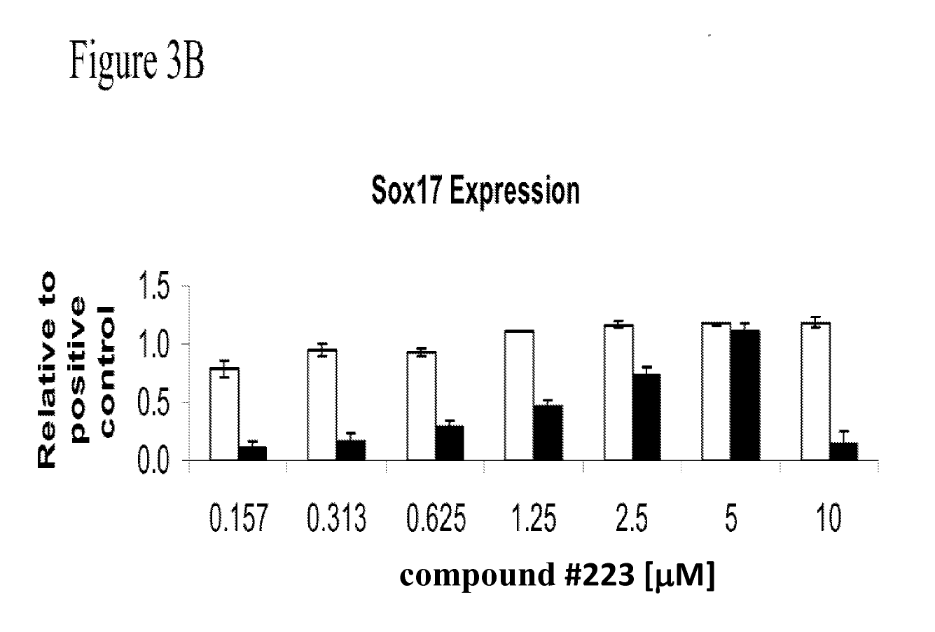

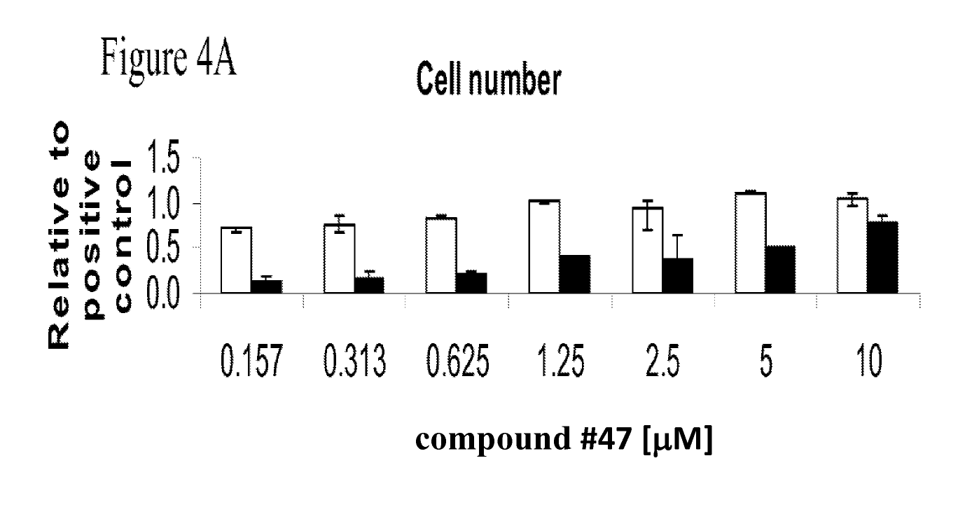

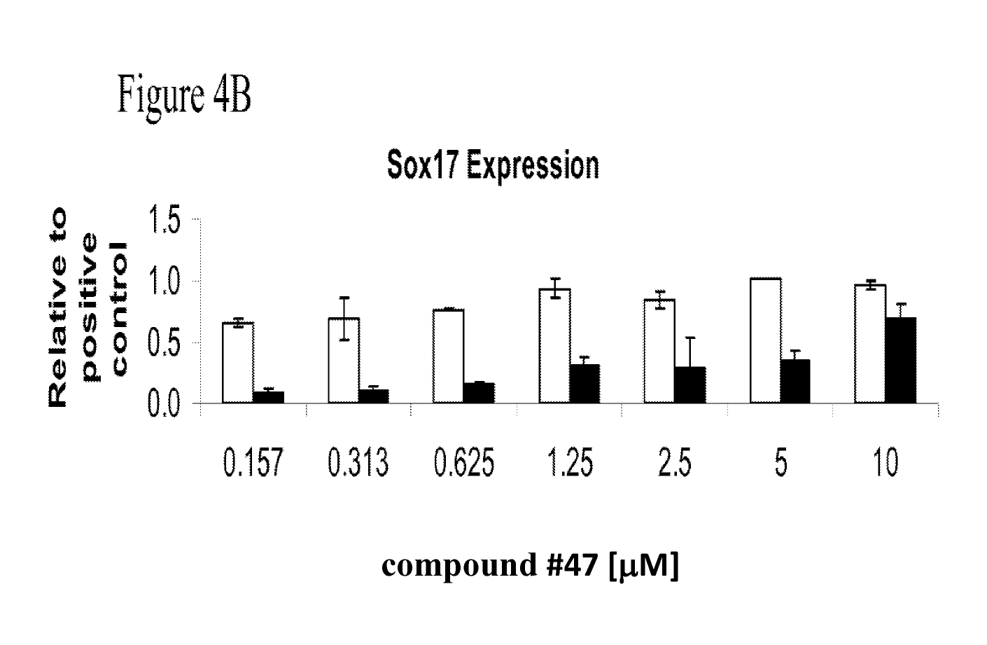

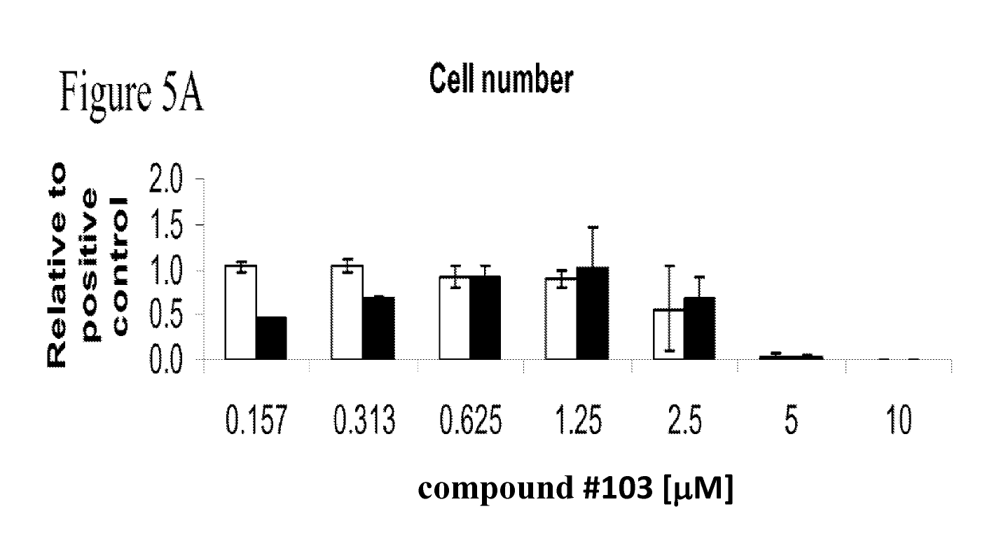

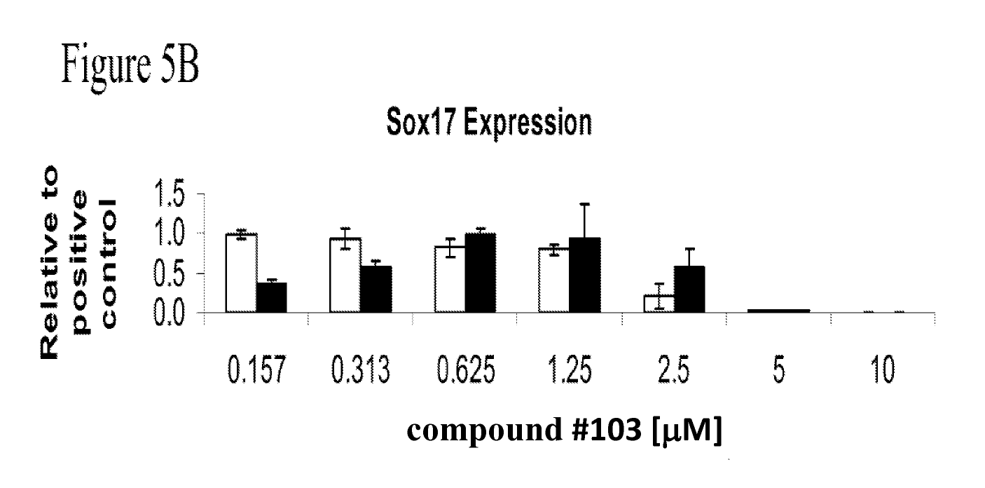

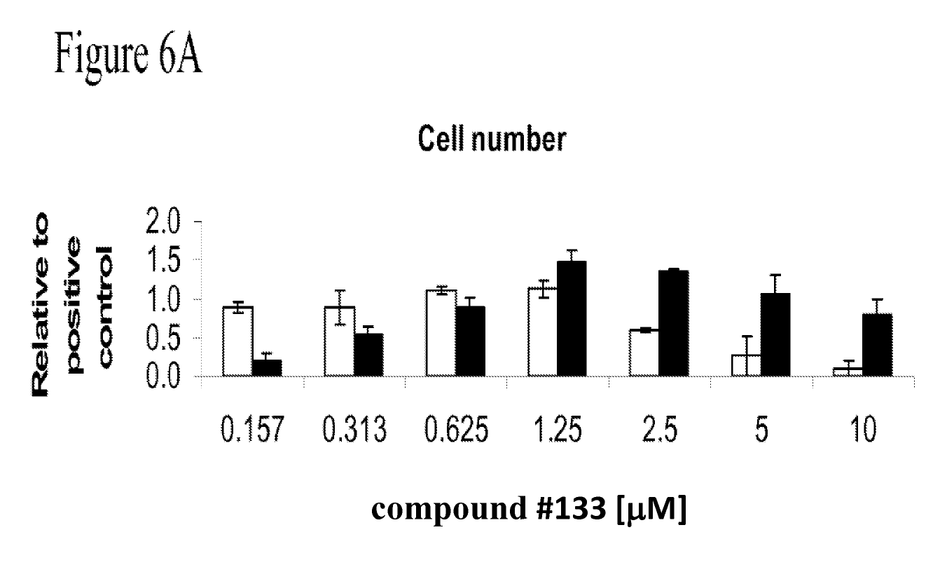

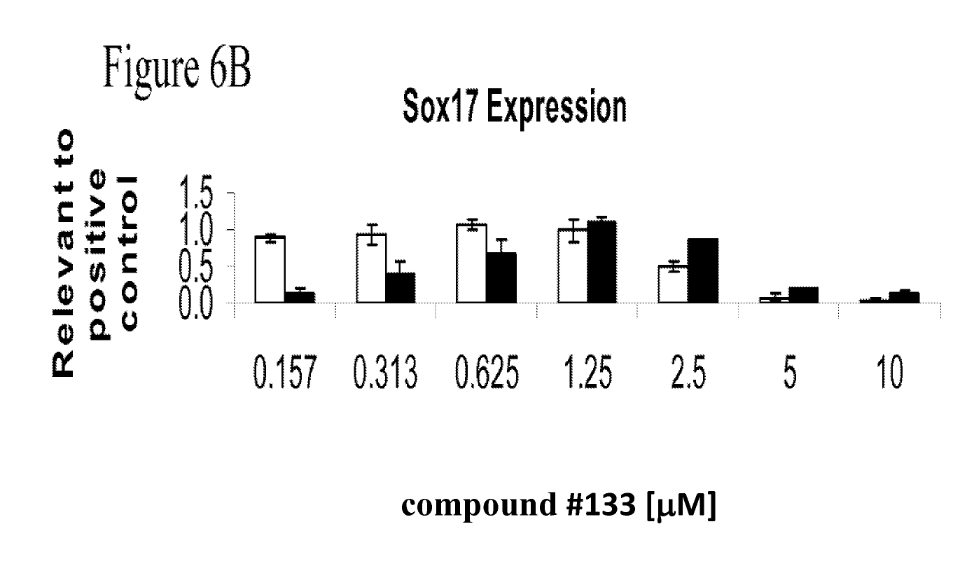

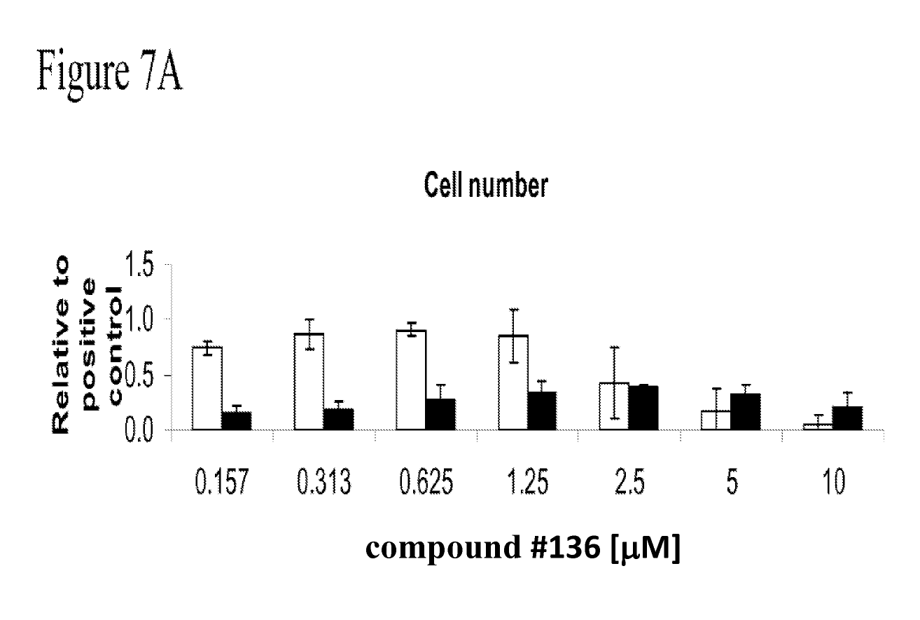

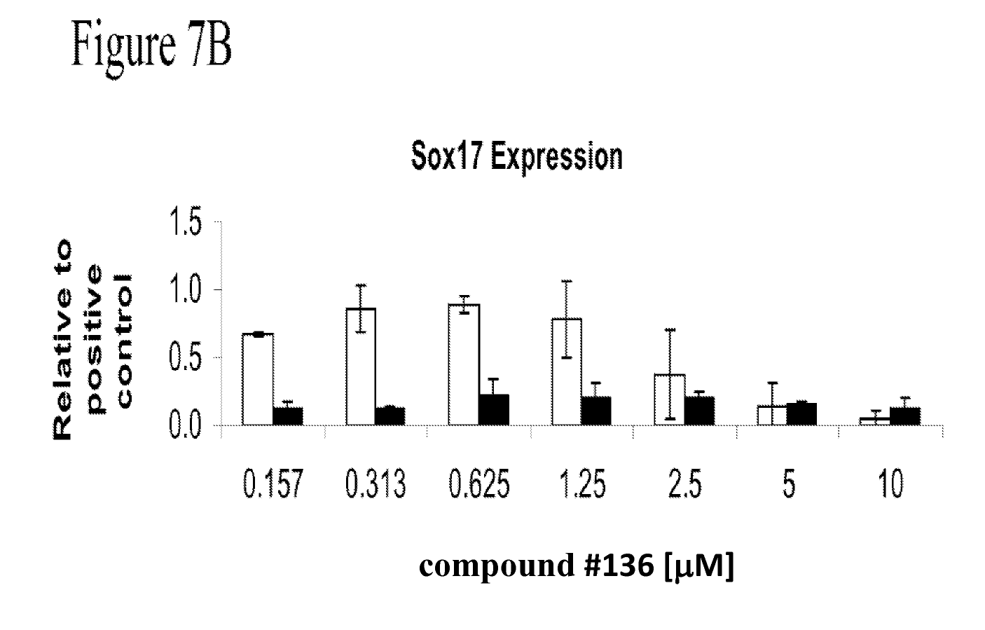

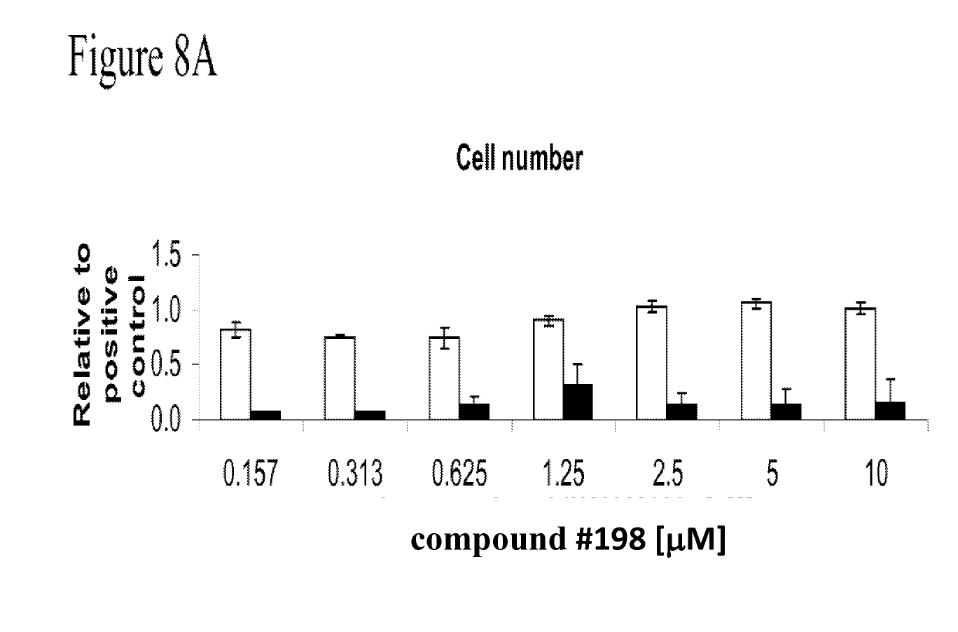

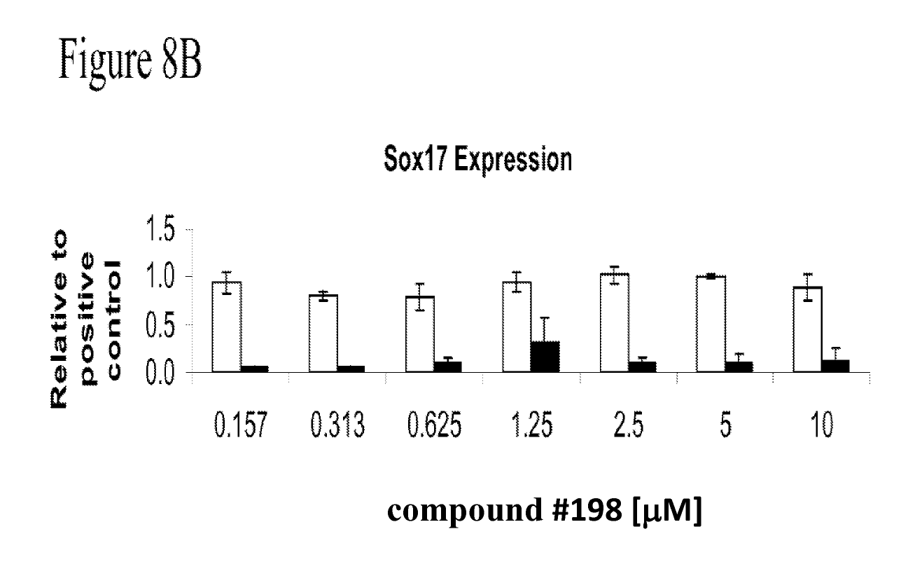

Results are shown for eight GSK-3B enzyme inhibitors where activity was confirmed and potency was determined by titration in this secondary assay. Data presented show compound effects on cell number and Sox17 intensity where respective data points were averaged from a duplicate set and mined for each parameter from identical fields and wells. In this example, Sox17 expression is indicative of definitive endoderm differentiation. Results for cell number and Sox17 intensity, respectively, using the H1 human embryonic stem cell line are shown in Tables VIII and IX. Results for the H9 human embryonic stem cell line are shown in Tables X and XI. Positive control values were normalized to 1.000 for cell number and Sox17 intensity. Negative control values were less-than 0.388 for cell number and less-than 0.065 for Sox17 intensity with both cell lines. A graphic portrayal of these data, comparing both human embryonic stem cell lines and including a dose titration of each compound, is provided in FIGS. 1 to 8. Cell number is presented in panel A; Sox 17 intensity is shown in panel B. These data confirm that each compound can promote hES cell proliferation and definitive endoderm differentiation and identify an optimal range of activity.

### Example 8

### Effects of GSK-3β Enzyme Inhibitors on the Expression of Additional Markers Associated with Definitive Endoderm

It was important to demonstrate that lead compounds could also induce other markers indicative of definitive endoderm differentiation, in addition to the transcription factor Sox17. A select subset of hits was tested for their ability to promote expression of CXCR4, a surface receptor protein, and HNF-3 beta, a transcription factor also associated with definitive endoderm differentiation.

Preparation of Cells for Assay:

Cell clusters from the H1 human embryonis stem cell line used in the assay were evenly resuspended in culture medium and plated onto MATRIGEL™-coated (1:30 dilution) 6-well plates (Corning) in volumes of 2 ml/well. MEF conditioned medium supplemented with 8 ng/ml bFGF was used for initial plating and expansion. Daily feeding was conducted by aspirating spent culture medium from each well and replacing with an equal volume of fresh medium. Cultures were allowed to expand one to three days after plating prior to initiating assay. Plates were maintained at 37° C., 5% CO₂ for the duration of assay.

Preparation of Compounds and Assay Medium:

A subset of seven hits resulting from primary screening was used for follow-up study and subsequent secondary assays. Neat compounds were solubilized as 10 mM stocks in DMSO and stored dessicated at 20° C. until use Immediately prior to assay, compound stocks were diluted to a final concentration ranging between 1 μM and 5 μM in DMEM:F12 base medium (Invitrogen) supplemented with 0.5% FCS (HyClone) and 100 ng/ml Activin A (R&D Biosystems).

Assay:

The assay was initiated by aspirating culture medium from cell monolayers in each well followed by three washes in PBS to remove residual growth factors and serum. Test volumes of 2 ml per well were added back containing medium with 0.5% FCS and different concentrations of inhibitor compounds with 100 ng/ml Activin A, without Wnt3a. Positive control wells contained the same base medium and 0.5% FCS with 100 ng/ml Activin A and 20 ng/ml Wnt3a (R&D Biosystems) in the absence of test compound. Negative control wells contained base medium with 0.5% FCS, in the absence of Activin A, Wnt3a, or test compound. Assay wells were aspirated and fed again with identical concentrations of test compound or control solutions on day 2 of assay. On days 3 and 4, all assay wells were aspirated and fed with DMEM:F12 supplemented with 2% FCS and 100 ng/ml Activin A in the absence of both test compound or Wnt3a. Parallel negative control wells were maintained on days 3 and 4 in DMEM:F12 base medium with 2% FCS.

Assay Evaluation:

At the end of culture, cell monolayers were washed with PBS and harvested from culture plates by incubating 5 minutes with TrypLE™ Express solution (Invitrogen, CA). Cells were resuspended in MEF conditioned medium and split into two equal samples. One set of samples was further stained with various fluorescent labeled antibodies and subjected to flow cytometric (FACS) analysis. A second parallel set of samples was subjected to quantitative PCR.

Cells for FACS analysis were washed into PBS and blocked for 15 minutes at 4° C. in 0.125% human gamma-globulin (Sigma cat#G-4386) diluted in PBS and BD FACS staining buffer. Aliquots of cells (approximately 10⁵ cells each) were stained for 30 minutes at 4° C. with antibodies directly conjugated to a fluorescent tag and having specificity for CD9 PE (BD#555372), CD99 PE (Catalog#MHCD9904), or CXCR-4 APC (R&D Systems cat#FAB173A). After a series of washes in BD FACS staining buffer, cells were stained with 7-AAD (BD#559925) to assess viability and analyzed on a BD FACS Array instrument (BD Biosciences), collecting at least 10,000 events. Mouse IgG₁k isotype control antibodies for both PE and APC were used to gate percent positive cells.

Cells for quantitative PCR were processed for RNA extraction, purification, and cDNA synthesis. RNA samples were purified by binding to a silica-gel membrane (Rneasy Mini Kit, Qiagen, CA) in the presence of an ethanol-containing, high-salt buffer followed by washing to remove contaminants. The RNA was further purified using a TURBO DNA-free kit (Ambion, Inc.), and high-quality RNA was eluted in water. Yield and purity were assessed by A260 and A280 readings on a spectrophotometer. cDNA copies were made from purified RNA using an Applied Biosystems, Inc. (ABI, CA) high capacity cDNA archive kit.

Unless otherwise stated, all reagents for real-time PCR amplification and quantitation were purchased from ABI. Real-time PCR reactions were performed using the ABI PRISM 7900 Sequence Detection System. TAQMAN UNIVERSAL PCR MASTER MIX (ABI, CA) was used with 20 ng of reverse transcribed RNA in a total reaction volume of 20 μA Each cDNA sample was run in duplicate to correct for pipetting errors. Primers and FAM-labeled TAQMAN probes were used at concentrations of 200 nM. The level of expression for each target gene was normalized using a human glyceraldehyde-3-phosphate dehydrogenase (GAPDH) endogenous control previously developed by ABI. Primer and probe sets are listed as follows: CXCR4 (Hs00237052), GAPDH (4310884E), HNF3b (Hs00232764), SOX17 (probe part #450025, forward and reverse part #4304971).

After an initial incubation at 50° C. for 2 min followed by 95° C. for 10 min, samples were cycled 40 times in two stages, a denaturation step at 95° C. for 15 sec followed by an annealing/extension step at 60° C. for 1 min. Data analysis was carried out using GENEAMP 7000 Sequence Detection System software. For each primer/probe set, a Ct value was determined as the cycle number at which the fluorescence intensity reached a specific value in the middle of the exponential region of amplification. Relative gene expression levels were calculated using the comparative Ct method. Briefly, for each cDNA sample, the endogenous control Ct value was subtracted from the gene of interest Ct to give the delta Ct value (ΔCt). The normalized amount of target was calculated as 2-ΔCt, assuming amplification to be 100% efficiency. Final data were expressed relative to a calibrator sample.

### Results

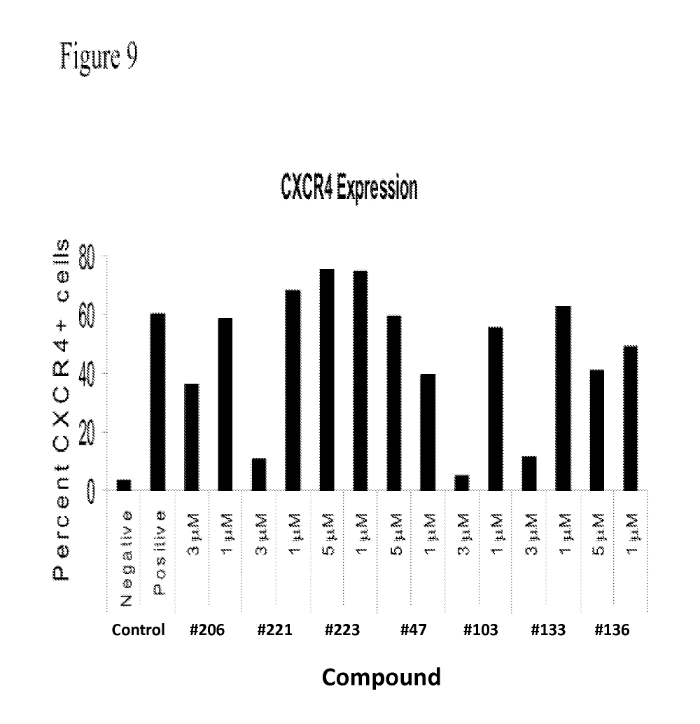

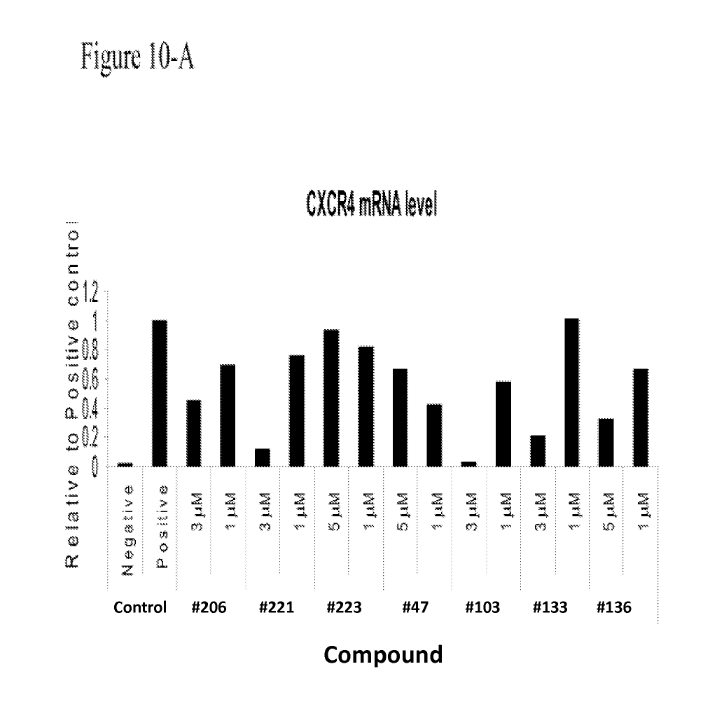

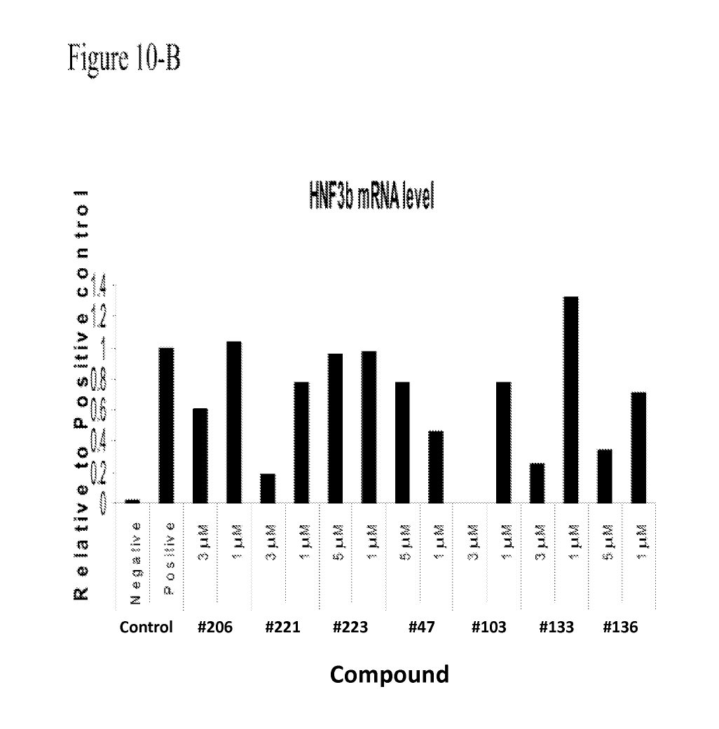

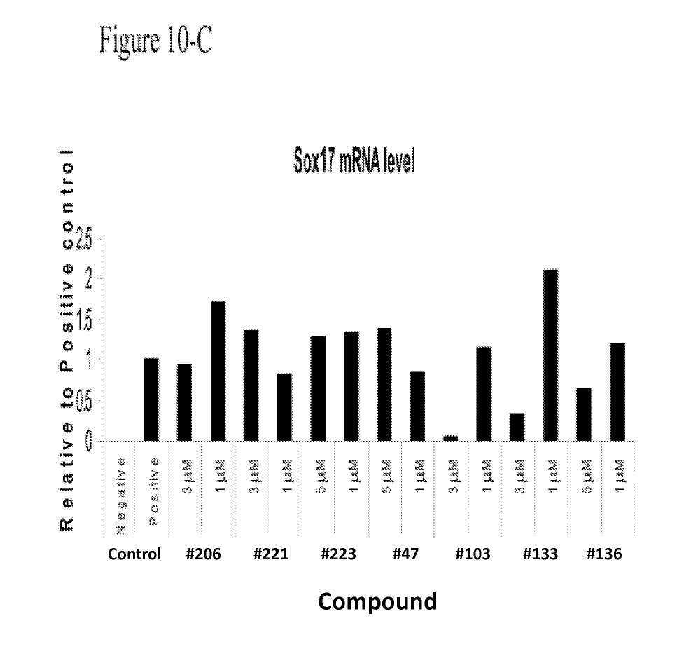

FIG. 9 displays the FACS analysis of percent positive cells expressing CXCR4 surface receptor after treatment with various GSK3 inhibitors. Two concentrations of each compound, ranging between 1 μM and 5 μM, are shown relative to an untreated population of cells (negative control) or cells treated with Activin A and Wnt3 (positive control). FIG. 10 panels a, b, and c show real-time PCR data for CXCR4, Sox17, and HNF3beta, which are also considered to be markers of definitive endoderm. Both FACS and real-time PCR analysis demonstrate a significant increase in each of these markers observed in differentiated cells relative to untreated control cells. Expression levels of these definitive endoderm markers were equivalent in some cases to the positive control, demonstrating that a GSK3 inhibitor can substitute for Wnt3a at this stage of differentiation.

### Example 9

### Effects of GSK-30 Enzyme Inhibitors on the Formation of Pancreatic Endoderm

It was important to demonstrate that treatment with GSK30 inhibitors during induction of definitive endoderm did not prevent the subsequent differentiation of other cell types, such as pancreatic endoderm, for example. A select subset of hits was tested for their ability to promote expression of PDX1 and HNF6, key transcription factors associated with pancreatic endoderm.

Maintenance of human embryonic stem cells (H1 and H9 lines) was conducted as described in Example 1. Colonies of cells were maintained in an undifferentiated, pluripotent state with passage on average every four days. Passage was performed by exposing cell cultures to a solution of collagenase (1 mg/ml; Sigma-Aldrich) for 10 to 30 minutes at 37° C., followed by gentle scraping with a pipette tip to recover cell clusters. Clusters were allowed to sediment by gravity, followed by washing to remove residual collagenase.

Cell clusters were split at a 1:3 ratio for routine maintenance culture or a 1:1 ratio for subsequent assay. The human embryonic stem cell lines used were maintained at less than passage 50 and routinely evaluated for normal karyotypic phenotype and absence of mycoplasma contamination.

Cell Preparation of Assay:

Cell clusters of the H1 human embryonis stem cell line used in the assay were evenly resuspended in culture medium and plated onto MATRIGEL™-coated (1:30 dilution) 24-well plates (black well; Arctic White) in volumes of 1 ml/well. MEF conditioned medium supplemented with 8 ng/ml bFGF was used for initial plating and expansion. In a second experiment, clusters of hES cells from the H9 line were plated in 96-well plates on mouse embryonic feeder (MEF) layers, previously inactivated by treating with mitomycin C (Sigma Chemical Co). Culture medium for hES cells on MEF monolayers consisted of DMEM:F12 with 20% Knockout Serum Replacer (Invitrogen) supplemented with minimal essential amino acids (Invitrogen), L-glutamine, and 2-mercaptoethanol. Daily feeding was conducted by aspirating spent culture medium from each well and replacing with an equal volume of fresh medium. Cultures were allowed to expand one to three days after plating prior to initiating assay. Plates were maintained at 37° C., 5% CO₂ for the duration of assay.

Preparation of Compounds and Assay Medium:

A subset of eight hits resulting from primary screening was used for follow-up study and subsequent secondary assays. Neat compounds were solubilized as 10 mM stocks in DMSO and stored dessicated at 20° C. until use. Immediately prior to assay, compound stocks were diluted to a final concentration ranging between 1 μM and 5 μM in base medium with additives.

Assay:

In this assay, GSK3 inhibitors were included only on days 1 and 2 of the definitive endoderm differentiation step, substituting for Wnt3a. Embryonic stem cell cultures on MATRIGEL™ were initiated as described in Examples 7 and 8 above by aspirating culture medium from cell monolayers in each well followed by three washes in PBS to remove residual growth factors and serum. For differentiation to definitive endoderm, test volumes (0.5 ml per well for 24-well plates, 100 μl per well for 96-well plates) were added containing DMEM:F12 medium with) 0.5% FCS and different concentrations of inhibitor compounds with 100 ng/ml Activin A, without Wnt3a. Positive control wells contained the same base medium with 0.5% FCS and with 100 ng/ml Activin A and 20 ng/ml Wnt3a (R&D Biosystems) in the absence of test compound. Negative control wells contained the same base medium with 0.5% FCS, in the absence of Activin A, Wnt3a, or test compound. Assay wells were aspirated and fed again with identical concentrations of test compound or control solutions on day 2 of assay. On days 3 and 4, all assay wells were aspirated and fed with DMEM:F12 supplemented with 2% FCS and 100 ng/ml Activin A in the absence of both test compound or Wnt3a. Parallel negative control wells were maintained on days 3 and 4 in DMEM:F 12 base medium with 2% FCS. For differentiation to pancreatic endoderm, cells were treated for three days, feeding daily with DMEM:F12 base medium containing 2% FCS with 0.25 μM KAAD cyclopamine (EMD Biosciences) and 20 ng/ml FGF7 (R&D Biosystems). Cells were then treated for an additional four days, feeding daily with DMEM:F12 containing 1% B27 (Invitrogen), 0.25 μM KAAD cyclopamine, 2 μM Retinoic Acid (RA; Sigma-Aldrich) and 20 ng/ml FGF7. Parallel negative control wells were maintained throughout in DMEM:F12 base medium with 2% FCS (stage 2) or 1% B27 (stage 3) and without any other additives.

Parallel cultures of H9 human embryonic cells were grown on MEF feeder layers, and differentiated to pancreatic endoderm. Definitive endoderm differentiation was achieved by culturing the cells in medium consisting of RPMI-1640 (Invitrogen) containing no serum on day 1 and 0.2% FCS on days 2 and 3 along with different concentrations of inhibitor compounds and 100 ng/ml Activin A. Positive control wells contained the same base medium (with or without serum) with 100 ng/ml Activin A and 20 ng/ml Wnt3a (R&D Biosystems) in the absence of test compound. Negative control wells contained the same base medium with or without serum, in the absence of Activin A, Wnt3a, or test compound. Assay wells were aspirated and fed again with identical concentrations of test compound or control solutions on day 2 of assay. On day 3, all assay wells were aspirated and fed with RPMI-1640 supplemented with 2% FCS and 100 ng/ml Activin A in the absence of both test compound and Wnt3a. Parallel negative control wells were maintained on day 3 in RPMI-1640 base medium with 2% FCS. Cells were differentiated into pancreatic endoderm by treating the cells for four days, feeding daily with RPMI-1640 base medium containing 2% FCS with 0.25 mM KAAD cyclopamine (EMD Biosciences) and 50 ng/ml FGF10 (R&D Biosystems). Subsequently, cells were treated for three days duration, feeding daily with RPMI-1640 containing 1% B27 (Invitrogen), 0.25 mM KAAD cyclopamine, 2 mM Retinoic Acid (RA; Sigma-Aldrich) and 50 ng/ml FGF10. Parallel negative control wells were maintained throughout in RPMI-1640 base medium with 2% FCS (stage 2) or 1% B27 (stage 3) and without any other additives.

Assay Evaluation:

At the end the differentiation, cells were examined as described in Example 8 for gene expression by real-time PCR. For high content fluorescence staining, cells in 96-well plates were washed twice with PBS then fixed with 4% paraformaldehyde at room temperature for 20 minutes, washed three times more with PBS, and then permeabilized with 0.5% Triton X-100 for 20 minutes at room temperature. After fixing and permeabilizing, cells were washed again three times with PBS and blocked with 4% chicken serum (Invitrogen) in PBS for 30 minutes at room temperature. Primary antibody (goat anti-human Pdx1; Santa Cruz) was diluted 1:100 in 4% chicken serum and added to cells for two hours at room temperature. Alexa Fluor 488 conjugated secondary antibody (chicken anti-goat IgG; Molecular Probes) was diluted 1:200 in PBS and added to each well after washing the cells three times with PBS. To counterstain nuclei, 2 μg/ml Hoechst 33342 (Invitrogen) was added for ten minutes at room temperature. Cells were washed once with PBS and left in 100 μl/well PBS for imaging.

Cells were imaged using an IN Cell Analyzer 1000 (GE Healthcare) utilizing the 51008bs dichroic for cells stained with Hoechst 33342 and Alexa Fluor 488. Exposure times were optimized using positive control wells and wells stained with secondary antibody alone. Images from 15 fields per well were acquired to compensate for any cell loss during the treatment and staining procedures. Measurements for total cell number and total Pdx1 intensity were obtained for each well using IN Cell Developer Toolbox 1.7 (GE Healthcare) software. Segmentation for the nuclei was determined based on grey-scale levels (baseline range 100-300) and nuclear size. Averages and standard deviations were calculated for each replicate data set. Total Pdx1 protein expression was reported as total intensity or integrated intensity, defined as total fluorescence of the cell times area of the cell. Background was eliminated based on acceptance criteria of grey-scale ranges between 300 to 3000. Total intensity data were normalized by dividing the total intensities for each well by the average total intensity for the Wnt3a/Activin A positive control. Normalized data were calculated for averages and standard deviations for each replicate set.

Cells for quantitative PCR were lysed in RLT buffer (Qiagen) and then processed for RNA extraction, purification, and cDNA synthesis. RNA samples were purified by binding to a silica-gel membrane (Rneasy Mini Kit, Qiagen, CA) in the presence of an ethanol-containing, high-salt buffer followed by washing to remove contaminants. The RNA was further purified using a TURBO DNA-free kit (Ambion, Inc.), and high-quality RNA was then eluted in water. Yield and purity were assessed by A260 and A280 readings on a spectrophotometer. cDNA copies were made from purified RNA using an Applied Biosystems, Inc. (ABI, CA) high capacity cDNA archive kit.

Unless otherwise stated, all reagents for real-time PCR amplification and quantitation were purchased from ABI. Real-time PCR reactions were performed using the ABI PRISM 7900 Sequence Detection System. TAQMAN UNIVERSAL PCR MASTER MIX was used with 20 ng of reverse transcribed RNA in a total reaction volume of 20 μl. Each cDNA sample was run in duplicate to correct for pipetting errors. Primers and FAM-labeled TAQMAN probes were used at concentrations of 200 nM. The level of expression for each target gene was normalized using a human glyceraldehyde-3-phosphate dehydrogenase (GAPDH) endogenous control previously developed by ABI. Primer and probe sets are listed as follows: PDX1 (Hs00236830_m1), GAPDH (4310884E), and HNF6 (Hs00413554_m1).

After an initial incubation at 50° C. for 2 min followed by 95° C. for 10 min, samples were cycled 40 times in two stages, a denaturation step at 95° C. for 15 sec followed by an annealing/extension step at 60° C. for 1 min. Data analysis was carried out using GENEAMPO7000 Sequence Detection System software. For each primer/probe set, a Ct value was determined as the cycle number at which the fluorescence intensity reached a specific value in the middle of the exponential region of amplification. Relative gene expression levels were calculated using the comparative Ct method. Briefly, for each cDNA sample, the endogenous control Ct value was subtracted from the gene of interest Ct to give the delta Ct value (ΔCt). The normalized amount of target was calculated as 2-ΔCt, assuming amplification to be 100% efficiency. Final data were expressed relative to a calibrator sample.

### Results

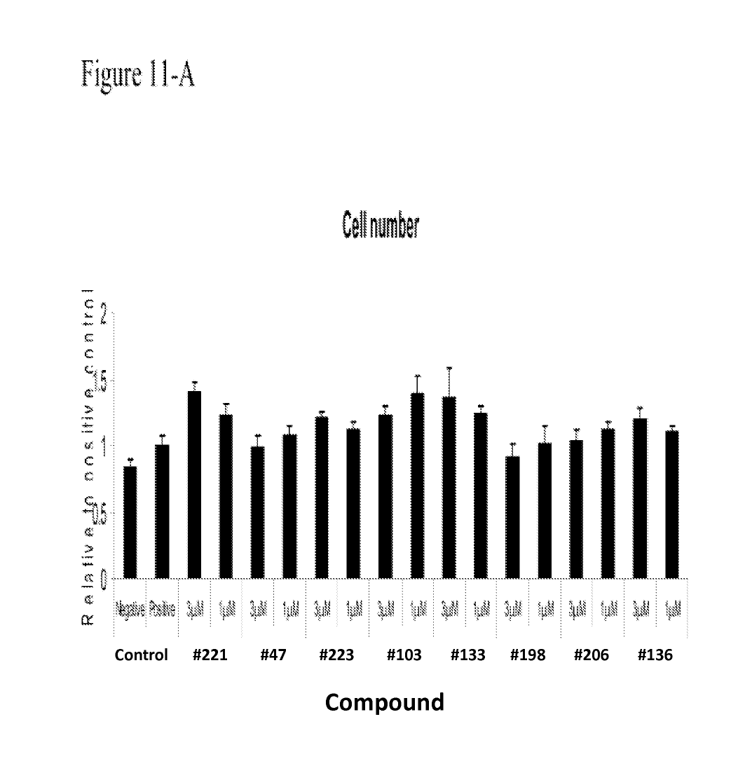

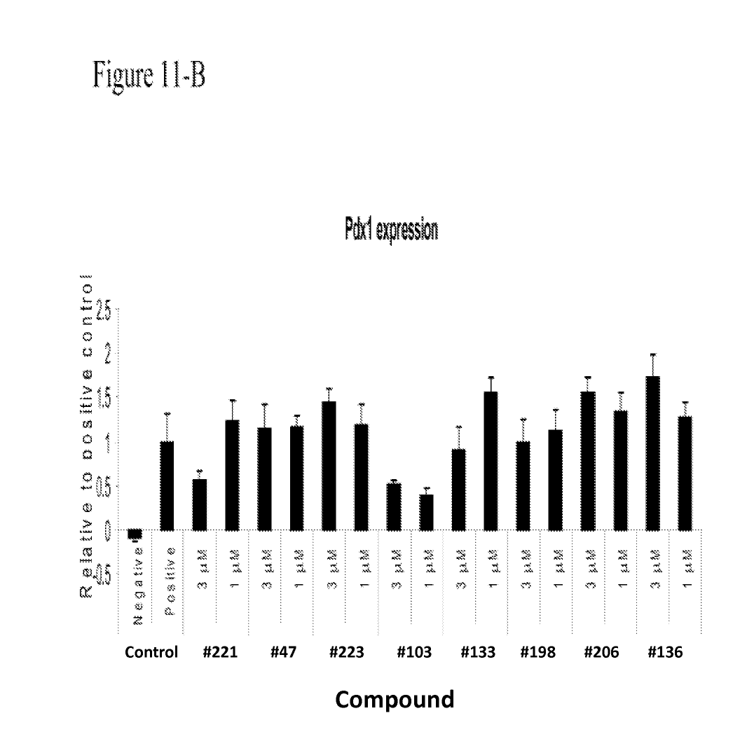

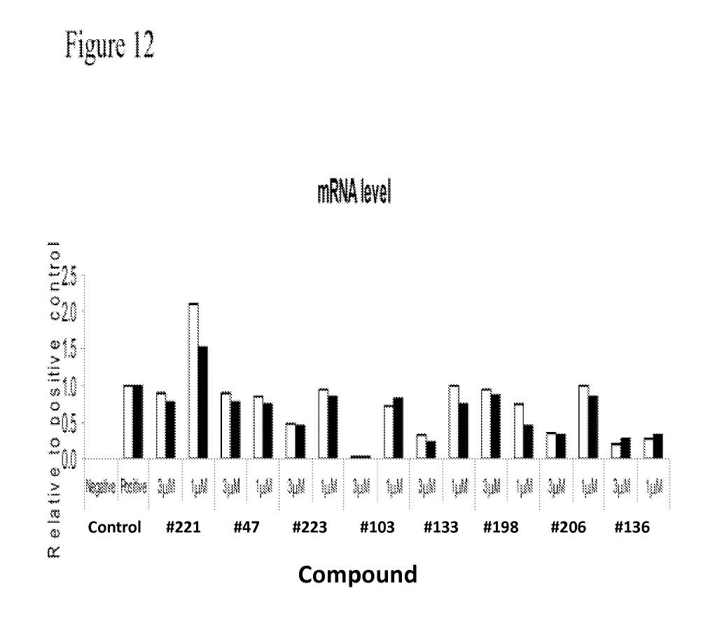

Results are shown for eight GSK-3β enzyme inhibitors. Data presented in FIG. 11 from high content analysis show effects on cell number (panel A) and Pdx1 intensity (panel B) for the H1 hES cell line, where respective data points were averaged from a duplicate sample set and mined for each parameter from identical fields and wells. Data presented in FIG. 12 from real-time PCR show effects of these small molecule inhibitors on induced expression of two transcription factors, Pdx1 and HNF6. In these examples, Pdx1 and HNF6 expression are indicative of pancreatic endoderm differentiation. GSK313 inhibitor compounds in these assays can substitute for Wnt3a during early stages of cell lineage commitment; resulting cells sustain a capacity to form pancreatic endoderm during later sequential stages of differentiation.

### Example 10

### Effects of GSK-30 Enzyme Inhibitors on the Formation of Pancreatic Endocrine Cells

It was important to demonstrate that treatment with GSK3 inhibitors during induction of definitive endoderm did not prevent the subsequent differentiation of other cell types, such as pancreatic endocrine cells, or insulin producing cells, for example. A select subset of hits was tested for their ability to promote expression of pancreatic hormones.

Cell Preparation for Assay:

Pancreatic endoderm cells obtained according to the methods described in Example 9 (cultured on 96-wellplates and 24-well plates) were subsequently subjected to agents that cause the cells to differentiate into pancreatic hormone expressing cells.

Assay for cultures of the H1 human embryonic stem cell line on MATRIGEL™ was initiated as described in Examples 7-9 above by aspirating culture medium from cell monolayers in each well followed by three washes in PBS to remove residual growth factors and serum. For differentiation to definitive endoderm, test volumes (0.5 ml per well for 24-well plates, 100 μl per well for 96-well plates) were added containing medium with 0.5% FCS and different concentrations of inhibitor compounds with 100 ng/ml Activin A, without Wnt3a. Positive control wells contained the same base medium and 0.5% FCS with 100 ng/ml Activin A and 20 ng/ml Wnt3a (R&D Biosystems) in the absence of test compound. Negative control wells contained the same base medium with 0.5% FCS, in the absence of Activin A, Wnt3a, or test compound. Assay wells were aspirated and fed again with identical concentrations of test compound or control solutions on day 2 of assay. On days 3, 4, and 5, all assay wells were aspirated and fed with DMEM:F12 supplemented with 2% FCS and 100 ng/ml Activin A in the absence of both test compound or Wnt3a. Parallel negative control wells were maintained on days 3, 4, and 5 in DMEM:F12 base medium with 2% FCS. For differentiation to pancreatic endoderm, cells were treated for three days, feeding daily with DMEM:F12 base medium containing 2% FCS with 0.25 μM KAAD cyclopamine (EMD Biosciences) and 20 ng/ml FGF7 (R&D Biosystems). Cells were subsequently treated for four days, feeding daily with DMEM:F12 containing 1% B27 (Invitrogen), 0.25 μM KAAD cyclopamine, 2 μM Retinoic Acid (RA; Sigma-Aldrich) and 20 ng/ml FGF7. Parallel negative control wells during stages 2 and 3 were maintained throughout in DMEM:F12 base medium with 2% FCS or 1% B27 and without any other additives. After formation of pancreatic endoderm, cells were treated further for six days duration, feeding daily with DMEM:F12 base medium containing 1% B27 with 1 μM DAPT (gamma secretase inhibitor: EMD Biosciences) and 50 ng/ml Exendin 4 (Sigma-Aldrich). Cells were then treated for another three days duration, feeding daily with DMEM:F12 base medium containing 1% B27, 50 ng/ml Exendin 4, 50 ng/ml IGF (R&D Biosystems) and 50 ng/ml HGF (R&D Biosystems). Parallel negative control wells were maintained throughout in DMEM:F12 base medium with 1% B27 and without any other additives.

Assay Evaluation:

At the end of culture, cells were treated as in Examples 7 and 8 above for evaluation by high content analysis or real-time PCR.

For high content fluorescence staining, cells in 96-well plates were washed twice with PBS then fixed with 4% paraformaldehyde at room temperature for 20 minutes, washed three times more with PBS, and then permeabilized with 0.5% Triton X-100 for 20 minutes at room temperature. After fixing and permeabilizing, cells were washed again three times with PBS and blocked with 4% chicken serum (Invitrogen) in PBS for 30 minutes at room temperature. Primary antibody (guinea pig anti-swine insulin, cross-reactive with human insulin; DakoCytomation) was diluted 1:500 in 4% goat serum and added to cells for one hour at room temperature. Cells were washed three times with PBS and then stained with Alexa Fluor 488 conjugated secondary antibody (goat anti-guinea pig IgG; Molecular Probes) diluted 1:100 in 4% goat serum. To counterstain nuclei, 2 μg/ml Hoechst 33342 (Invitrogen) was added for ten minutes at room temperature. Cells were washed once with PBS and left in 100 μl/well PBS for imaging.

Cells were imaged using an IN Cell Analyzer 1000 (GE Healthcare) utilizing the 51008bs dichroic for cells stained with Hoechst 33342 and Alexa Fluor 488. Exposure times were optimized using positive control wells and wells stained with secondary antibody alone. Images from 15 fields per well were acquired to compensate for any cell loss during the treatment and staining procedures. Measurements for total cell number and total insulin intensity were obtained for each well using IN Cell Developer Toolbox 1.7 (GE Healthcare) software. Segmentation for the nuclei was determined based on grey-scale levels (baseline range 100-300) and nuclear size. Averages and standard deviations were calculated for each replicate data set. Total insulin protein expression was reported as total intensity or integrated intensity, defined as total fluorescence of the cell times area of the cell. Background was eliminated based on acceptance criteria of grey-scale ranges between 300 to 3000. Total intensity data were normalized by dividing the total intensities for each well by the average total intensity for the Wnt3a/Activin A positive control. Normalized data were calculated for averages and standard deviations for each triplicate set.

Cells for quantitative PCR were lysed in RLT buffer (Qiagen) and then processed for RNA extraction, purification, and cDNA synthesis. RNA samples were purified by binding to a silica-gel membrane (Rneasy Mini Kit, Qiagen, CA) in the presence of an ethanol-containing, high-salt buffer followed by washing to remove contaminants. The RNA was further purified using a TURBO DNA-free kit (Ambion, INC), and high-quality RNA was eluted in water. Yield and purity were assessed by A260 and A280 readings on a spectrophotometer. cDNA copies were made from purified RNA using an Applied Biosystems, Inc. (ABI, CA) high capacity cDNA archive kit.

Unless otherwise stated, all reagents for real-time PCR amplification and quantitation were purchased from ABI. Real-time PCR reactions were performed using the ABI PRISM® 7900 Sequence Detection System. TAQMAN® UNIVERSAL PCR MASTER MIX® (ABI, CA) was used with 20 ng of reverse transcribed RNA in a total reaction volume of 20 μA Each cDNA sample was run in duplicate to correct for pipetting errors. Primers and FAM-labeled TAQMAN®probes were used at concentrations of 200 nM. The level of expression for each target gene was normalized using a human glyceraldehyde-3-phosphate dehydrogenase (GAPDH) endogenous control previously developed by ABI. Primer and probe sets are listed as follows: PDX1 (Hs00236830_m1), Insulin (Hs00355773), and GAPDH (4310884E).

After an initial incubation at 50° C. for 2 min followed by 95° C. for 10 min, samples were cycled 40 times in two stages, a denaturation step at 95° C. for 15 sec followed by an annealing/extension step at 60° C. for 1 min. Data analysis was carried out using GENEAMP®7000 Sequence Detection System software. For each primer/probe set, a Cₜ value was determined as the cycle number at which the fluorescence intensity reached a specific value in the middle of the exponential region of amplification. Relative gene expression levels were calculated using the comparative Cₜ method. Briefly, for each cDNA sample, the endogenous control Cₜ value was subtracted from the gene of interest Cₜ to give the delta Cₜ value (ΔCₜ). The normalized amount of target was calculated as 2^{-ΔCt}, assuming amplification to be 100% efficiency. Final data were expressed relative to a calibrator sample.

### Results

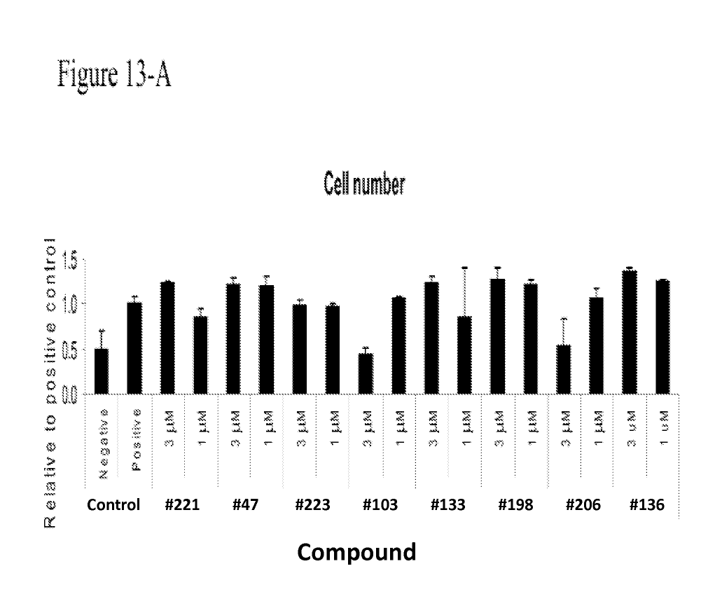

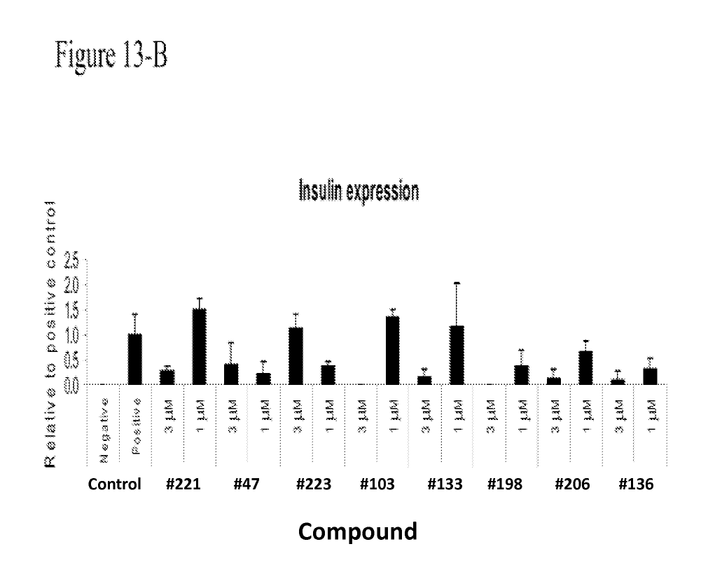

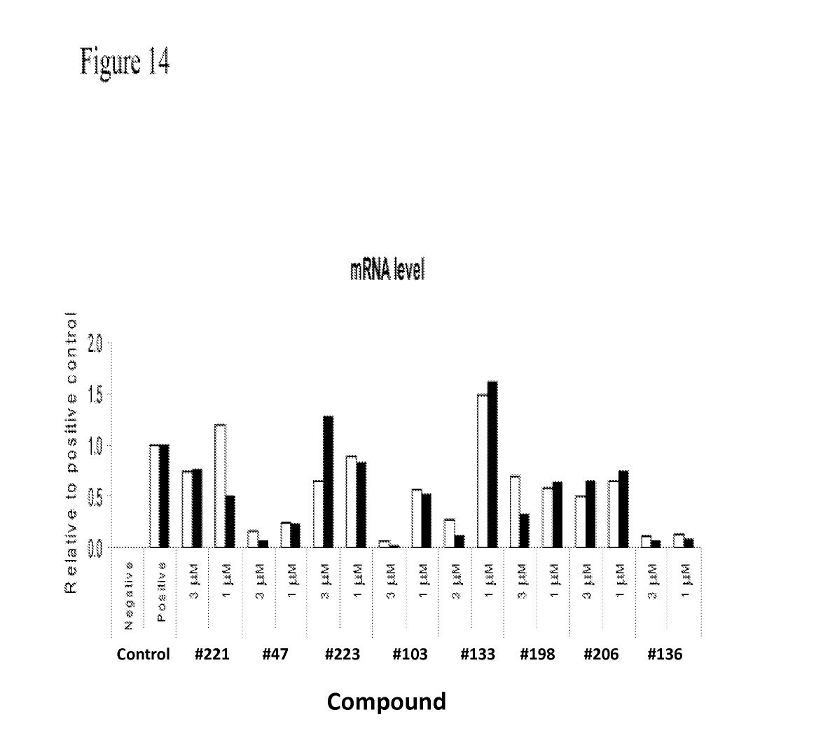

Results are shown for eight GSK-3B enzyme inhibitors. Data presented in FIG. 13 from high content analysis show compound effects on cell number (panel A) and insulin intensity (panel B) for the H1 hES cell line where respective data points were averaged from a triplicate set and mined for each parameter from identical fields and wells. Data presented in FIG. 14 from real-time PCR show compound effects for Pdx1 and insulin. In these examples, Pdx1 and insulin expression are indicative of pancreatic endoderm differentiation and generation of hormonal positive cells. Selective GSK3β inhibitor compounds in these assays can substitute for Wnt3a during early stages of cell lineage commitment and can induce and sustain pancreatic beta cell formation during later sequential stages of differentiation, as evident from both insulin immunostaining and real-time PCR.

### Example 11

### Additive Effects of GSK-30 Enzyme Inhibitors on the Formation of Pancreatic Endocrine Cells

It was important to demonstrate that treatment with GSK30 inhibitors could improve pancreatic beta cell differentiation if added during multiple phases of cell fate commitment. A select subset of hits was tested by sequential timed addition to enhance insulin expression associated with pancreatic hormonal positive cells.

Preparation of Cells for Assay:

Cell preparation for assay: Pancreatic endoderm cells obtained according to the methods described in Example 9 and 10 (cultured on 96-wellplates) were subsequently subjected to agents that cause the cells to differentiate into pancreatic hormone expressing cells.

Assay for cultures of the H1 human embryonic stem cell line on MATRIGEL™ was initiated as described in Examples 7-9 above by aspirating culture medium from cell monolayers in each well followed by three washes in PBS to remove residual growth factors and serum. For differentiation to definitive endoderm, test volumes (100 μl per well for 96-well plates) were added containing medium with 0.5% FCS and different concentrations of inhibitor compounds with 100 ng/ml Activin A, without Wnt3a. Positive control wells contained the same base medium and 0.5% FCS with 100 ng/ml Activin A and 20 ng/ml Wnt3a (R&D Biosystems) in the absence of test compound. Negative control wells contained the same base medium with 0.5% FCS, in the absence of Activin A, Wnt3a, or test compound. Assay wells were aspirated and fed again with identical concentrations of test compound or control solutions on day 2 of assay. On days 3, 4, and 5, all assay wells were aspirated and fed with DMEM:F12 supplemented with 2% FCS and 100 ng/ml Activin A in the absence of both test compound or Wnt3a. Parallel negative control wells were maintained on days 3, 4, and 5 in DMEM:F12 base medium with 2% FCS. For differentiation to pancreatic endoderm, cells were treated for three days, feeding daily with DMEM:F12 base medium containing 2% FCS with 0.25 μM KAAD cyclopamine (EMD Biosciences) and 20 ng/ml FGF7 (R&D Biosystems). Cells were subsequently treated for four days, feeding daily with DMEM:F12 containing 1% B27 (Invitrogen), 0.25 μM KAAD cyclopamine, 2 μM Retinoic Acid (RA; Sigma-Aldrich) and 20 ng/ml FGF7. Parallel negative control wells were maintained throughout in DMEM:F12 base medium with 2% FCS or 1% B27 and without any other additives. After formation of pancreatic endoderm, cells were treated further for six days duration, feeding alternating days with DMEM:F12 base medium containing 1% B27 with 1 μM DAPT (gamma secretase inhibitor: EMD Biosciences) and 50 ng/ml Exendin 4 (Sigma-Aldrich) and 1 μM TGFbeta R1 inhibitor II (ALKS inhibitor; EMD Biosciences). During this six day period, GSK313 inhibitors were added back to respective wells, using the same concentration as previous treatment at the initiation of differentiation. Cells were then treated for another three days duration, feeding alternating days with DMEM:F12 base medium containing 1% B27, 50 ng/ml Exendin 4, 50 ng/ml IGF (R&D Biosystems) and 50 ng/ml HGF (R&D Biosystems), and 1 μM TGFbeta R1 inhibitor II (ALKS inhibitor; EMD Biosciences). During this three day period, GSK313 inhibitors were added back to respective wells, using the same concentration as previous treatment at the initiation of differentiation. Parallel sets of positive control wells were treated in the presence or absence of 20 ng/ml Wnt3a. Parallel negative control wells were maintained throughout in DMEM:F12 base medium with 1% B27 and without any other additives.

Assay Evaluation:

At the end of culture, cells were treated as in Examples 10 above for evaluation by high content analysis.

For high content fluorescence staining, cells in 96-well plates were washed twice with PBS then fixed with 4% paraformaldehyde at room temperature for 20 minutes, washed three times more with PBS, and then permeabilized with 0.5% Triton X-100 for 20 minutes at room temperature. After fixing and permeabilizing, cells were washed again three times with PBS and blocked with 4% chicken serum (Invitrogen) in PBS for 30 minutes at room temperature. Primary antibody (guinea pig anti-swine insulin, cross-reactive with human insulin; DakoCytomation) was diluted 1:500 in 4% goat serum and added to cells for one hour at room temperature. Cells were washed three times with PBS and then stained with Alexa Fluor 488 conjugated secondary antibody (goat anti-guinea pig IgG; Molecular Probes) diluted 1:100 in 4% goat serum. To counterstain nuclei, 2 μg/ml Hoechst 33342 (Invitrogen) was added for ten minutes at room temperature. Cells were washed once with PBS and left in 100 μl/well PBS for imaging.

Cells were imaged using an IN Cell Analyzer 1000 (GE Healthcare) utilizing the 51008bs dichroic for cells stained with Hoechst 33342 and Alexa Fluor 488. Exposure times were optimized using positive control wells and wells stained with secondary antibody alone. Images from 15 fields per well were acquired to compensate for any cell loss during the treatment and staining procedures. Measurements for total cell number and total insulin intensity were obtained for each well using IN Cell Developer Toolbox 1.7 (GE Healthcare) software. Segmentation for the nuclei was determined based on grey-scale levels (baseline range 100-300) and nuclear size. Averages and standard deviations were calculated for each replicate data set. Total insulin protein expression was reported as total intensity or integrated intensity, defined as total fluorescence of the cell times area of the cell. Background was eliminated based on acceptance criteria of grey-scale ranges between 300 to 3000. Total intensity data were normalized by dividing the total intensities for each well by the average total intensity for the Wnt3a/Activin A positive control. Normalized data were calculated for averages and standard deviations for each triplicate set.

### Results

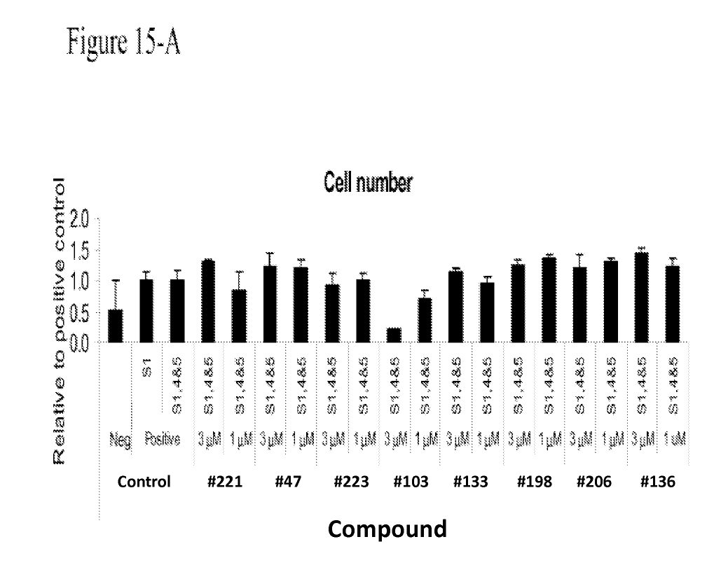

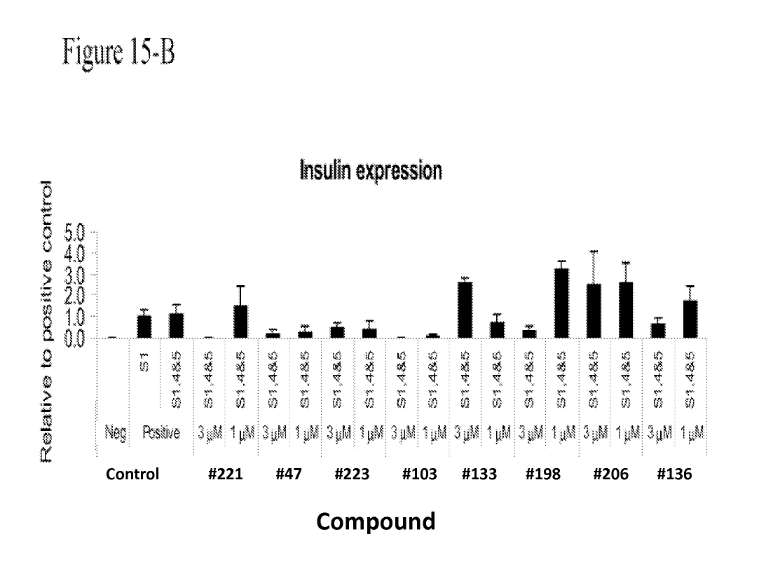

Results are shown for eight GSK-3B enzyme inhibitors. Data presented in FIG. 15 from high content analysis show compound effects on cell number (panel A) and insulin intensity (panel B) for the H1 hES cell line, where respective data points were averaged from a triplicate set and mined for each parameter from identical fields and wells. In this example, insulin expression is indicative of differentiation to hormonal positive pancreatic cells. Selective GSK313 inhibitor compounds in these assays can substitute for Wnt3a during early stages of cell lineage commitment and, when added at later stages of differentiation, appear to promote enhanced insulin expression relative to a positive control sample.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

TABLE IA
LIST OF PRIMARY ANTIBODIES USED FOR
FACS AND IMMUNOSTAINININGANALYSIS.

| Antibody | Supplier | Isotype | Clone |
|---|---|---|---|
| SSEA-1 | Chemicon (CA) | Mouse IgM | MC-480 |
| SSEA-3 | Chemicon (CA) | Mouse IgG3 | MC-631 |
| SSEA-4 | Chemicon (CA) | Rat IgM | MC-813-70 |
| TRA 1-60 | Chemicon (CA) | Mouse IgM | TRA 1-60 |
| TRA 1-81 | Chemicon (CA) | Mouse IgM | TRA 1-81 |
| TRA 1-85 | Chemicon (CA) | Mouse IgG1 | TRA 1-85 |
| AP | R&D Systems | Mouse IgG1 | B4-78 |
| HNF3β | R&D Systems | Goat IgG | |
| PDX1 | Santa Cruz | Goat IgG | A-17 |
| | Biotechnology, | | |
| | INC | | |
| GATA4 | R&D Systems | Goat IgG | |
| Sox 17 | R&D Systems | Goat IgG | |
| CD 9 | BD | Mouse IgG1 | M-L13 |

TABLE IB
LIST OF SECONDARY CONJUGATED ANTIBODIES
USED FOR FACS AND IMMUNOSTAINININGANALYSIS.

| Secondary Conjugated | | |
|---|---|---|
| Antibody | Supplier | Dilution |
| Goat Anti-Mouse IgG APC | Jackson ImmunoResearch (PA) | 1:200 |
| conjugated | | |
| Goat Anti-Mouse IgG PE | Jackson ImmunoResearch (PA) | 1:200 |
| conjugated | | |
| Donkey anti-rabbit PE or— | Jackson ImmunoResearch (PA) | 1:200 |
| APC conjugated | | |
| Donkey anti-goat PE or— | Jackson ImmunoResearch (PA) | 1:200 |
| APC conjugated | | |
| Goat anti-mouse IgM PE | SouthernBiotech (AL) | 1:200 |
| Goat anti-Rat IgM PE | SouthernBiotech (AL) | 1:200 |
| Goat anti-mouse IgG3 PE | SouthernBiotech (AL) | 1:200 |

TABLE II
EFFECTS OF INHIBITORS OF GSK-3B ENZYME
ACTIVITY ON THE VIABILITY OF CELLS
EXPRESSING PLURIPOTENCY MARKERS.

| | Raw data | | | | | |
|---|---|---|---|---|---|---|
| Compound # | (duplicate) | | Average | S.D. | % CV | % Control |
| 2 | 0.785 | 0.790 | 0.788 | 0.00382 | 0.48 | 94.0 |
| 12 | 0.148 | 0.152 | 0.150 | 0.00247 | 1.65 | 4.8 |
| 20 | 0.427 | 0.462 | 0.444 | 0.02496 | 5.62 | 46.0 |
| 28 | 0.643 | 0.638 | 0.641 | 0.00368 | 0.57 | 73.5 |
| 1 | 0.762 | 0.762 | 0.762 | 0.00007 | 0.01 | 90.4 |
| 46 | 0.850 | 0.824 | 0.837 | 0.01824 | 2.18 | 101.0 |
| 52 | 0.911 | 0.884 | 0.898 | 0.01881 | 2.10 | 109.5 |
| 61 | 0.723 | 0.743 | 0.733 | 0.01421 | 1.94 | 86.4 |
| 3 | 0.161 | 0.169 | 0.165 | 0.00559 | 3.39 | 6.9 |
| 13 | 0.767 | 0.789 | 0.778 | 0.01556 | 2.00 | 92.6 |
| 20 | 0.512 | 0.555 | 0.533 | 0.03048 | 5.72 | 58.4 |
| 28 | 0.282 | 0.293 | 0.288 | 0.00792 | 2.75 | 24.1 |
| 37 | 0.764 | 0.723 | 0.743 | 0.02892 | 3.89 | 87.9 |
| 47 | 0.853 | 0.858 | 0.855 | 0.00382 | 0.45 | 103.5 |
| 53 | 0.832 | 0.837 | 0.834 | 0.00361 | 0.43 | 100.6 |
| 62 | 0.726 | 0.725 | 0.725 | 0.00042 | 0.06 | 85.3 |
| 4 | 0.132 | 0.137 | 0.134 | 0.00368 | 2.74 | 2.6 |
| 14 | 0.797 | 0.793 | 0.795 | 0.00346 | 0.44 | 95.1 |
| 21 | 0.776 | 0.787 | 0.782 | 0.00792 | 1.01 | 93.2 |
| 29 | 0.164 | 0.148 | 0.156 | 0.01131 | 7.24 | 5.6 |
| 38 | 0.475 | 0.383 | 0.429 | 0.06548 | 15.26 | 43.8 |
| 47 | 0.823 | 0.774 | 0.798 | 0.03444 | 4.31 | 95.6 |
| 54 | 0.781 | 0.729 | 0.755 | 0.03649 | 4.83 | 89.5 |
| 63 | 0.143 | 0.149 | 0.146 | 0.00396 | 2.72 | 4.2 |
| 5 | 0.716 | 0.716 | 0.716 | 0.00014 | 0.02 | 84.1 |
| 14 | 0.804 | 0.802 | 0.803 | 0.00148 | 0.18 | 96.2 |
| 22 | 0.900 | 0.877 | 0.888 | 0.01626 | 1.83 | 108.2 |
| 30 | 0.824 | 0.799 | 0.812 | 0.01725 | 2.13 | 97.4 |
| 39 | 0.744 | 0.819 | 0.781 | 0.05261 | 6.73 | 93.2 |
| 48 | 0.952 | 0.966 | 0.959 | 0.00933 | 0.97 | 118.1 |
| 55 | 0.952 | 0.919 | 0.935 | 0.02277 | 2.43 | 114.8 |
| 64 | 0.776 | 0.777 | 0.777 | 0.00042 | 0.05 | 92.5 |
| 5 | 0.691 | 0.617 | 0.654 | 0.05254 | 8.03 | 75.4 |
| 15 | 0.162 | 0.134 | 0.148 | 0.02022 | 13.66 | 4.5 |
| 9 | 0.791 | 0.608 | 0.700 | 0.12947 | 18.50 | 81.8 |
| 31 | 0.153 | 0.129 | 0.141 | 0.01676 | 11.87 | 3.5 |
| 40 | 0.731 | 0.585 | 0.658 | 0.10317 | 15.68 | 75.9 |
| DMSO | 0.789 | 0.700 | 0.744 | 0.06279 | 8.44 | 88.0 |
| 56 | 0.909 | 0.675 | 0.792 | 0.16546 | 20.88 | 94.7 |
| 65 | 0.164 | 0.151 | 0.157 | 0.00926 | 5.89 | 5.8 |
| 6 | 0.706 | 0.672 | 0.689 | 0.02404 | 3.49 | 83.9 |
| 16 | 0.641 | 0.601 | 0.621 | 0.02878 | 4.63 | 73.7 |
| 23 | 0.882 | 0.748 | 0.815 | 0.09504 | 11.66 | 102.5 |
| 32 | 0.822 | 0.802 | 0.812 | 0.01400 | 1.72 | 102.1 |
| 41 | 0.777 | 0.764 | 0.771 | 0.00919 | 1.19 | 95.9 |
| DMSO | 0.798 | 0.771 | 0.785 | 0.01916 | 2.44 | 98.0 |
| 57 | 0.791 | 0.789 | 0.790 | 0.00134 | 0.17 | 98.7 |
| 66 | 0.628 | 0.640 | 0.634 | 0.00806 | 1.27 | 75.6 |
| 7 | 0.149 | 0.135 | 0.142 | 0.00969 | 6.81 | 2.7 |
| 17 | 0.803 | 0.782 | 0.792 | 0.01492 | 1.88 | 99.1 |
| 24 | 0.125 | 0.129 | 0.127 | 0.00318 | 2.51 | 0.4 |
| 33 | 0.315 | 0.542 | 0.428 | 0.15995 | 37.34 | 45.2 |
| 42 | 0.820 | 0.748 | 0.784 | 0.05091 | 6.49 | 97.9 |
| 48 | 0.154 | 0.165 | 0.160 | 0.00806 | 5.05 | 5.3 |
| 58 | 0.737 | 0.730 | 0.734 | 0.00481 | 0.66 | 90.4 |
| 67 | 0.659 | 0.647 | 0.653 | 0.00813 | 1.25 | 78.5 |
| 8 | 0.165 | 0.154 | 0.159 | 0.00785 | 4.93 | 5.2 |
| 18 | 0.637 | 0.554 | 0.595 | 0.05876 | 9.87 | 69.9 |
| 25 | 0.684 | 0.588 | 0.636 | 0.06809 | 10.71 | 76.0 |
| 34 | 0.750 | 0.624 | 0.687 | 0.08945 | 13.02 | 83.5 |
| 43 | 0.678 | 0.618 | 0.648 | 0.04285 | 6.61 | 77.8 |
| 49 | 0.777 | 0.667 | 0.722 | 0.07757 | 10.74 | 88.7 |
| DMSO | 0.799 | 0.649 | 0.724 | 0.10564 | 14.59 | 89.0 |
| 68 | 0.648 | 0.625 | 0.636 | 0.01662 | 2.61 | 76.0 |
| 10 | 0.601 | 0.620 | 0.611 | 0.01308 | 2.14 | 72.2 |
| 19 | 0.695 | 0.702 | 0.698 | 0.00552 | 0.79 | 85.2 |
| 26 | 0.568 | 0.709 | 0.639 | 0.09956 | 15.59 | 76.4 |
| 35 | 0.623 | 0.765 | 0.694 | 0.10041 | 14.46 | 84.6 |
| 44 | 0.758 | 0.762 | 0.760 | 0.00297 | 0.39 | 94.3 |
| 50 | 0.487 | 0.434 | 0.461 | 0.03769 | 8.18 | 49.9 |
| 59 | 0.690 | 0.686 | 0.688 | 0.00262 | 0.38 | 83.7 |
| 69 | 0.535 | 0.550 | 0.543 | 0.01089 | 2.01 | 62.1 |
| 11 | 0.743 | 0.638 | 0.691 | 0.07446 | 10.78 | 84.1 |
| 19 | 0.694 | 0.603 | 0.649 | 0.06449 | 9.94 | 77.8 |
| 27 | 0.160 | 0.186 | 0.173 | 0.01824 | 10.56 | 7.2 |
| 36 | 0.662 | 0.566 | 0.614 | 0.06788 | 11.05 | 72.7 |
| 45 | 0.600 | 0.514 | 0.557 | 0.06102 | 10.96 | 64.2 |
| 51 | 0.685 | 0.524 | 0.605 | 0.11427 | 18.90 | 71.3 |
| 60 | 0.731 | 0.525 | 0.628 | 0.14552 | 23.18 | 74.7 |
| 74 | 0.715 | 0.596 | 0.655 | 0.08436 | 12.87 | 78.8 |
| 74 | 0.592 | 0.572 | 0.582 | 0.01393 | 2.39 | 70.0 |
| 80 | 0.614 | 0.611 | 0.613 | 0.00177 | 0.29 | 74.6 |
| 90 | 0.766 | 0.849 | 0.807 | 0.05869 | 7.27 | 104.3 |
| 99 | 0.830 | 0.813 | 0.822 | 0.01195 | 1.45 | 106.5 |
| 108 | 0.727 | 0.730 | 0.728 | 0.00198 | 0.27 | 92.2 |
| 117 | 0.713 | 0.836 | 0.774 | 0.08733 | 11.28 | 99.3 |
| 126 | 0.726 | 0.719 | 0.722 | 0.00523 | 0.72 | 91.3 |
| 138 | 0.646 | 0.681 | 0.663 | 0.02510 | 3.78 | 82.4 |
| 71 | 0.651 | 0.649 | 0.650 | 0.00120 | 0.19 | 80.3 |
| 81 | 0.642 | 0.622 | 0.632 | 0.01407 | 2.23 | 77.5 |
| 91 | 0.843 | 0.672 | 0.758 | 0.12099 | 15.97 | 96.7 |
| 100 | 0.734 | 0.815 | 0.774 | 0.05728 | 7.40 | 99.3 |
| 109 | 0.823 | 0.743 | 0.783 | 0.05699 | 7.28 | 100.6 |
| 118 | 0.871 | 0.874 | 0.872 | 0.00219 | 0.25 | 114.2 |
| 152 | 0.652 | 0.642 | 0.647 | 0.00721 | 1.12 | 79.8 |
| 134 | 0.617 | 0.633 | 0.625 | 0.01174 | 1.88 | 76.5 |
| 72 | 0.657 | 0.655 | 0.656 | 0.00134 | 0.20 | 81.2 |
| 82 | 0.684 | 0.809 | 0.746 | 0.08803 | 11.80 | 95.0 |
| 92 | 0.901 | 0.735 | 0.818 | 0.11731 | 14.34 | 106.0 |
| 101 | 0.791 | 0.768 | 0.779 | 0.01591 | 2.04 | 100.1 |
| 110 | 0.948 | 0.764 | 0.856 | 0.12982 | 15.17 | 111.7 |
| 119 | 0.821 | 0.608 | 0.714 | 0.15033 | 21.05 | 90.1 |
| 127 | 0.745 | 0.685 | 0.715 | 0.04243 | 5.94 | 90.2 |
| 135 | 0.624 | 0.618 | 0.621 | 0.00417 | 0.67 | 76.0 |
| 73 | 0.652 | 0.624 | 0.638 | 0.01916 | 3.00 | 78.5 |
| 83 | 0.773 | 0.662 | 0.718 | 0.07792 | 10.86 | 90.6 |
| 93 | 0.856 | 0.834 | 0.845 | 0.01570 | 1.86 | 110.1 |
| 102 | 0.828 | 0.800 | 0.814 | 0.02008 | 2.47 | 105.4 |
| 111 | 0.821 | 0.841 | 0.831 | 0.01421 | 1.71 | 108.0 |
| 120 | 0.816 | 0.787 | 0.802 | 0.02072 | 2.58 | 103.5 |
| 127 | 0.744 | 0.737 | 0.741 | 0.00453 | 0.61 | 94.1 |
| 136 | 0.699 | 0.679 | 0.689 | 0.01464 | 2.12 | 86.3 |
| 76 | 0.186 | 0.208 | 0.197 | 0.01541 | 7.83 | 11.3 |
| 84 | 0.665 | 0.699 | 0.682 | 0.02432 | 3.57 | 85.2 |
| 94 | 0.810 | 0.683 | 0.746 | 0.09030 | 12.10 | 95.0 |
| 103 | 0.141 | 0.162 | 0.151 | 0.01506 | 9.95 | 4.3 |
| DMSO | 0.784 | 0.605 | 0.695 | 0.12671 | 18.25 | 87.1 |
| 121 | 0.726 | 0.590 | 0.658 | 0.09624 | 14.63 | 81.5 |
| 128 | 0.635 | 0.620 | 0.628 | 0.01068 | 1.70 | 76.9 |
| 136 | 0.697 | 0.695 | 0.696 | 0.00113 | 0.16 | 87.3 |
| 75 | 0.154 | 0.153 | 0.154 | 0.00042 | 0.28 | 4.5 |
| 85 | 0.616 | 0.645 | 0.630 | 0.02072 | 3.29 | 82.1 |
| 70 | 0.909 | 0.830 | 0.869 | 0.05614 | 6.46 | 121.0 |
| 104 | 0.150 | 0.150 | 0.150 | 0.00028 | 0.19 | 3.9 |
| 112 | 0.981 | 1.056 | 1.018 | 0.05303 | 5.21 | 145.3 |
| 122 | 0.166 | 0.189 | 0.177 | 0.01626 | 9.19 | 8.3 |
| 129 | 0.718 | 0.451 | 0.584 | 0.18887 | 32.34 | 74.6 |
| 137 | 0.652 | 0.647 | 0.649 | 0.00389 | 0.60 | 85.2 |
| 77 | 0.503 | 0.529 | 0.516 | 0.01860 | 3.61 | 63.5 |
| 86 | 0.603 | 0.609 | 0.606 | 0.00424 | 0.70 | 78.1 |
| 95 | 0.856 | 0.793 | 0.824 | 0.04419 | 5.36 | 113.7 |
| 154 | 0.883 | 0.848 | 0.866 | 0.02503 | 2.89 | 120.5 |
| 113 | 0.779 | 0.784 | 0.781 | 0.00368 | 0.47 | 106.7 |
| 123 | 0.892 | 0.914 | 0.903 | 0.01591 | 1.76 | 126.6 |
| 130 | 0.544 | 0.537 | 0.540 | 0.00460 | 0.85 | 67.5 |
| 139 | 0.532 | 0.682 | 0.607 | 0.10543 | 17.37 | 78.3 |
| 77 | 0.665 | 0.645 | 0.655 | 0.01400 | 2.14 | 86.1 |
| 87 | 0.676 | 0.677 | 0.677 | 0.00035 | 0.05 | 89.7 |
| 96 | 0.935 | 0.807 | 0.871 | 0.09115 | 10.47 | 121.3 |
| 105 | 0.916 | 0.859 | 0.887 | 0.03981 | 4.49 | 124.0 |
| 114 | 0.907 | 0.891 | 0.899 | 0.01124 | 1.25 | 125.9 |
| 124 | 0.909 | 0.896 | 0.902 | 0.00919 | 1.02 | 126.4 |
| 131 | 0.682 | 0.797 | 0.740 | 0.08118 | 10.98 | 99.9 |
| 140 | 0.679 | 0.644 | 0.661 | 0.02510 | 3.80 | 87.2 |
| 78 | 0.300 | 0.223 | 0.261 | 0.05452 | 20.88 | 22.0 |
| 88 | 0.183 | 0.175 | 0.179 | 0.00573 | 3.20 | 8.6 |
| 97 | 0.741 | 0.728 | 0.734 | 0.00884 | 1.20 | 99.1 |
| 106 | 0.935 | 0.906 | 0.921 | 0.02051 | 2.23 | 129.4 |
| 115 | 0.131 | 0.128 | 0.129 | 0.00212 | 1.64 | 0.5 |
| 125 | 0.138 | 0.137 | 0.138 | 0.00092 | 0.67 | 1.9 |
| 132 | 0.241 | 0.227 | 0.234 | 0.01032 | 4.41 | 17.6 |
| 155 | 0.604 | 0.639 | 0.622 | 0.02475 | 3.98 | 80.7 |
| 79 | 0.247 | 0.182 | 0.215 | 0.04617 | 21.52 | 14.4 |
| 89 | 0.659 | 0.634 | 0.647 | 0.01718 | 2.66 | 84.8 |
| 98 | 0.758 | 0.575 | 0.667 | 0.12961 | 19.44 | 88.1 |
| 107 | 0.166 | 0.170 | 0.168 | 0.00276 | 1.64 | 6.9 |
| 116 | 0.651 | 0.559 | 0.605 | 0.06541 | 10.81 | 78.0 |
| 126 | 0.803 | 0.694 | 0.748 | 0.07693 | 10.28 | 101.3 |
| 133 | 0.823 | 0.634 | 0.728 | 0.13378 | 18.37 | 98.1 |
| 141 | 0.624 | 0.618 | 0.621 | 0.00431 | 0.69 | 80.6 |
| 161 | 0.639 | 0.603 | 0.621 | 0.02553 | 4.11 | 73.6 |
| 171 | 0.143 | 0.149 | 0.146 | 0.00403 | 2.76 | 2.9 |
| 251 | 0.817 | 0.818 | 0.818 | 0.00071 | 0.09 | 102.8 |
| 188 | 0.742 | 0.752 | 0.747 | 0.00679 | 0.91 | 92.2 |
| 198 | 0.856 | 0.905 | 0.881 | 0.03479 | 3.95 | 112.1 |
| 207 | 0.650 | 0.576 | 0.613 | 0.05268 | 8.59 | 72.4 |
| 216 | 0.768 | 0.724 | 0.746 | 0.03097 | 4.15 | 92.2 |
| 225 | 0.556 | 0.549 | 0.553 | 0.00537 | 0.97 | 63.4 |
| 162 | 0.227 | 0.242 | 0.235 | 0.01103 | 4.70 | 16.1 |
| 172 | 0.634 | 0.663 | 0.649 | 0.02044 | 3.15 | 77.7 |
| 180 | 0.141 | 0.128 | 0.135 | 0.00919 | 6.83 | 1.3 |
| 189 | 0.847 | 0.832 | 0.840 | 0.01110 | 1.32 | 106.0 |
| 199 | 0.803 | 0.845 | 0.824 | 0.02998 | 3.64 | 103.7 |
| 208 | 0.860 | 0.860 | 0.860 | 0.00035 | 0.04 | 109.1 |
| 217 | 0.528 | 0.497 | 0.513 | 0.02227 | 4.34 | 57.5 |
| 226 | 0.683 | 0.688 | 0.686 | 0.00332 | 0.48 | 83.1 |
| 180 | 0.611 | 0.628 | 0.620 | 0.01202 | 1.94 | 73.3 |
| 173 | 0.719 | 0.749 | 0.734 | 0.02143 | 2.92 | 90.3 |
| 181 | 0.916 | 0.838 | 0.877 | 0.05487 | 6.26 | 111.6 |
| 190 | 0.771 | 0.740 | 0.755 | 0.02178 | 2.88 | 93.5 |
| 200 | 0.820 | 0.852 | 0.836 | 0.02305 | 2.76 | 105.5 |
| 209 | 0.971 | 0.913 | 0.942 | 0.04137 | 4.39 | 121.2 |
| 221 | 0.839 | 0.743 | 0.791 | 0.06746 | 8.53 | 98.8 |
| 227 | 0.562 | 0.527 | 0.544 | 0.02440 | 4.48 | 62.2 |
| 163 | 0.678 | 0.661 | 0.670 | 0.01195 | 1.78 | 80.8 |
| 174 | 0.722 | 0.713 | 0.717 | 0.00658 | 0.92 | 87.9 |
| 182 | 0.802 | 0.801 | 0.802 | 0.00106 | 0.13 | 100.4 |
| 191 | 0.854 | 0.857 | 0.855 | 0.00205 | 0.24 | 108.4 |
| 201 | 0.767 | 0.798 | 0.782 | 0.02157 | 2.76 | 97.5 |
| 210 | 0.789 | 0.776 | 0.782 | 0.00870 | 1.11 | 97.5 |
| 218 | 0.720 | 0.709 | 0.714 | 0.00764 | 1.07 | 87.4 |
| 228 | 0.641 | 0.618 | 0.630 | 0.01619 | 2.57 | 74.9 |
| 164 | 0.603 | 0.584 | 0.593 | 0.01372 | 2.31 | 69.4 |
| 175 | 0.135 | 0.158 | 0.146 | 0.01633 | 11.18 | 3.0 |
| 183 | 0.792 | 0.572 | 0.682 | 0.15563 | 22.83 | 82.6 |
| 192 | 0.752 | 0.593 | 0.673 | 0.11292 | 16.79 | 81.2 |
| 202 | 0.805 | 0.598 | 0.702 | 0.14644 | 20.87 | 85.5 |
| 211 | 0.599 | 0.504 | 0.552 | 0.06682 | 12.11 | 63.2 |
| 219 | 0.714 | 0.593 | 0.654 | 0.08549 | 13.08 | 78.4 |
| 229 | 0.699 | 0.698 | 0.698 | 0.00099 | 0.14 | 85.0 |
| 165 | 0.690 | 0.674 | 0.682 | 0.01131 | 1.66 | 83.3 |
| 176 | 0.616 | 0.634 | 0.625 | 0.01301 | 2.08 | 74.8 |
| 184 | 0.809 | 0.817 | 0.813 | 0.00552 | 0.68 | 103.0 |
| 193 | 0.128 | 0.133 | 0.131 | 0.00361 | 2.76 | 0.7 |
| 203 | 0.821 | 0.811 | 0.816 | 0.00721 | 0.88 | 103.4 |
| 212 | 0.456 | 0.474 | 0.465 | 0.01223 | 2.63 | 50.8 |
| 220 | 0.762 | 0.766 | 0.764 | 0.00304 | 0.40 | 95.7 |
| 230 | 0.680 | 0.663 | 0.671 | 0.01195 | 1.78 | 81.8 |
| 166 | 0.615 | 0.635 | 0.625 | 0.01400 | 2.24 | 74.8 |
| 169 | 0.681 | 0.698 | 0.689 | 0.01266 | 1.84 | 84.5 |
| 185 | 0.830 | 0.807 | 0.818 | 0.01584 | 1.94 | 103.8 |
| 194 | 0.869 | 0.849 | 0.859 | 0.01442 | 1.68 | 109.9 |
| 204 | 0.821 | 0.841 | 0.831 | 0.01428 | 1.72 | 105.7 |
| 213 | 0.819 | 0.840 | 0.830 | 0.01485 | 1.79 | 105.5 |
| 221 | 0.795 | 0.793 | 0.794 | 0.00078 | 0.10 | 100.1 |
| 231 | 0.640 | 0.636 | 0.638 | 0.00283 | 0.44 | 76.7 |
| 168 | 0.610 | 0.628 | 0.619 | 0.01266 | 2.05 | 73.9 |
| 177 | 0.143 | 0.144 | 0.144 | 0.00035 | 0.25 | 2.6 |
| 167 | 0.804 | 0.903 | 0.853 | 0.07000 | 8.20 | 109.0 |
| 195 | 0.918 | 0.854 | 0.886 | 0.04483 | 5.06 | 113.9 |
| 205 | 0.105 | 1.080 | 0.593 | 0.68971 | 116.37 | 70.0 |
| 214 | 0.877 | 0.860 | 0.868 | 0.01209 | 1.39 | 111.3 |
| 222 | 0.808 | 0.695 | 0.751 | 0.07941 | 10.57 | 93.8 |
| 232 | 0.720 | 0.697 | 0.709 | 0.01648 | 2.33 | 87.3 |
| 169 | 0.636 | 0.621 | 0.629 | 0.01054 | 1.68 | 75.4 |
| 178 | 0.640 | 0.634 | 0.637 | 0.00474 | 0.74 | 76.6 |
| 186 | 0.833 | 0.833 | 0.833 | 0.00000 | 0.00 | 106.0 |
| 196 | 0.887 | 0.846 | 0.866 | 0.02934 | 3.39 | 111.0 |
| 206 | 0.845 | 0.877 | 0.861 | 0.02326 | 2.70 | 110.2 |
| 214 | 0.794 | 0.784 | 0.789 | 0.00686 | 0.87 | 99.4 |
| 223 | 0.770 | 0.786 | 0.778 | 0.01138 | 1.46 | 97.8 |
| 158 | 0.629 | 0.659 | 0.644 | 0.02128 | 3.30 | 77.7 |
| 170 | 0.584 | 0.558 | 0.571 | 0.01817 | 3.18 | 66.8 |
| 179 | 0.707 | 0.679 | 0.693 | 0.01987 | 2.87 | 85.0 |
| 187 | 0.727 | 0.578 | 0.652 | 0.10536 | 16.15 | 78.9 |
| 197 | 0.742 | 0.629 | 0.685 | 0.07969 | 11.63 | 83.8 |
| DMSO | 0.653 | 0.507 | 0.580 | 0.10310 | 17.78 | 68.0 |
| 215 | 0.722 | 0.568 | 0.645 | 0.10904 | 16.90 | 77.9 |
| 224 | 0.643 | 0.581 | 0.612 | 0.04384 | 7.16 | 72.9 |
| 233 | 0.608 | 0.590 | 0.599 | 0.01245 | 2.08 | 70.9 |
| 142 | 0.597 | 0.610 | 0.603 | 0.00926 | 1.54 | 71.2 |
| 143 | 0.687 | 0.668 | 0.677 | 0.01336 | 1.97 | 82.4 |
| 144 | 0.840 | 0.832 | 0.836 | 0.00594 | 0.71 | 106.1 |
| 145 | 0.831 | 0.822 | 0.826 | 0.00587 | 0.71 | 104.7 |
| 146 | 0.863 | 0.856 | 0.860 | 0.00509 | 0.59 | 109.7 |
| 147 | 0.886 | 0.802 | 0.844 | 0.05954 | 7.05 | 107.3 |
| 148 | 0.753 | 0.687 | 0.720 | 0.04660 | 6.47 | 88.8 |
| 149 | 0.455 | 0.463 | 0.459 | 0.00587 | 1.28 | 49.6 |
| 150 | 0.668 | 0.678 | 0.673 | 0.00764 | 1.13 | 81.7 |
| 151 | 0.181 | 0.171 | 0.176 | 0.00658 | 3.74 | 7.2 |
| 152 | 0.832 | 0.842 | 0.837 | 0.00658 | 0.79 | 106.3 |
| 153 | 0.795 | 0.802 | 0.798 | 0.00445 | 0.56 | 100.5 |
| 70 | 0.157 | 0.140 | 0.148 | 0.01202 | 8.11 | 3.0 |
| 154 | 0.153 | 0.153 | 0.153 | 0.00035 | 0.23 | 3.7 |
| 155 | 0.168 | 0.154 | 0.161 | 0.00969 | 6.02 | 4.9 |
| 156 | 0.670 | 0.641 | 0.655 | 0.02079 | 3.17 | 79.1 |
| 159 | 0.706 | 0.679 | 0.693 | 0.01888 | 2.73 | 84.7 |
| 234 | 0.788 | 0.666 | 0.727 | 0.08627 | 11.86 | 89.8 |
| 235 | 0.879 | 0.785 | 0.832 | 0.06640 | 7.98 | 105.6 |
| 236 | 0.168 | 0.176 | 0.172 | 0.00537 | 3.13 | 6.6 |
| 237 | 0.946 | 0.848 | 0.897 | 0.06972 | 7.77 | 115.3 |
| 238 | 0.187 | 0.202 | 0.194 | 0.01089 | 5.61 | 9.9 |
| 239 | 0.906 | 0.688 | 0.797 | 0.15394 | 19.31 | 100.3 |
| 240 | 0.715 | 0.674 | 0.694 | 0.02850 | 4.10 | 84.9 |
| 241 | 0.695 | 0.700 | 0.697 | 0.00339 | 0.49 | 85.3 |
| 241 | 0.665 | 0.631 | 0.648 | 0.02369 | 3.66 | 78.0 |
| 242 | 0.590 | 0.613 | 0.601 | 0.01655 | 2.75 | 71.0 |
| 243 | 0.681 | 0.687 | 0.684 | 0.00382 | 0.56 | 83.3 |
| 244 | 0.829 | 0.821 | 0.825 | 0.00530 | 0.64 | 104.5 |
| 245 | 0.822 | 0.790 | 0.806 | 0.02270 | 2.82 | 101.6 |
| 246 | 0.671 | 0.684 | 0.677 | 0.00912 | 1.35 | 82.3 |
| 247 | 0.686 | 0.668 | 0.677 | 0.01266 | 1.87 | 82.3 |
| 248 | 0.212 | 0.197 | 0.204 | 0.01047 | 5.12 | 11.5 |
| 249 | 0.666 | 0.666 | 0.666 | 0.00007 | 0.01 | 80.7 |
| 250 | 0.736 | 0.656 | 0.696 | 0.05643 | 8.11 | 85.1 |
| 160 | 0.726 | 0.610 | 0.668 | 0.08217 | 12.30 | 81.0 |
| 157 | 0.303 | 0.310 | 0.306 | 0.00488 | 1.59 | 26.7 |
| DMSO | 0.786 | 0.659 | 0.722 | 0.09001 | 12.46 | 89.1 |
| DMSO | 0.673 | 0.649 | 0.661 | 0.01676 | 2.53 | 79.9 |
| DMSO | 0.701 | 0.686 | 0.693 | 0.01011 | 1.46 | 84.8 |

TABLE III
EFFECTS OF INHIBITORS OF GSK-3B ENZYME
ACTIVITY ON THE VIABILITY OF CELLS
EXPRESSING PLURIPOTENCY MARKERS.

| Com- | cmpd | | | | | | % |
|---|---|---|---|---|---|---|---|
| pound | conc | Raw data | | Aver- | | % | Con- |
| # | (μM) | (duplicate) | | age | S.D. | CV | trol |
| EXPRES | | 0.6379 | 0.6180 | 0.6280 | 0.0141 | 2.2 | 74.6 |
| 01 medium | | | | | | | |
| no | | 0.7412 | 0.7038 | 0.7225 | 0.0264 | 3.7 | 88.7 |
| treatment | | | | | | | |
| AA only | | 0.7674 | 0.8047 | 0.7861 | 0.0264 | 3.4 | 98.3 |
| AA + | | 0.7754 | 0.8200 | 0.7977 | 0.0315 | 4.0 | 100.0 |
| Wnt3a | | | | | | | |
| 144 | 10 | 0.1412 | 0.1515 | 0.1464 | 0.0073 | 5.0 | 2.4 |
| 144 | 5 | 0.1501 | 0.1444 | 0.1473 | 0.0040 | 2.7 | 2.5 |
| 144 | 2.5 | 0.1541 | 0.4254 | 0.2898 | 0.1918 | 66.2 | 23.9 |
| 145 | 10 | 0.1272 | 0.1282 | 0.1277 | 0.0007 | 0.6 | −0.4 |
| 145 | 5 | 0.5862 | 0.5880 | 0.5871 | 0.0013 | 0.2 | 68.4 |
| 145 | 2.5 | 0.7613 | 0.7603 | 0.7608 | 0.0007 | 0.1 | 94.5 |
| 148 | 10 | 0.1481 | 0.1592 | 0.1537 | 0.0078 | 5.1 | 3.5 |
| 148 | 5 | 0.1479 | 0.1639 | 0.1559 | 0.0113 | 7.3 | 3.8 |
| 148 | 2.5 | 0.2861 | 0.2477 | 0.2669 | 0.0272 | 10.2 | 20.4 |
| 150 | 10 | 0.2092 | 0.2426 | 0.2259 | 0.0236 | 10.5 | 14.3 |
| 150 | 5 | 0.6815 | 0.7128 | 0.6972 | 0.0221 | 3.2 | 84.9 |
| 150 | 2.5 | 0.7389 | 0.7870 | 0.7630 | 0.0340 | 4.5 | 94.8 |
| 101 | 10 | 0.1381 | 0.1398 | 0.1390 | 0.0012 | 0.9 | 1.3 |
| 101 | 5 | 0.7826 | 0.7578 | 0.7702 | 0.0175 | 2.3 | 95.9 |
| 101 | 2.5 | 0.8231 | 0.7742 | 0.7987 | 0.0346 | 4.3 | 100.1 |
| 103 | 10 | 0.1352 | 0.1326 | 0.1339 | 0.0018 | 1.4 | 0.5 |
| 103 | 5 | 0.2632 | 0.2604 | 0.2618 | 0.0020 | 0.8 | 19.7 |
| 103 | 2.5 | 0.4160 | 0.5314 | 0.4737 | 0.0816 | 17.2 | 51.4 |
| 198 | 10 | 0.4447 | 0.4791 | 0.4619 | 0.0243 | 5.3 | 49.7 |
| 198 | 5 | 0.6902 | 0.6884 | 0.6893 | 0.0013 | 0.2 | 83.8 |
| 198 | 2.5 | 0.7476 | 0.7483 | 0.7480 | 0.0005 | 0.1 | 92.5 |
| 110 | 10 | 0.6790 | 0.6704 | 0.6747 | 0.0061 | 0.9 | 81.6 |
| 110 | 5 | 0.7833 | 0.7924 | 0.7879 | 0.0064 | 0.8 | 98.5 |
| 110 | 2.5 | 0.8155 | 0.8389 | 0.8272 | 0.0165 | 2.0 | 104.4 |
| 111 | 10 | 0.6533 | 0.6884 | 0.6709 | 0.0248 | 3.7 | 81.0 |
| 111 | 5 | 0.7697 | 0.7738 | 0.7718 | 0.0029 | 0.4 | 96.1 |
| 111 | 2.5 | 0.8119 | 0.8219 | 0.8169 | 0.0071 | 0.9 | 102.9 |
| 112 | 10 | 0.1242 | 0.1323 | 0.1283 | 0.0057 | 4.5 | −0.4 |
| 112 | 5 | 0.1263 | 0.1303 | 0.1283 | 0.0028 | 2.2 | −0.3 |
| 112 | 2.5 | 0.8480 | 0.7725 | 0.8103 | 0.0534 | 6.6 | 101.9 |
| 206 | 10 | 0.1695 | 0.1890 | 0.1793 | 0.0138 | 7.7 | 7.3 |
| 206 | 5 | 0.7217 | 0.7435 | 0.7326 | 0.0154 | 2.1 | 90.2 |
| 206 | 2.5 | 0.7812 | 0.7221 | 0.7517 | 0.0418 | 5.6 | 93.1 |
| EXPRES | | 0.6294 | 0.6363 | 0.6329 | 0.0049 | 0.8 | 70.3 |
| 01medium | | | | | | | |
| no | | 0.7156 | 0.7356 | 0.7256 | 0.0141 | 1.9 | 83.3 |
| treatment | | | | | | | |
| AA only | | 0.8732 | 0.9046 | 0.8889 | 0.0222 | 2.5 | 106.0 |
| AA + | | 0.8415 | 0.8500 | 0.8458 | 0.0060 | 0.7 | 100.0 |
| Wnt3a | | | | | | | |
| 52 | 10 | 0.1403 | 0.1493 | 0.1448 | 0.0064 | 4.4 | 2.3 |
| 52 | 5 | 0.4434 | 0.3878 | 0.4156 | 0.0393 | 9.5 | 40.1 |
| 52 | 2.5 | 0.7734 | 0.8038 | 0.7886 | 0.0215 | 2.7 | 92.0 |
| 133 | 10 | 0.2993 | 0.3026 | 0.3010 | 0.0023 | 0.8 | 24.1 |
| 133 | 5 | 0.7023 | 0.6299 | 0.6661 | 0.0512 | 7.7 | 75.0 |
| 133 | 2.5 | 0.7835 | 0.8043 | 0.7939 | 0.0147 | 1.9 | 92.8 |
| 223 | 10 | 0.7205 | 0.7369 | 0.7287 | 0.0116 | 1.6 | 83.7 |
| 223 | 5 | 0.7769 | 0.8272 | 0.8021 | 0.0356 | 4.4 | 93.9 |
| 223 | 2.5 | 0.8214 | 0.8640 | 0.8427 | 0.0301 | 3.6 | 99.6 |
| 221 | 10 | 0.6275 | 0.5980 | 0.6128 | 0.0209 | 3.4 | 67.5 |
| 221 | 5 | 0.7159 | 0.7222 | 0.7191 | 0.0045 | 0.6 | 82.3 |
| 221 | 2.5 | 0.9245 | 0.9403 | 0.9324 | 0.0112 | 1.2 | 112.1 |
| 226 | 10 | 0.7220 | 0.6670 | 0.6945 | 0.0389 | 5.6 | 78.9 |
| 226 | 5 | 0.7526 | 0.7486 | 0.7506 | 0.0028 | 0.4 | 86.7 |
| 226 | 2.5 | 0.7557 | 0.7390 | 0.7474 | 0.0118 | 1.6 | 86.3 |
| 136 | 10 | 0.8214 | 0.8636 | 0.8425 | 0.0298 | 3.5 | 99.5 |
| 136 | 5 | 0.7996 | 0.7873 | 0.7935 | 0.0087 | 1.1 | 92.7 |
| 136 | 2.5 | 0.8669 | 0.8195 | 0.8432 | 0.0335 | 4.0 | 99.6 |
| 158 | 10 | 0.6195 | 0.5908 | 0.6052 | 0.0203 | 3.4 | 66.5 |
| 158 | 5 | 0.8047 | 0.8319 | 0.8183 | 0.0192 | 2.4 | 96.2 |
| 158 | 2.5 | 0.8041 | 0.7900 | 0.7971 | 0.0100 | 1.3 | 93.2 |
| 233 | 10 | 0.1261 | 0.1520 | 0.1391 | 0.0183 | 13.2 | 1.5 |
| 233 | 5 | 0.1303 | 0.1263 | 0.1283 | 0.0028 | 2.2 | 0.0 |
| 233 | 2.5 | 0.4482 | 0.4051 | 0.4267 | 0.0305 | 7.1 | 41.6 |

TABLE IV
EFFECTS OF INHIBITORS OF GSK-3B ENZYME ACTIVITY ON THE DIFFERENTIATION
AND PROLIFERATION OF HUMAN EMBRYONIC STEM CELLS.

| | Proliferative Response | | SOX-17 Expression | | Proliferative Response | | HNF-3b Expression | |
|---|---|---|---|---|---|---|---|---|
| | | Fold over | | Fold over | | Fold over | | Fold over |
| | Total | Wnt 3a/AA | Total | Wnt 3a/AA | Total | Wnt 3a/AA | Total | Wnt 3a/AA |
| Compound # | cells | control | Intensity | control | cells | control | Intensity | control |
| 142 | 1723 | 0.11244207 | 68870409 | 0.0708 | 1645 | 0.10460717 | 50143628 | 0.0453 |
| 143 | 1110 | 0.07245904 | 42978557 | 0.0442 | 94 | 0.00597755 | 0 | 0.0000 |
| 144 | 7990 | 0.52154188 | 339840000 | 0.3494 | 6833 | 0.43448539 | 231745000 | 0.2092 |
| 145 | 4914 | 0.32074548 | 238555000 | 0.2453 | 2907 | 0.18485899 | 82808745 | 0.0747 |
| 146 | 3056 | 0.19945819 | 153145000 | 0.1575 | 2643 | 0.16807097 | 122246784 | 0.1103 |
| 147 | 3960 | 0.25850251 | 47669463 | 0.0490 | 4641 | 0.29512575 | 210730000 | 0.1902 |
| 148 | 12243 | 0.79917096 | 699160000 | 0.7189 | 6536 | 0.41559887 | 248855000 | 0.2246 |
| 149 | 401 | 0.02614400 | 25580022 | 0.0263 | 27 | 0.00168516 | 0 | 0.0000 |
| 150 | 7958 | 0.51948561 | 351070000 | 0.3610 | 6992 | 0.44459636 | 288075000 | 0.2600 |
| 151 | 277 | 0.01808212 | 6558563 | 0.0067 | 12 | 0.00073130 | 535481 | 0.0005 |
| 152 | 1327 | 0.08662445 | 69037756 | 0.0710 | 1194 | 0.07589584 | 40478497 | 0.0365 |
| 153 | 791 | 0.05160259 | 24732475 | 0.0254 | 64 | 0.00406982 | 1092011 | 0.0010 |
| 70 | 0 | 0.00000000 | 0 | 0.0000 | 3 | 0.00019077 | 95784 | 0.0001 |
| 154 | 2 | 0.00013056 | 0 | 0.0000 | 0 | 0.00000000 | 0 | 0.0000 |
| 155 | 6 | 0.00035903 | 1092432 | 0.0011 | 2 | 0.00009539 | 150222 | 0.0001 |
| 156 | 2742 | 0.17899341 | 122926199 | 0.1264 | 3166 | 0.20132905 | 120729987 | 0.1090 |
| 157 | 33 | 0.00212155 | 3855900 | 0.0040 | 8 | 0.00050873 | 208129 | 0.0002 |
| 213 | 2000 | 0.13055682 | 110080123 | 0.1132 | 116 | 0.00737655 | 4290889 | 0.0039 |
| 214 | 3495 | 0.22814805 | 110559816 | 0.1137 | 438 | 0.02782105 | 24450647 | 0.0221 |
| 214 | 3107 | 0.20278739 | 120998421 | 0.1244 | 6177 | 0.39276971 | 273965000 | 0.2473 |
| 215 | 658 | 0.04295320 | 37841044 | 0.0389 | 646 | 0.04107977 | 31352380 | 0.0283 |
| 216 | 5991 | 0.39108297 | 252690000 | 0.2598 | 8479 | 0.53915615 | 306520000 | 0.2767 |
| 217 | 1953 | 0.12745610 | 88653625 | 0.0912 | 641 | 0.04076182 | 18162585 | 0.0164 |
| 218 | 2024 | 0.13209087 | 128395000 | 0.1320 | 4923 | 0.31302661 | 232020000 | 0.2094 |
| 219 | 2979 | 0.19446439 | 93454696 | 0.0961 | 3582 | 0.22775110 | 137054653 | 0.1237 |
| 220 | 3703 | 0.24169332 | 138180000 | 0.1421 | 3980 | 0.25306032 | 139550000 | 0.1260 |
| 221 | 21070 | 1.37538351 | 1089750000 | 1.1205 | 21203 | 1.34831961 | 1281000000 | 1.1562 |
| 222 | 1297 | 0.08466610 | 47445962 | 0.0488 | 30 | 0.00190773 | 0 | 0.0000 |
| 223 | 14529 | 0.94839741 | 1013360000 | 1.0419 | 9871 | 0.62767480 | 540725000 | 0.4881 |
| 224 | 4063 | 0.26522619 | 207891758 | 0.2137 | 3973 | 0.25264697 | 177190000 | 0.1599 |
| 225 | 1 | 0.00006528 | 0 | 0.0000 | 7 | 0.00041334 | 0 | 0.0000 |
| 226 | 9716 | 0.63421242 | 572520000 | 0.5887 | 7650 | 0.48643922 | 329425000 | 0.2973 |
| 227 | 916 | 0.05979503 | 0 | 0.0000 | 1076 | 0.06839210 | 40211776 | 0.0363 |
| 228 | 738 | 0.04817547 | 30943000 | 0.0318 | 503 | 0.03198626 | 0 | 0.0000 |
| 229 | 8367 | 0.54618448 | 373185000 | 0.3837 | 7976 | 0.50720168 | 260000000 | 0.2347 |
| 230 | 20079 | 1.31069260 | 1104750000 | 1.1359 | 16884 | 1.07363836 | 1052345000 | 0.9499 |
| 231 | 13789 | 0.90012403 | 789085000 | 0.8113 | 11369 | 0.72296588 | 547055000 | 0.4938 |
| 232 | 16652 | 1.08698348 | 1045395000 | 1.0749 | 14950 | 0.95065340 | 854325000 | 0.7711 |
| 158 | 6376 | 0.41618252 | 324450000 | 0.3336 | 6058 | 0.38523417 | 269025000 | 0.2428 |
| 233 | 6470 | 0.42231869 | 327055000 | 0.3363 | 4357 | 0.27706591 | 109160000 | 0.0985 |
| No treatment | 3891 | 0.25396566 | 97657703 | 0.1004 | 6091 | 0.38733268 | 109336609 | 0.0987 |
| AA | 4348 | 0.28379790 | 104735084 | 0.1077 | 122 | 0.00775810 | 5341271 | 0.0048 |
| AA/3a | 15319 | 1.00000000 | 972595000 | 1.0000 | 15726 | 1.00000000 | 1107900000 | 1.0000 |
| 161 | 738 | 0.44211577 | 0 | 0.0000 | 0 | 0.00000000 | 0 | 0.0000 |
| 162 | 0 | 0.00000000 | 0 | 0.0000 | 0 | 0.00000000 | 0 | 0.0000 |
| DMSO | 56 | 0.03353293 | 454796 | 0.0148 | 211 | 0.16644754 | 4455058 | 0.1626 |
| 163 | 1313 | 0.78642715 | 28506437 | 0.9266 | 5485 | 4.32684722 | 85245671 | 3.1115 |
| 164 | 12 | 0.00738523 | 85949 | 0.0028 | 67 | 0.05259006 | 1300640 | 0.0475 |
| 165 | 2899 | 1.73612774 | 32703235 | 1.0630 | 7460 | 5.88456482 | 149772525 | 5.4668 |
| 166 | 562 | 0.33632735 | 11388240 | 0.3702 | 787 | 0.62108861 | 10743082 | 0.3921 |
| 168 | 118 | 0.07045908 | 2574279 | 0.0837 | 57 | 0.04522745 | 2584708 | 0.0943 |
| 169 | 136 | 0.08163673 | 410648 | 0.0133 | 0 | 0.00000000 | 0 | 0.0000 |
| 170 | 19 | 0.01137725 | 0 | 0.0000 | 0 | 0.00000000 | 0 | 0.0000 |
| 171 | 3 | 0.00159681 | 431883 | 0.0140 | 31 | 0.02419143 | 847186 | 0.0309 |
| 172 | 33 | 0.01976048 | 0 | 0.0000 | 225 | 0.17749145 | 5223879 | 0.1907 |
| 173 | 16 | 0.00978044 | 0 | 0.0000 | 496 | 0.39127005 | 8966327 | 0.3273 |
| 174 | 26 | 0.01556886 | 459801 | 0.0149 | 189 | 0.14935577 | 1819533 | 0.0664 |
| 175 | 1 | 0.00039920 | 0 | 0.0000 | 42 | 0.03339469 | 1605538 | 0.0586 |
| 176 | 22 | 0.01297405 | 82062 | 0.0027 | 311 | 0.24506968 | 5749996 | 0.2099 |
| 177 | 0 | 0.00000000 | 0 | 0.0000 | 0 | 0.00000000 | 0 | 0.0000 |
| 178 | 26 | 0.01556886 | 0 | 0.0000 | 0 | 0.00000000 | 0 | 0.0000 |
| 179 | 202 | 0.12095808 | 627280 | 0.0204 | 1079 | 0.85143308 | 14326715 | 0.5229 |
| 180 | 3 | 0.00179641 | 0 | 0.0000 | 4 | 0.00315540 | 101114 | 0.0037 |
| 181 | 1310 | 0.78423154 | 24382455 | 0.7926 | 3249 | 2.56323955 | 75834631 | 2.7680 |
| 182 | 20 | 0.01177645 | 0 | 0.0000 | 425 | 0.33526164 | 8880858 | 0.3242 |
| 184 | 9 | 0.00538922 | 37140 | 0.0012 | 134 | 0.10570602 | 2144545 | 0.0783 |
| 183 | 7 | 0.00419162 | 48154 | 0.0016 | 5 | 0.00420720 | 170177 | 0.0062 |
| 185 | 70 | 0.04191617 | 589594 | 0.0192 | 0 | 0.00000000 | 0 | 0.0000 |
| 186 | 1215 | 0.72774451 | 7568849 | 0.2460 | 0 | 0.00000000 | 0 | 0.0000 |
| no Treatment | 1145 | 0.68542914 | 6979814 | 0.2269 | not done | | | |
| AA | 100 | 0.05988024 | 1264807 | 0.0411 | 51 | 0.04049435 | 923625 | 0.0337 |
| AA/3a | 1670 | 1.00000000 | 30764293 | 1.0000 | 1268 | 1.00000000 | 27396787 | 1.0000 |
| 187 | 43 | 0.00510815 | 706614 | 0.0055 | 0 | 0.00000000 | 0 | 0.0000 |
| 188 | 7 | 0.00079815 | 102445 | 0.0008 | 0 | 0.00000000 | 0 | 0.0000 |
| 189 | 46 | 0.00546732 | 0 | 0.0000 | 46 | 0.00548446 | 818478 | 0.0044 |
| 190 | 5 | 0.00059861 | 284777 | 0.0022 | 32 | 0.00385502 | 2309043 | 0.0124 |
| 191 | 258 | 0.03092825 | 4009395 | 0.0312 | 391 | 0.04665766 | 14340307 | 0.0769 |
| 192 | 62 | 0.00742278 | 782261 | 0.0061 | 112 | 0.01335347 | 2792473 | 0.0150 |
| 193 | 36 | 0.00431000 | 312039 | 0.0024 | 2 | 0.00027820 | 1731575 | 0.0093 |
| 194 | 59 | 0.00702371 | 397711 | 0.0031 | 103 | 0.01232017 | 3561761 | 0.0191 |
| 195 | 22 | 0.00267380 | 770128 | 0.0060 | 0 | 0.00000000 | 0 | 0.0000 |
| 196 | 77 | 0.00925852 | 1631067 | 0.0127 | 0 | 0.00000000 | 0 | 0.0000 |
| 197 | 129 | 0.01540426 | 997629 | 0.0078 | 98 | 0.01164454 | 4138261 | 0.0222 |
| 198 | 2386 | 0.28565728 | 20866647 | 0.1625 | 2594 | 0.30931563 | 61161468 | 0.3280 |
| 199 | 172 | 0.02063213 | 625299 | 0.0049 | 133 | 0.01589699 | 3578458 | 0.0192 |
| 200 | 8 | 0.00099769 | 394948 | 0.0031 | 530 | 0.06319053 | 16678849 | 0.0894 |
| 201 | 17 | 0.00207519 | 0 | 0.0000 | 53 | 0.00627931 | 2270954 | 0.0122 |
| 202 | 11 | 0.00127704 | 0 | 0.0000 | 36 | 0.00433193 | 2287281 | 0.0123 |
| 203 | 2 | 0.00023944 | 0 | 0.0000 | 0 | 0.00000000 | 0 | 0.0000 |
| 204 | 174 | 0.02087158 | 1451727 | 0.0113 | 0 | 0.00000000 | 0 | 0.0000 |
| 205 | 80 | 0.00961769 | 940367 | 0.0073 | 333 | 0.03970273 | 5586343 | 0.0300 |
| 206 | 11886 | 1.42305850 | 223646667 | 1.7415 | 10331 | 1.23173834 | 309900000 | 1.6618 |
| 207 | 545 | 0.06524862 | 5849381 | 0.0455 | 404 | 0.04820761 | 6738305 | 0.0361 |
| 208 | 10 | 0.00115732 | 315367 | 0.0025 | 35 | 0.00421270 | 3072013 | 0.0165 |
| 209 | 2473 | 0.29603320 | 80676667 | 0.6282 | 4209 | 0.50182815 | 143916667 | 0.7718 |
| 210 | 8 | 0.00091787 | 233687 | 0.0018 | 6 | 0.00071536 | 0 | 0.0000 |
| 211 | 1 | 0.00007981 | 1309298 | 0.0102 | 0 | 0.00000000 | 0 | 0.0000 |
| 212 | 0 | 0.00003991 | 0 | 0.0000 | 0 | 0.00000000 | 0 | 0.0000 |
| No treatment | 7653 | 0.91619443 | 26272707 | 0.2046 | 12050 | 1.43665050 | 74453588 | 0.3993 |
| AA | 15 | 0.00175593 | 0 | 0.0000 | 210 | 0.02503776 | 3777945 | 0.0203 |
| AA/3a | 8353 | 1.00000000 | 128424304 | 1.0000 | 8387 | 1.00000000 | 186480000 | 1.0000 |
| 169 | 7319 | 0.91843393 | 387695000 | 1.0342 | 5436 | 1.07644321 | 437495000 | 0.9520 |
| 185 | 6620 | 0.83065629 | 333205000 | 0.8889 | 4767 | 0.94395485 | 397435000 | 0.8649 |
| 167 | 6217 | 0.78014807 | 337920000 | 0.9014 | 5013 | 0.99277156 | 437235000 | 0.9515 |
| reference compound | 5934 | 0.74463546 | 363935000 | 0.9708 | 4122 | 0.81621943 | 348135000 | 0.7576 |
| 47 | 10447 | 1.31089221 | 382680000 | 1.0208 | 6908 | 1.36805624 | 560475000 | 1.2196 |
| 2 | 10963 | 1.37570586 | 296920000 | 0.7921 | 5679 | 1.12456679 | 463525000 | 1.0087 |
| 3 | 1766 | 0.22160873 | 162790000 | 0.4343 | 2184 | 0.43241905 | 189875000 | 0.4132 |
| 4 | 2914 | 0.36566696 | 230965000 | 0.6161 | 2776 | 0.54975740 | 125125000 | 0.2723 |
| 5 | 3600 | 0.45175053 | 276080000 | 0.7365 | 4121 | 0.81612041 | 294665000 | 0.6412 |
| 5 | 1977 | 0.24808633 | 164760000 | 0.4395 | 2266 | 0.44865828 | 152060000 | 0.3309 |
| 6 | 9964.5 | 1.25040783 | 363855000 | 0.9706 | 9728 | 1.92642836 | 635655000 | 1.3832 |
| 7 | 2536.5 | 0.31829590 | 179185000 | 0.4780 | 2397 | 0.47460145 | 150600000 | 0.3277 |
| 8 | 5706.5 | 0.71608734 | 319930000 | 0.8534 | 5096 | 1.00920883 | 341360000 | 0.7428 |
| 10 | 4645.5 | 0.58294642 | 257295000 | 0.6864 | 4507 | 0.89256362 | 312605000 | 0.6803 |
| 11 | 2892.5 | 0.36296900 | 213165000 | 0.5686 | 3043 | 0.60253490 | 269570000 | 0.5866 |
| 12 | 2460.5 | 0.30875894 | 203350000 | 0.5425 | 2410 | 0.47727498 | 209795000 | 0.4565 |
| 13 | 4783 | 0.60020078 | 306085000 | 0.8165 | 4556 | 0.90226755 | 326475000 | 0.7104 |
| 14 | 6916.5 | 0.86792571 | 377885000 | 1.0080 | 4504 | 0.89196950 | 365090000 | 0.7945 |
| 14 | 7370.5 | 0.92489647 | 365075000 | 0.9739 | 5300 | 1.04950985 | 399265000 | 0.8688 |
| 15 | 10533 | 1.32174677 | 475250000 | 1.2678 | 5186 | 1.02693336 | 404710000 | 0.8807 |
| 16 | 3513 | 0.44083323 | 242750000 | 0.6476 | 2522 | 0.49945539 | 214575000 | 0.4669 |
| No Treatment | not done | | | | not done | | | |
| AA | not done | | | | not done | | | |
| AA/3a | 7969 | 1.00000000 | 374870000 | 1.0000 | 5050 | 1.00000000 | 459540000 | 1.0000 |
| 16 | 563 | 0.31250000 | 57351132 | 0.3295 | 1744 | 0.03386884 | 165365000 | 1.1010 |
| 17 | 158 | 0.08777778 | 14786632 | 0.0850 | 83 | 0.00161234 | 14201404 | 0.0946 |
| 18 | 3 | 0.00166667 | 0 | 0.0000 | 4 | 0.00007770 | 28439 | 0.0002 |
| 19 | 5 | 0.00277778 | 0 | 0.0000 | 10 | 0.00019426 | 0 | 0.0000 |
| 19 | 15 | 0.00805556 | 548982 | 0.0032 | 0 | 0.00000000 | 0 | 0.0000 |
| 20 | 24 | 0.01305556 | 689535 | 0.0040 | 11 | 0.00021368 | 0 | 0.0000 |
| 20 | 94 | 0.05194444 | 11142426 | 0.0640 | 12 | 0.00022340 | 1767033 | 0.0118 |
| 21 | 15 | 0.00805556 | 0 | 0.0000 | 21 | 0.00039823 | 4567590 | 0.0304 |
| 22 | 33 | 0.01805556 | 2188847 | 0.0126 | 69 | 0.00134038 | 13689421 | 0.0911 |
| 9 | 4 | 0.00194444 | 0 | 0.0000 | 3 | 0.00005828 | 291660 | 0.0019 |
| 23 | 88 | 0.04888889 | 7121122 | 0.0409 | 399 | 0.00774117 | 65100086 | 0.4335 |
| 24 | 11 | 0.00583333 | 1073763 | 0.0062 | 5 | 0.00008742 | 0 | 0.0000 |
| 25 | 8 | 0.00444444 | 0 | 0.0000 | 9 | 0.00016512 | 0 | 0.0000 |
| 26 | 109 | 0.06027778 | 15714170 | 0.0903 | 136 | 0.00263219 | 15725984 | 0.1047 |
| 27 | 5 | 0.00250000 | 125443 | 0.0007 | 5 | 0.00009713 | 0 | 0.0000 |
| 28 | 20 | 0.01083333 | 3135653 | 0.0180 | 8 | 0.00015541 | 0 | 0.0000 |
| 28 | 9 | 0.00472222 | 72387 | 0.0004 | 17 | 0.00033024 | 736311 | 0.0049 |
| 29 | 6 | 0.00305556 | 644015 | 0.0037 | 4 | 0.00007770 | 0 | 0.0000 |
| 30 | 77 | 0.04277778 | 12632849 | 0.0726 | 28 | 0.00054392 | 9312311 | 0.0620 |
| 31 | 14 | 0.00750000 | 887585 | 0.0051 | 1 | 0.00001943 | 52047 | 0.0003 |
| 32 | 23 | 0.01277778 | 2117429 | 0.0122 | 13 | 0.00024282 | 0 | 0.0000 |
| No Treatment | not done | | | | 432 | 0.00838222 | 42987388 | 0.2862 |
| AA | 147 | 0.08138889 | 20330009 | 0.1168 | 8 | 0.00014569 | 87206 | 0.0006 |
| AA/3a | 1800 | 1.00000000 | 174052346 | 1.0000 | 1478 | 0.02870158 | 150190000 | 1.0000 |

TABLE V
EFFECTS OF INHIBITORS OF GSK-3B ENZYME ACTIVITY ON THE DIFFERENTIATION
AND PROLIFERATION OF HUMAN EMBRYONIC STEM CELLS.

| | Fold over | | Fold over | | Fold over |
|---|---|---|---|---|---|
| | Wnt 3a/AA | | Wnt 3a/AA | | Wnt 3a/AA |
| Compound # | control | Compound # | control | Compound # | control |
| Proliferative Response—Strong Hits | | SOX 17 Strong Hits | | HNF3β Expression—Strong Hits | |
| 165 | 5.8846 | 206 | 1.7415 | 165 | 5.4668 |
| 163 | 4.3268 | 15 | 1.2678 | 163 | 3.1115 |
| 181 | 2.5632 | SOX17 Expression—Moderate Hits | | 181 | 2.7680 |
| 6 | 1.9264 | 230 | 1.1359 | 206 | 1.6618 |
| 206 | 1.4231 | 221 | 1.1205 | 6 | 1.3832 |
| 2 | 1.3757 | 232 | 1.0749 | 47 | 1.2196 |
| 221 | 1.3754 | 165 | 1.0630 | HNF3β Expression—Moderate Hits | |
| 47 | 1.3681 | 223 | 1.0419 | 221 | 1.1562 |
| 15 | 1.3217 | 169 | 1.0342 | 16 | 1.1010 |
| 230 | 1.3107 | 47 | 1.0208 | 2 | 1.0087 |
| Proliferative Response—Moderate Hits | | 14 | 1.0080 | 169 | 0.9520 |
| 2 | 1.1246 | reference cmpd | 0.9708 | 167 | 0.9515 |
| 232 | 1.0870 | 6 | 0.9706 | 230 | 0.9499 |
| 169 | 1.0764 | 163 | 0.9266 | 15 | 0.8807 |
| 230 | 1.0736 | 167 | 0.9014 | 14 | 0.8688 |
| 14 | 1.0495 | 185 | 0.8889 | 185 | 0.8649 |
| 15 | 1.0269 | 8 | 0.8534 | 209 | 0.7718 |
| 8 | 1.0092 | 13 | 0.8165 | 232 | 0.7711 |
| 167 | 0.9928 | 231 | 0.8113 | reference cmpd | 0.7576 |
| 223 | 0.9484 | 181 | 0.7926 | 8 | 0.7428 |
| 185 | 0.9440 | SOX 17 Moderate Hits | | 13 | 0.7104 |
| 14 | 0.9249 | 2 | 0.7921 | 10 | 0.6803 |
| 13 | 0.9023 | 5 | 0.7365 | 5 | 0.6412 |
| 231 | 0.9001 | 148 | 0.7189 | 11 | 0.5866 |
| 10 | 0.8926 | 10 | 0.6864 | | |
| 179 | 0.8514 | 16 | 0.6476 | | |
| reference cmpd | 0.8162 | 209 | 0.6282 | | |
| 5 | 0.8161 | 4 | 0.6161 | | |
| 148 | 0.7992 | 226 | 0.5887 | | |
| 163 | 0.7864 | 11 | 0.5686 | | |
| 181 | 0.7842 | | | | |
| 186 | 0.7277 | | | | |
| 226 | 0.6342 | | | | |
| 166 | 0.6211 | | | | |
| 11 | 0.6025 | | | | |

TABLE VI
EFFECTS OF INHIBITORS OF GSK-3B ENZYME ACTIVITY ON
THE PROLIFERATION OF HUMAN EMBRYONIC STEM CELLS.

| Compound # | Raw Data | | | Average | S.D. | % CV | % Control |
|---|---|---|---|---|---|---|---|
| conditioned medium | 1.1348 | 1.0099 | 1.1092 | 1.0846 | 0.0660 | 6.1 | 116.5 |
| no treatment | 0.9344 | 0.5977 | 0.8454 | 0.7925 | 0.1745 | 22.0 | 85.2 |
| AA/DMSO | 0.3878 | 0.2434 | 0.2252 | 0.2855 | 0.0891 | 31.2 | 30.7 |
| AA/Wnt3a/DMSO | 0.6098 | 1.0804 | 0.7635 | 0.8179 | 0.2403 | 25.8 | 100.0 |
| 161 | 0.3418 | 0.4276 | 0.5751 | 0.4482 | 0.1180 | 26.3 | 48.2 |
| 162 | 0.1362 | 0.1531 | 0.1532 | 0.1475 | 0.0098 | 6.6 | 15.8 |
| 163 | 1.3764 | 1.2753 | 1.3208 | 1.3242 | 0.0506 | 3.8 | 142.3 |
| 164 | 0.6923 | 0.5994 | 0.6134 | 0.6350 | 0.0501 | 7.9 | 68.2 |
| 165 | 1.7896 | 1.4721 | 2.1908 | 1.8175 | 0.3602 | 19.8 | 195.3 |
| 166 | 1.7591 | 1.6274 | 1.6518 | 1.6794 | 0.0701 | 4.2 | 180.4 |
| 168 | 0.3702 | 0.3193 | 0.3368 | 0.3421 | 0.0259 | 7.6 | 36.8 |
| 169 | 0.5876 | 0.6384 | 0.9154 | 0.7138 | 0.1764 | 24.7 | 76.7 |
| 170 | 0.3074 | 0.2328 | 0.2920 | 0.2774 | 0.0394 | 14.2 | 29.8 |
| 171 | 0.1311 | 0.1245 | 0.1288 | 0.1281 | 0.0034 | 2.6 | 13.8 |
| 172 | 0.1270 | 0.2778 | 0.1916 | 0.1988 | 0.0757 | 38.1 | 21.4 |
| 173 | 0.2166 | 0.3062 | 0.2915 | 0.2714 | 0.0481 | 17.7 | 29.2 |
| 174 | 0.4362 | 0.3728 | 0.2481 | 0.3524 | 0.0957 | 27.2 | 37.9 |
| 175 | 0.1560 | 0.1481 | 0.1359 | 0.1467 | 0.0101 | 6.9 | 15.8 |
| 176 | 0.2932 | 0.3883 | 0.6258 | 0.4358 | 0.1713 | 39.3 | 46.8 |
| 177 | 0.1362 | 0.1479 | 0.1298 | 0.1380 | 0.0092 | 6.7 | 14.8 |
| 178 | 0.2198 | 0.2159 | 0.2300 | 0.2219 | 0.0073 | 3.3 | 23.8 |
| 179 | 0.7624 | 0.2705 | 0.2478 | 0.4269 | 0.2908 | 68.1 | 45.9 |
| 180 | 0.1239 | 0.1233 | 0.1269 | 0.1247 | 0.0019 | 1.5 | 13.4 |
| 181 | 0.1277 | 0.1254 | 0.6980 | 0.3170 | 0.3299 | 104.1 | 34.1 |
| 182 | 0.2665 | 0.3215 | 0.2605 | 0.2828 | 0.0336 | 11.9 | 30.4 |
| 183 | 0.2395 | 0.3235 | 0.1333 | 0.2321 | 0.0953 | 41.1 | 24.9 |
| 184 | 0.2646 | 0.1873 | 0.1293 | 0.1937 | 0.0679 | 35.0 | 20.8 |
| 185 | 0.3590 | 0.2790 | 0.1515 | 0.2632 | 0.1047 | 39.8 | 28.3 |
| 186 | 0.4690 | 0.5805 | 0.3349 | 0.4615 | 0.1230 | 26.6 | 49.6 |
| conditioned medium | 1.1525 | 1.1269 | 1.1140 | 1.1311 | 0.0196 | 1.7 | 71.0 |
| no treatment | 1.2057 | 1.2358 | 1.3132 | 1.2516 | 0.0555 | 4.4 | 78.6 |
| AA/DMSO | 0.2622 | 0.2073 | 0.2830 | 0.2508 | 0.0391 | 15.6 | 15.8 |
| AA/Wnt3a/DMSO | 1.3943 | 1.7976 | 1.8000 | 1.5922 | 0.2136 | 13.4 | 100.0 |
| 187 | 0.1930 | 0.2223 | 0.2167 | 0.2107 | 0.0156 | 7.4 | 13.2 |
| 188 | 0.1757 | 0.1813 | 0.1835 | 0.1802 | 0.0040 | 2.2 | 11.3 |
| 189 | 0.1473 | 0.1880 | 0.1732 | 0.1695 | 0.0206 | 12.2 | 10.6 |
| 190 | 0.1330 | 0.1362 | 0.1867 | 0.1520 | 0.0301 | 19.8 | 9.5 |
| 191 | 0.8191 | 0.5493 | 0.6526 | 0.6737 | 0.1361 | 20.2 | 42.3 |
| 192 | 0.4008 | 0.2779 | 0.3869 | 0.3552 | 0.0673 | 18.9 | 22.3 |
| 193 | 0.1220 | 0.1248 | 0.1251 | 0.1240 | 0.0017 | 1.4 | 7.8 |
| 194 | 0.2883 | 0.3308 | 0.5503 | 0.3898 | 0.1406 | 36.1 | 24.5 |
| 195 | 0.2835 | 0.4024 | 0.5698 | 0.4186 | 0.1438 | 34.4 | 26.3 |
| 196 | 0.3704 | 0.6073 | 0.5280 | 0.5019 | 0.1206 | 24.0 | 31.5 |
| 197 | 0.2266 | 0.1815 | 0.2289 | 0.2123 | 0.0267 | 12.6 | 13.3 |
| 198 | 1.0820 | 1.1862 | 1.1076 | 1.1253 | 0.0543 | 4.8 | 70.7 |
| 199 | 0.3590 | 0.5457 | 0.6123 | 0.5057 | 0.1313 | 26.0 | 31.8 |
| 200 | 0.2198 | 0.3564 | 0.3202 | 0.2988 | 0.0708 | 23.7 | 18.8 |
| 201 | 0.2928 | 0.2920 | 0.3659 | 0.3169 | 0.0424 | 13.4 | 19.9 |
| 202 | 0.3349 | 0.3013 | 0.3507 | 0.3290 | 0.0252 | 7.7 | 20.7 |
| 203 | 0.1852 | 0.1924 | 0.2349 | 0.2042 | 0.0269 | 13.2 | 12.8 |
| 204 | 0.2170 | 0.3003 | 0.1877 | 0.2350 | 0.0584 | 24.9 | 14.8 |
| 205 | 0.3094 | 0.2515 | 0.1881 | 0.2497 | 0.0607 | 24.3 | 15.7 |
| 206 | 1.8452 | 1.7710 | 1.5591 | 1.7251 | 0.1485 | 8.6 | 108.3 |
| 207 | 0.7305 | 0.7067 | 0.6250 | 0.6874 | 0.0553 | 8.0 | 43.2 |
| 208 | 0.2113 | 0.1800 | 0.1547 | 0.1820 | 0.0284 | 15.6 | 11.4 |
| 209 | 1.5225 | 1.5912 | 0.1081 | 1.0739 | 0.8371 | 78.0 | 67.4 |
| 210 | 0.4006 | 1.2807 | 0.1162 | 0.5992 | 0.6071 | 101.3 | 37.6 |
| 211 | 0.1972 | 0.1839 | 0.1162 | 0.1658 | 0.0434 | 26.2 | 10.4 |
| 212 | 0.1351 | 0.1318 | 0.1169 | 0.1279 | 0.0097 | 7.6 | 8.0 |

| | | | | | | |
|---|---|---|---|---|---|---|
| conditioned medium | 1.0568 | 1.0604 | 1.0586 | 0.0025 | 0.2 | 71.9 |
| no treatment | 1.1544 | 0.9576 | 1.0560 | 0.1392 | 13.2 | 71.7 |
| AA only + DMSO | 0.6329 | 0.8434 | 0.7382 | 0.1488 | 20.2 | 47.1 |
| AA + Wnt3a + DMSO | 1.2704 | 1.8669 | 1.4229 | 0.2960 | 20.8 | 100.0 |
| 213 | 0.5617 | 0.2098 | 0.3858 | 0.2488 | 64.5 | 19.9 |
| 214 | 0.6850 | 0.5853 | 0.6352 | 0.0705 | 11.1 | 39.2 |
| 214 | 0.7496 | 0.9187 | 0.8342 | 0.1196 | 14.3 | 54.5 |
| 215 | 0.2320 | 0.2124 | 0.2222 | 0.0139 | 6.2 | 7.3 |
| 216 | 0.8079 | 1.4391 | 1.1235 | 0.4463 | 39.7 | 76.9 |
| 217 | 0.8310 | 0.7318 | 0.7814 | 0.0701 | 9.0 | 50.5 |
| 218 | 1.0646 | 1.1384 | 1.1015 | 0.0522 | 4.7 | 75.2 |
| 219 | 0.6344 | 1.0400 | 0.8372 | 0.2868 | 34.3 | 54.8 |
| no cells | 0.1335 | 0.2070 | 0.1703 | 0.0520 | 30.5 | 3.3 |
| 220 | 0.8643 | 0.4060 | 0.6352 | 0.3241 | 51.0 | 39.2 |
| 221 | 1.7922 | 1.8533 | 1.8228 | 0.0432 | 2.4 | 130.9 |
| 222 | 0.1914 | 0.2371 | 0.2143 | 0.0323 | 15.1 | 6.7 |
| 223 | 1.8401 | 1.7563 | 1.7982 | 0.0593 | 3.3 | 129.0 |
| 224 | 1.0301 | 1.0356 | 1.0329 | 0.0039 | 0.4 | 69.9 |
| 225 | 0.1306 | 0.1338 | 0.1322 | 0.0023 | 1.7 | 0.3 |
| 226 | 1.7143 | 1.6506 | 1.6825 | 0.0450 | 2.7 | 120.0 |
| 227 | 0.4170 | 0.4956 | 0.4563 | 0.0556 | 12.2 | 25.4 |
| 228 | 0.1772 | 0.2348 | 0.2060 | 0.0407 | 19.8 | 6.0 |
| 229 | 1.0231 | 1.2392 | 1.1312 | 0.1528 | 13.5 | 77.5 |
| 230 | 1.9718 | 2.0997 | 2.0358 | 0.0904 | 4.4 | 147.3 |
| 231 | 1.5168 | 1.6872 | 1.6020 | 0.1205 | 7.5 | 113.8 |
| 232 | 1.6935 | 1.9710 | 1.8323 | 0.1962 | 10.7 | 131.6 |
| 158 | 1.2655 | 1.1829 | 1.2242 | 0.0584 | 4.8 | 84.7 |
| 233 | 1.3481 | 1.3168 | 1.3325 | 0.0221 | 1.7 | 93.0 |
| 142 | 0.6444 | 0.7239 | 0.6842 | 0.0562 | 8.2 | 43.0 |
| 143 | 0.2046 | 0.3076 | 0.2561 | 0.0728 | 28.4 | 9.9 |
| 144 | 1.3627 | 1.0693 | 1.2160 | 0.2075 | 17.1 | 84.0 |
| 145 | 0.8722 | 0.9660 | 0.9191 | 0.0663 | 7.2 | 61.1 |
| 146 | 1.0332 | 0.4554 | 0.7443 | 0.4086 | 54.9 | 47.6 |
| 147 | 0.8775 | 0.7347 | 0.8061 | 0.1010 | 12.5 | 52.4 |
| 148 | 1.7865 | 1.2008 | 1.4937 | 0.4142 | 27.7 | 105.5 |
| 149 | 0.2396 | 0.1584 | 0.1990 | 0.0574 | 28.9 | 5.5 |
| 150 | 0.8122 | 1.0827 | 0.9475 | 0.1913 | 20.2 | 63.3 |
| 151 | 0.1342 | 0.1363 | 0.1353 | 0.0015 | 1.1 | 0.6 |
| 152 | 1.0462 | 0.5838 | 0.8150 | 0.3270 | 40.1 | 53.1 |
| 153 | 0.4586 | 0.2903 | 0.3745 | 0.1190 | 31.8 | 19.0 |
| 70 | 0.1277 | 0.1402 | 0.1340 | 0.0088 | 6.6 | 0.5 |
| 154 | 0.1258 | 0.1324 | 0.1291 | 0.0047 | 3.6 | 0.1 |
| 155 | 0.1219 | 0.1216 | 0.1218 | 0.0002 | 0.2 | −0.5 |
| 156 | 0.4223 | 0.4721 | 0.4472 | 0.0352 | 7.9 | 24.7 |
| 157 | 0.1514 | 0.1396 | 0.1455 | 0.0083 | 5.7 | 1.4 |
| conditioned medium | 0.7423 | 0.7081 | 0.7252 | 0.0242 | 3.3 | 87.7 |
| no treatment | 0.4936 | 0.5689 | 0.5313 | 0.0532 | 10.0 | 59.8 |
| AA only + DMSO | 0.1433 | 0.1939 | 0.1686 | 0.0358 | 21.2 | 7.6 |
| AA + Wnt3a + DMSO | 0.6808 | 0.9406 | 0.8107 | 0.1837 | 22.7 | 100.0 |
| 33 | 0.2447 | 0.1331 | 0.1889 | 0.0789 | 41.8 | 10.6 |
| 34 | 0.1537 | 0.1302 | 0.1420 | 0.0166 | 11.7 | 3.8 |
| no cells | 0.1163 | 0.1147 | 0.1155 | 0.0011 | 1.0 | 0.0 |
| 35 | 0.2994 | 0.2592 | 0.2793 | 0.0284 | 10.2 | 23.6 |
| 36 | 0.1353 | 0.2121 | 0.1737 | 0.0543 | 31.3 | 8.4 |
| 1 | 0.1267 | 0.1419 | 0.1343 | 0.0107 | 8.0 | 2.7 |
| 37 | 0.1376 | 0.1676 | 0.1526 | 0.0212 | 13.9 | 5.3 |
| 38 | 0.1134 | 0.1103 | 0.1119 | 0.0022 | 2.0 | −0.5 |
| 39 | 0.1318 | 0.1478 | 0.1398 | 0.0113 | 8.1 | 3.5 |
| 40 | 0.2569 | 0.2124 | 0.2347 | 0.0315 | 13.4 | 17.1 |
| 41 | 0.2674 | 0.2636 | 0.2655 | 0.0027 | 1.0 | 21.6 |
| 42 | 0.4357 | 0.3467 | 0.3912 | 0.0629 | 16.1 | 39.7 |
| 43 | 0.1265 | 0.1588 | 0.1427 | 0.0228 | 16.0 | 3.9 |
| 44 | 0.1662 | 0.2521 | 0.2092 | 0.0607 | 29.0 | 13.5 |
| 45 | 0.1596 | 0.1566 | 0.1581 | 0.0021 | 1.3 | 6.1 |
| 46 | 0.2725 | 0.1636 | 0.2181 | 0.0770 | 35.3 | 14.8 |
| 48 | 1.2256 | 1.0636 | 1.1446 | 0.1146 | 10.0 | 148.0 |
| 48 | 0.1134 | 0.1070 | 0.1102 | 0.0045 | 4.1 | −0.8 |
| 49 | 0.1469 | 0.1495 | 0.1482 | 0.0018 | 1.2 | 4.7 |
| 50 | 0.1169 | 0.1122 | 0.1146 | 0.0033 | 2.9 | −0.1 |
| 51 | 0.1595 | 0.1422 | 0.1509 | 0.0122 | 8.1 | 5.1 |
| 52 | 1.0484 | 1.0749 | 1.0617 | 0.0187 | 1.8 | 136.1 |
| 53 | 0.3012 | 0.2347 | 0.2680 | 0.0470 | 17.5 | 21.9 |
| 54 | 0.1267 | 0.1510 | 0.1389 | 0.0172 | 12.4 | 3.4 |
| 55 | 1.1902 | 1.1487 | 1.1695 | 0.0293 | 2.5 | 151.6 |
| 56 | 0.6400 | 0.7076 | 0.6738 | 0.0478 | 7.1 | 80.3 |
| 57 | 0.1701 | 0.1752 | 0.1727 | 0.0036 | 2.1 | 8.2 |
| 58 | 0.3435 | 0.3488 | 0.3462 | 0.0037 | 1.1 | 33.2 |
| 59 | 0.4032 | 0.3548 | 0.3790 | 0.0342 | 9.0 | 37.9 |
| 60 | 0.1602 | 0.1502 | 0.1552 | 0.0071 | 4.6 | 5.7 |
| 61 | 0.1604 | 0.2079 | 0.1842 | 0.0336 | 18.2 | 9.9 |
| 62 | 0.1646 | 0.1592 | 0.1619 | 0.0038 | 2.4 | 6.7 |
| 63 | 0.1779 | 0.2273 | 0.2026 | 0.0349 | 17.2 | 12.5 |
| 64 | 0.1225 | 0.1443 | 0.1334 | 0.0154 | 11.6 | 2.6 |
| 65 | 0.1300 | 0.1291 | 0.1296 | 0.0006 | 0.5 | 2.0 |
| 66 | 0.1263 | 0.1336 | 0.1300 | 0.0052 | 4.0 | 2.1 |
| 67 | 0.2778 | 0.1326 | 0.2052 | 0.1027 | 50.0 | 12.9 |
| 68 | 0.2569 | 0.1219 | 0.1894 | 0.0955 | 50.4 | 10.6 |
| 69 | 0.1640 | 0.1158 | 0.1399 | 0.0341 | 24.4 | 3.5 |
| 74 | 1.1486 | 0.8970 | 1.0228 | 0.1779 | 17.4 | 130.5 |
| 74 | 0.1358 | 0.1201 | 0.1280 | 0.0111 | 8.7 | 1.8 |
| 71 | 0.1257 | 0.1257 | 0.1257 | 0.0000 | 0.0 | 1.5 |
| 72 | 0.4676 | 0.4803 | 0.4740 | 0.0090 | 1.9 | 51.6 |
| conditioned medium | 0.6935 | 0.7803 | 0.7369 | 0.0614 | 8.3 | 104.8 |
| no treatment | 0.4735 | 0.6069 | 0.5402 | 0.0943 | 17.5 | 71.5 |
| AA only + DMSO | 0.1428 | 0.1656 | 0.1542 | 0.0161 | 10.5 | 6.3 |
| AA + Wnt3a + DMSO | 0.5702 | 0.8468 | 0.7085 | 0.1956 | 27.6 | 100.0 |
| 73 | 0.1599 | 0.2380 | 0.1990 | 0.0552 | 27.8 | 13.8 |
| 76 | 0.1287 | 0.1244 | 0.1266 | 0.0030 | 2.4 | 1.6 |
| no cells | 0.1241 | 0.1100 | 0.1171 | 0.0100 | 8.5 | 0.0 |
| 75 | 0.1235 | 0.1152 | 0.1194 | 0.0059 | 4.9 | 0.4 |
| 77 | 0.1199 | 0.1278 | 0.1239 | 0.0056 | 4.5 | 1.1 |
| 77 | 0.1174 | 0.1162 | 0.1168 | 0.0008 | 0.7 | −0.1 |
| 78 | 1.1100 | 0.9464 | 1.0282 | 0.1157 | 11.3 | 154.1 |
| 79 | 0.1247 | 0.1115 | 0.1181 | 0.0093 | 7.9 | 0.2 |
| 80 | 0.2640 | 0.1688 | 0.2164 | 0.0673 | 31.1 | 16.8 |
| 81 | 0.2313 | 0.1307 | 0.1810 | 0.0711 | 39.3 | 10.8 |
| 82 | 0.8639 | 0.9218 | 0.8929 | 0.0409 | 4.6 | 131.2 |
| 83 | 0.2540 | 0.2320 | 0.2430 | 0.0156 | 6.4 | 21.3 |
| 84 | 0.1809 | 0.3077 | 0.2443 | 0.0897 | 36.7 | 21.5 |
| 85 | 0.1892 | 0.1872 | 0.1882 | 0.0014 | 0.8 | 12.0 |
| 86 | 0.1967 | 0.2492 | 0.2230 | 0.0371 | 16.7 | 17.9 |
| 87 | 0.3346 | 0.1619 | 0.2483 | 0.1221 | 49.2 | 22.2 |
| 88 | 0.1106 | 0.1138 | 0.1122 | 0.0023 | 2.0 | −0.8 |
| 89 | 0.1224 | 0.1445 | 0.1335 | 0.0156 | 11.7 | 2.8 |
| 90 | 0.1312 | 0.1270 | 0.1291 | 0.0030 | 2.3 | 2.0 |
| 91 | 0.1653 | 0.2114 | 0.1884 | 0.0326 | 17.3 | 12.0 |
| 92 | 0.1732 | 0.1467 | 0.1600 | 0.0187 | 11.7 | 7.2 |
| 93 | 0.1618 | 0.2754 | 0.2186 | 0.0803 | 36.7 | 17.2 |
| 94 | 1.0006 | 0.9631 | 0.9819 | 0.0265 | 2.7 | 146.2 |
| 95 | 0.6472 | 0.4319 | 0.5396 | 0.1522 | 28.2 | 71.4 |
| 96 | 0.1539 | 0.1469 | 0.1504 | 0.0049 | 3.3 | 5.6 |
| 97 | 0.1127 | 0.1309 | 0.1218 | 0.0129 | 10.6 | 0.8 |
| 98 | 0.6887 | 0.5860 | 0.6374 | 0.0726 | 11.4 | 88.0 |
| 99 | 0.1141 | 0.1094 | 0.1118 | 0.0033 | 3.0 | −0.9 |
| 100 | 0.2774 | 0.1690 | 0.2232 | 0.0767 | 34.3 | 17.9 |
| 101 | 0.9482 | 1.1150 | 1.0316 | 0.1179 | 11.4 | 154.6 |
| 102 | 0.7687 | 0.6804 | 0.7246 | 0.0624 | 8.6 | 102.7 |
| 103 | 0.7125 | 0.3347 | 0.5236 | 0.2671 | 51.0 | 68.7 |
| 104 | 0.1446 | 0.1221 | 0.1334 | 0.0159 | 11.9 | 2.7 |
| 105 | 1.0968 | 1.3108 | 1.2038 | 0.1513 | 12.6 | 183.8 |
| 106 | 0.3167 | 0.3415 | 0.3291 | 0.0175 | 5.3 | 35.8 |
| 107 | 0.1261 | 0.1144 | 0.1203 | 0.0083 | 6.9 | 0.5 |
| 108 | 0.2223 | 0.2930 | 0.2577 | 0.0500 | 19.4 | 23.8 |
| 109 | 0.1265 | 0.1236 | 0.1251 | 0.0021 | 1.6 | 1.3 |
| 110 | 1.1940 | 0.9431 | 1.0686 | 0.1774 | 16.6 | 160.9 |
| 111 | 1.0689 | 0.6879 | 0.8784 | 0.2694 | 30.7 | 128.7 |
| 112 | 1.0444 | 0.7603 | 0.9024 | 0.2009 | 22.3 | 132.8 |
| 113 | 0.1443 | 0.1209 | 0.1326 | 0.0165 | 12.5 | 2.6 |
| 114 | 0.1152 | 0.1309 | 0.1231 | 0.0111 | 9.0 | 1.0 |
| conditioned medium | 0.7590 | 0.7451 | 0.7521 | 0.0098 | 1.3 | 98.0 |
| no treatment | 0.5687 | 0.4490 | 0.5089 | 0.0846 | 16.6 | 60.4 |
| AA only + DMSO | 0.1988 | 0.1522 | 0.1755 | 0.0330 | 18.8 | 8.9 |
| AA + Wnt3a + DMSO | 0.6837 | 0.8460 | 0.7649 | 0.1148 | 15.0 | 100.0 |
| 115 | 0.1911 | 0.1101 | 0.1506 | 0.0573 | 38.0 | 5.0 |
| 116 | 0.2772 | 0.1151 | 0.1962 | 0.1146 | 58.4 | 12.1 |
| no cells | 0.1278 | 0.1084 | 0.1181 | 0.0137 | 11.6 | 0.0 |
| 117 | 0.1443 | 0.2120 | 0.1782 | 0.0479 | 26.9 | 9.3 |
| 118 | 0.4413 | 0.2238 | 0.3326 | 0.1538 | 46.2 | 33.2 |
| 119 | 0.1098 | 0.1085 | 0.1092 | 0.0009 | 0.8 | −1.4 |
| 120 | 0.1389 | 0.2147 | 0.1768 | 0.0536 | 30.3 | 9.1 |
| 121 | 0.1852 | 0.1342 | 0.1597 | 0.0361 | 22.6 | 6.4 |
| 122 | 0.1114 | 0.1295 | 0.1205 | 0.0128 | 10.6 | 0.4 |
| 123 | 0.5375 | 0.6158 | 0.5767 | 0.0554 | 9.6 | 70.9 |
| 124 | 0.1259 | 0.1441 | 0.1350 | 0.0129 | 9.5 | 2.6 |
| 125 | 0.1206 | 0.1312 | 0.1259 | 0.0075 | 6.0 | 1.2 |
| 126 | 0.2269 | 0.2857 | 0.2563 | 0.0416 | 16.2 | 21.4 |
| 126 | 0.1140 | 0.1079 | 0.1110 | 0.0043 | 3.9 | −1.1 |
| 127 | 0.9589 | 0.8868 | 0.9229 | 0.0510 | 5.5 | 124.4 |
| 127 | 1.0442 | 0.9622 | 1.0032 | 0.0580 | 5.8 | 136.8 |
| 128 | 0.1961 | 0.1735 | 0.1848 | 0.0160 | 8.6 | 10.3 |
| 129 | 0.5732 | 0.5216 | 0.5474 | 0.0365 | 6.7 | 66.4 |
| 130 | 0.1273 | 0.1217 | 0.1245 | 0.0040 | 3.2 | 1.0 |
| 131 | 0.5932 | 0.6671 | 0.6302 | 0.0523 | 8.3 | 79.2 |
| 132 | 0.1444 | 0.1368 | 0.1406 | 0.0054 | 3.8 | 3.5 |
| 133 | 1.0786 | 1.0891 | 1.0839 | 0.0074 | 0.7 | 149.3 |
| 138 | 0.5418 | 0.2338 | 0.3878 | 0.2178 | 56.2 | 41.7 |
| 134 | 0.1268 | 0.2052 | 0.1660 | 0.0554 | 33.4 | 7.4 |
| 135 | 0.1169 | 0.1184 | 0.1177 | 0.0011 | 0.9 | −0.1 |
| 136 | 0.8618 | 1.0400 | 0.9509 | 0.1260 | 13.3 | 128.8 |
| 136 | 0.8430 | 1.0187 | 0.9309 | 0.1242 | 13.3 | 125.7 |
| 137 | 0.3659 | 0.3168 | 0.3414 | 0.0347 | 10.2 | 34.5 |
| 139 | 0.9184 | 0.8116 | 0.8650 | 0.0755 | 8.7 | 115.5 |
| 140 | 0.2384 | 0.3156 | 0.2770 | 0.0546 | 19.7 | 24.6 |
| 141 | 0.2297 | 0.1469 | 0.1883 | 0.0585 | 31.1 | 10.9 |
| 159 | 0.1955 | 0.1256 | 0.1606 | 0.0494 | 30.8 | 6.6 |
| 234 | 0.1658 | 0.1704 | 0.1681 | 0.0033 | 1.9 | 7.7 |
| 235 | 0.1399 | 0.1303 | 0.1351 | 0.0068 | 5.0 | 2.6 |
| 236 | 0.1234 | 0.1236 | 0.1235 | 0.0001 | 0.1 | 0.8 |
| 237 | 0.1397 | 0.2147 | 0.1772 | 0.0530 | 29.9 | 9.1 |
| 238 | 0.1218 | 0.1310 | 0.1264 | 0.0065 | 5.1 | 1.3 |
| 239 | 0.1456 | 0.1981 | 0.1719 | 0.0371 | 21.6 | 8.3 |
| 240 | 0.5412 | 0.1898 | 0.3655 | 0.2485 | 68.0 | 38.2 |
| 241 | 0.1996 | 0.1245 | 0.1621 | 0.0531 | 32.8 | 6.8 |
| 241 | 0.1418 | 0.2014 | 0.1716 | 0.0421 | 24.6 | 8.3 |
| 242 | 0.1106 | 0.1197 | 0.1152 | 0.0064 | 5.6 | −0.5 |
| 243 | 0.1159 | 0.1272 | 0.1216 | 0.0080 | 6.6 | 0.5 |
| conditioned medium | 0.8077 | 0.7210 | 0.7644 | 0.0613 | 8.0 | 74.7 |
| no treatment + DMSO | 0.4638 | 0.4073 | 0.4356 | 0.0400 | 9.2 | 36.7 |
| AA/Wnt3a | 0.8466 | 0.9935 | 0.9830 | 0.2592 | 26.4 | 100.0 |
| 16 | 0.8095 | 0.9055 | 0.8575 | 0.0679 | 7.9 | 85.5 |
| 17 | 0.3519 | 0.4708 | 0.4114 | 0.0841 | 20.4 | 33.9 |
| 18 | 0.1609 | 0.1275 | 0.1442 | 0.0236 | 16.4 | 3.1 |
| 19 | 0.5020 | 0.2733 | 0.3877 | 0.1617 | 41.7 | 31.2 |
| 19 | 0.3413 | 0.4146 | 0.3780 | 0.0518 | 13.7 | 30.1 |
| 20 | 0.1176 | 0.1174 | 0.1175 | 0.0001 | 0.1 | 0.0 |
| 20 | 0.1148 | 0.1410 | 0.1279 | 0.0185 | 14.5 | 1.2 |
| 21 | 0.2394 | 0.2450 | 0.2422 | 0.0040 | 1.6 | 14.4 |
| 22 | 0.3672 | 0.3098 | 0.3385 | 0.0406 | 12.0 | 25.5 |
| 9 | 0.2722 | 0.1593 | 0.2158 | 0.0798 | 37.0 | 11.3 |
| 23 | 0.5079 | 0.4349 | 0.4714 | 0.0516 | 11.0 | 40.9 |
| 24 | 0.1076 | 0.1168 | 0.1122 | 0.0065 | 5.8 | −0.6 |
| 25 | 0.2569 | 0.2151 | 0.2360 | 0.0296 | 12.5 | 13.7 |
| 26 | 0.2846 | 0.4376 | 0.3611 | 0.1082 | 30.0 | 28.1 |
| 27 | 0.1168 | 0.1136 | 0.1152 | 0.0023 | 2.0 | −0.3 |
| 28 | 0.1168 | 0.1152 | 0.1160 | 0.0011 | 1.0 | −0.2 |
| 28 | 0.1137 | 0.1195 | 0.1166 | 0.0041 | 3.5 | −0.1 |
| 29 | 0.1154 | 0.1152 | 0.1153 | 0.0001 | 0.1 | −0.3 |
| 30 | 0.2188 | 0.2353 | 0.2271 | 0.0117 | 5.1 | 12.6 |
| 31 | 0.4588 | 0.2521 | 0.3555 | 0.1462 | 41.1 | 27.5 |
| 32 | 0.3081 | 0.1961 | 0.2521 | 0.0792 | 31.4 | 15.5 |
| conditioned medium | 0.7914 | 1.1189 | 0.9552 | 0.2316 | 24.2 | 93.3 |
| no treatment | 0.4215 | 0.5259 | 0.4737 | 0.0738 | 15.6 | 39.8 |
| no cells | 0.1152 | 0.1160 | 0.1156 | 0.0006 | 0.5 | 0.0 |
| AA/Wnt3a | 0.7168 | 0.8836 | 1.0151 | 0.2016 | 19.9 | 100.0 |
| 244 | 0.2882 | 0.2308 | 0.2844 | 0.0499 | 17.6 | 18.8 |
| 245 | 0.3049 | 0.2845 | 0.3127 | 0.0282 | 9.0 | 21.9 |
| 246 | 0.5403 | 0.2570 | 0.3855 | 0.1332 | 34.6 | 30.0 |
| 247 | 0.7323 | 0.3034 | 0.4388 | 0.2041 | 46.5 | 35.9 |
| 248 | 0.1185 | 0.1216 | 0.1199 | 0.0018 | 1.5 | 0.5 |
| 249 | 0.2496 | 0.2683 | 0.2302 | 0.0376 | 16.3 | 12.7 |
| 250 | 0.1548 | 0.1356 | 0.1513 | 0.0134 | 8.8 | 4.0 |
| 160 | 0.1555 | 0.1450 | 0.1581 | 0.0161 | 10.2 | 4.7 |
| 251 | 0.2347 | 0.1920 | 0.3785 | 0.2589 | 68.4 | 29.2 |
| 180 | 0.1842 | 0.2093 | 0.3793 | 0.2585 | 68.2 | 29.3 |
| 221 | 0.7223 | 0.8707 | 0.4291 | 0.2452 | 57.2 | 34.8 |
| 169 | 0.6268 | 0.3192 | 0.3354 | 0.1667 | 49.7 | 24.4 |

TABLE VII
EFFECTS OF INHIBITORS OF GSK-3B
ENZYME ACTIVITY ON THE PROLIFERATION
OF HUMAN EMBRYONIC STEM CELLS.

| List Strong Hits | | List Moderate Hits | |
|---|---|---|---|
| >=120% control | | 60-120% control | |
| Compound # | % Control Value | Compound # | % Control Value |
| 165 | 195.3 | 139 | 115.5 |
| 105 | 183.8 | 231 | 113.8 |
| 166 | 180.4 | 206 | 108.3 |
| 110 | 160.9 | 148 | 105.5 |
| 101 | 154.6 | 102 | 102.7 |
| 78 | 154.1 | 233 | 93.0 |
| 55 | 151.6 | 98 | 88.0 |
| 133 | 149.3 | 16 | 85.5 |
| 48 | 148.0 | 158 | 84.7 |
| 230 | 147.3 | 144 | 84.0 |
| 94 | 146.2 | 56 | 80.3 |
| 163 | 142.3 | 131 | 79.2 |
| 127 | 136.8 | 229 | 77.5 |
| 52 | 136.1 | 216 | 76.9 |
| 112 | 132.8 | 169 | 76.7 |
| 232 | 131.6 | 218 | 75.2 |
| 82 | 131.2 | 95 | 71.4 |
| 221 | 130.9 | 123 | 70.9 |
| 74 | 130.5 | 198 | 70.7 |
| 223 | 129.0 | 224 | 69.9 |
| 136 | 128.8 | 103 | 68.7 |
| 111 | 128.7 | 164 | 68.2 |
| 136 | 125.7 | 209 | 67.4 |
| 127 | 124.4 | 129 | 66.4 |
| 226 | 120.0 | 150 | 63.3 |
| | | 145 | 61.1 |

TABLE VIII
DOSE-DEPENDANT EFFECTS OF INHIBITORS OF GSK-3B ENZYME ACTIVITY ON THE
PROLIFERATION OF CELLS OF THE HUMAN EMBRYONIC STEM CELL LINE H1.

| | compound # 198 | | compound # 206 | | compound # 221 | | compound # 223 | | compound # 47 | |
|---|---|---|---|---|---|---|---|---|---|---|
| Conc | Cell | | Cell | | Cell | | Cell | | Cell | |
| [μM] | number | SD | number | SD | number | SD | number | SD | number | SD |
| 10 | 1.006 | 0.051 | 0.039 | 0.049 | 0.193 | 0.147 | 1.280 | 0.014 | 1.049 | 0.062 |
| 5 | 1.058 | 0.047 | 1.164 | 0.018 | 0.889 | 0.035 | 1.348 | 0.007 | 1.104 | 0.014 |
| 2.5 | 1.031 | 0.054 | 1.022 | 0.023 | 0.896 | 0.035 | 1.318 | 0.028 | 0.932 | 0.087 |
| 1.25 | 0.899 | 0.040 | 1.121 | 0.023 | 1.120 | 0.072 | 1.159 | 0.041 | 1.006 | 0.023 |
| 0.625 | 0.742 | 0.095 | 1.092 | 0.044 | 1.107 | 0.093 | 1.029 | 0.018 | 0.832 | 0.026 |
| 0.313 | 0.754 | 0.010 | 0.931 | 0.056 | 1.132 | 0.018 | 1.018 | 0.044 | 0.742 | 0.127 |
| 0.156 | 0.822 | 0.074 | 0.804 | 0.002 | 1.082 | 0.041 | 0.776 | 0.054 | 0.712 | 0.020 |
| | compound # 103 | | compound # 133 | | compound # 136 | | compound # 226 | | compound # 233 | |
| Conc | Cell | | Cell | | Cell | | Cell | | Cell | |
| [μM] | number | SD | number | SD | number | [uM] | number | SD | number | SD |
| 10 | 0.001 | 0.001 | 0.096 | 0.103 | 0.058 | 0.074 | 0.290 | 0.307 | 0.000 | 0.000 |
| 5 | 0.034 | 0.035 | 0.262 | 0.268 | 0.173 | 0.207 | 0.458 | 0.263 | 0.089 | 0.067 |
| 2.5 | 0.566 | 0.461 | 0.592 | 0.019 | 0.428 | 0.326 | 0.640 | 0.104 | 0.438 | 0.050 |
| 1.25 | 0.897 | 0.103 | 1.124 | 0.101 | 0.850 | 0.238 | 0.739 | 0.129 | 0.636 | 0.016 |
| 0.625 | 0.921 | 0.122 | 1.106 | 0.056 | 0.910 | 0.061 | 0.805 | 0.036 | 0.736 | 0.025 |
| 0.313 | 1.028 | 0.069 | 0.888 | 0.213 | 0.868 | 0.131 | 0.785 | 0.094 | 0.791 | 0.038 |
| 0.156 | 1.027 | 0.067 | 0.890 | 0.079 | 0.742 | 0.051 | 0.774 | 0.027 | 0.832 | 0.005 |
| | compound # 52 | | compound # 101 | | compound # 110 | | compound # 111 | | compound # 112 | |
| Conc | Cell | | Cell | | Cell | | Cell | | Cell | |
| [μM] | number | SD | number | SD | number | [uM] | number | SD | number | SD |
| 10 | 0.000 | 0.000 | 0.496 | 0.690 | 0.129 | 0.170 | 0.412 | 0.081 | 0.996 | 0.246 |
| 5 | 0.024 | 0.034 | 0.768 | 0.490 | 0.530 | 0.080 | 1.128 | 0.026 | 0.908 | 0.179 |
| 2.5 | 1.097 | 0.294 | 1.001 | 0.129 | 1.174 | 0.016 | 1.031 | 0.217 | 1.005 | 0.086 |
| 1.25 | 1.446 | 0.076 | 1.158 | 0.043 | 1.113 | 0.057 | 0.914 | 0.100 | 1.200 | 0.085 |
| 0.625 | 1.296 | 0.183 | 0.699 | 0.248 | 1.188 | 0.041 | 0.801 | 0.136 | 1.111 | 0.300 |
| 0.313 | 1.034 | 0.197 | 0.617 | 0.232 | 1.158 | 0.102 | 0.785 | 0.121 | 0.959 | 0.094 |
| 0.156 | 0.826 | 0.030 | 0.812 | 0.120 | 0.974 | 0.065 | 0.659 | 0.068 | 0.912 | 0.059 |
| | compound # 144 | | compound # 145 | | compound # 148 | | compound # 150 | | compound # 158 | |
| Conc | Cell | | Cell | | Cell | | Cell | | Cell | |
| [μM] | number | SD | number | SD | number | [uM] | number | SD | number | SD |
| 10 | 0.000 | 0.000 | 0.021 | 0.027 | 0.002 | 0.002 | 0.052 | 0.067 | 0.053 | 0.024 |
| 5 | 0.000 | 0.000 | 0.339 | 0.254 | 1.011 | 0.499 | 1.161 | 0.134 | 0.905 | 0.036 |
| 2.5 | 0.192 | 0.233 | 1.350 | 0.170 | 1.724 | 0.042 | 1.293 | 0.020 | 1.019 | 0.015 |
| 1.25 | 0.552 | 0.458 | 1.277 | 0.101 | 1.652 | 0.032 | 1.213 | 0.087 | 1.163 | 0.062 |
| 0.625 | 0.895 | 0.054 | 0.713 | 0.151 | 1.357 | 0.023 | 1.025 | 0.045 | 1.231 | 0.152 |
| 0.313 | 0.734 | 0.075 | 0.665 | 0.207 | 1.213 | 0.177 | 1.241 | 0.031 | 1.216 | 0.007 |
| 0.156 | 0.594 | 0.078 | 0.469 | 0.465 | 1.206 | 0.142 | 1.041 | 0.007 | 1.103 | 0.065 |

TABLE IX
DOSE-DEPENDANT EFFECTS OF INHIBITORS OF GSK-3B ENZYME ACTIVITY ON THE
DIFFERENTIATION OF CELLS OF THE HUMAN EMBRYONIC STEM CELL LINE H1.

| | compound # 198 | | compound # 206 | | compound # 221 | | compound # 223 | | compound # 47 | |
|---|---|---|---|---|---|---|---|---|---|---|
| Conc. | Sox17 | | Sox17 | | Sox17 | | Sox17 | | Sox17 | |
| [μM] | Intensity | SD | Intensity | SD | Intensity | SD | Intensity | SD | Intensity | SD |
| 10 | 0.889 | 0.144 | 0.029 | 0.034 | 0.140 | 0.095 | 1.183 | 0.044 | 0.969 | 0.040 |
| 5 | 1.004 | 0.021 | 0.824 | 0.035 | 0.785 | 0.077 | 1.171 | 0.010 | 1.013 | 0.002 |
| 2.5 | 1.023 | 0.092 | 0.849 | 0.003 | 0.842 | 0.032 | 1.169 | 0.031 | 0.838 | 0.068 |
| 1.25 | 0.954 | 0.100 | 0.985 | 0.082 | 1.028 | 0.043 | 1.106 | 0.006 | 0.940 | 0.071 |
| 0.625 | 0.793 | 0.135 | 0.986 | 0.059 | 1.016 | 0.000 | 0.931 | 0.033 | 0.767 | 0.014 |
| 0.313 | 0.803 | 0.048 | 0.916 | 0.028 | 1.058 | 0.017 | 0.943 | 0.056 | 0.692 | 0.167 |
| 0.156 | 0.941 | 0.106 | 0.822 | 0.036 | 1.039 | 0.015 | 0.789 | 0.074 | 0.651 | 0.032 |
| | compound # 103 | | compound # 133 | | compound # 136 | | compound # 226 | | compound # 233 | |
| Conc. | Sox17 | | Sox17 | | Sox17 | | Sox17 | | Sox17 | |
| [μM] | Intensity | SD | Intensity | SD | Intensity | SD | Intensity | SD | Intensity | SD |
| 10 | 0.001 | 0.001 | 0.034 | 0.027 | 0.054 | 0.063 | 0.267 | 0.280 | 0.000 | 0.001 |
| 5 | 0.017 | 0.020 | 0.071 | 0.054 | 0.141 | 0.169 | 0.402 | 0.229 | 0.056 | 0.035 |
| 2.5 | 0.200 | 0.157 | 0.497 | 0.076 | 0.373 | 0.326 | 0.605 | 0.041 | 0.286 | 0.034 |
| 1.25 | 0.792 | 0.066 | 0.993 | 0.144 | 0.783 | 0.282 | 0.686 | 0.185 | 0.587 | 0.023 |
| 0.625 | 0.824 | 0.118 | 1.061 | 0.066 | 0.887 | 0.062 | 0.786 | 0.061 | 0.695 | 0.001 |
| 0.313 | 0.934 | 0.127 | 0.937 | 0.136 | 0.859 | 0.176 | 0.780 | 0.132 | 0.753 | 0.098 |
| 0.156 | 0.986 | 0.055 | 0.888 | 0.062 | 0.666 | 0.015 | 0.782 | 0.061 | 0.816 | 0.043 |
| | compound # 52 | | compound # 101 | | compound # 110 | | compound # 111 | | compound # 112 | |
| Conc. | Sox17 | | Sox17 | | Sox17 | | Sox17 | | Sox17 | |
| [μM] | Intensity | SD | Intensity | SD | Intensity | SD | Intensity | SD | Intensity | SD |
| 10 | 0.000 | 0.000 | 0.491 | 0.681 | 0.281 | 0.358 | 0.330 | 0.059 | 0.701 | 0.307 |
| 5 | 0.035 | 0.049 | 0.158 | 0.224 | 0.460 | 0.189 | 0.846 | 0.036 | 0.728 | 0.146 |
| 2.5 | 1.336 | 0.192 | 0.800 | 0.201 | 1.018 | 0.139 | 0.887 | 0.191 | 0.928 | 0.019 |
| 1.25 | 1.238 | 0.030 | 0.910 | 0.045 | 0.960 | 0.106 | 0.819 | 0.179 | 1.159 | 0.093 |
| 0.625 | 0.997 | 0.095 | 0.567 | 0.190 | 1.050 | 0.038 | 0.755 | 0.126 | 1.136 | 0.186 |
| 0.313 | 0.791 | 0.172 | 0.515 | 0.276 | 1.032 | 0.063 | 0.667 | 0.125 | 1.006 | 0.009 |
| 0.156 | 0.669 | 0.037 | 0.708 | 0.148 | 0.950 | 0.087 | 0.628 | 0.053 | 0.922 | 0.096 |
| | compound # 144 | | compound # 145 | | compound # 148 | | compound # 150 | | compound # 158 | |
| Conc. | Sox17 | | Sox17 | | Sox17 | | Sox17 | | Sox17 | |
| [μM] | Intensity | SD | Intensity | SD | Intensity | SD | Intensity | SD | Intensity | SD |
| 10 | 0.000 | 0.000 | 0.018 | 0.021 | 0.002 | 0.001 | 0.054 | 0.062 | 0.074 | 0.048 |
| 5 | 0.000 | 0.000 | 0.235 | 0.174 | 1.052 | 0.281 | 1.250 | 0.177 | 1.006 | 0.070 |
| 2.5 | 0.270 | 0.382 | 1.153 | 0.223 | 1.459 | 0.074 | 1.186 | 0.069 | 1.120 | 0.038 |
| 1.25 | 0.678 | 0.434 | 1.055 | 0.046 | 1.322 | 0.078 | 1.112 | 0.038 | 1.122 | 0.009 |
| 0.625 | 0.978 | 0.021 | 0.569 | 0.124 | 1.173 | 0.015 | 0.913 | 0.005 | 1.241 | 0.230 |
| 0.313 | 0.742 | 0.048 | 0.555 | 0.118 | 1.102 | 0.165 | 1.140 | 0.036 | 1.231 | 0.012 |
| 0.156 | 0.508 | 0.049 | 0.451 | 0.443 | 1.060 | 0.126 | 0.998 | 0.006 | 1.034 | 0.008 |

TABLE X
DOSE-DEPENDANT EFFECTS OF INHIBITORS OF GSK-3B ENZYME ACTIVITY ON THE
PROLIFERATION OF CELLS OF THE HUMAN EMBRYONIC STEM CELL LINE H9.

| | compound # 198 | | compound # 206 | | compound # 221 | | compound # 223 | | compound # 47 | |
|---|---|---|---|---|---|---|---|---|---|---|
| Conc. | Cell | | Cell | | Cell | | Cell | | Cell | |
| [μM] | number | SD | number | SD | number | SD | number | SD | number | SD |
| 10 | 0.164 | 0.209 | 0.001 | 0.000 | 0.049 | 0.028 | 0.123 | 0.106 | 0.770 | 0.077 |
| 5 | 0.147 | 0.141 | 0.616 | 0.497 | 0.583 | 0.155 | 0.954 | 0.146 | 0.496 | 0.011 |
| 2.5 | 0.140 | 0.112 | 1.295 | 0.402 | 1.108 | 0.170 | 0.795 | 0.101 | 0.384 | 0.247 |
| 1.25 | 0.307 | 0.198 | 1.233 | 0.058 | 1.195 | 0.147 | 0.541 | 0.051 | 0.395 | 0.002 |
| 0.625 | 0.138 | 0.071 | 0.606 | 0.121 | 1.100 | 0.014 | 0.332 | 0.049 | 0.221 | 0.009 |
| 0.313 | 0.063 | 0.008 | 0.397 | 0.020 | 0.887 | 0.078 | 0.206 | 0.085 | 0.172 | 0.071 |
| 0.156 | 0.069 | 0.001 | 0.214 | 0.025 | 0.699 | 0.109 | 0.142 | 0.039 | 0.138 | 0.048 |
| | compound # 103 | | compound # 133 | | compound # 136 | | compound # 226 | | compound # 233 | |
| Conc. | Cell | | Cell | | Cell | | Cell | | Cell | |
| [μM] | number | SD | number | SD | number | SD | number | SD | number | SD |
| 10 | 0.001 | 0.000 | 0.785 | 0.192 | 0.208 | 0.134 | 0.377 | 0.040 | 0.000 | 0.000 |
| 5 | 0.023 | 0.024 | 1.067 | 0.236 | 0.320 | 0.087 | 0.336 | 0.081 | 0.052 | 0.009 |
| 2.5 | 0.681 | 0.223 | 1.368 | 0.025 | 0.388 | 0.019 | 0.296 | 0.016 | 0.089 | 0.003 |
| 1.25 | 1.011 | 0.461 | 1.477 | 0.147 | 0.334 | 0.113 | 0.222 | 0.035 | 0.106 | 0.003 |
| 0.625 | 0.927 | 0.108 | 0.899 | 0.108 | 0.267 | 0.148 | 0.282 | 0.096 | 0.169 | 0.041 |
| 0.313 | 0.686 | 0.022 | 0.540 | 0.094 | 0.192 | 0.056 | 0.208 | 0.003 | 0.119 | 0.026 |
| 0.156 | 0.458 | 0.001 | 0.206 | 0.089 | 0.147 | 0.067 | 0.174 | 0.051 | 0.067 | 0.015 |
| | compound # 52 | | compound # 101 | | compound # 110 | | compound # 111 | | compound # 112 | |
| Conc. | Cell | | Cell | | Cell | | Cell | | Cell | |
| [μM] | number | SD | number | SD | number | SD | number | SD | number | SD |
| 10 | 0.000 | 0.000 | 0.452 | 0.094 | 0.002 | 0.001 | 1.117 | 0.043 | 1.022 | 0.422 |
| 5 | 0.002 | 0.000 | 0.433 | 0.050 | 1.325 | 0.015 | 0.793 | 0.030 | 1.281 | 0.109 |
| 2.5 | 0.668 | 0.059 | 0.521 | 0.229 | 1.355 | 0.026 | 0.600 | 0.122 | 1.197 | 0.068 |
| 1.25 | 0.988 | 0.032 | 0.293 | 0.038 | 1.182 | 0.076 | 0.442 | 0.018 | 1.039 | 0.213 |
| 0.625 | 0.390 | 0.032 | 0.200 | 0.122 | 0.928 | 0.127 | 0.371 | 0.072 | 0.686 | 0.014 |
| 0.313 | 0.250 | 0.090 | 0.072 | 0.025 | 0.772 | 0.050 | 0.100 | 0.008 | 0.437 | 0.066 |
| 0.156 | 0.095 | 0.020 | 0.057 | 0.044 | 0.336 | 0.056 | 0.072 | 0.015 | 0.276 | 0.043 |
| | compound # 144 | | compound # 145 | | compound # 148 | | compound # 150 | | compound # 158 | |
| Conc. | Cell | | Cell | | Cell | | Cell | | Cell | |
| [μM] | number | SD | number | SD | number | SD | number | SD | number | SD |
| 10 | 0.007 | 0.002 | 0.000 | 0.000 | 0.000 | 0.000 | 0.044 | 0.038 | 0.004 | 0.001 |
| 5 | 0.002 | 0.001 | 0.127 | 0.069 | 0.415 | 0.023 | 0.382 | 0.110 | 0.017 | 0.003 |
| 2.5 | 0.001 | 0.001 | 0.151 | 0.059 | 0.425 | 0.082 | 0.345 | 0.001 | 0.033 | 0.037 |
| 1.25 | 0.090 | 0.097 | 0.108 | 0.051 | 0.325 | 0.042 | 0.284 | 0.076 | 0.044 | 0.028 |
| 0.625 | 0.248 | 0.058 | 0.230 | 0.168 | 0.314 | 0.062 | 0.266 | 0.021 | 0.100 | 0.099 |
| 0.313 | 0.264 | 0.048 | 0.086 | 0.033 | 0.267 | 0.098 | 0.347 | 0.084 | 0.057 | 0.032 |
| 0.156 | 0.133 | 0.069 | 0.063 | 0.004 | 0.218 | 0.012 | 0.192 | 0.014 | 0.070 | 0.048 |

TABLE XI
DOSE-DEPENDANT EFFECTS OF INHIBITORS OF GSK-3B ENZYME ACTIVITY ON THE
DIFFERENTIATION OF CELLS OF THE HUMAN EMBRYONIC STEM CELL LINE H9.

| | compound # 198 | | compound # 206 | | compound # 221 | | compound # 223 | | compound # 47 | |
|---|---|---|---|---|---|---|---|---|---|---|
| Conc. | Sox17 | | Sox17 | | Sox17 | | Sox17 | | Sox17 | |
| [μM] | Intensity | SD | Intensity | SD | Intensity | SD | Intensity | SD | Intensity | SD |
| 10 | 0.121 | 0.141 | 0.002 | 0.002 | 0.022 | 0.005 | 0.140 | 0.110 | 0.694 | 0.123 |
| 5 | 0.105 | 0.089 | 0.480 | 0.423 | 0.432 | 0.111 | 1.114 | 0.066 | 0.353 | 0.080 |
| 2.5 | 0.100 | 0.062 | 0.986 | 0.269 | 0.869 | 0.158 | 0.726 | 0.079 | 0.297 | 0.235 |
| 1.25 | 0.312 | 0.255 | 1.012 | 0.051 | 1.042 | 0.134 | 0.459 | 0.066 | 0.317 | 0.062 |
| 0.625 | 0.103 | 0.058 | 0.453 | 0.076 | 1.160 | 0.013 | 0.277 | 0.061 | 0.154 | 0.013 |
| 0.313 | 0.052 | 0.008 | 0.311 | 0.005 | 0.951 | 0.010 | 0.155 | 0.071 | 0.110 | 0.030 |
| 0.156 | 0.051 | 0.003 | 0.132 | 0.003 | 0.678 | 0.093 | 0.116 | 0.047 | 0.095 | 0.025 |
| | compound # 103 | | compound # 133 | | compound # 136 | | compound # 226 | | compound # 233 | |
| Conc. | Sox17 | | Sox17 | | Sox17 | | Sox17 | | Sox17 | |
| [μM] | Intensity | SD | Intensity | SD | Intensity | SD | Intensity | SD | Intensity | SD |
| 10 | 0.001 | 0.001 | 0.129 | 0.037 | 0.129 | 0.067 | 0.200 | 0.022 | 0.000 | 0.000 |
| 5 | 0.019 | 0.019 | 0.194 | 0.007 | 0.154 | 0.023 | 0.174 | 0.070 | 0.038 | 0.001 |
| 2.5 | 0.559 | 0.238 | 0.857 | 0.012 | 0.209 | 0.045 | 0.177 | 0.030 | 0.053 | 0.005 |
| 1.25 | 0.943 | 0.419 | 1.110 | 0.042 | 0.202 | 0.103 | 0.129 | 0.029 | 0.075 | 0.017 |
| 0.625 | 0.985 | 0.072 | 0.678 | 0.197 | 0.212 | 0.134 | 0.196 | 0.084 | 0.137 | 0.049 |
| 0.313 | 0.577 | 0.062 | 0.398 | 0.166 | 0.129 | 0.018 | 0.146 | 0.005 | 0.070 | 0.027 |
| 0.156 | 0.364 | 0.044 | 0.149 | 0.058 | 0.125 | 0.051 | 0.132 | 0.063 | 0.039 | 0.010 |
| | compound # 52 | | compound # 101 | | compound # 110 | | compound # 111 | | compound # 112 | |
| Conc. | Sox17 | | Sox17 | | Sox17 | | Sox17 | | Sox17 | |
| [μM] | Intensity | SD | Intensity | SD | Intensity | SD | Intensity | SD | Intensity | SD |
| 10 | 0.000 | 0.000 | 0.262 | 0.068 | 0.000 | 0.000 | 0.822 | 0.024 | 0.759 | 0.328 |
| 5 | 0.001 | 0.001 | 0.251 | 0.092 | 1.185 | 0.012 | 0.543 | 0.004 | 1.127 | 0.121 |
| 2.5 | 0.914 | 0.038 | 0.408 | 0.279 | 1.305 | 0.066 | 0.432 | 0.154 | 1.146 | 0.137 |
| 1.25 | 0.981 | 0.075 | 0.155 | 0.010 | 1.119 | 0.045 | 0.332 | 0.006 | 0.936 | 0.186 |
| 0.625 | 0.246 | 0.036 | 0.150 | 0.095 | 0.941 | 0.111 | 0.268 | 0.050 | 0.563 | 0.019 |
| 0.313 | 0.170 | 0.046 | 0.051 | 0.016 | 0.746 | 0.088 | 0.080 | 0.006 | 0.342 | 0.068 |
| 0.156 | 0.074 | 0.024 | 0.040 | 0.030 | 0.291 | 0.086 | 0.054 | 0.014 | 0.186 | 0.040 |
| | compound # 144 | | compound # 145 | | compound # 148 | | compound # 150 | | compound # 158 | |
| Conc. | Sox17 | | Sox17 | | Sox17 | | Sox17 | | Sox17 | |
| [μM] | Intensity | SD | Intensity | SD | Intensity | SD | Intensity | SD | Intensity | SD |
| 10 | 0.009 | 0.003 | 0.000 | 0.000 | 0.000 | 0.000 | 0.042 | 0.028 | 0.004 | 0.003 |
| 5 | 0.001 | 0.001 | 0.087 | 0.036 | 0.300 | 0.095 | 0.234 | 0.078 | 0.016 | 0.001 |
| 2.5 | 0.001 | 0.001 | 0.120 | 0.066 | 0.299 | 0.019 | 0.205 | 0.002 | 0.042 | 0.049 |
| 1.25 | 0.114 | 0.134 | 0.076 | 0.034 | 0.202 | 0.002 | 0.165 | 0.030 | 0.053 | 0.035 |
| 0.625 | 0.165 | 0.043 | 0.222 | 0.201 | 0.220 | 0.070 | 0.202 | 0.013 | 0.073 | 0.066 |
| 0.313 | 0.240 | 0.030 | 0.068 | 0.010 | 0.203 | 0.061 | 0.282 | 0.135 | 0.054 | 0.040 |
| 0.156 | 0.085 | 0.041 | 0.049 | 0.011 | 0.173 | 0.009 | 0.146 | 0.041 | 0.059 | 0.051 |

TABLE XII
RELATIONSHIP BETWEEN COMPOUNDS
ON TABLES AND COMPOUNDS

| COMPOUND NO. | TABLE-COMPOUND |
|---|---|
| 15 | C-12 |
| 230 | D-13a |
| 221 | D-6a |
| 232 | D-8a |
| 165 | C-6 |
| 223 | D-11a |
| 47 | B-40 |
| 6 | C-2 |
| 163 | C-5 |
| 185 | B-16 |
| 231 | D-7a |
| 2 | C-1 |
| 148 | D-31a |
| 10 | C-29 |
| 16 | B-11 |
| 226 | D-12a |
| 11 | C-26 |
| 165 | C-6 |
| 163 | C-5 |
| 206 | D-4a |
| 6 | C-2 |
| 47 | B-40 |
| 2 | C-1 |
| 230 | D-13a |
| 15 | C-12 |
| 185 | B-16 |
| 232 | D-8a |
| 10 | C-29 |
| 11 | C-26 |
| 3 | C-31 |
| 11 | C-26 |
| 12 | B-2 |
| 17 | B-14 |
| 18 | B-15 |
| 19 | B-21 |
| 22 | B-30 |
| 23 | B-33 |
| 26 | B-34 |
| 27 | B-36 |
| 28 | D-9a |
| 33 | B-29 |
| 34 | B-28 |
| 48 | B-41 |
| 52 | D-15a |
| 55 | D-16a |
| 74 | B-42 |
| 78 | D-17a |
| 82 | D-18a |
| 94 | B-43 |
| 98 | B-44 |
| 101 | D-19a |
| 103 | D-20a |
| 105 | D-21a |
| 110 | D-22a |
| 111 | D-23a |
| 112 | D-24a |
| 127 | D-25a |
| 133 | D-26a |
| 136 | D-27a |
| 139 | D-28a |
| 144 | D-29a |
| 145 | D-30a |
| 150 | D32a |
| 158 | A-5 |
| 164 | C-4 |
| 168 | C-3 |
| 175 | B-4 |
| 180 | C-28 |
| 182 | B-18 |
| 183 | B-19 |
| 198 | D-2A |
| 216 | D-5A |
| 233 | D-14a |
| 241 | B-25 |
| 242 | B-24 |
| 2 | C-1 |
| 6 | C-2 |
| 10 | C-29 |
| 11 | C-26 |
| 15 | C-12 |
| 16 | B-11 |
| 47 | B-40 |
| 148 | D-31A |
| 163 | C-5 |
| 165 | C-6 |
| 166 | D-3a |
| 185 | B-16 |
| 206 | D-4A |
| 221 | D-6a |
| 223 | D-11a |
| 226 | D-12a |
| 230 | D-13a |
| 231 | D-7a |
| 232 | D-8a |

TABLE XIII
CHEMICAL FORMULAS OF OTHER COMPOUNDS TESTED

| COMPOUND NO. | CHEMICAL FORMULA |
|---|---|
| 206 | 3-[1-(2-Hydroxyethyl)-1H-indol-3-yl]-4-(1- |
| | pyridin-3-yl-1H-indol-3-yl)-1H-pyrrole-2,5- |
| | dione |
| 8 | 3-{1-[3-(Dimethylamino)propyl]-1H-indazol-3- |
| | yl}-4-(1-naphthalen-1-yl-1H-indol-3-yl)-1H- |
| | pyrrole-2,5-dione |
| 181 | blocked |
| 209 | 3-[1-(3-Hydroxypropyl)-1H-pyrrolo[2,3- |
| | b]pyridin-3-yl]-4-(1-methyl-1H-pyrazol-3-yl)- |
| | 1H-pyrrole-2,5-dione |
| 4 | 3-[1-(3-Aminopropyl)-1H-indazol-3-yl]-4-[1- |
| | (1-benzothiophen-3-yl)-1H-indol-3-yl]-1H- |
| | pyrrole-2,5-dione |
| 221 | 6-[(2-{[4-(2,4-Dichlorophenyl)-5-(4-methyl- |
| | 1H-imidazol-2-yl)pyrimidin-2- |
| | yl]amino}ethyl)amino]pyridine-3-carbonitrile |
| 16 | 3-[1-(3-Hydroxypropyl)-1H-pyrrolo[2,3- |
| | b]pyridin-3-yl]-4-[2-(trifluoromethyl)phenyl]- |
| | 1H-pyrrole-2,5-dione |
| 169 | 10,11,13,14,16,17,19,20,22,23-Decahydro-1H- |
| | 9,4:24,29-di(metheno)dipyrido[2,3-n:3′,2′- |
| | t]pyrrolo[3,4- |
| | q][1,4,7,10,13,22]tetraoxadiazacyclotetracosine- |
| | 1,3(2H)-dione |
| 14 | 3-(1H-Indazol-3-yl)-4-(1-pyridin-3-yl-1H- |
| | indol-3-yl)-1H-pyrrole-2,5-dione |
| 167 | 6,7,9,10,12,13,15,16-Octahydro-23H- |
| | 5,26:17,22-di(metheno)dipyrido[2,3-k:3′,2′- |
| | q]pyrrolo[3,4- |
| | n][1,4,7,10,19]trioxadiazacyclohenicosine- |
| | 23,25(24H)-dione |
| 13 | 3-[1-(3-Hydroxypropyl)-1H-indazol-3-yl]-4-(1- |
| | naphthalen-2-yl-1H-indol-3-yl)-1H-pyrrole-2,5- |
| | dione |
| 5 | 3-[1-(1-Benzothiophen-3-yl)-1H-indol-3-yl]-4- |
| | [1-(3-hydroxypropyl)-1H-indol-3-yl]-1H- |
| | pyrrole-2,5-dione |

What is claimed is:

1. A method to expand and differentiate pluripotent cells, comprising the steps of:
a. Culturing pluripotent cells, and
b. Treating the pluripotent cells with an inhibitor of GSK-3B enzyme activity.

2. The method of claim 1, wherein the pluripotent cells are embryonic stem cells.

3. The method of claim 1, wherein the pluripotent cells are cells expressing pluripotency markers derived from embryonic stem cells.

4. The method of claim 3, wherein the cells expressing pluripotency markers express at least one of the following pluripotency markers selected from the group consisting of: ABCG2, cripto, FoxD3, Connexin43, Connexin45, Oct4, SOX-2, Nanog, hTERT, UTF-1, ZFP42, SSEA-3, SSEA-4, Tra1-60, and Tra1-81.

5. The method of claim 1, wherein the pluripotent cells are differentiated into cells expressing markers characteristic of the definitive endoderm lineage.

6. The method of claim 1, wherein the pluripotent cells are treated with the inhibitor of GSK-3B enzyme activity for about one to about 72 hours.

7. The method of claim 1, wherein the pluripotent cells are treated with the inhibitor of GSK-3B enzyme activity for about 12 to about 48 hours.

8. The method of claim 1, wherein the pluripotent cells are treated with the inhibitor of GSK-3B enzyme activity for about 48 hours.

9. The method of claim 1, wherein the inhibitor of GSK-3B enzyme activity is used at a concentration of about 100 nM to about 100 μM.

10. The method of claim 1, wherein the inhibitor of GSK-3B enzyme activity is used at a concentration of about 1 μM to about 10 μM.

11. The method of claim 1, wherein the inhibitor of GSK-3B enzyme activity is used at a concentration of about 10 μM.

12. The method of claim 1, wherein the inhibitor of GSK-3B enzyme activity is a compound of the Formula (I):

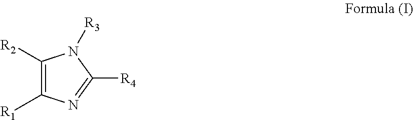

13. The method of claim 12, wherein R₁ is phenyl, substituted phenyl wherein the phenyl substituents are selected from the group consisting of C₁₋₅alkyl, halogen, nitro, trifluoromethyl and nitrile, or pyrimidinyl.

14. The method of claim 12, wherein R₂ is phenyl, substituted phenyl wherein the phenyl substituents are selected from the group consisting of C₁₋₅alkyl, halogen, nitro, trifluoromethyl and nitrile, or pyrimidinyl which is optionally C₁₋₄alkyl substituted, and at least one of R₁ and R₂ is pyrimidinyl.

15. The method of claim 12, wherein R₃ is hydrogen, 2-(trimethylsilyl)ethoxymethyl, C₁₋₅alkoxycarbonyl, aryloxycarbonyl, arylC₁₋₅alkyloxycarbonyl, arylC₁₋₅alkyl, substituted arylC₁₋₅alkyl wherein the one or more aryl substituents are independently selected from the group consisting of C₁₋₅alkyl, C₁₋₅alkoxy, halogen, amino, C₁₋₅alkylamino, and diC₁₋₅alkylamino, phthalimidoC₁₋₅alkyl, aminoC₁₋₅alkyl, diaminoC₁₋₅alkyl, succinimidoC₁₋₅alkyl, C₁₋₅alkylcarbonyl, arylcarbonyl, C₁₋₅alkylcarbonylC₁₋₅alkyl and aryloxycarbonylC₁₋₅alkyl.

16. The method of claim 12, wherein R₄ is -(A)-(CH₂)_{q}—X.

17. The method of claim 16, wherein A is vinylene, ethynylene or

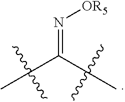

18. The method of claim 17, wherein R₅ is selected from the group consisting of hydrogen, C₁₋₅alkyl, phenyl and phenylC₁₋₅alkyl.

19. The method of claim 16, wherein q is 0-9.

20. The method of claim 16, wherein X is selected from the group consisting of hydrogen, hydroxy, vinyl, substituted vinyl wherein one or more vinyl substituents are each selected from the group consisting of fluorine, bromine, chlorine and iodine, ethynyl, substituted ethynyl wherein the ethynyl substituents are selected from the group consisting of fluorine, bromine chlorine and iodine, C₁₋₅alkyl, substituted C₁₋₅alkyl wherein the one or more alkyl substituents are each selected from the group consisting of C₁₋₅alkoxy, trihaloalkyl, phthalimido and amino, C₃₋₇cycloalkyl, C₁₋₅alkoxy, substituted C₁₋₅alkoxy wherein the alkyl substituents are selected from the group consisting of phthalimido and amino, phthalimidooxy, phenoxy, substituted phenoxy wherein the one or more phenyl substituents are each selected from the group consisting of C₁₋₅alkyl, halogen and C₁₋₅alkoxy, phenyl, substituted phenyl wherein the one or more phenyl substituents are each selected from the group consisting of C₁₋₅alkyl, halogen and C₁₋₅alkoxy, arylC₁₋₅alkyl, substituted arylC₁₋₅alkyl wherein the one or more aryl substituents are each selected from the group consisting of C₁₋₅alkyl, halogen and C₁₋₅alkoxy, aryloxyC₁₋₅alkylamino, C₁₋₅alkylamino, diC₁₋₅alkylamino, nitrile, oxime, benxyloxyimino, C₁₋₅alkyloxyimino, phthalimido, succinimido, C₁₋₅alkylcarbonyloxy, phenylcarbonyloxy, substituted phenylcarbonyloxy wherein the one or more phenyl substituents are each selected from the group consisting of C₁₋₅alkyl, halogen and C₁₋₅alkoxy, phenylC₁₋₅alkylcarbonyloxy wherein the one or more phenyl substituents are each selected from the group consisting of C₁₋₅alkyl, halogen and C₁₋₅alkoxy, aminocarbonyloxy, C₁₋₅alkylaminocarbonyloxy, diC₁₋₅alkylaminocarbonyloxy, C₁₋₅alkoxycarbonyloxy, substituted C₁₋₅alkoxycarbonyloxy wherein the one or more alkyl substituents are each selected from the group consisting of methyl, ethyl, isopropyl and hexyl, phenoxycarbonyloxy, substituted phenoxycarbonyloxy wherein the one or more phenyl substituents are each selected from the group consisting of C₁₋₅alkyl, C₁₋₅alkoxy and halogen, C₁₋₅alkylthio, substituted C₁₋₅alkylthio wherein the alkyl substituents are selected from the group consisting of hydroxy and phthalimido, C₁₋₅alkylsulfonyl, phenylsulfonyl, substituted phenylsulfonyl wherein the one or more phenyl substituents are each selected from the group consisting of bromine, fluorine, chloride, C₁₋₅alkoxy and trifluoromethyl; with the proviso that if A is 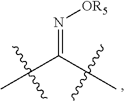
q is 0 and X is H, then R₃ may not be 2-(trimethylsilyl)ethoxymethyl; and pharmaceutically acceptable salts thereof.

21. The method of claim 12, wherein R₁ is substituted phenyl and R₂ is pyrimidin-3-yl.

22. The method of claim 12, wherein R₁ is 4-fluorophenyl.

23. The method of claim 12, wherein R₃ is hydrogen, arylC₁₋₅alkyl, or substituted arylC₁₋₅alkyl.

24. The method of claim 12, wherein R₃ is hydrogen or phenylC₁₋₅alkyl.

25. The method of claim 16, wherein A is ethynylene and q is 0-5.

26. The method of claim 16, wherein X is succinimido, hydroxy, methyl, phenyl, C₁₋₅alkylsulfonyl, C₃₋₆cycloalkyl, C₁₋₅alkylcarbonyloxy, C₁₋₅alkoxy, phenylcarbonyloxy, C₁₋₅alkylamino, diC₁₋₅alkylamino or nitrile.

27. The method of claim 12, wherein the compound of the Formula I is 4-(4-fluorophenyl)-2-(4-hydroxybutyn-1-yl)-1-(3-phenylpropyl)-5-(4-pyridyl)imidazole.

28. The method of claim 1, wherein the inhibitor of GSK-3B enzyme activity is a compound of the Formula (II):

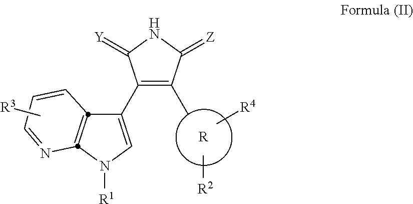

29. The method of claim 28, wherein R is selected from the group consisting of Rₐ, —C₁₋₈alkyl-Rₐ, —C₂₋₈alkenyl-Rₐ, —C₂₋₈alkynyl-Rₐ and cyano.

30. The method of claim 29, wherein Rₐ is selected from the group consisting of cycloalkyl, heterocyclyl, aryl and heteroaryl.

31. The method of claim 28, wherein R¹ is selected from the group consisting of hydrogen, —C₁₋₈alkyl-R⁵, —C₂₋₈alkenyl-R⁵, —C₂₋₈alkynyl-R⁵, —C(O)—(C₁₋₈)alkyl-R⁹, —C(O)-aryl-R⁸, —C(O)—O—(C₁₋₈)alkyl-R⁹, —C(O)—O-aryl-R⁸, —C(O)—NH(C₁₋₈alkyl-R⁹), —C(O)—NH(aryl-R⁸), —C(O)—N(C₁₋₈alkyl-R⁹)₂, —SO₂—(C₁₋₈)alkyl-R⁹, —SO₂-aryl-R⁸, -cycloalkyl-R⁶, -heterocyclyl-R⁶, -aryl-R⁶ and -heteroaryl-R⁶; wherein heterocyclyl and heteroaryl are attached to the azaindole nitrogen atom in the one position via a heterocyclyl or heteroaryl ring carbon atom.

32. The method of claim 31, wherein R⁵ is 1 to 2 substituents independently selected from the group consisting of hydrogen, —O—(C₁₋₈)alkyl, —O—(C₁₋₈)alkyl-OH, —O—(C₁₋₈)alkyl-O—(C₁₋₈)alkyl, —O—(C₁₋₈)alkyl-NH₂, —O—(C₁₋₈)alkyl-NH(C₁₋₈alkyl), —O—(C₁₋₈)alkyl-N(C₁₋₅alkyl)₂, —O—(C₁₋₈)alkyl-S—(C₁₋₈)alkyl, —O—(C₁₋₈)alkyl-SO₂—(C₁₋₈)alkyl, —O—(C₁₋₈)alkyl-SO₂—NH₂, —O—(C₁₋₈)alkyl-SO₂—NH(C₁₋₈alkyl), —O—(C₁₋₈)alkyl-SO₂—N(C₁₋₈alkyl)₂, —O—C(O)H, —O—C(O)—(C₁₋₈)alkyl, —O—C(O)—NH₂, —O—C(O)—NH(C₁₋₈alkyl), —O—C(O)—N(C₁₋₈alkyl)₂, —O—(C₁₋₈)alkyl-C(O)H, —O—(C₁₋₈)alkyl-C(O)—(C₁₋₈)alkyl, —O—(C₁₋₈)alkyl-CO₂H, —O—(C₁₋₈)alkyl-C(O)—O—(C₁₋₈)alkyl, —O—(C₁₋₈)alkyl-C(O)—NH₂, —O—(C₁₋₈)alkyl-C(O)—NH(C₁₋₈alkyl), —O—(C₁₋₈)alkyl-C(O)—N(C₁₋₈alkyl)₂, —C(O)H, —C(O)—(C₁₋₈)alkyl, —CO₂H, —C(O)—O—(C₁₋₈)alkyl, —C(O)—NH₂, —C(NH)—NH₂, —C(O)—NH(C₁₋₈alkyl), —C(O)—N(C₁₋₈alkyl)₂, —SH, —S—(C₁₋₅)alkyl, —S—(C₁₋₈)alkyl-S—(C₁₋₈)alkyl, —S—(C₁₋₅)alkyl-O—(C₁₋₅)alkyl, —S—(C₁₋₈)alkyl-O—(C₁₋₈)alkyl-OH, —S—(C₁₋₈)alkyl-O—(C₁₋₈)alkyl-NH₂, —S—(C₁₋₈)alkyl-O—(C₁₋₈)alkyl-NH(C₁₋₈alkyl), —S—(C₁₋₈)alkyl-O—(C₁₋₈)alkyl-N(C₁₋₅alkyl)₂, —S—(C₁₋₈)alkyl-NH(C₁₋₈alkyl), —SO₂—(C₁₋₈)alkyl, —SO₂—NH₂, —SO₂—NH(C₁₋₈alkyl), —SO₂—N(C₁₋₈alkyl)₂, —N—R⁷, cyano, (halo)₁₋₃, hydroxy, nitro, oxo, -cycloalkyl-R⁶, -heterocyclyl-R⁶, -aryl-R⁶ and -heteroaryl-R⁶.

33. The method of claim 31, wherein R⁶ is 1 to 4 substituents attached to a carbon or nitrogen atom independently selected from the group consisting of hydrogen, —C₁₋₈alkyl, —C₂₋₈alkenyl, —C₂₋₈alkynyl, —C(O)H, —C(O)—(C₁₋₈)alkyl, —CO₂H, —C(O)—O—(C₁₋₈)alkyl, —C(O)—NH₂, —C(NH)—NH₂, —C(O)—NH(C₁₋₈alkyl), —C(O)—N(C₁₋₈)alkyl)₂, —SO₂—(C₁₋₈)alkyl, —SO₂—NH₂, —SO₂—NH(C₁₋₈alkyl), —SO₂—N(C₁₋₈alkyl)₂, —(C₁₋₈)alkyl-N—R⁷, —(C₁₋₈)alkyl-(halo)₁₋₃, —(C₁₋₈)alkyl-OH, -aryl-R⁸, —(C₁₋₈)alkyl-aryl-R⁸ and —(C₁₋₅)alkyl-heteroaryl-R⁸; with the proviso that, when R⁶ is attached to a carbon atom, R⁶ is further selected from the group consisting of —C₁₋₅alkoxy, —(C₁₋₈)alkoxy-(halo)₁₋₃, —SH, —S—(C₁₋₈)alkyl, —N—R⁷, cyano, halo, hydroxy, nitro, oxo and -heteroaryl-R⁸.

34. The method of claim 33, wherein R² is 2 substituents independently selected from the group consisting of hydrogen, —C₁₋₈alkyl, —C₂₋₈alkenyl, —C₂₋₈alkynyl, —(C₁₋₈)alkyl-OH, —(C₁₋₈)alkyl-O—(C₁₋₈)alkyl, —(C₁₋₈)alkyl-NH₂, —(C₁₋₈)alkyl-NH(C₁₋₅alkyl), —(C₁₋₈)alkyl-N(C₁₋₅alkyl)₂, —(C₁₋₈)alkyl-S—(C₁₋₈)alkyl, —C(O)H, —C(O)—(C₁₋₈)alkyl, —C(O)—O—(C₁₋₈)alkyl, —C(O)—NH₂, —C(O)—NH(C₁₋₅alkyl), —C(O)—N(C₁₋₈alkyl)₂, —SO₂—(C₁₋₈)alkyl, —SO₂—NH₂, —SO₂—NH(C₁₋₈alkyl), —SO₂—N(C₁₋₈alkyl)₂, —C(N)—NH₂, -cycloalkyl-R⁸, —(C₁₋₈)alkyl-heterocyclyl-R⁸, -aryl-R⁸, —(C₁₋₈)alkyl-aryl-R⁸ and —(C₁₋₈)alkyl-heteroaryl-R⁸.

35. The method of claim 31, wherein R⁸ is 1 to 4 substituents attached to a carbon or nitrogen atom independently selected from the group consisting of hydrogen, —C₁₋₈alkyl, —(C₁₋₈)alkyl-(halo)₁₋₃ and —(C₁₋₈)alkyl-OH; with the proviso that, when R⁸ is attached to a carbon atom, R⁸ is further selected from the group consisting of —C₁₋₈alkoxy, —NH₂, —NH(C₁₋₅alkyl), —N(C₁₋₈alkyl)₂, cyano, halo, —(C₁₋₈)alkoxy-(halo)₁₋₃, hydroxy and nitro.

36. The method of claim 31, wherein R⁹ is 1 to 2 substituents independently selected from the group consisting of hydrogen, —C₁₋₈alkoxy, —NH₂, —NH(C₁₋₅alkyl), —N(C₁₋₅alkyl)₂, cyano, (halo)₁₋₃, hydroxy and nitro.

37. The method of claim 28, wherein R² is one substituent attached to a carbon or nitrogen atom selected from the group consisting of hydrogen, —C₁₋₈alkyl-R⁵, —C₂₋₈alkenyl-R⁵, —C₂₋₈alkynyl-R⁵, —C(O)H, —C(O)—(C₁₋₈)alkyl-R⁹, —C(O)—NH₂, —C(O)—NH(C₁₋₈alkyl-R⁹), —C(O)—N(C₁₋₈alkyl-R⁹)₂, —C(O)—NH(aryl-R⁸), —C(O)-cycloalkyl-R⁸, —C(O)-heterocyclyl-R⁸, —C(O)-aryl-R⁸, —C(O)-heteroaryl-R⁸, —CO₂H, —C(O)—O—(C₁₋₈)alkyl-R⁹, —C(O)—O-aryl-R⁸, —SO₂—(C₁₋₈)alkyl-R⁹, —SO₂-aryl-R⁸, -cycloalkyl-R⁶, -aryl-R⁶ and —(C₁₋₈)alkyl-N—R²; with the proviso that, when R² is attached to a carbon atom, R² is further selected from the group consisting of —C₁₋₈alkoxy-R⁵, —N—R², cyano, halogen, hydroxy, nitro, oxo, -heterocyclyl-R⁶ and -heteroaryl-R⁶.

38. The method of claim 28, wherein R³ is 1 to 3 substituents attached to a carbon atom independently selected from the group consisting of hydrogen, —C₁₋₅alkyl-R¹⁰, —C₂₋₈alkenyl-R¹⁰, —C₂₋₈alkynyl-R¹⁰, —C₁₋₈alkoxy-R¹⁰, —C(O)H, —C(O)—(C₁₋₈)alkyl-R⁹, —C(O)—NH₂, —C(O)—NH(C₁₋₈alkyl-R⁹), —C(O)—N(C₁₋₈alkyl-R⁹)₂, —C(O)-cycloalkyl-R⁸, —C(O)-heterocyclyl-R⁸, —C(O)-aryl-R⁸, —C(O)-heteroaryl-R⁸, —C(NH)—NH₂, —CO₂H, —C(O)—O—(C₁₋₈)alkyl-R⁹, —C(O)—O-aryl-R⁸, —SO₂—(C₁₋₈)alkyl-R⁹, —SO₂-aryl-R⁸, —N—R⁷, cyano, halogen, hydroxy, nitro, -cycloalkyl-R⁸, -heterocyclyl-R⁸, -aryl-R⁸ and -heteroaryl-R⁸.

39. The method of claim 38, wherein R¹⁰ is 1 to 2 substituents independently selected from the group consisting of hydrogen, —NH₂, —NH(C₁₋₅alkyl), —N(C₁₋₈alkyl)₂, cyano, (halo)₁₋₃, hydroxy, nitro and oxo.

40. The method of claim 28, wherein R⁴ is 1 to 4 substituents attached to a carbon atom independently selected from the group consisting of hydrogen, —C₁₋₅alkyl-R¹⁰, —C₂₋₈alkenyl-R¹⁰, —C₂₋₈alkynyl-R¹⁰, —C₁₋₈alkoxy-R¹⁰, —C(O)H, —C(O)—(C₁₋₈)alkyl-R⁹, —C(O)—NH₂, —C(O)—NH(C₁₋₈alkyl-R⁹), —C(O)—N(C₁₋₈alkyl-R⁹)₂, —C(O)-cycloalkyl-R⁸, —C(O)-heterocyclyl-R⁸, —C(O)-aryl-R⁸, —C(O)-heteroaryl-R⁸, —C(NH)—NH₂, —CO₂H, —C(O)—O—(C₁₋₈)alkyl-R⁹, —C(O)—O-aryl-R⁸, —SH, —S—(C₁₋₈)alkyl-R¹⁰, —SO₂—(C₁₋₈)alkyl-R⁹, —SO₂-aryl-R⁸, —SO₂—NH₂, —SO₂—NH(C₁₋₈alkyl-R⁹), —SO₂—N(C₁₋₈alkyl-R⁹)₂, —N—R⁷, cyano, halogen, hydroxy, nitro, -cycloalkyl-R⁸, -heterocyclyl-R⁸, -aryl-R⁸ and -heteroaryl-R⁸.

41. The method of claim 40, wherein R¹⁰ is 1 to 2 substituents independently selected from the group consisting of hydrogen, —NH₂, —NH(C₁₋₅alkyl), —N(C₁₋₈alkyl)₂, cyano, (halo)₁₋₃, hydroxy, nitro and oxo.

42. The method of claim 28, wherein Y and Z are independently selected from the group consisting of O, S, (H,OH) and (H,H); with the proviso that one of Y and Z is O and the other is selected from the group consisting of O, S, (H,OH) and (H,H); and pharmaceutically acceptable salts thereof.

43. The method of claim 28, wherein R is selected from the group consisting of Rₐ, —C₁₋₄alkyl-Rₐ, —C₂₋₄alkenyl-Rₐ, —C₂₋₄alkynyl-Rₐ and cyano.

44. The method of claim 29, wherein Rₐ is selected from the group consisting of heterocyclyl, aryl and heteroaryl.

45. The method of claim 29, Rₐ is selected from the group consisting of dihydro-pyranyl, phenyl, naphthyl, thienyl, pyrrolyl, imidazolyl, pyrazolyl, pyridinyl, azaindolyl, indazolyl, benzofuryl, benzothienyl, dibenzofuryl and dibenzothienyl.

46. The method of claim 28, wherein R¹ is selected from the group consisting of hydrogen, —C₁₋₄alkyl-R⁵, —C₂₋₄alkenyl-R⁵, —C₂₋₄alkynyl-R⁵, —C(O)—(C₁₋₄)alkyl-R⁹, —C(O)-aryl-R⁸, —C(O)—O—(C₁₋₄)alkyl-R⁹, —C(O)—O-aryl-R⁸, —C(O)—NH(C₁₋₄)alkyl-R⁹), —C(O)—NH(aryl-R⁸), —C(O)—N(C₁₋₄)alkyl-R⁹)₂, —SO₂—(C₁₋₄)alkyl-R⁹, —SO₂-aryl-R⁸, -cycloalkyl-R⁶, -heterocyclyl-R⁶, -aryl-R⁶ and -heteroaryl-R⁶; wherein heterocyclyl and heteroaryl are attached to the azaindole nitrogen atom in the one position via a heterocyclyl or heteroaryl ring carbon atom.

47. The method of claim 28, wherein R¹ is selected from the group consisting of hydrogen, —C₁₋₄alkyl-R⁵, -aryl-R⁶ and -heteroaryl-R⁶; wherein heteroaryl is attached to the azaindole nitrogen atom in the one position via a heteroaryl ring carbon atom.

48. The method of claim 28, wherein R¹ is selected from the group consisting of hydrogen, —C₁₋₄alkyl-R⁵ and -naphthyl-R⁶.

49. The method of claim 31, wherein R⁵ is 1 to 2 substituents independently selected from the group consisting of hydrogen, —O—(C₁₋₄)alkyl, —O—(C₁₋₄)alkyl-OH, —O—(C₁₋₄)alkyl-O—(C₁₋₄)alkyl, —O—(C₁₋₄)alkyl-NH₂, —O—(C₁₋₄)alkyl-NH(C₁₋₄alkyl), —O—(C₁₋₄)alkyl-N(C₁₋₄alkyl)₂, —O—(C₁₋₄)alkyl-S—(C₁₋₄)alkyl, —O—(C₁₋₄)alkyl-SO₂—(C₁₋₄)alkyl, —O—(C₁₋₄alkyl-SO₂—NH₂, —O—(C₁₋₄)alkyl-SO₂—NH(C₁₋₄alkyl), —O—(C₁₋₄)alkyl-SO₂—N(C₁₋₄alkyl)₂, —O—C(O)H, —O—C(O)—(C₁₋₄)alkyl, —O—C(O)—NH₂, —O—C(O)—NH(C₁₋₄alkyl), —O—C(O)—N(C₁₋₄alkyl)₂, —O—(C₁₋₄alkyl-C(O)H, —O—(C₁₋₄)alkyl-C(O)—(C₁₋₄alkyl, —O—(C₁₋₄)alkyl-CO₂H, —O—(C₁₋₄)alkyl-C(O)—O—(C₁₋₄)alkyl, —O—(C₁₋₄)alkyl-C(O)—NH₂, —O—(C₁₋₄)alkyl-C(O)—NH(C₁₋₄alkyl), —O—(C₁₋₄)alkyl-C(O)—N(C₁₋₄alkyl)₂, —C(O)H, —C(O)—(C₁₋₄)alkyl, —CO₂H, —C(O)—O—(C₁₋₄alkyl, —C(O)—NH₂, —C(NH)—NH₂, —C(O)—NH(C₁₋₄alkyl), —C(O)—N(C₁₋₄alkyl)₂, —SH, —S—(C₁₋₄)alkyl, —S—(C₁₋₄)alkyl-S—(C₁₋₄)alkyl, —S—(C₁₋₄)alkyl-O—(C₁₋₄)alkyl, —S—(C₁₋₄)alkyl-O—(C₁₋₄)alkyl-OH, —S—(C₁₋₄)alkyl-O—(C₁₋₄)alkyl-NH₂, —S—(C₁₋₄)alkyl-O—(C₁₋₄)alkyl-NH(C₁₋₄alkyl), —S—(C₁₋₄)alkyl-O—(C₁₋₄)alkyl-N(C₁₋₄alkyl)₂, —S—(C₁₋₄)alkyl-NH(C₁₋₄alkyl), —SO₂—(C₁₋₄alkyl, —SO₂—NH₂, —SO₂—NH(C₁₋₄alkyl), —SO₂—N(C₁₋₄alkyl)₂, —N—R⁷, cyano, (halo)₁₋₃, hydroxy, nitro, oxo, -cycloalkyl-R⁶, -heterocyclyl-R⁶, -aryl-R⁶ and -heteroaryl-R⁶.

50. The method of claim 31, wherein R⁵ is 1 to 2 substituents independently selected from the group consisting of hydrogen, —O—(C₁₋₄)alkyl, —N—R⁷, hydroxy and -heteroaryl-R⁶.

51. The method of claim 31, wherein R⁵ is 1 to 2 substituents independently selected from the group consisting of hydrogen, —O—(C₁₋₄)alkyl, —N—R⁷, hydroxy, -imidazolyl-R⁶, -triazolyl-R⁶ and -tetrazolyl-R⁶.

52. The method of claim 31, wherein R⁶ is 1 to 4 substituents attached to a carbon or nitrogen atom independently selected from the group consisting of hydrogen, —C₁₋₄alkyl, —C₂₋₄alkenyl, —C₂₋₄alkynyl, —C(O)H, —C(O)—(C₁₋₄alkyl, —CO₂H, —C(O)—O—(C₁₋₄)alkyl, —C(O)—NH₂, —C(NH)—NH₂, —C(O)—NH(C₁₋₄alkyl), —C(O)—N(C₁₋₄alkyl)₂, —SO₂—(C₁₋₄)alkyl, —SO₂—NH₂, —SO₂—NH(C₁₋₄alkyl), —SO₂—N(C₁₋₄alkyl)₂, —(C₁₋₄)alkyl-N—R⁷, —(C₁₋₄)alkyl-(halo)₁₋₃, —(C₁₋₄)alkyl-OH, -aryl-R⁸, —(C₁₋₄)alkyl-aryl-R⁸ and —(C₁₋₄)alkyl-heteroaryl-R⁸; with the proviso that, when R⁶ is attached to a carbon atom, R⁶ is further selected from the group consisting of —C₁₋₄alkoxy, —(C₁₋₄alkoxy-(halo)₁₋₃, —SH, —S—(C₁₋₄)alkyl, —N—R⁷, cyano, halo, hydroxy, nitro, oxo and -heteroaryl-R⁸.

53. The method of claim 31, wherein R⁶ is hydrogen.

54. The method of claim 33, wherein R⁷ is 2 substituents independently selected from the group consisting of hydrogen, —C₁₋₄alkyl, —C₂₋₄alkenyl, —C₂₋₄alkynyl, —(C₁₋₄)alkyl-OH, —(C₁₋₄)alkyl-O—(C₁₋₄)alkyl, —(C₁₋₄)alkyl-NH₂, —(C₁₋₄)alkyl-NH(C₁₋₄alkyl), —(C₁₋₄)alkyl-N(C₁₋₄alkyl)₂, —(C₁₋₄)alkyl-S—(C₁₋₄)alkyl, —C(O)H, —C(O)—(C₁₋₄)alkyl, —C(O)—O—(C₁₋₄)alkyl, —C(O)—NH₂, —C(O)—NH(C₁₋₄alkyl), —C(O)—N(C₁₋₄alkyl)₂, —SO₂—(C₁₋₄)alkyl, —SO₂—NH₂, —SO₂—NH(C₁₋₄alkyl), —SO₂—N(C₁₋₄alkyl)₂, —C(N)—NH₂, -cycloalkyl-R⁸, —(C₁₋₄)alkyl-heterocyclyl-R⁸, -aryl-R⁸, —(C₁₋₄)alkyl-aryl-R⁸ and —(C₁₋₄)alkyl-heteroaryl-R⁸.

55. The method of claim 33, wherein R⁷ is 2 substituents independently selected from the group consisting of hydrogen, —C₁₋₄alkyl, —C(O)H, —C(O)—(C₁₋₄)alkyl, —C(O)—O—(C₁₋₄)alkyl, —SO₂—NH₂, —SO₂—NH(C₁₋₄alkyl) and —SO₂—N(C₁₋₄alkyl)₂.

56. The method of claim 31, wherein R⁸ is 1 to 4 substituents attached to a carbon or nitrogen atom independently selected from the group consisting of hydrogen, —C₁₋₄alkyl, —(C₁₋₄)alkyl-(halo)₁₋₃ and —(C₁₋₄)alkyl-OH; with the proviso that, when R⁸ is attached to a carbon atom, R⁸ is further selected from the group consisting of —C₁₋₄alkoxy, —NH₂, —NH(C₁₋₄alkyl), —N(C₁₋₄alkyl)₂, cyano, halo, —(C₁₋₄alkoxy-(halo)₁₋₃, hydroxy and nitro.

57. The method of claim 31, wherein R⁸ is hydrogen.

58. The method of claim 31, wherein R⁹ is 1 to 2 substituents independently selected from the group consisting of hydrogen, —C₁₋₄alkoxy, —NH₂, —NH(C₁₋₄alkyl), —N(C₁₋₄alkyl)₂, cyano, (halo)₁₋₃, hydroxy and nitro.

59. The method of claim 31, wherein R⁹ is hydrogen.

60. The method of claim 28, wherein R² is one substituent attached to a carbon or nitrogen atom selected from the group consisting of hydrogen, —C₁₋₄alkyl-R⁵, —C₂₋₄alkenyl-R⁵, —C₂₋₄alkynyl-R⁵, —C(O)H, —C(O)—(C₁₋₄)alkyl-R⁹, —C(O)—NH₂, —C(O)—NH(C₁₋₄)alkyl-R⁹), —C(O)—N(C₁₋₄)alkyl-R⁹)₂, —C(O)—NH(aryl-R⁸), —C(O)-cycloalkyl-R⁸, —C(O)-heterocyclyl-R⁸, —C(O)-aryl-R⁸, —C(O)-heteroaryl-R⁸, —CO₂H, —C(O)—O—(C₁₋₄)alkyl-R⁹, —C(O)—O-aryl-R⁸, —SO₂—(C₁₋₄)alkyl-R⁹, —SO₂-aryl-R⁸, -cycloalkyl-R⁶, -aryl-R⁶ and —(C₁₋₄)alkyl-N—R²; with the proviso that, when R² is attached to a carbon atom, R² is further selected from the group consisting of —C₁₋₄alkoxy-R⁵, —N—R², cyano, halogen, hydroxy, nitro, oxo, -heterocyclyl-R⁶ and -heteroaryl-R⁶.

61. The method of claim 28, wherein R² is one substituent attached to a carbon or nitrogen atom selected from the group consisting of hydrogen, —C₁₋₄alkyl-R⁵, —C₂₋₄alkenyl-R⁵, —C₂₋₄alkynyl-R⁵, —CO₂H, —C(O)—O—(C₁₋₄)alkyl-R⁹, -cycloalkyl-R⁶, -aryl-R⁶ and —(C₁₋₄)alkyl-N—R²; with the proviso that, when R² is attached to a nitrogen atom, a quaternium salt is not formed; and, with the proviso that, when R² is attached to a carbon atom, R² is further selected from the group consisting of —C₁₋₄alkoxy-R⁵, —N—R⁷, cyano, halogen, hydroxy, nitro, oxo, -heterocyclyl-R⁶ and -heteroaryl-R⁶.

62. The method of claim 28, wherein R² is one substituent attached to a carbon or nitrogen atom selected from the group consisting of hydrogen, —C₁₋₄alkyl-R⁵ and -aryl-R⁶; with the proviso that, when R² is attached to a nitrogen atom, a quaternium salt is not formed; and, with the proviso that when R² is attached to a carbon atom, R² is further selected from the group consisting of —N—R², halogen, hydroxy and -heteroaryl-R⁶.

63. The method of claim 28, wherein R³ is 1 to 3 substituents attached to a carbon atom independently selected from the group consisting of hydrogen, —C₁₋₄alkyl-R¹⁰, —C₂₋₄alkenyl-R¹⁰, —C₂₋₄alkynyl-R¹⁰, —C₁₋₄alkoxy-R¹⁰, —C(O)H, —C(O)—(C₁₋₄)alkyl-R⁹, —C(O)—NH₂, —C(O)—NH(C₁₋₄alkyl-R⁹), —C(O)—N(C₁₋₄alkyl-R⁹)₂, —C(O)-cycloalkyl-R⁸, —C(O)-heterocyclyl-R⁸, —C(O)-aryl-R⁸, —C(O)-heteroaryl-R⁸, —C(NH)—NH₂, —CO₂H, —C(O)—O—(C₁₋₄alkyl-R⁹, —C(O)—O-aryl-R⁸, —SO₂—(C₁₋₈)alkyl-R⁹, —SO₂-aryl-R⁸, —N—R⁷, —(C₁₋₄)alkyl-N—R⁷, cyano, halogen, hydroxy, nitro, -cycloalkyl-R⁸, -heterocyclyl-R⁸, -aryl-R⁸ and -heteroaryl-R⁸.

64. The method of claim 28, wherein R³ is one substituent attached to a carbon atom selected from the group consisting of hydrogen, —C₁₋₄alkyl-R¹⁰, —C₂₋₄alkenyl-R¹⁰, —C₂₋₄alkynyl-R¹⁰, —C₁₋₄alkoxy-R¹⁰, —C(O)H, —CO₂H, —NH₂, —NH(C₁₋₄alkyl), —N(C₁₋₄alkyl)₂, cyano, halogen, hydroxy and nitro.

65. The method of claim 28, wherein R³ is one substituent attached to a carbon atom selected from the group consisting of hydrogen, —C₁₋₄alkyl-R¹⁰, —NH₂, —NH(C₁₋₄alkyl), —N(C₁₋₄alkyl)₂, halogen and hydroxy.

66. The method of claim 28, wherein R⁴ is 1 to 4 substituents attached to a carbon atom independently selected from the group consisting of hydrogen, —C₁₋₄alkyl-R¹⁰, —C₂₋₄alkenyl-R¹⁰, —C₂₋₄alkynyl-R¹⁰, —C₁₋₄alkoxy-R¹⁰, —C(O)H, —C(O)—(C₁₋₄alkyl-R⁹, —C(O)—NH₂, —C(O)—NH(C₁₋₄alkyl-R⁹), —C(O)—N(C₁₋₄alkyl-R⁹)₂, —C(O)-cycloalkyl-R⁸, —C(O)-heterocyclyl-R⁸, —C(O)-aryl-R⁸, —C(O)-heteroaryl-R⁸, —C(NH)—NH₂, —CO₂H, —C(O)—O—(C₁₋₄alkyl-R⁹, —C(O)—O-aryl-R⁸, —SH, —S—(C₁₋₄)alkyl-R¹⁰, —SO₂—(C₁₋₄)alkyl-R⁹, —SO₂-aryl-R⁸, —SO₂—NH₂, —SO₂—NH(C₁₋₄alkyl-R⁹), —SO₂—N(C₁₋₄)alkyl-R⁹)₂, —N—R⁷, cyano, halogen, hydroxy, nitro, -cycloalkyl-R⁸, -heterocyclyl-R⁸, -aryl-R⁸ and -heteroaryl-R⁸.

67. The method of claim 28, wherein R⁴ is 1 to 4 substituents attached to a carbon atom independently selected from the group consisting of hydrogen, —C₁₋₄alkyl-R¹⁰, —C₂₋₄alkenyl-R¹⁰, —C₂₋₄alkynyl-R¹⁰, —C₁₋₄alkoxy-R¹⁰, —C(O)H, —CO₂H, —NH₂, —NH(C₁₋₄alkyl), —N(C₁₋₄alkyl)₂, cyano, halogen, hydroxy, nitro, -cycloalkyl, -heterocyclyl, -aryl and -heteroaryl.

68. The method of claim 28, wherein R⁴ is 1 to 4 substituents attached to a carbon atom independently selected from the group consisting of hydrogen, C₁₋₄alkyl-R¹⁰, C₁₋₄alkoxy-R¹⁰, —NH₂, —NH(C₁₋₄alkyl), —N(C₁₋₄alkyl)₂, halogen and hydroxy.

69. The method of claim 28, wherein R⁴ is 1 to 4 substituents attached to a carbon atom independently selected from the group consisting of hydrogen, C₁₋₄alkyl-R¹⁰, C₁₋₄alkoxy-R¹⁰, —NH₂, —NH(C₁₋₄alkyl), —N(C₁₋₄alkyl)₂, chlorine, fluorine and hydroxy.

70. The method of claims 38 and 41, wherein R¹⁰ is 1 to 2 substituents independently selected from the group consisting of hydrogen, —NH₂, —NH(C₁₋₄alkyl), —N(C₁₋₄alkyl)₂, cyano, (halo)₁₋₃, hydroxy, nitro and oxo.

71. The method of claims 38 and 41, wherein R¹⁰ is 1 to 2 substituents independently selected from the group consisting of hydrogen and (halo)₁₋₃.

72. The method of claims 38 and 41, wherein R¹⁰ is 1 to 2 substituents independently selected from the group consisting of hydrogen and (fluoro)₃.

73. The method of claim 28, wherein Y and Z are independently selected from the group consisting of O, S, (H,OH) and (H,H); with the proviso that one of Y and Z is O and the other is selected from the group consisting of O, S, (H,OH) and (H,H).

74. The method of claim 28, wherein Y and Z are independently selected from the group consisting of O and (H,H); with the proviso that one of Y and Z is O, and the other is selected from the group consisting of O and (H,H).

75. The method of claim 28, wherein Y and Z are independently selected from O.

76. The method of claim 28, where the compound of the Formula II is 3-[1-(3-hydroxypropyl)-1H-pyrrolo[2,3-b]pyridin-3-yl]-4-[2-(trifluoromethyl)phenyl]-1H-pyrrole-2,5-dione.

77. The method of claim 28, where the compound of the Formula II is 3-[1-(3-hydroxypropyl)-1H-pyrrolo[2,3-b]pyridin-3-yl]-4-(1-methyl-1H-pyrazol-3-yl)-1H-pyrrole-2,5-dione.

78. The method of claim 28, where the compound of the Formula II is 3-[1-(3-hydroxy-propyl)-1H-pyrrolo[2,3-b]pyridin-3-yl]-4-pyrazin-2-yl-pyrrole-2,5-dione.

79. The method of claim 28, where the compound of the Formula II is 3-(2,4-dimethoxy-pyrimidin-5-yl)-4-[1-(3-hydroxy-propyl)-1H-pyrrolo[2,3-b]pyridin-3-yl]-pyrrole-2,5-dione.

80. The method of claim 28, where the compound of the Formula II is 4-{3-[4-(2,4-dimethoxy-pyrimidin-5-yl)-2,5-dioxo-2,5-dihydro-1H-pyrrol-3-yl]-pyrrolo[2,3-b]pyridin-1-yl}-butyronitrile.

81. The method of claim 28, where the compound of the Formula II is 4-{3-[4-(1-methyl-1H-pyrazol-3-yl)-2,5-dioxo-2,5-dihydro-1H-pyrrol-3-yl]-pyrrolo[2,3-b]pyridin-1-yl}-butyronitrile.

82. The method of claim 28, where the compound of the Formula II is 3-(2,4-dimethoxy-pyrimidin-5-yl)-4-(1-phenethyl-1H-pyrrolo[2,3-b]pyridine-3-yl)-pyrrole-2,5-dione.

83. The method of claim 1, wherein the inhibitor of GSK-3B enzyme activity is a compound of the Formula (III):

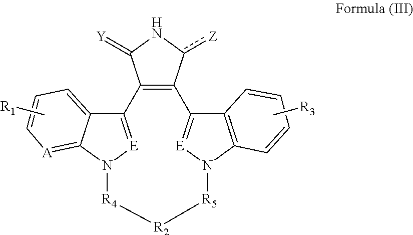

84. The method of claim 83, wherein A and E are independently selected from the group consisting of a hydrogen substituted carbon atom and a nitrogen atom; wherein 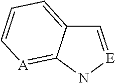
is independently selected from the group consisting of 1H-indole, 1H-pyrrolo[2,3-b]pyridine, 1H-pyrazolo[3,4-b]pyridine and 1H-indazole.

85. The method of claim 83, wherein Z is selected from O; alternatively, Z is selected from dihydro; wherein each hydrogen atom is attached by a single bond.

86. The method of claim 83, wherein R₄ and R₅ are independently selected from C₁₋₈alkyl, C₂₋₈alkenyl and C₂₋₈alkynyl optionally substituted with oxo.

87. The method of claim 83, wherein R₂ is selected from the group consisting of —C₁₋₈alkyl-, —C₂₋₈alkenyl-, —C₂₋₈alkynyl-, —O—(C₁₋₈)alkyl-O—, —O—(C₂₋₈)alkenyl-O—, —O—(C₂₋₈)alkynyl-O—, —C(O)—(C₁₋₈)alkyl-C(O)— (wherein any of the foregoing alkyl, alkenyl and alkynyl linking groups are straight carbon chains optionally substituted with one to four substituents independently selected from the group consisting of C₁₋₈alkyl, C₁₋₈alkoxy, C₁₋₅alkoxy(C₁₋₈)alkyl, carboxyl, carboxyl(C₁₋₈)alkyl, —C(O)O—(C₁₋₈)alkyl, —C₁₋₈alkyl-C(O)O—(C₁₋₈)alkyl, amino (substituted with a substituent independently selected from the group consisting of hydrogen and C₁₋₄alkyl), amino(C₁₋₅)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy, hydroxy(C₁₋₈)alkyl and oxo; and, wherein any of the foregoing alkyl, alkenyl and alkynyl linking groups are optionally substituted with one to two substituents independently selected from the group consisting of heterocyclyl, aryl, heteroaryl, heterocyclyl(C₁₋₈)alkyl, aryl(C₁₋₈)alkyl, heteroaryl(C₁₋₈)alkyl, spirocycloalkyl and spiroheterocyclyl (wherein any of the foregoing cycloalkyl, heterocyclyl, aryl and heteroaryl substituents are optionally substituted with one to four substituents independently selected from the group consisting of C₁₋₅alkyl, C₁₋₅alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₅)alkyl (wherein amino is substituted with a substituent independently selected from the group consisting of hydrogen and C₁₋₄alkyl), halogen, (halo)₁₋₃(C₁₋₈)alkyl, (halo)₁₋₃ (C₁₋₅)alkoxy, hydroxy and hydroxy(C₁₋₅)alkyl; and, wherein any of the foregoing heterocyclyl substituents are optionally substituted with oxo)), cycloalkyl, heterocyclyl, aryl, heteroaryl (wherein cycloalkyl, heterocyclyl, aryl and heteroaryl are optionally substituted with one to four substituents independently selected from the group consisting of C₁₋₅alkyl, C₁₋₅alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₅)alkyl (wherein amino is substituted with a substituent independently selected from the group consisting of hydrogen and C₁₋₄alkyl), halogen, (halo)₁₋₃(C₁₋₈)alkyl, (halo)₁₋₃ (C₁₋₈)alkoxy, hydroxy and hydroxy(C₁₋₈)alkyl; and, wherein heterocyclyl is optionally substituted with oxo), —(O—(CH₂)₁₋₆)₀₋₅—O—, —O—(CH₂)₁₋₆—O—(CH₂)₁₋₆—O—, —O—(CH₂)₁₋₆—O—(CH₂)₁₋₆—O—(CH₂)₁₋₆—O—, —(O—(CH₂)₁₋₆)₀₋₅—NR₆—, —O—(CH₂)₁₋₆—NR₆—(CH₂)₁₋₆—O—, —O—(CH₂)₁₋₆—O—(CH₂)₁₋₆—NR₆—, —(O—(CH₂)₁₋₆)₀₋₅—S—, —O—(CH₂)₁₋₆—S—(CH₂)₁₋₆—O—, —O—(CH₂)₁₋₆—O—(CH₂)₁₋₆—S—, —NR₆—, —NR₆—NR₇—, —NR₆—(CH₂)₁₋₆—NR₂—, —NR₆—(CH₂)₁₋₆—NR₇—(CH₂)₁₋₆—NR₈—, —NR₆—C(O)—, —C(O)—NR₆—, —C(O)—(CH₂)₀₋₆—NR₆—(CH₂)₀₋₆—C(O)—, —NR₆—(CH₂)₀₋₆—C(O)—(CH₂)₁₋₆—C(O)—(CH₂)₀₋₆—NR₂—, —NR₆—C(O)—NR₇—, —NR₆—C(NR₇)—NR₈—, —O—(CH₂)₁₋₆—NR₆—(CF₁₂)₁₋₆—S—, —S—(CH₂)₁₋₆—NR₆—(CH₂)₁₋₆—O—, —S—(CH₂)₁₋₆—NR₆—(CH₂)₁₋₆—S—, —NR₆—(CH₂)₁₋₆—S—(CH₂)₁₋₆—NR₂— and —SO₂— (wherein R₆, R₂ and R₈ are independently selected from the group consisting of hydrogen, C₁₋₈alkyl, C₁₋₈alkoxy(C₁₋₈)alkyl, carboxyl(C₁₋₈)alkyl, amino(C₁₋₈)alkyl (wherein amino is substituted with a substituent independently selected from the group consisting of hydrogen and C₁₋₄alkyl), hydroxy(C₁₋₈)alkyl, heterocyclyl(C₁₋₈)alkyl, aryl(C₁₋₈)alkyl and heteroaryl(C₁₋₈)alkyl (wherein the foregoing heterocyclyl, aryl and heteroaryl substituents are optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl; and, wherein heterocyclyl is optionally substituted with oxo)); with the proviso that, if A and E are selected from a hydrogen substituted carbon atom, then R₂ is selected from the group consisting of —C₂₋₈alkynyl-, —O—(C₁₋₈)alkyl-O—, —O—(C₂₋₈)alkenyl-O—, —O—(C₂₋₈)alkynyl-O—, —C(O)—(C₁₋₈)alkyl-C(O)— (wherein any of the foregoing alkyl, alkenyl and alkynyl linking groups are straight carbon chains optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, —C(O)O—(C₁₋₈)alkyl, —C₁₋₈alkyl-C(O)O—(C₁₋₈)alkyl, amino (substituted with a substituent independently selected from the group consisting of hydrogen and C₁₋₄alkyl), amino(C₁₋₅)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy, hydroxy(C₁₋₈)alkyl and oxo; and, wherein any of the foregoing alkyl, alkenyl and alkynyl linking groups are optionally substituted with one to two substituents independently selected from the group consisting of heterocyclyl, aryl, heteroaryl, heterocyclyl(C₁₋₈)alkyl, aryl(C₁₋₈)alkyl, heteroaryl(C₁₋₈)alkyl, spirocycloalkyl and spiroheterocyclyl (wherein any of the foregoing cycloalkyl, heterocyclyl, aryl and heteroaryl substituents are optionally substituted with one to four substituents independently selected from the group consisting of C₁₋₅alkyl, C₁₋₅alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₅)alkyl (wherein amino is substituted with a substituent independently selected from the group consisting of hydrogen and C₁₋₄alkyl), halogen, (halo)₁₋₃(C₁₋₈)alkyl, (halo)₁₋₃ (C₁₋₈)alkoxy, hydroxy and hydroxy(C₁₋₈)alkyl; and, wherein any of the foregoing heterocyclyl substituents are optionally substituted with oxo)), cycloalkyl (wherein cycloalkyl is optionally substituted with one to four substituents independently selected from the group consisting of C₁₋₅alkyl, C₁₋₅alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₅)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl), —(O—(CH₂)₁₋₆)₁₋₅—O—, —O—(CH₂)₁₋₆—O—(CH₂)₁₋₆—O—, —O—(CH₂)₁₋₆—O—(CH₂)₁₋₆—O—(CH₂)₁₋₆—O—, —(O—(CH₂)₁₋₆)₁₋₅—NR₆—, —O—(CH₂)₁₋₆—NR₆—(CH₂)₁₋₆—O—, —O—(CH₂)₁₋₆—O—(CH₂)₁₋₆—NR₆—, —(O—(CH₂)₁₋₆)₀₋₅—S—, —O—(CH₂)₁₋₆—S—(CH₂)₁₋₆—O—, —O—(CH₂)₁₋₆—O—(CH₂)₁₋₆—S—, —NR₆—NR₇—, —NR₆—(CH₂)₁₋₆—NR₇—, —NR₆—(CH₂)₁₋₆—NR₇—(CH₂)₁₋₆—NR₈—, —NR₉—C(O)—, —C(O)—NR₉—, —C(O)—(CH₂)₀₋₆—NR₆—(CH₂)₀₋₆—C(O)—, —NR₆—(CH₂)₀₋₆—C(O)—(CH₂)₁₋₆—C(O)—(CH₂)₀₋₆—NR₇—, —NR₆—C(O)—NR₇—, —NR₆—C(NR₇)—NR₈—, —O—(CH₂)₁₋₆—NR₆—(CH₂)₁₋₆—S—, —S—(CH₂)₁₋₆—NR₆—(CH₂)₁₋₆—O—, —S—(CH₂)₁₋₆—NR₆—(CH₂)₁₋₆—S— and —NR₆—(CH₂)₁₋₆—S—(CH₂)₁₋₆—NR₇— (wherein R₆, R₇ and R₈ are independently selected from the group consisting of hydrogen, C₁₋₈alkyl, C₁₋₈alkoxy(C₁₋₈)alkyl, carboxyl(C₁₋₈)alkyl, amino(C₁₋₈)alkyl (wherein amino is substituted with a substituent independently selected from the group consisting of hydrogen and C₁₋₄alkyl), hydroxy(C₁₋₈)alkyl, heterocyclyl(C₁₋₈)alkyl, aryl(C₁₋₈)alkyl and heteroaryl(C₁₋₈)alkyl (wherein the foregoing heterocyclyl, aryl and heteroaryl substituents are optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃ (C₁₋₈)alkoxy, hydroxy and hydroxy(C₁₋₈)alkyl; and, wherein heterocyclyl is optionally substituted with oxo); and, wherein R₉ is selected from the group consisting of C₁₋₈alkyl, C₁₋₈alkoxy(C₁₋₈)alkyl, carboxyl(C₁₋₈)alkyl, amino(C₁₋₈)alkyl (wherein amino is substituted with a substituent independently selected from the group consisting of hydrogen and C₁₋₄alkyl), hydroxy(C₁₋₈)alkyl, heterocyclyl(C₁₋₈)alkyl, aryl(C₁₋₈)alkyl and heteroaryl(C₁₋₈)alkyl (wherein the foregoing heterocyclyl, aryl and heteroaryl substituents are optionally substituted with one to four substituents independently selected from the group consisting of C₁₋₈alkyl, C₁₋₈alkoxy, C₁₋₈alkoxy(C₁₋₈)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃(C₁₋₈)alkoxy, hydroxy and hydroxy(C₁₋₈)alkyl; and, wherein heterocyclyl is optionally substituted with oxo)).

88. The method of claim 83, wherein R₁ and R₃ are independently selected from the group consisting of hydrogen, C₁₋₈alkyl, C₂₋₈alkenyl, C₂₋₈alkynyl (wherein alkyl, alkenyl and alkynyl are optionally substituted with a substituent selected from the group consisting of C₁₋₈alkoxy, alkoxy(C₁₋₅)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), (halo)₁₋₃, (halo)₁₋₃(C₁₋₈)alkyl, (halo)₁₋₃(C₁₋₈)alkoxy, hydroxy, hydroxy(C₁₋₈)alkyl and oxo), C₁₋₈alkoxy, C₁₋₈alkoxycarbonyl, (halo)₁₋₃(C₁₋₈)alkoxy, C₁₋₈alkylthio, aryl, heteroaryl (wherein aryl and heteroaryl are optionally substituted with a substituent selected from the group consisting of C₁₋₈alkyl, C₁₋₈alkoxy, alkoxy(C₁₋₅)alkyl, carboxyl, carboxyl(C₁₋₈)alkyl, amino (substituted with a substituent independently selected from the group consisting of hydrogen and C₁₋₄alkyl), amino(C₁₋₈)alkyl (wherein amino is substituted with a substituent independently selected from the group consisting of hydrogen and C₁₋₄alkyl), halogen, (halo)₁₋₃(C₁₋₈)alkyl, (halo)₁₋₃ (C₁₋₈)alkoxy, hydroxy and hydroxy(C₁₋₈)alkyl), amino (substituted with a substituent independently selected from the group consisting of hydrogen and C₁₋₄alkyl), cyano, halogen, hydroxy and nitro; and pharmaceutically acceptable salts thereof.

89. The method of claim 83, wherein the compound of the Formula (III) is 6,7,9,10,12,13,15,16-octahydro-23H-5,26:17,22-dimetheno-5H-dipyrido[2,3-k:3′,2′-q]pyrrolo[3,4-n][1,4,7,10,19]trioxadiazacyclohenicosine-23,25(24H)-dione.

90. The method of claim 83, wherein the compound of the Formula (III) is 10,11,13,14,16,17,19,20,22,23-decahydro-9,4:24,29-dimetheno-1H-dipyrido[2,3-n:3′,2′-t]pyrrolo[3,4-q][1,4,7,10,13,22]tetraoxadiazacyclotetracosine-1,3(2H)-dione.

91. The method of claim 83, wherein the compound of the Formula (III) is 10,11,13,14,16,17,19,20,22,23,25,26-dodecahydro-9, 4:27,32-dimetheno-1H-dipyrido[2,3-q:3′,2′-w]pyrrolo[3,4-t][1,4,7,10,13,16,25]pentaoxadiazacycloheptacosine-1,3(2H)-dione.

92. The method of claim 83, wherein the compound of the Formula (III) is 6,7,9,10,12,13-hexahydro-20H-5, 23:14,19-dimetheno-5H-dibenzo[h,n]pyrrolo[3,4-k][1,4,7,16]dioxadiazacyclooctadecine-20,22(21H)-dione.

93. The method of claim 83, wherein the compound of the Formula (III) is 6,7,9,10,12,13,15,16-octahydro-23H-5, 26:17,22-dimetheno-5H-dibenzo[k,q]pyrrolo[3,4-n][1,4,7,10,19]trioxadiazacycloheneicosine-23,25(24H)-dione.

94. The method of claim 83, wherein the compound of the Formula (III) is 10,11,13,14,16,17,19,20,22,23-decahydro-9, 4:24,29-dimetheno-1H-dibenzo[n,t]pyrrolo[3,4-q][1,4,7,10,13,22]tetraoxadiazacyclotetracosine-1,3 (2H)-dione.

95. The method of claim 83, wherein the compound of the Formula (III) is Compound 1a.

96. The method of claim 83, wherein the compound of the Formula (III) is 3-[1-[3-[(2-hydroxyethyl)methylamino]propyl]-1H-indazol-3-yl]-4-[1-(3-pyridinyl)-1H-indol-3-yl]-1H-pyrrole-2,5-dione.

97. The method of claim 83, wherein the compound of the Formula (III) is 3,5-dichloro-N-[3-chloro-4-[(3,4,12,12a-tetrahydro-1H-[1,4]thiazino[3,4-c][1,4]benzodiazepin-11(6H)-yl)carbonyl]phenyl]-benzamide.

98. The method of claim 83, wherein the compound of the Formula (III) is 3-[1-(2-hydroxy-ethyl)-1H-indol-3-yl]-4-(1-pyridin-3-yl-1H-indol-3-yl)-pyrrole-2,5-dione.

99. The method of claim 83, wherein the compound of the Formula (III) is 3-(2-methoxy-phenyl)-4-(1-pyridin-3-yl-1H-indol-3-yl)-pyrrole-2,5-dione.

100. The method of claim 83, wherein the compound of the Formula (III) is 6-[[2-[[4-(2,4-dichlorophenyl)-5-(4-methyl-1H-imidazol-2-yl)-2-pyrimidinyl]amino]ethyl]amino]-3-pyridinecarbonitrile.

101. The method of claim 83, wherein the compound of the Formula (III) is 3-(5-chloro-1-methyl-1H-indol-3-yl)-4-[1-(3-imidazol-1-yl-propyl)-1H-indazol-3-yl]-pyrrole-2,5-dione.

102. The method of claim 83, wherein the compound of the Formula (III) is 3-(5-chloro-1-methyl-1H-indol-3-yl)-4-[1-(3-[1,2,3]triazol-1-yl-propyl)-1H-indazol-3-yl]-pyrrole-2,5-dione.

103. The method of claim 83, wherein the compound of the Formula (III) is 3-[1-(3-hydroxy-propyl)-1H-pyrrolo[2,3-b]pyridin-3-yl]-4-(1-methyl-1H-pyrazol-3-yl)-pyrrole-2,5-dione.

104. The method of claim 83, wherein the compound of the Formula (III) is Compound 10a.

105. The method of claim 83, wherein the compound of the Formula (III) is 3-[1-(3-hydroxy-3-methyl-butyl)-1H-indazol-3-yl]-4-(1-pyridin-3-yl-1H-indol-3-yl)-pyrrole-2,5-dione.

106. The method of claim 83, wherein the compound of the Formula (III) is 3-[1-(2-hydroxy-ethyl)-1H-indazol-3-yl]-4-(1-pyrimidin-5-yl-1H-indol-3-yl)-pyrrole-2,5-dione.

107. The method of claim 83, wherein the compound of the Formula (III) is 3-[1-(2-hydroxy-ethyl)-1H-indol-3-yl]-4-(1-pyrimidin-5-yl-1H-indol-3-yl)-pyrrole-2,5-dione.

108. The method of claim 83, wherein the compound of the Formula (III) is (11Z)-8,9,10,13,14,15-hexahydro-2,6:17,21-di(metheno)pyrrolo[3,4-h][1,15,7]dioxazacyclotricosine-22,24(1H,23H)-dione.

109. The method of claim 83, wherein the compound of the Formula (III) is 3-(5-chloro-1-pyridin-3-yl-1H-indol-3-yl)-4-[1-(3-hydroxy-propyl)-1H-indazol-3-yl]-pyrrole-2,5-dione.

110. The method of claim 83, wherein the compound of the Formula (III) is 3-(2-methoxy-phenyl)-4-[1-(3-methoxy-propyl)-1H-pyrrolo[3,2-c]pyridin-3-yl]-pyrrole-2,5-dione.

111. The method of claim 83, wherein the compound of the Formula (III) is 3-[1-(3-hydroxy-propyl)-1H-indazol-3-yl]-4-[1-(tetrahydro-pyran-4-yl)-1H-indol-3-yl]-pyrrole-2,5-dione.

112. The method of claim 83, wherein the compound of the Formula (III) is 2-{3-[4-(5-chloro-1-methyl-1H-indol-3-yl)-2,5-dioxo-2,5-dihydro-1H-pyrrol-3-yl]-indazol-1-yl}-N-(2-hydroxy-ethyl)-acetamide.

113. The method of claim 83, wherein the compound of the Formula (III) is 4-(3-chloro-phenyl)-6-(3-dimethylamino-propyl)-5,6-dihydro-4H-2,4,6-triaza-cyclopenta[c]fluorine-1,3-dione.

114. The method of claim 83, wherein the compound of the Formula (III) is 14-ethyl-6,7,9,10,13,14,15,16-octahydro-12H,23H-5,26:17,22-dimethenodibenzo[k,q]pyrrolo[3,4-n][1,4,7,10,19]dioxatriazacycloheneicosine-23,25(24H)-dione.

115. The method of claim 83, wherein the compound of the Formula (III) is 14-benzyl-6,7,9,10,13,14,15,16-octahydro-12H,23H-5, 26:17,22-di(metheno)dibenzo[k,q]pyrrolo[3,4-n][1,4,7,10,19]dioxatriazacyclohenicosine-23,25(24H)-dione.

116. The method of claim 83, wherein the compound of the Formula (III) is 3-(1-{2-[2-(2-hydroxy-ethoxy)-ethoxy]-ethyl}-1H-indol-3-yl)-4-[1-(2-hydroxy-ethyl)-1H-indol-3-yl]-pyrrole-2,5-dione.

117. The method of claim 83, wherein the compound of the Formula (III) is 6,7,8,9,10,11,12,13-octahydro-8,11-dimethyl-5,23:14,19-dimetheno-20H-dibenzo[k,q]pyrrolo[3,4-n][1,4,7,10]tetraazacyclooctadecine-20,22(21H)-dione.

118. The method of claim 83, wherein the compound of the Formula (III) is 7,8,9,10,12,13,16,17,18,19-decahydro-8,17-dimethyl-15H,26H-5,29:20,25-dimetheno-6H-dibenzo[k,q]pyrrolo[3,4-n][1,4,7,10,19,22]dioxatetraazacyclotetracosine-26,28(27H)-dione.

119. The method of claim 83, wherein the compound of the Formula (III) is 14-(2-furylmethyl)-6,7,9,10,13,14,15,16-octahydro-12H,23H-5,26:17,22-di(metheno)dibenzo[k,q]pyrrolo[3,4-n][1,4,7,10,19]dioxatriazacyclohenicosine-23,25(24H)-dione.

120. The method of claim 83, wherein the compound of the Formula (III) is 14-(2-thienylmethyl)-6,7,9,10,13,14,15,16-octahydro-12H,23H-5,26:17,22-di(metheno)dibenzo[k,q]pyrrolo[3,4-n][1,4,7,10,19]dioxatriazacyclohenicosine-23,25(24H)-dione.

121. The method of claim 83, wherein the compound of the Formula (III) is 14-(1-naphthylmethyl)-6,7,9,10,13,14,15,16-octahydro-12H,23H-5,26:17,22-di(metheno)dibenzo[k,q]pyrrolo[3,4-n][1,4,7,10,19]dioxatriazacyclohenicosine-23,25(24H)-dione.

122. The method of claim 83, wherein the compound of the Formula (III) is 14-(pyridin-4-ylmethyl)-6,7,9,10,13,14,15,16-octahydro-12H,23H-5,26:17,22-di(metheno)dibenzo[k,q]pyrrolo[3,4-n][1,4,7,10,19]dioxatriazacyclohenicosine-23,25(24H)-dione.

123. The method of claim 83, wherein the compound of the Formula (III) is 3-[1-(2-{2-[2-(1,2,3,4-tetrahydro-naphthalen-1-ylamino)-ethoxy]-ethoxy}-ethyl)-1H-indol-3-yl]-4-{1-[2-(1,2,3,4-tetrahydro-naphthalen-1-ylamino)-ethyl]-1H-indol-3-yl}-pyrrole-2,5-dione.

124. The method of claim 83, wherein the compound of the Formula (III) is 3-[1-(3-dimethylamino-phenyl)-1H-indol-3-yl]-4-[1-(2-hydroxy-ethyl)-1H-indazol-3-yl]-pyrrole-2,5-dione.

125. The method of claim 83, wherein the compound of the Formula (III) is 3-[5-chloro-1-(6-dimethylamino-pyridin-3-yl)-1H-indol-3-yl]-4-[1-(2-hydroxy-ethyl)-1H-indazol-3-yl]-pyrrole-2,5-dione.

126. The method of claim 83, wherein the compound of the Formula (III) is 5-(5-chloro-3-{4-[1-(2-hydroxy-ethyl)-1H-indazol-3-yl]-2,5-dioxo-2,5-dihydro-1H-pyrrol-3-yl}-indol-1-yl)-nicotinic acid methyl ester.
